(19) United States
(12) Patent Application Publication
(10) Pub. No.: US 2024/0299179 A1
(43) Pub. Date: Sep. 12, 2024

(54) IMPLANTABLE INTERPOSITIONAL ORTHOPEDIC PAIN MANAGEMENT

(71) Applicant: Ensemble Orthopedics, Inc., West Lake Hills, TX (US)

(72) Inventor: William F. Ogilvie, West Lake Hills, TX (US)

(73) Assignee: Ensemble Orthopedics, Inc., West Lake Hills, TX (US)

(21) Appl. No.: 18/118,078

(22) Filed: Mar. 6, 2023

Related U.S. Application Data
(63) Continuation-in-part of application No. 16/865,297, filed on May 1, 2020.

(51) Int. Cl. A61F 2/42; A61F 2/30

(52) U.S. Cl. CPC A61F 2/4241; A61F 2/30; A61F 2/30771; A61F 2002/30301; A61F 2002/30891

(57) ABSTRACT

Implantable orthopedic pain management devices are disclosed, including a body with a top surface having a radius of curvature, and a bottom surface with another radius of curvature, configured to be surgically implanted in a joint and to prevent contact between one or more bones in the joint, the body not being coupled to the one or more bones, and a peripheral protrusion disposed substantially about a perimeter of the body and having a substantially circular cross section, the peripheral protrusion being configured to prevent expulsion of the body from the joint.

### CROSS-REFERENCE TO RELATED APPLICATIONS

This application is a continuation-in-part application of U.S. patent application Ser. No. 16/865,297, filed May 1, 2020 and titled, “IMPLANTABLE INTERPOSITIONAL ORTHOPEDIC PAIN MANAGEMENT,” all of which is herein incorporated by reference in its entirety for all purposes.

### FIELD

The present invention relates generally to implantable devices for medical and health-related purposes. More specifically, techniques for implantable interpositional orthopedic pain management are described.

### BACKGROUND

Orthopedic pain is created by many different conditions including rheumatoid arthritis, traumatic arthritis, osteoarthritis, overuse, post-fracture deformation, bone loss (due to aging or use-related injuries) and others, all of which can result in substantial pain, loss of strength, or decreased ranges of motion. Pain can be caused due to severely weakened or degraded cartilage between bones, including those found in knee, hip, finger, metacarpal, carpometacarpal, and many other joints throughout the human body. Friction or physical contact between bones with missing, weakened, or degraded cartilage is a frequent source of pain, particularly in elderly persons. Consequently, orthopedic pain relief is a major field of research, endeavor, and investment.

Conventional techniques for relieving pain and restoring motion or improving a range of motion in some bodily joints typically require the removal of degraded cartilage and bones, fusing of joints, or other highly invasive surgical procedures that can often be counterproductive to biomechanical restoration of movement and future usefulness of degraded bodily joints. Fusion of bones in some bodily joints is also problematic because further use and range of motion are eliminated or severely limited. Also, conventional solutions are problematic and can lead to the transmission of pain from a joint through bones that are fused, which is typically a time (due to surgical operating time and post-surgical rehabilitation) less costly procedure than attempting joint repair or restoration. Further, conventional surgical techniques to alleviate pain in a joint often point away from removing bones or portions thereof, which limits effectiveness, minimizes pain relief and pain management, and restoration of joint stability and ranges of motion. Other conventional solutions often remove bone or parts thereof, such as ligament removal and tendon interpositioning (“LRTI”) remove bone or portions thereof such as removing a trapezium when attempting to surgically effect pain relief in joints such as the carpometacarpal (“CMC”) joint, which can leave gaps and undesirable large joint spaces. These conventional solutions do not typically achieve joint stability or restore range of motion or strength to a degree of normal articulation and use.

Other problematic conventional solutions rely upon the use of implantable devices. Due to large numbers of ligaments, connective tissue, and bone presence, conventional techniques for the placement of implantable devices can result in cutting connective and non-connective tissue, tendons, muscle, and ligaments that do not naturally restore easily post-surgery. Many implantable devices are made of materials that are problematic as long-term options for effecting orthopedic pain relief and range of motion restoration. For example, implantable devices such as ball-and-socket artificial joints made of cobalt chrome or other metals or alloys can erode surrounding bone or cause friction resulting in bone erosion over time. Further, conventional placement of implantable devices in bodily joints typically require substantial alterations to bones and bone structures by cutting off ends, portions, or removing bones entirely. Conventional techniques are hampered due to the sacrifice of function in exchange for pain relief and are limited in offerings to a user seeking to regain normal strength and range of motion.

Thus, what is needed is a solution for orthopedic pain management using implantable medical devices without the limitations of conventional techniques.

### BRIEF DESCRIPTION OF THE DRAWINGS

Various embodiments or examples (“examples”) of the invention are disclosed in the following detailed description and the accompanying drawings:

FIG. 1A illustrates a top view of an exemplary implantable interpositional orthopedic pain management apparatus;

FIG. 1B illustrates a bottom view of an exemplary implantable interpositional orthopedic pain management apparatus;

FIG. 1C illustrates an anterior view of an exemplary implantable interpositional orthopedic pain management apparatus;

FIG. 1D illustrates a posterior view of an exemplary implantable interpositional orthopedic pain management apparatus;

FIG. 1E illustrates a left view of an exemplary implantable interpositional orthopedic pain management apparatus;

FIG. 1F illustrates a right view of an exemplary implantable interpositional orthopedic pain management apparatus;

FIG. 1G illustrates a perspective view of an exemplary implantable interpositional orthopedic pain management apparatus;

FIG. 2A illustrates a top view of an exemplary implantable interpositional orthopedic pain management apparatus;

FIG. 2B illustrates a bottom view of an exemplary implantable interpositional orthopedic pain management apparatus;

FIG. 2C illustrates an anterior view of an exemplary implantable interpositional orthopedic pain management apparatus;

FIG. 2D illustrates a posterior view of an exemplary implantable interpositional orthopedic pain management apparatus;

FIG. 2E illustrates a left view of an exemplary implantable interpositional orthopedic pain management apparatus;

FIG. 2F illustrates a right view of an exemplary implantable interpositional orthopedic pain management apparatus;

FIG. 2G illustrates a perspective view of an exemplary implantable interpositional orthopedic pain management apparatus;

FIG. 3A illustrates a top view of an exemplary implantable interpositional orthopedic pain management apparatus;

FIG. 3B illustrates a bottom view of an exemplary implantable interpositional orthopedic pain management apparatus;

FIG. 3C illustrates an anterior view of an exemplary implantable interpositional orthopedic pain management apparatus;

FIG. 3D illustrates a posterior view of an exemplary implantable interpositional orthopedic pain management apparatus;

FIG. 3E illustrates a left view of an exemplary implantable interpositional orthopedic pain management apparatus;

FIG. 3F illustrates a right view of an exemplary implantable interpositional orthopedic pain management apparatus;

FIG. 3G illustrates a perspective view of an exemplary implantable interpositional orthopedic pain management apparatus;

FIG. 4A illustrates a top view of an exemplary implantable interpositional orthopedic pain management apparatus;

FIG. 4B illustrates a bottom view of an exemplary implantable interpositional orthopedic pain management apparatus;

FIG. 4C illustrates an anterior view of an exemplary implantable interpositional orthopedic pain management apparatus;

FIG. 4D illustrates a posterior view of an exemplary implantable interpositional orthopedic pain management apparatus;

FIG. 4E illustrates a left view of an exemplary implantable interpositional orthopedic pain management apparatus;

FIG. 4F illustrates a right view of an exemplary implantable interpositional orthopedic pain management apparatus;

FIG. 4G illustrates a perspective view of an exemplary implantable interpositional orthopedic pain management apparatus;

FIG. 5A illustrates a top view of an exemplary rasping instrument used for implantable interpositional orthopedic pain management;

FIG. 5B illustrates a bottom view of an exemplary rasping instrument used for implantable interpositional orthopedic pain management;

FIG. 5C illustrates an anterior view of an exemplary rasping instrument used for implantable interpositional orthopedic pain management;

FIG. 5D illustrates a posterior view of an exemplary rasping instrument used for implantable interpositional orthopedic pain management;

FIG. 5E illustrates a left view of an exemplary rasping instrument used for implantable interpositional orthopedic pain management;

FIG. 5F illustrates a right view of an exemplary rasping instrument used for implantable interpositional orthopedic pain management;

FIG. 5G illustrates a perspective view of an exemplary rasping instrument used for implantable interpositional orthopedic pain management;

FIG. 6A illustrates an exemplary rasping instrument used for implantable interpositional orthopedic pain management;

FIG. 6B illustrates an exemplary placement of an implantable interpositional orthopedic pain management device in a carpometacarpal joint;

FIG. 6C illustrates an exemplary placement of an implantable interpositional orthopedic pain management device in a carpometacarpal joint;

FIG. 7A illustrates another exemplary placement of an implantable interpositional orthopedic pain management device in a carpometacarpal joint;

FIG. 7B illustrates a further exemplary placement of an implantable interpositional orthopedic pain management device in a carpometacarpal joint;

FIG. 7C illustrates yet another exemplary placement of an implantable interpositional orthopedic pain management device in a bone joint;

FIG. 8 illustrates an exemplary surgical technique for implantable interpositional orthopedic pain management;

FIG. 9 illustrates another exemplary surgical technique for implantable interpositional orthopedic pain management;

FIG. 10 illustrates an exemplary surgical technique for implantable interpositional orthopedic pain management; and

FIG. 11 illustrates yet another exemplary surgical technique for implantable interpositional orthopedic pain management.

FIG. 12A illustrates a top view of an exemplary implantable interpositional orthopedic pain management apparatus;

FIG. 12B illustrates a bottom view of an exemplary implantable interpositional orthopedic pain management apparatus;

FIG. 12C illustrates an anterior view of an exemplary implantable interpositional orthopedic pain management apparatus;

FIG. 12D illustrates a posterior view of an exemplary implantable interpositional orthopedic pain management apparatus;

FIG. 12E illustrates a left view of an exemplary implantable interpositional orthopedic pain management apparatus;

FIG. 12F illustrates a right view of an exemplary implantable interpositional orthopedic pain management apparatus;

FIG. 12G illustrates a perspective view of an exemplary implantable interpositional orthopedic pain management apparatus;

FIG. 13A illustrates a top view of an exemplary implantable interpositional orthopedic pain management apparatus;

FIG. 13B illustrates a bottom view of an exemplary implantable interpositional orthopedic pain management apparatus;

FIG. 13C illustrates an anterior view of an exemplary implantable interpositional orthopedic pain management apparatus;

FIG. 13D illustrates a posterior view of an exemplary implantable interpositional orthopedic pain management apparatus;

FIG. 13E illustrates a left view of an exemplary implantable interpositional orthopedic pain management apparatus;

FIG. 13F illustrates a right view of an exemplary implantable interpositional orthopedic pain management apparatus;

FIG. 13G illustrates a perspective view of an exemplary implantable interpositional orthopedic pain management apparatus;

FIG. 14A illustrates a top view of an exemplary implantable interpositional orthopedic pain management apparatus;

FIG. 14B illustrates a bottom view of an exemplary implantable interpositional orthopedic pain management apparatus;

FIG. 14C illustrates an anterior view of an exemplary implantable interpositional orthopedic pain management apparatus;

FIG. 14D illustrates a posterior view of an exemplary implantable interpositional orthopedic pain management apparatus;

FIG. 14E illustrates a left view of an exemplary implantable interpositional orthopedic pain management apparatus;

FIG. 14F illustrates a right view of an exemplary implantable interpositional orthopedic pain management apparatus;

FIG. 14G illustrates a perspective view of an exemplary implantable interpositional orthopedic pain management apparatus;

FIG. 15A illustrates a top view of an exemplary implantable interpositional orthopedic pain management apparatus;

FIG. 15B illustrates a bottom view of an exemplary implantable interpositional orthopedic pain management apparatus;

FIG. 15C illustrates an anterior view of an exemplary implantable interpositional orthopedic pain management apparatus;

FIG. 15D illustrates a posterior view of an exemplary implantable interpositional orthopedic pain management apparatus;

FIG. 15E illustrates a left view of an exemplary implantable interpositional orthopedic pain management apparatus;

FIG. 15F illustrates a right view of an exemplary implantable interpositional orthopedic pain management apparatus;

FIG. 15G illustrates a perspective view of an exemplary implantable interpositional orthopedic pain management apparatus;

FIG. 16A illustrates a top view of an exemplary implantable interpositional orthopedic pain management apparatus;

FIG. 16B illustrates a bottom view of an exemplary implantable interpositional orthopedic pain management apparatus;

FIG. 16C illustrates an anterior view of an exemplary implantable interpositional orthopedic pain management apparatus;

FIG. 16D illustrates a posterior view of an exemplary implantable interpositional orthopedic pain management apparatus;

FIG. 16E illustrates a left view of an exemplary implantable interpositional orthopedic pain management apparatus;

FIG. 16F illustrates a right view of an exemplary implantable interpositional orthopedic pain management apparatus;

FIG. 16G illustrates a perspective view of an exemplary implantable interpositional orthopedic pain management apparatus;

FIG. 17A illustrates a top view of an exemplary implantable interpositional orthopedic pain management apparatus;

FIG. 17B illustrates a bottom view of an exemplary implantable interpositional orthopedic pain management apparatus;

FIG. 17C illustrates an anterior view of an exemplary implantable interpositional orthopedic pain management apparatus;

FIG. 17D illustrates a posterior view of an exemplary implantable interpositional orthopedic pain management apparatus;

FIG. 17E illustrates a left view of an exemplary implantable interpositional orthopedic pain management apparatus;

FIG. 17F illustrates a right view of an exemplary implantable interpositional orthopedic pain management apparatus;

FIG. 17G illustrates a perspective view of an exemplary implantable interpositional orthopedic pain management apparatus;

FIG. 18A illustrates a top view of an exemplary implantable interpositional orthopedic pain management apparatus;

FIG. 18B illustrates a bottom view of an exemplary implantable interpositional orthopedic pain management apparatus;

FIG. 18C illustrates an anterior view of an exemplary implantable interpositional orthopedic pain management apparatus;

FIG. 18D illustrates a posterior view of an exemplary implantable interpositional orthopedic pain management apparatus;

FIG. 18E illustrates a left view of an exemplary implantable interpositional orthopedic pain management apparatus;

FIG. 18F illustrates a right view of an exemplary implantable interpositional orthopedic pain management apparatus;

FIG. 18G illustrates a perspective view of an exemplary implantable interpositional orthopedic pain management apparatus;

FIG. 19A illustrates a top view of an exemplary implantable interpositional orthopedic pain management apparatus;

FIG. 19B illustrates a bottom view of an exemplary implantable interpositional orthopedic pain management apparatus;

FIG. 19C illustrates an anterior view of an exemplary implantable interpositional orthopedic pain management apparatus;

FIG. 19D illustrates a posterior view of an exemplary implantable interpositional orthopedic pain management apparatus;

FIG. 19E illustrates a left view of an exemplary implantable interpositional orthopedic pain management apparatus;

FIG. 19F illustrates a right view of an exemplary implantable interpositional orthopedic pain management apparatus;

FIG. 19G illustrates a perspective view of an exemplary implantable interpositional orthopedic pain management apparatus;

FIG. 20A illustrates a top view of an exemplary implantable interpositional orthopedic pain management apparatus;

FIG. 20B illustrates a bottom view of an exemplary implantable interpositional orthopedic pain management apparatus;

FIG. 20C illustrates an anterior view of an exemplary implantable interpositional orthopedic pain management apparatus;

FIG. 20D illustrates a posterior view of an exemplary implantable interpositional orthopedic pain management apparatus;

FIG. 20E illustrates a left view of an exemplary implantable interpositional orthopedic pain management apparatus;

FIG. 20F illustrates a right view of an exemplary implantable interpositional orthopedic pain management apparatus;

FIG. 20G illustrates a perspective view of an exemplary implantable interpositional orthopedic pain management apparatus;

FIG. 21A illustrates a top view of an exemplary implantable interpositional orthopedic pain management apparatus;

FIG. 21B illustrates a bottom view of an exemplary implantable interpositional orthopedic pain management apparatus;

FIG. 21C illustrates an anterior view of an exemplary implantable interpositional orthopedic pain management apparatus;

FIG. 21D illustrates a posterior view of an exemplary implantable interpositional orthopedic pain management apparatus;

FIG. 21E illustrates a left view of an exemplary implantable interpositional orthopedic pain management apparatus;

FIG. 21F illustrates a right view of an exemplary implantable interpositional orthopedic pain management apparatus;

FIG. 21G illustrates a perspective view of an exemplary implantable interpositional orthopedic pain management apparatus;

FIG. 22A illustrates a top view of an exemplary implantable interpositional orthopedic pain management apparatus;

FIG. 22B illustrates a bottom view of an exemplary implantable interpositional orthopedic pain management apparatus;

FIG. 22C illustrates an anterior view of an exemplary implantable interpositional orthopedic pain management apparatus;

FIG. 22D illustrates a posterior view of an exemplary implantable interpositional orthopedic pain management apparatus;

FIG. 22E illustrates a left view of an exemplary implantable interpositional orthopedic pain management apparatus;

FIG. 22F illustrates a right view of an exemplary implantable interpositional orthopedic pain management apparatus;

FIG. 22G illustrates a perspective view of an exemplary implantable interpositional orthopedic pain management apparatus;

FIG. 23A illustrates a top view of an exemplary implantable interpositional orthopedic pain management apparatus;

FIG. 23B illustrates a bottom view of an exemplary implantable interpositional orthopedic pain management apparatus;

FIG. 23C illustrates an anterior view of an exemplary implantable interpositional orthopedic pain management apparatus;

FIG. 23D illustrates a posterior view of an exemplary implantable interpositional orthopedic pain management apparatus;

FIG. 23E illustrates a left view of an exemplary implantable interpositional orthopedic pain management apparatus;

FIG. 23F illustrates a right view of an exemplary implantable interpositional orthopedic pain management apparatus;

FIG. 23G illustrates a perspective view of an exemplary implantable interpositional orthopedic pain management apparatus;

FIG. 24A illustrates a top view of an exemplary implantable interpositional orthopedic pain management apparatus;

FIG. 24B illustrates a bottom view of an exemplary implantable interpositional orthopedic pain management apparatus;

FIG. 24C illustrates an anterior view of an exemplary implantable interpositional orthopedic pain management apparatus;

FIG. 24D illustrates a posterior view of an exemplary implantable interpositional orthopedic pain management apparatus;

FIG. 24E illustrates a left view of an exemplary implantable interpositional orthopedic pain management apparatus;

FIG. 24F illustrates a right view of an exemplary implantable interpositional orthopedic pain management apparatus;

FIG. 24G illustrates a perspective view of an exemplary implantable interpositional orthopedic pain management apparatus;

FIG. 25A illustrates a top view of an exemplary implantable interpositional orthopedic pain management apparatus;

FIG. 25B illustrates a bottom view of an exemplary implantable interpositional orthopedic pain management apparatus;

FIG. 25C illustrates an anterior view of an exemplary implantable interpositional orthopedic pain management apparatus;

FIG. 25D illustrates a posterior view of an exemplary implantable interpositional orthopedic pain management apparatus;

FIG. 25E illustrates a left view of an exemplary implantable interpositional orthopedic pain management apparatus;

FIG. 25F illustrates a right view of an exemplary implantable interpositional orthopedic pain management apparatus;

FIG. 25G illustrates a perspective view of an exemplary implantable interpositional orthopedic pain management apparatus;

FIG. 26A illustrates a top view of an exemplary implantable interpositional orthopedic pain management apparatus;

FIG. 26B illustrates a bottom view of an exemplary implantable interpositional orthopedic pain management apparatus;

FIG. 26C illustrates an anterior view of an exemplary implantable interpositional orthopedic pain management apparatus;

FIG. 26D illustrates a posterior view of an exemplary implantable interpositional orthopedic pain management apparatus;

FIG. 26E illustrates a left view of an exemplary implantable interpositional orthopedic pain management apparatus;

FIG. 26F illustrates a right view of an exemplary implantable interpositional orthopedic pain management apparatus;

FIG. 26G illustrates a perspective view of an exemplary implantable interpositional orthopedic pain management apparatus;

FIG. 27A illustrates a top view of an exemplary implantable interpositional orthopedic pain management apparatus;

FIG. 27B illustrates a bottom view of an exemplary implantable interpositional orthopedic pain management apparatus;

FIG. 27C illustrates an anterior view of an exemplary implantable interpositional orthopedic pain management apparatus;

FIG. 27D illustrates a posterior view of an exemplary implantable interpositional orthopedic pain management apparatus;

FIG. 27E illustrates a left view of an exemplary implantable interpositional orthopedic pain management apparatus;

FIG. 27F illustrates a right view of an exemplary implantable interpositional orthopedic pain management apparatus;

FIG. 27G illustrates a perspective view of an exemplary implantable interpositional orthopedic pain management apparatus;

FIG. 28A illustrates a top view of an exemplary implantable interpositional orthopedic pain management apparatus;

FIG. 28B illustrates a bottom view of an exemplary implantable interpositional orthopedic pain management apparatus;

FIG. 28C illustrates an anterior view of an exemplary implantable interpositional orthopedic pain management apparatus;

FIG. 28D illustrates a posterior view of an exemplary implantable interpositional orthopedic pain management apparatus;

FIG. 28E illustrates a left view of an exemplary implantable interpositional orthopedic pain management apparatus;

FIG. 28F illustrates a right view of an exemplary implantable interpositional orthopedic pain management apparatus; and

FIG. 28G illustrates a perspective view of an exemplary implantable interpositional orthopedic pain management apparatus.

### DETAILED DESCRIPTION

Various embodiments or examples may be implemented in numerous ways, including as a device, a system, a process, an apparatus, or an article of manufacture. Processes generally may be varied in individual operations, processes, or sub-processes may be performed in an arbitrary order, unless otherwise provided in the claims.

A detailed description of one or more examples is provided below along with accompanying figures. This detailed description is provided in connection with such examples, but is not limited to any particular example. The scope is limited only by the claims and numerous alternatives, modifications, and equivalents. Numerous specific details are set forth in the following description in order to provide a thorough understanding. These details are provided for the purpose of illustrating various examples and the described techniques may be practiced according to the claims without some or all of these specific details. For clarity, technical material that is known in the technical, medical, and industrial fields and related to the exemplary subject matter has not been described in detail to avoid unnecessarily obscuring the description or providing unnecessary details that may be already known to those of ordinary skill in the art.

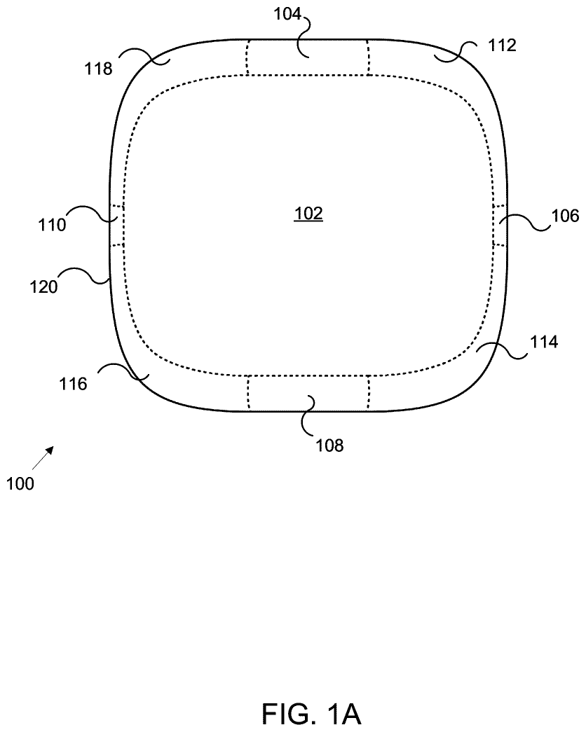

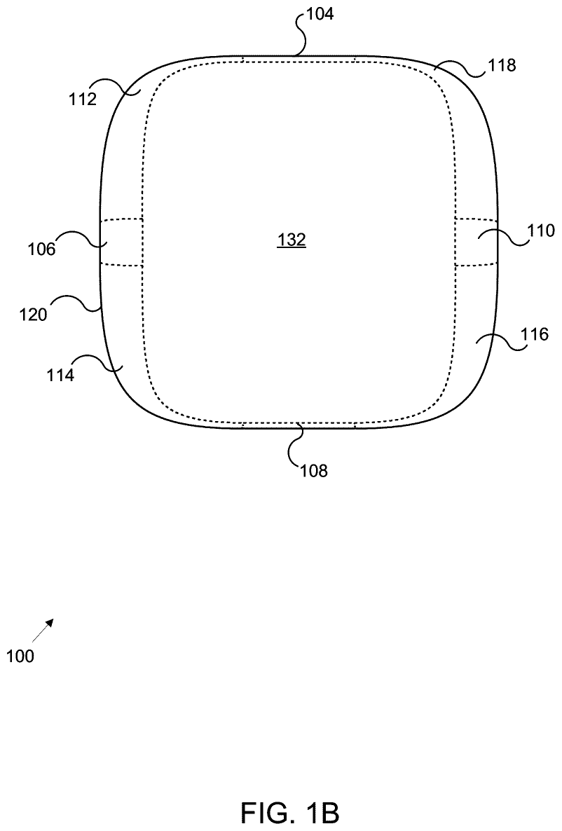

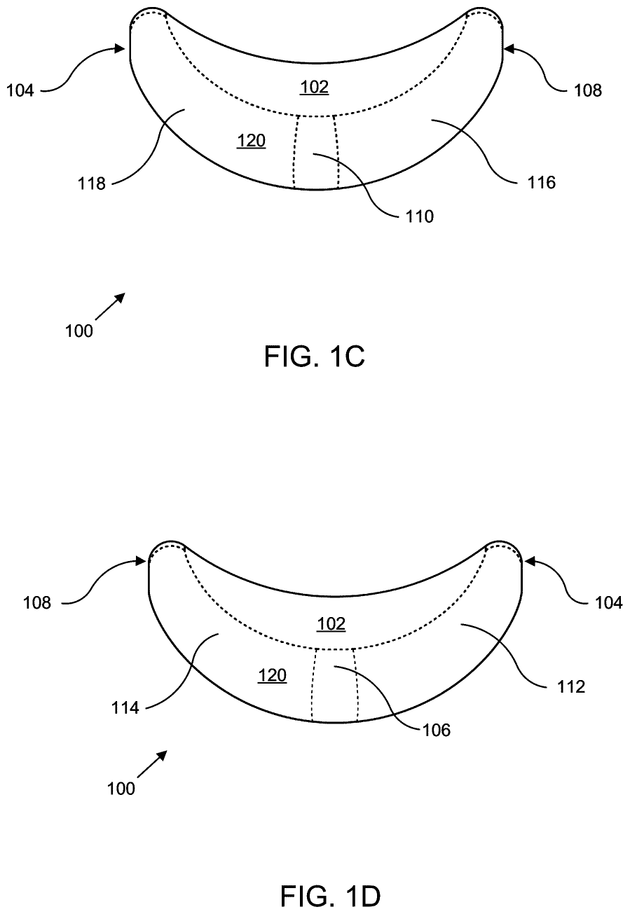

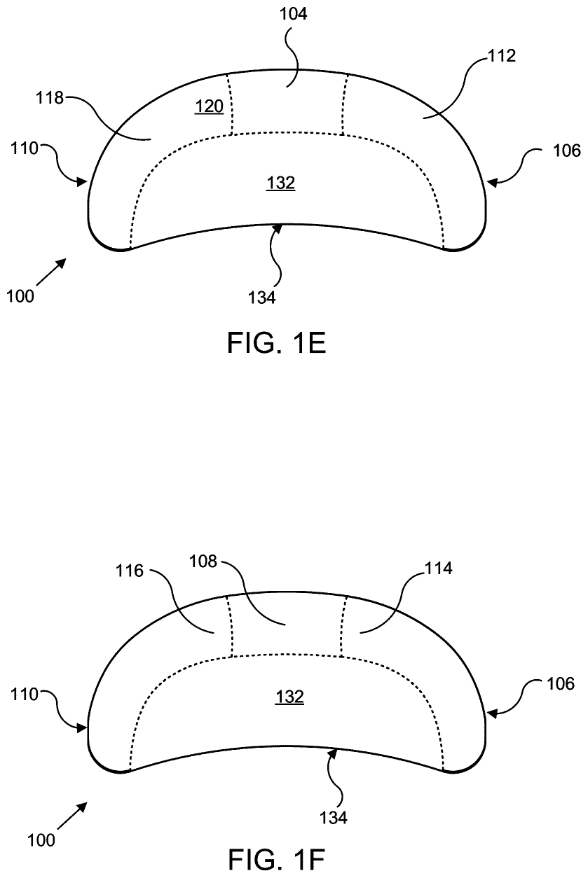

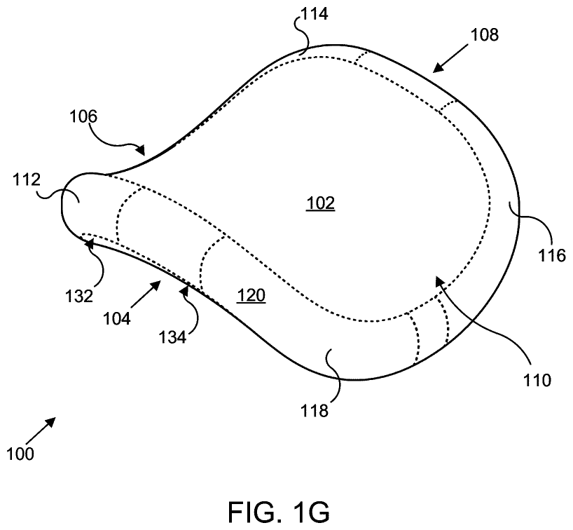

FIG. 1A illustrates a top view of an exemplary implantable interpositional orthopedic pain management apparatus. Here, a top view is shown of device 100, which includes interpositional saddle surface 102, saddle channel openings 104-110, peripheral protrusions 112-118, and periphery 120 (collectively “elements 102-120”). As used herein, like numbered and/or like named elements are assumed to be referencing the same or a substantially similar element having the same or substantially similar function or structure. As used herein, the term “saddle” may be used to refer to any implementation of device 100, which is not limited to symmetrical, asymmetrical, off-centered, centered, aligned, or other specific geometric shapes or properties. A “saddle” may be implemented using various shapes that may be concave or convex and are not required to be aligned symmetrically around any dimensional axis (not shown) shown or described throughout, but can be axially aligned, anatomically aligned (i.e., aligned about a joint, bone, or other anatomical structure, without limitation or restriction), or aligned differently, without limitation or restriction. The term “saddle” may be used to refer, in some examples, to a body of device 100 that is configured to receive, fit, conform, or otherwise be positioned or placed on, over, under, or between one or more bones. Some of these shapes may be varied such that device is saddle-shaped or substantially saddle-shaped implementation. In some examples, device 100 may be an implantable medical device (as used herein, “implantable medical device,” “implantable device,” “implant device,” “implant,” and “device” are interchangeable terms that may be used to refer to, for example, device 100, without limitation or restriction) configured for orthopedic pain relief in various types of joints such as the carpometacarpal joint, knee joint, elbow joint, or any other type of joint where two or more bones form a cartilaginous joint in which device 100 may be implanted or disposed (hereafter “implanted,” “inserted,” “surgically inserted,” “positioned,” “oriented,” or “placed,” may be used interchangeably with “disposed”). As shown, device 100 may be formed as a monolithic structure or as a composite of structures and/or sub-structures that, when combined, form an integrated implantable device that may be appropriately sized for a particular joint (or type of joints) to distract bones adjacent to a joint to prevent direct or indirect physical contact, thus alleviating pain and providing dynamic stability to the joint as well as restoring or maintaining a desired range of motion. Also, degraded (due to age-related wear or other biological deterioration) or damaged cartilage (e.g., resulting from a traumatic injury) may be replaced partially or fully using device 100.

Here, device 100 is configured structurally to contour to bone ends (e.g., distal or proximal ends of bones that have various contoured surfaces that may be convex, concave, or otherwise). For example, in a carpometacarpal joint (hereafter referred to as the “CMC joint”), a trapezium bone (“trapezium”) and a metacarpal bone (“metacarpal”) form a joint surrounded by a synovial capsule in which there may be cartilage. When used to replace degraded, deteriorated, or missing cartilage in the CMC joint, device 100 may be structurally configured with a concave surface such as interpositional saddle surface 102, which may be configured with a curvature attribute (e.g., one or more types of radii (e.g., spline, toroidal (e.g., elliptical, spline, donut, or other toroidal shapes), ellipse, or others, without limitation or restriction)) that is larger than that of, for example, a bone associated with a joint targeted for the implant within a given patient. In other examples, a curvature attribute (e.g., radius or radii of curvature) may be substituted with other units or attributes of measurement such as splines, As used herein, “larger” may refer to an attribute associated with the sizes and shapes of device 100 and interpositional saddle surface 102 (or other interpositional saddle surfaces or other features of device 100 and others such as those described herein) as determined by a range of size measurements of various bones forming different types of joints and standards of deviation. In some examples, the term “interpositional” as used herein may refer to an intended function and/or structure of device 100 as a “spacer” or intermediate implantable medical device that is designed to interpose between bones in a joint in order to alleviate pain, prevent distraction of bones from each other, and prevent or reduce loss of articulating motion and/or ranges of motion associated with a joint having missing and/or damaged cartilage. Here, device 100 may be configured for insertion into joints such as the CMC in order to relieve pain (e.g., due to injury, wounds, degradation, aging, arthritis, or other causes) while avoiding invasive and destructive procedures such as bone removal and/or joint fusion.

For example, device 100 may be formed (i.e., made, manufactured, molded, synthesized, built, generated, or the like) to different sizes of joints using various types of materials. Materials such as alloys, ceramics, ceramic-like, or polymers (e.g., thermoplastics, polycarbonate, polycarbonate urethanes (“PCU”), carbon chrome, pyrolytic carbon, polycarbon, polyetheretherketone (“PEEK”), polyetherketone (“PEK”), polyetherketoneketone (“PEKK”), polyurethane, or others, without limitation or restriction) may be used to form device 100. Also, materials may be organic, inorganic, synthetic, or natural when forming device 100, elements 102-120, and the other examples provided herein, regardless of manufacturing processes or techniques. In some examples, other attributes of device 100 may be varied before, during, or after formation. For example, device 100 may be implemented with attributes to determine the type of material to be used. In some examples, materials having an elastic modulus or moduli that is similar or substantially similar to that of bone may be used. In other words, some implementation examples of device 100 (and the other examples of devices similar or substantially similar to device 100 as described below) may be designed, formed, or otherwise implemented using materials that present attributes, characteristics, and properties (e.g., elastic modulus (i.e., elasticity), flexibility, permeability, porosity, strength, tensile strength, tensile/compressive moduli or others, without limitation or restriction) that are similar or substantially similar to those of cortical bone and bone matrix (e.g., materials that exhibit properties such as elastic moduli similar or substantially similar to cortical bone). In other examples, materials for forming device 100 may be selected based on attributes and properties of materials that are similar, substantially similar, or less than that of cortical bone and cortical bone matrix in order to prevent damage to surrounding bones during implantation. For example, material for forming device 100 may be selected with a low enough elastic moduli such that implantation may occur by folding or partially deforming device 100 for insertion through an incision in a synovial capsule and an enlarged opening into a joint. Once inserted, device 100 made of materials such as those described above, may have a sufficient low modulus (property of elastic moduli such that it recovers and restores into its originally formed shape once positioned within a joint. The above examples are provided solely for purposes of illustrating a variety of materials that may be used to implement device 100 and the other implementation examples provided herein, without limitation or restriction to any particular type of material, whether found in nature or synthesized in artificial manufacturing processes. As used herein, different types of materials may be used and are not limited to the examples above.

Sizes may be determined based on median measurements of bone structures and features such as radii of curvature of torii found at either the distal or proximal ends of the bone structures. To provide tolerance for sizing implants for various joint sizes, one or multiple standards of deviation of measurements may also be taken into account when forming device 100. For a CMC joint of a given patient, in some examples, interpositional saddle surface 102 may be formed having a radius of curvature that is designed to fit over the proximal or distal end of a trapezium, metacarpal, tibia, femur, or other type of bone that couples to another bone over one or more joints. A “channel” or length of curvature traversing horizontally through interpositional saddle surface 102 may, in some examples, extend between saddle channel openings 106 and 110. Thus, curved surfaces rising up the sides of a saddle channel between saddle channel openings 106-110 along interpositional saddle surface 102 may be formed with a radius of curvature that, when measured, may be one or two standard deviations apart from the measured radius of curvature of a bone end (i.e., distal or proximal end of a bone or bone structures such as those mentioned above) over which device 100 has been configured to “fit” when disposed within a joint. In other words, when device 100 is implanted within a synovial capsule (the term “synovial capsule” may be used interchangeably with “capsule” and may refer to an anatomical structure or feature surrounding a joint in which bone, cartilage, or other anatomical elements or features may be found), interpositional saddle surface 102 is designed (i.e., structured, formed, or configured) to contour to a corresponding surface of a bone. As an example, device 100 may be implanted into a CMC joint (not shown). When inserted, interpositional saddle surface 102 may be positioned over the end (e.g., proximal or distal) of the metacarpal or trapezium bones (not shown) adjacent to and forming the CMC joint. Formed having a radius of curvature that is one, two, or other standard deviations from a measured radius of curvature of a patient's metacarpal or trapezium, interpositional saddle surface 102 may be a concave channel traversing the distance between saddle channel openings 106 and 110 and configured to receive the end (e.g., distal or proximal) of a metacarpal, trapezium, or other bone of a joint. For example, anthropometric data may be used to determine a given radius of curvature and 1, 2, 3, or more standard deviations when forming device 100 in order to ensure interpositional saddle surface 102 has a radius of curvature that exceeds the radius of curvature of any bone adjacent to the joint.

In some examples, a channel (e.g., as formed by interpositional saddle surface 102 and saddle channel openings 106 and 110) may be disposed on one side of device 100 instead of having multiple channels on opposite sides. In other words, device 100 may be varied to have a single channel to receive the head or torus of a single bone of a joint as opposed to multiple channels configured to receive opposing bones. As shown in the present example, FIG. 1A illustrates an example of device 100 having multiple channels, but there may be examples where cartilage is present within a synovial capsule and joint in such quantity as to require only a single channel implementation of device 100.

Alternatively, another channel, an example of which is described below in connection with FIG. 1B, may be formed between saddle channel openings 104 and 108. Saddle channel openings 104 and 108, in some examples, may be disposed at either end of another saddle channel (not shown) that may be axially offset from the saddle channel formed by interpositional saddle surface 102 between saddle channel openings 106 and 110. Another saddle channel (not shown) may be disposed on the opposite surface of interpositional saddle surface 102 and configured to receive the end of an opposing bone (e.g., trapezium or metacarpal) forming a joint (not shown) with the bone received by interpositional saddle surface 102, as described in greater detail below in connection with FIG. 1B.

Referring back to FIG. 1A, an outer perimeter of device 102 (e.g., periphery 120) may have one or more peripheral protrusions 112-118 formed (e.g., molded, made, integrated, incorporated, included, or otherwise disposed) and disposed at different locations along periphery 120. As shown from a top view, a cross section (not shown) of device 100 along a horizontal (i.e., x-axis) plane may, in some examples, be substantially rectangular in shape, particularly if a cross-sectional view is of a horizontal plane disposed between interpositional saddle surface 102 and interpositional saddle surface 132 (FIG. 1)). Axes, as presented throughout this description, may be aligned along the length, width, cross-axially, anatomically aligned, or aligned otherwise, without limitation or restriction. In some examples, axes (as referred to herein) may refer to one or more axes that may run the length, width, diagonal, or in other directions aligned with one or more bones of a joint. For example, one axis may run lengthwise through a tibia, while another axis may also run lengthwise through a femur, and still another set of axes may be used for orientation associated with a joint or bone(s) (i.e., “anatomical alignment”). In other words, axes may or may not correspond to a set of three (3) dimensional axes that are orthogonal to each other, but are instead determined based on a joint and the bones that form said joint. An axis associated with a given bone may or may not necessarily also be aligned with another one of a joint. Axes may also be determined differently and are not limited to examples described.

In other examples, the positions of peripheral protrusions 112-118 may be varied, without limitation or restriction. For example, here, peripheral protrusions 112-118 are substantially positioned at the corner regions of device 100 along periphery 120. Peripheral protrusions 112-118 may be configured (i.e., when device 100 is inserted into a synovial capsule of a joint) to be positioned at non-articulating regions of a joint (not shown), but may be shaped in various configurations to maintain and prevent expulsion of device 100 from within the joint. In other examples, the positioning, number, shape, configuration, design, material, or other aspects, without limitation or restriction to the examples described. In other examples, device 100 and elements 102-120 may be designed, configured, formed, modified, or implemented apart from the examples shown or described and are not limited to those provided.

FIG. 1B illustrates a bottom view of an exemplary implantable interpositional orthopedic pain management apparatus. Here, a bottom view is shown of device 100, which includes saddle channel openings 104-110, peripheral protrusions 112-118, and periphery 120, and interpositional saddle surface 132, (collectively “elements 102-132”). As used herein, like numbered and/or like named elements are assumed to be referencing the same or a substantially similar element having the same or substantially similar function or structure. Differences in function or structure may be described separately with regard to a specific feature described. In some examples, another saddle channel may be formed with interpositional saddle surface 132, and saddle channel openings 104 and 108. As described above, an interpositional saddle channel disposed between saddle channel openings 104 and 108 along interpositional saddle surface 132 may be a partially or entirely concave feature of device 100 that is configured to receive a bone (opposite to another bone received by interpositional saddle surface 102 ((FIG. 1A)). In some examples, the heads (e.g., torus) of each bone, when received by interpositional saddle surfaces 102 and 132, may be axially offset from or aligned with each other, without limitation or restriction. Interpositional saddle surface 102 and 132 may be orthogonal or substantially orthogonal to each other. In other examples, interpositional saddle surface 102 and 132 may be axially offset, along one or more different axes, by more or less than 90 degrees.

As shown, interpositional saddle surface 102, in some examples, may have a cross sectional shape with a radius of curvature (which may be of various sizes) that is structured to receive a bone adjacent to a joint into which device 100 may be inserted. Also, as described above, a cross-section of a plane disposed substantially parallel to an axis of each interpositional saddle channel running between saddle channel openings 104 and 108 and 106 and 110, respectively. A cross-section of a plane that is substantially orthogonal to a plane lying parallel to interpositional saddle surface 102 (FIG. 1A) and interpositional saddle surface 132 (FIG. 1B) may be substantially rectangular in shape. In other examples, the shape, cross-section, or other dimensional attributes of device 100 may be varied and is not limited to those shown and described.

In some examples, peripheral protrusions 112-118 may be disposed at substantially corner positions of periphery 120 to provide sub-structures molded into periphery 120 that are configured to maintain a given position and/or orientation of device 100 within a joint. Although shown disposed at substantially corner positions of periphery 120, peripheral protrusions 112-118 may be formed at different positions, angles, attitudes, or other varying attributes along periphery 120. Here, device 100 includes peripheral protrusions 112-118 that are configured for disposition within non-articulating regions when device 100 is inserted and oriented within a synovial capsule and joint. Peripheral protrusions 112-118, in some examples, are configured to maintain device 100 within one or more tolerances of fit within a joint to prevent physical contact (i.e., relieving or lessening pain by doing so) between one or more bones of a joint, but also to maintain position within a joint and prevent expulsion, partially or fully, of device 100. In other examples, device 100 and elements 102-132 may be designed, configured, formed, modified, or implemented apart from the examples shown or described and are not limited to those provided.

FIG. 1C illustrates an anterior view of an exemplary implantable interpositional orthopedic pain management apparatus. Here, an anterior or frontal view of device 100 includes interpositional saddle channel surface 102 (showing a substantially concave contour), channel openings 104, 108 and 110 (peripheral protrusions 104 and 108 being used to form a saddle channel (i.e., a channel or contoured surface, concave or convex, configured to receive the end, head, torus, or other portion of a bone adjacent to a joint into which device 100 is surgically implanted, inserted, or otherwise disposed, regardless of surgical technique) with surface 132 (as described in connection with FIG. 1B above)), and peripheral protrusions 116-118 disposed along periphery 120. As used herein, like numbered and/or like named elements are assumed to be referencing the same or a substantially similar element having the same or substantially similar function or structure. Differences in function or structure may be described separately with regard to a specific feature described. In some examples, when device 100 is inserted into a synovial capsule or into a joint, a bone adjacent to the joint may be received by interpositional saddle surface 102. As an example, saddle surface 102 may be disposed on one side or another of device 100 in order to provide a channel (i.e., a regularly, irregularly, symmetrical, asymmetrical, or other contoured surface that may or may not be substantially concave, convex, or shaped differently) configured to receive a torus or other portion of a bone in order to provide an intermediate structure designed to prevent one bone of a joint from contact another bone in the same joint in order to prevent or alleviate pain (i.e., pain management).

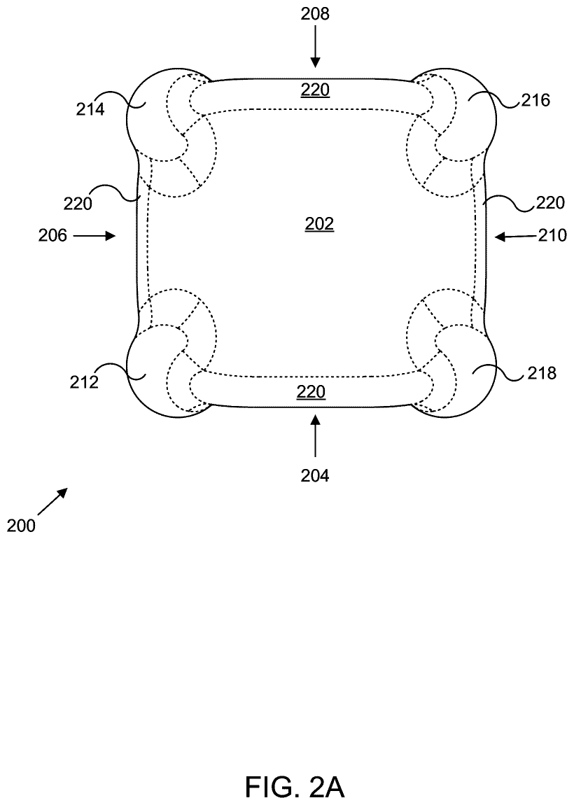

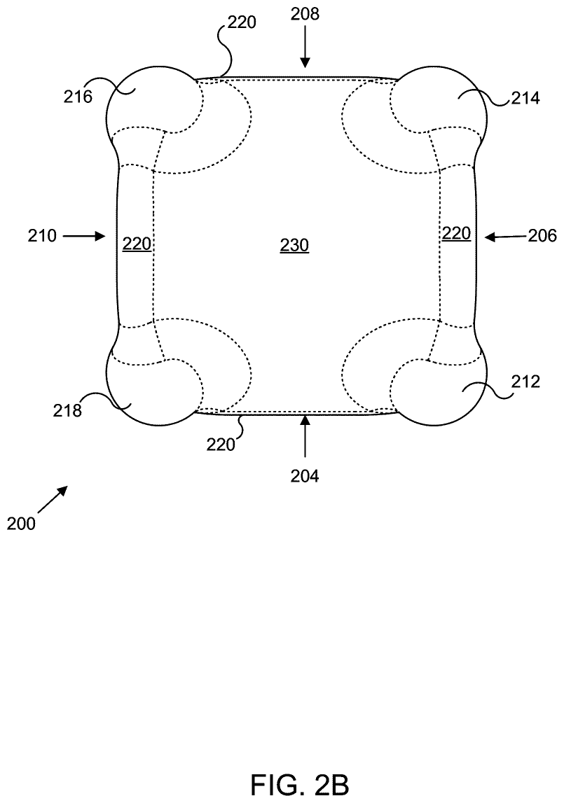

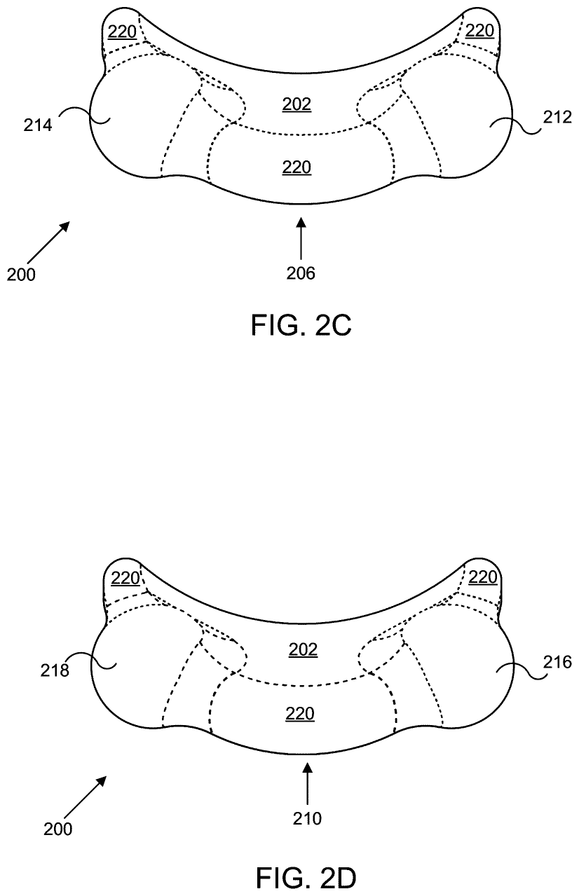

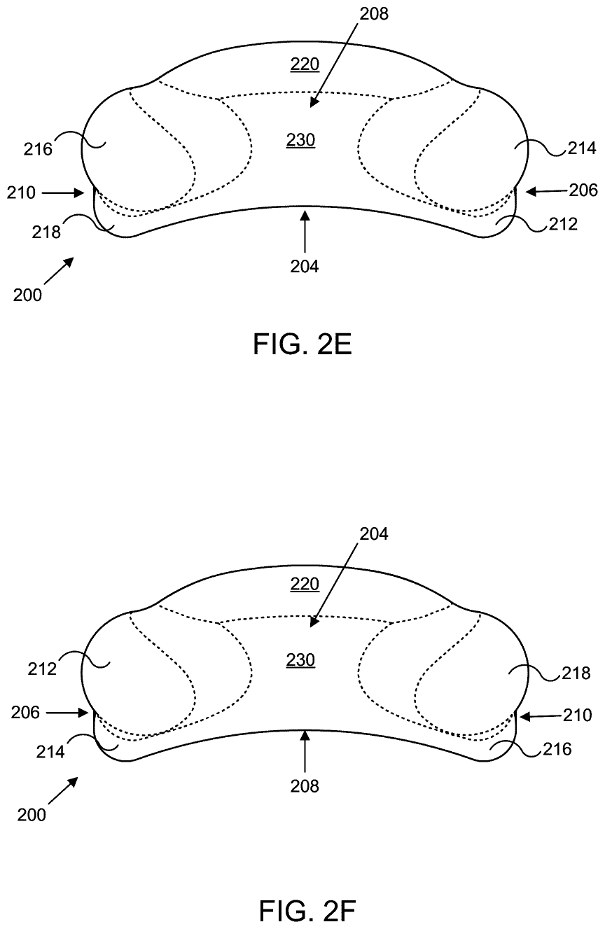

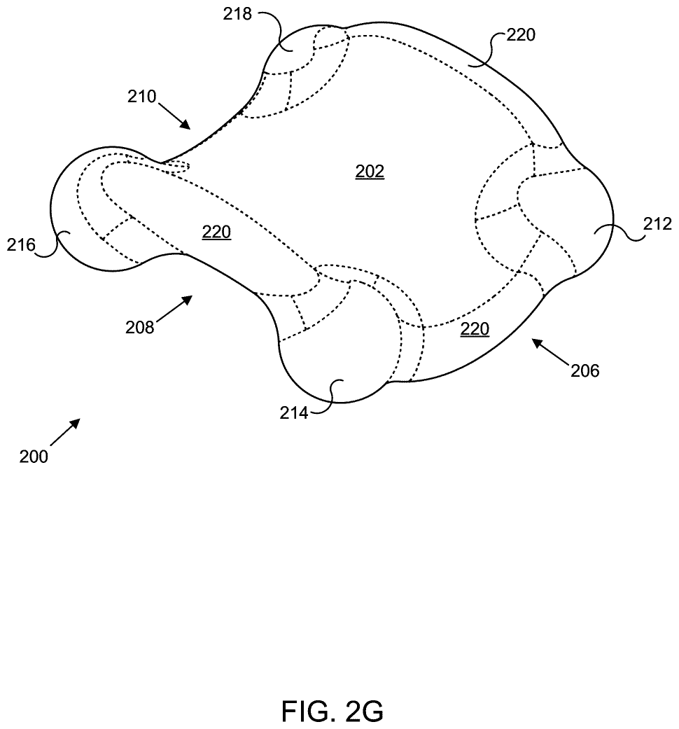

Once inserted and positioned within a joint, device 100 may be configured to remain in place by disposing peripheral protrusions 116-118 in non-articulating regions of a joint (i.e., between two or more bones). As a joint is articulated (not shown), device 100, as shown in FIG. 1C, may be used to receive the end of a bone in direct or indirect or “floating” (i.e., intermittent) contact with interpositional saddle surface 102. Periphery 120, which forms an integrate perimeter of device 100, including peripheral protrusions 116-118, which may be substantially smooth or shaped with structures (as shown in FIGS. 2-4 below) configured to maintain device 100 within a joint (i.e., prevent expulsion of device 100 by providing structures (e.g., peripheral protrusion 116-118) that are configured to maintain the position of device 100 between two or more articulating bones. Saddle channel opening 110 may be shaped (e.g., configured with a radius of curvature that is larger than that of a bone intended to be received within interpositional saddle surface 102) to receive the end, head, torus, or other portion of a bone when surgically implanted into a joint (e.g., inserting through a surgically-created opening in a synovial capsule surrounding a joint). Likewise, saddle channel openings 104 and 108 may be implemented similarly or differently.

Also shown in FIG. 1C are saddle channel openings 104 and 108, which may be axially (e.g., symmetrically) or not axially (e.g., asymmetrically) aligned to provide another saddle channel (as described below in connection with FIG. 1D) to receive the head, end, torus or other portion of an opposing or another bone when device 100 is surgically implanted. For example, when device 100 is surgically implanted in a CMC joint, interpositional saddle surface 102 and saddle channel opening 110 may be configured to receive a torus of a metacarpal bone. In some examples, saddle channel openings 104 (not shown) and 108 may be configured to receive an opposing end, head, torus, or portion of a trapezium bone and, once implanted, device 100 may be configured in function and shape to provide an intermediate implantable device (e.g., device 100) to manage, relieve, or prevent pain by preventing a trapezium and metacarpal bones from direct or indirect contact. As described herein, device 100 may be used to replace deteriorated, damaged, injured, worn, partially or wholly lost cartilage in a CMC or other joint, without restriction or limitation.

In other examples, device 100 and the elements shown and described may be designed, configured, formed, modified, or implemented apart from the examples shown or described and are not limited to those provided.

FIG. 1D illustrates a posterior or rear view of an exemplary implantable interpositional orthopedic pain management apparatus. Here, a posterior or rear view of device 100 includes interpositional saddle channel surface 102 (showing a substantially concave contour substantially opposite to that shown in FIG. 1C), channel openings 104-108 (channel openings 106-108 may be used to implement a saddle channel with interpositional saddle channel surface 102 (as described above in connection with FIG. 1A)), and peripheral protrusions 112-114 disposed along periphery 120. As used herein, like numbered and/or like named elements are assumed to be referencing the same or a substantially similar element having the same or substantially similar function or structure. Differences in function or structure may be described separately with regard to a specific feature described. For example, although interpositional saddle surface 102 is shown as substantially concave, in other examples, different shapes, contours, structures, or features may instead be implemented. As an example, interpositional saddle surfaces 102 and 132 (FIG. 1B) may be convex and/or concave, in entirety or partially. In other examples, interpositional saddle surfaces 102 and 132 (FIG. 1B) may also be substantially flat or planar or the degree of concavity or convex curvature (i.e., radii of curvature) may be altered to varying degrees. Still further, concave, convex, flat, planar, or other surface contouring may be symmetrically or asymmetrically oriented around a vertical axis (not shown) of device 100.

As shown and described, the posterior or rear view of device 100 also illustrates interpositional saddle surface 102, saddle channel openings 104-108 (for interpositional saddle surface 132 (not shown)), and peripheral protrusions 112-114 disposed along periphery 120. As described above, device 100 and the elements shown may be implemented in structure and function similarly to device 100 as shown and described above in connection with FIGS. 1A-1C. In other examples, device 100 may include variations in function and/or structure such as having a saddle channel (i.e., interpositional saddle surface 102 extending between saddle channel openings 106 and 110 (FIG. 1C)) on a single side of device 100. In other examples, device 100 may be implemented such that saddle channels may be disposed on adjacent sides or surfaces instead of being disposed on substantially opposite sides of device 100. In still other examples, device 100 and the elements shown and described may be designed, configured, formed, modified, or implemented apart from the examples shown or described and are not limited to those provided.

FIG. 1E illustrates a left view of an exemplary implantable interpositional orthopedic pain management apparatus. Here, a left view is shown of device 100 (i.e., an example of an implantable interpositional orthopedic pain management apparatus), which includes saddle channel openings 104-106 and 110, peripheral protrusions 112 and 118 disposed along and formed with periphery 120, interpositional saddle surface 132, and outer surface 134 (collectively “elements 102-132”). As described herein, like numbered and/or like named elements are assumed to be referencing the same or a substantially similar element having the same or substantially similar function or structure. Differences in function or structure may be described separately with regard to a specific feature described. In some examples, outer surface 134 may be shaped, curved, contoured, or otherwise formed to have a radius of curvature that is configured to receive a bone adjacent to a joint in which device 100 is surgically implanted. As shown, interpositional saddle surface 132 may be configured substantially orthogonal and opposing to interpositional saddle surface 102 (FIG. 1A) and used to receive a bone other than that received by a channel formed by interpositional saddle surface 102 and saddle channel openings 106 and 110. If implanted in a CMC joint, for example, interpositional saddle surface 132 may be configured with outer surface 134 having a radius of curvature that is one, two, or more standard deviations of width, depth, or other dimensions in order to receive a trapezium bone. A trapezium, when inserted into a channel formed by interpositional saddle surface 132 and saddle channel openings 104 and 108, may be configured to receive a trapezium bone. Further, another channel on substantially an opposing side of device 100 may be formed with interpositional saddle surface 102 and saddle channel openings 106 and 110 and configured to receive a metacarpal bone. In other examples, device 100 and elements 102-134 may be designed, configured, formed, modified, or implemented apart from the examples shown or described and are not limited to those provided.

FIG. 1F illustrates a right view of an exemplary implantable interpositional orthopedic pain management apparatus. Here, a right view is shown of device 100, which includes saddle channel openings 106-110, peripheral protrusions 114-116 formed in periphery 120, and interpositional saddle surface 132 with outer surface 134 (collectively “elements 102-132”). As used herein, like numbered and/or like named elements are assumed to be referencing the same or a substantially similar element having the same or substantially similar function or structure. Differences in function or structure may be described separately with regard to a specific feature described. In some examples, the above-described elements are similar to those previously described and device 100 is shown from a right-side view illustrating interpositional saddle surface 132 with saddle channel opening 108 being configured to receive a bone (e.g., trapezium, metacarpal, tibia, ulna, or others, without limitation or restriction). As described above in connection with FIG. 1E, outer surface 134 may be formed as part of interpositional saddle surface 132 with a radius of curvature configured to receive another bone. In other examples, the radius of curvature of outer surface 134 and interpositional saddle surface 132 may have a different or no curvature of radius. In other words, device 100 may be implemented with a channel on a single side and used to instead have a substantially flat surface for outer surface 134 when inserted into a joint. In other examples, device 100 and elements 102-134 may be designed, configured, formed, modified, or implemented apart from the examples shown or described and are not limited to those provided.

FIG. 1G illustrates a perspective view of an exemplary implantable interpositional orthopedic pain management apparatus. Here, a perspective view of device 100 is shown including interpositional saddle surface 102, saddle channel openings 104-110, peripheral protrusions 112-118, periphery 120, interpositional saddle surface 132, and outer surface 134 of interpositional saddle surface 132. As used herein, like numbered and/or like named elements are assumed to be referencing the same or a substantially similar element having the same or substantially similar function or structure. Differences in function or structure may be described separately with regard to a specific feature described. In some examples, saddle channel openings 104-110 may refer to those portions or regions of device 100 that have substantially concave surfaces and are designed to act as openings for receiving, for example, heads, portion, torus, or other parts of bones into interpositional saddle surfaces 102 and 132. In other examples, as described above, the radius of curvature of interpositional saddle surfaces 102 and/or 132 may be varied and alternatively have a flat or substantially flat surface on one side. In some examples, when device 100 is formed, interpositional saddle surfaces 102 and 132 may be configured to have flat, curved, concave, convex, or multi-faceted (i.e., having concave and convex surfaces disposed over interpositional saddle surfaces 102 or 132) structures or sub-structures. As described in greater detail below, peripheral protrusions 112-118 may be configured for placement within a joint to receive adjacent bones (and prevent them from contact). Device 100, in some examples, is substantially maintained in position and prevented from expulsion due to peripheral protrusions 112-118 disposed at various points along periphery 120. Thus, regardless of how a joint or bones are manipulated along various axes (e.g., abduction-adduction, flexion-extension, supination-pronation, and others) whether due to active manipulation of a joint (e.g., active motion) or passive motion (i.e., motion that may occur when the joint or bones adjacent thereto are not being directly manipulated, but instead have motion imparted to them due to other proximal or distal anatomical motion or activity), device 100 may be prevent from expulsion by one or more of peripheral protrusions 112-118 coming into contact with an articulating or non-articulating structure that keeps device 100 in position. In other examples, device 100 and elements 102-134 may be designed, configured, formed, modified, or implemented apart from the examples shown or described and are not limited to those provided.

FIG. 2A illustrates a top view of an exemplary implantable interpositional orthopedic pain management apparatus. Here, device 200 includes interpositional saddle surface 202, saddle channel openings 204-210, peripheral protrusions 212-218, and periphery 220. As used herein, like numbered and/or like named elements are assumed to be referencing the same or a substantially similar element having the same or substantially similar function or structure. Differences in function or structure may also be described separately without limitation, restriction, or regard to a specific feature previously described. For example, interpositional saddle surface 202 may be designed and implemented in function and structure similarly or substantially similarly to interpositional saddle surface 102 (FIGS. 1A, 1C-1G). In some examples, interpositional saddle surface 202 may be, with saddle channel openings 206 and 210, configured to be substantially concave in shape and receive a bone (e.g., metacarpal, trapezium, tibia, femur, or others, without limitation or restriction) and function similarly to the examples shown and described above in connection with FIGS. 1A-1G. Alternatively, peripheral protrusions 212-218 may be formed and integrated with periphery 220 to provide pronounced structures that, when device 202 is surgically implanted in a joint, may be configured to interact within articulating or non-articulating regions (or, in some examples, in a combination of articulating and non-articulating regions) of a joint.

In some examples, peripheral protrusions 212-218 may be implemented as spherical or substantially spherical (or other shapes) structures that are integrated (i.e., formed) with or along periphery 220 of device 202. When device 202 is surgically implanted, peripheral protrusions 212-218 may be positioned (i.e., disposed) within a joint so as to contact or provide structures that are configured to interact with bones or portions thereof adjacent or joining within a joint. Here, a channel may be formed as a concave or substantially concave surface of interpositional saddle surface 202 and saddle channel openings 206 and 210 that are configured to receive, as an example, a metacarpal bone in a CMC joint. As the CMC joint (not shown) is articulated, spherically-shaped (as shown in this example, but in others, different shapes, sizes, and quantities may be used and are not restricted or limited to the examples presented herein) peripheral protrusions 212-218 formed as part of periphery 220 are configured to prevent expulsion of device 200 from the joint. In other examples, the size, dimensions, and shape of device 200 may be configured for placement in different types of joints, including wrist, elbow, shoulder, knee, ankle, or others, without limitation or restriction. Further, peripheral protrusions may be extended to an opposing surface (i.e., an opposing interpositional saddle surface, as described in greater detail below) of interposition saddle surface 202.

As described herein, including in connection with FIGS. 1A-1G, device 200 may be surgically implanted to achieve stability and regain hand strength in a carpometacarpal (“CMC”), basal, or other type of joint and is not limited to any particular joint. When implanted, device 200 using peripheral protrusions 212-218 disposed about periphery 220 may be used to prevent expulsion of device 200 from a joint while also providing dynamic stability and pain relief in cases where cartilage has been worn away, destroyed, damaged, or is otherwise missing from a joint. Further, peripheral protrusions 212-218 are configured to not interfere with motion of joint in order to provide maximum range of extension and motion associated with individual bones forming a joint. Prevents migration of implantable device (i.e., implant or device) from joint. As described herein, device 200 may be surgically implanted (i.e., placed) in a joint at a point, position, and/or orientation where device 202 is least likely to be expulsed when the joint is articulated. As described above, device 200 is configured to permit motion or articulation of a joint without dislocation or expulsion of device 200 from the joint. In other words, when bones in a given joint are articulated, device 200 when implanted may be configured and implemented to prevent one or more bones from dislocation or expulsing device 200 from the joint.

In some examples, device 200 with peripheral protrusions 212-218 is configured to permit motion such as pivoting about one or more axes (e.g., abduction-adduction, flexion-extension, supination-pronation, and others) without dislocation of bones or displacement of device 200 from a joint. In some examples, surgical clamps or any type of implant holder may be configured for surgical insertion of implantable devices such as device 200 in human or animal joints, which may include carpometacarpal, trapeziometacarpal, or others, without limitation or restriction. In other examples, device 200 and the elements shown and described may be designed, configured, formed, modified, or implemented apart from the examples shown or described and are not limited to those provided.

FIG. 2B illustrates a bottom view of an exemplary implantable interpositional orthopedic pain management apparatus. Here, device 200 includes saddle channel openings 204-210, peripheral protrusions 212-218, periphery 220, and interpositional saddle surface 230. In some examples, interpositional saddle surface 230 may be contoured substantially opposite to that of interpositional saddle surface 202 (FIG. 2A). For example, interpositional saddle surface 202 (FIG. 2A) may be concave or substantially concave while interpositional saddle surface 230 may be formed to provide a convex or substantially convex channel for receiving an opposing bone in a joint. As an example, interpositional saddle surface 202 (FIG. 2A) may be formed as a substantially concave surface configured to receive a portion (e.g., central ridge, radial facet volar tubercle, or other structure disposed toward the proximal end of a metacarpal) of a bone while interpositional saddle surface 230 may be configured to fit a concave feature formed at the distal end of a trapezium bone. In other examples, interpositional saddle surfaces 202 (FIG. 2A) and 230 may be implemented differently and are not limited to the examples shown and described.

As used herein, like numbered and/or like named elements are assumed to be referencing the same or a substantially similar element having the same or substantially similar function or structure. Differences in function or structure may be described separately with regard to a specific feature described. In other examples, device 200 and the elements shown and described may be designed, configured, formed, modified, or implemented apart from the examples shown or described and are not limited to those provided.

FIG. 2C illustrates an anterior view of an exemplary implantable interpositional orthopedic pain management apparatus. Here, an anterior or front view of device 200 is shown, including interpositional saddle surface 202, saddle channel opening 206, peripheral protrusions 212-214, and periphery 220. As used herein, like numbered and/or like named elements are assumed to be referencing the same or a substantially similar element having the same or substantially similar function or structure. Differences in function or structure may be described separately with regard to a specific feature described. Here, a directional arrow points toward saddle channel opening 206, which is an opening into a saddle channel formed with interpositional saddle surface 202 and saddle channel opening 206 and saddle channel opening 210 (not shown) in which a bone (e.g., metacarpal, trapezium, femur, tibia, or the like) may be received. In some examples, peripheral protrusions 212-214 may be formed as substantially spherical structures that are integrated with periphery 220. In some examples, peripheral protrusions 212-214 may be configured to provide structures on one side (not shown) of device 200 or on multiple sides (as shown and described). In other examples, peripheral protrusions 212-214 may be formed differently and are not limited to the substantially spherical examples shown and described. Alternatively, peripheral protrusions 212-214 may be implemented using non-spherical or partially-spherical shapes. In other examples, device 200 and the elements shown and described may be designed, configured, formed, modified, or implemented apart from the examples shown or described and are not limited to those provided.

FIG. 2D illustrates a posterior view of an exemplary implantable interpositional orthopedic pain management apparatus. Here, a posterior or rear view of device 200 is shown, including interpositional saddle surface 202, saddle channel opening 210, peripheral protrusions 216-218, and periphery 220. In some examples, saddle channel opening 210 is configured to form, with interpositional saddle surface 202 and saddle channel opening 206 (FIG. 2C) a saddle-shaped channel configured to receive a bone in interpositional saddle surface 202 when device 200 is surgically implanted into a synovial capsule and joint. Disposed at an opposite end of a saddle channel formed with interpositional saddle surface 202 and saddle channel opening 206, saddle channel opening 210 is configured to receive a bone or a portion thereof (e.g., metacarpal) onto interpositional saddle surface 202. As used herein, like numbered and/or like named elements are assumed to be referencing the same or a substantially similar element having the same or substantially similar function or structure. Differences in function or structure may be described separately with regard to a specific feature described.

In other examples, device 200 and the elements shown and described may be designed, configured, formed, modified, or implemented apart from the examples shown or described and are not limited to those provided.

FIG. 2E illustrates a left view of an exemplary implantable interpositional orthopedic pain management apparatus. Here, device 200 is shown from a left view, including saddle channel openings 204-210, peripheral protrusions 212-218, periphery 220, and interpositional saddle surface 230. From a side view, interpositional saddle surface 230 is visible substantially underneath device 200 with saddle channel openings 204 and 208 at either end. In some examples, interpositional saddle surface 230 is configured, with saddle channel openings 204 and 208, to receive a bone or a portion thereof when device 200 is surgically implanted. For example, if device 200 is surgically implanted into a CMC joint, interpositional saddle surface 230 may be configured, with saddle channel openings 204 and 208, to receive (i.e., interpositional saddle surface 230 may have a radius of curvature that is larger than that of a trapezium bone) a trapezium bone, or other bone adjacent to the joint. As described above, a saddle channel consisting of interpositional saddle surface 202 (not shown; FIG. 2A) is also disposed between saddle channel openings 206 and 210. As used herein, like numbered and/or like named elements are assumed to be referencing the same or a substantially similar element having the same or substantially similar function or structure. Differences in function or structure may be described separately with regard to a specific feature described. In other examples, device 200 and the elements shown and described may be designed, configured, formed, modified, or implemented apart from the examples shown or described and are not limited to those provided.

FIG. 2F illustrates a right view of an exemplary implantable interpositional orthopedic pain management apparatus. Here, a right view of device 200 is shown including saddle channel openings 204-210, peripheral protrusions 212-218, periphery 220, and interpositional saddle surface 230. As used herein, like numbered and/or like named elements are assumed to be referencing the same or a substantially similar element having the same or substantially similar function or structure. Differences in function or structure may be described separately with regard to a specific feature described. In other examples, device 200 and the elements shown and described may be designed, configured, formed, modified, or implemented apart from the examples shown or described and are not limited to those provided.

FIG. 2G illustrates a perspective view of an exemplary implantable interpositional orthopedic pain management apparatus. Here, device 200 includes interpositional saddle surface 202, saddle channel openings 206-210, peripheral protrusions 212-218, and periphery 220. In some examples, a channel configured to receive a bone (e.g., a metacarpal bone in a CMC joint or a bone adjacent to another joint) may be implemented using interpositional saddle surface 202, saddle channel openings 206 and 210. As described above, when device 200 is surgically implanted in a joint (e.g., through an incision or opening in a synovial capsule), peripheral protrusions 212-218 may be oriented by positioning device 200 in a joint to provide dynamic stability to a joint with weakened, degraded, or missing cartilage while preventing dislocation of bones from a joint and expulsion of device 200 when a given joint is articulated. As shown, another saddle channel may be implemented on the opposite side of device 200 by using interpositional saddle surface 230 (FIG. 2B; not shown) and saddle channel openings 204 (FIG. 2B; not shown) and 208. As used herein, like numbered and/or like named elements are assumed to be referencing the same or a substantially similar element having the same or substantially similar function or structure. Differences in function or structure may be described separately with regard to a specific feature described. In other examples, device 200 and the elements shown and described may be designed, configured, formed, modified, or implemented apart from the examples shown or described and are not limited to those provided.

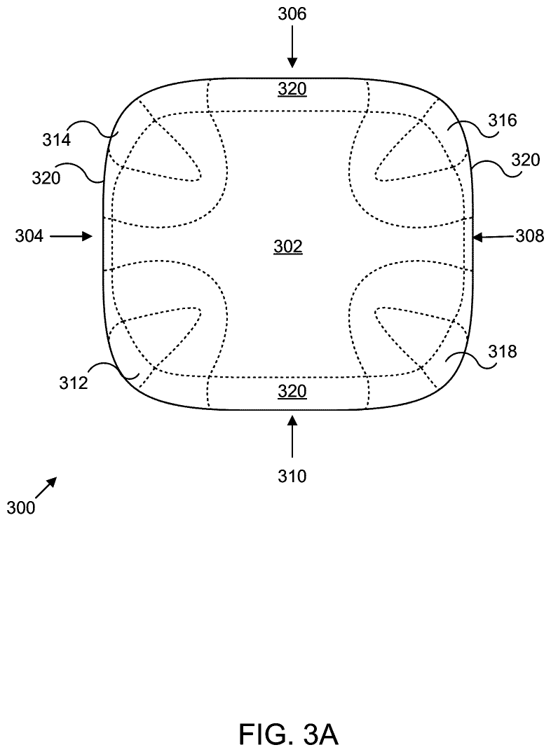

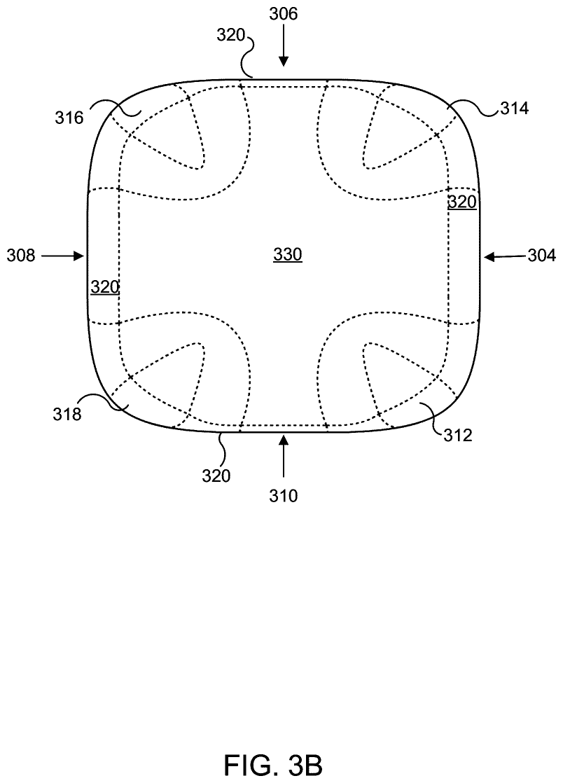

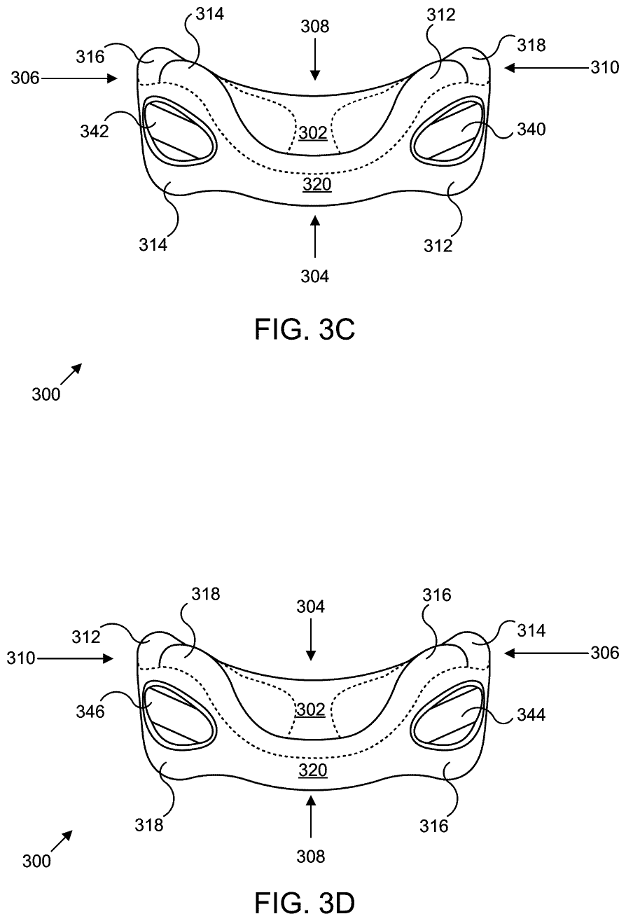

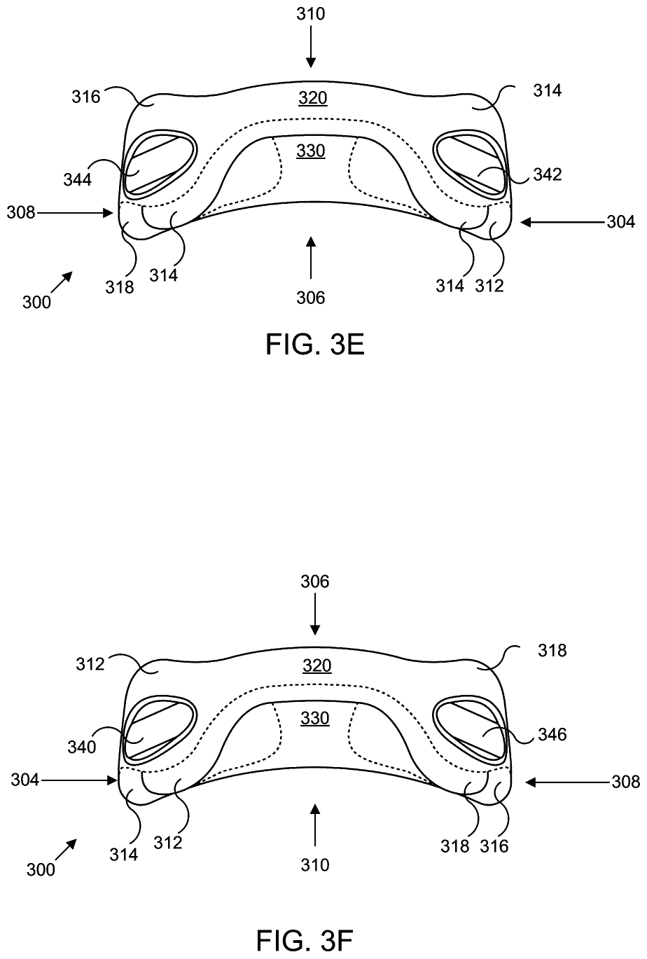

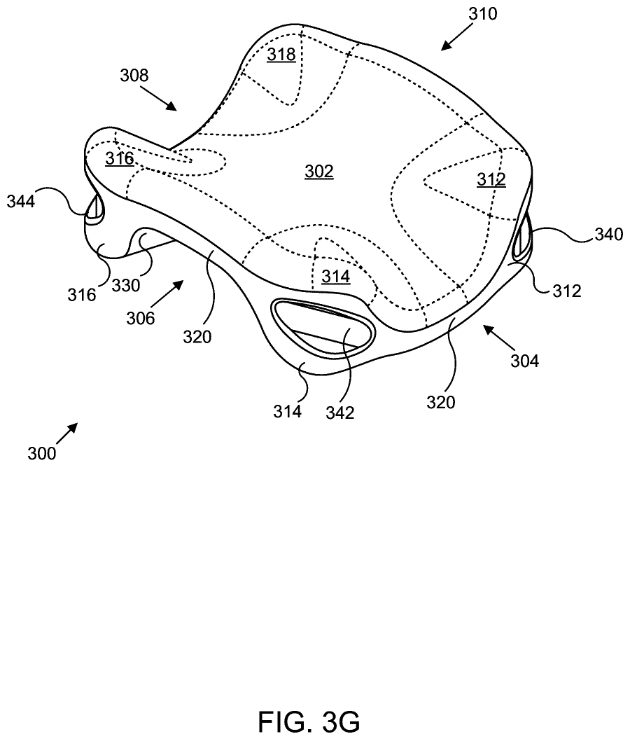

FIG. 3A illustrates a top view of an exemplary implantable interpositional orthopedic pain management apparatus. Here, device 300 includes interpositional saddle surface 302, saddle channel openings 304-310, peripheral protrusions 312-318, and periphery 320. As used herein, like numbered and/or like named elements are assumed to be referencing the same or a substantially similar element having the same or substantially similar function or structure. Differences in function or structure may be described separately with regard to a specific feature described. As shown in this drawing and others above and below, dotted lines are presented for purposes of illustrating contours and features that may include convex or concave ridges, structural or ornamental features, or other attributes as described herein.

In some examples, interpositional saddle surface 302 is configured to provide a concave surface of device 300 that is contoured and integrated with peripheral protrusions 312-318, which may be configured to “rise” from the channel formed between saddle channel openings 304 and 308. As used throughout the description of this drawing and others described above and below, the term “channel” may refer to a concave or convex feature using interpositional saddle surfaces such as interpositional saddle surface 302 or 330 (see FIG. 3B below), which functions to provide a structural feature that is configured to fit within a joint as described herein.

Here, dotted lines are presented to illustrate contouring of peripheral protrusions 312-318 as corner features of periphery 320, the latter of which may describe a perimeter portion of device 300. While formed as part or integrated with device 300, periphery 300 may be an outermost perimeter of device 300 into which features are shaped such as peripheral protrusions 312-318. As shown, periphery 320 can be found on the outer edges (i.e., perimeter) of device 300 and, disposed at various points are peripheral protrusions 312-318. In other examples, as in the previously and following described figures, the number, shape, type, quantity, and disposition of peripheral protrusions 312-318 may be varied and are not limited to those shown and described.

Here, peripheral protrusions 312-318 are illustrated in contrast to spherical or substantially spherical (e.g., peripheral protrusions 212-218 (FIGS. 2A-2G)), flat or substantially flat (e.g., 112-118 (FIGS. 1A-1G)) shapes such as those previously shown and described. In some examples, peripheral protrusions 212-218 are shaped to provide additional features and contours to improve placement, positioning, and expulsion resistance when device 300 is surgically implanted. Cavities formed within the proximal or distal ends of bones may have structures that device 300 is configured to contour to fit. The dotted lines shown in device 300 may represent contours of peripheral protrusions 212-218 that are configured to be disposed within non-articulating portions of a synovial capsule and/or joint and, when motion occurs that articulates a given joint, device 300 alleviates pain by preventing distal and proximal ends of bones in a joint from contact while peripheral protrusions also prevent dislocation (i.e., one or more bones distracting or dislocating from a joint) and expulsion of device 300. In other examples, device 300 and the elements shown and described may be designed, configured, formed, modified, or implemented apart from the examples shown or described and are not limited to those provided.

FIG. 3B illustrates a bottom view of an exemplary implantable interpositional orthopedic pain management apparatus. Here, device 300 includes saddle channel openings 304-310, peripheral protrusions 312-318, periphery 320, and interpositional saddle surface 330. As used herein, like numbered and/or like named elements are assumed to be referencing the same or a substantially similar element having the same or substantially similar function or structure. Differences in function or structure may be described separately with regard to a specific feature described. As shown herein, an “underside” view of device 300 is shown with interpositional saddle surface 330 forming a channel or contour that is configured to be disposed over or fit within a cavity formed with or within a bone, bone structure, bone joint, or portion(s) thereof, functioning similar or substantially similar to the other examples shown and described above and below in connection with various drawings throughout. In some examples, interpositional saddle surface 330 may be convex, concave, or having a radius of curvature that is substantially opposing to that of interpositional saddle surface 302. For example, interpositional saddle surface 302 (FIG. 3A) may be concave or substantially concave while interpositional saddle surface 330 may be convex or substantially convex.

Here, the radius of curvature of each of interpositional saddle surfaces 302 and 330 are configured such that a layer of material such as those described above is disposed between interpositional saddle surfaces 302 and 330 in such a manner that a cross sectional area that is coplanar between the two is substantially rectangular in shape or configuration. While the various examples shown herein may exhibit symmetry around various axes, in other examples device 300 and others described above and below may be asymmetrically formed or off-axially aligned, including when surgically implanted within a joint. In still other examples, device 300 and the elements shown and described may be designed, configured, formed, modified, or implemented apart from the examples shown or described and are not limited to those provided.

FIG. 3C illustrates an anterior view of an exemplary implantable interpositional orthopedic pain management apparatus. Here, an anterior or frontal view of device 300 includes interpositional saddle surface 302, saddle channel openings 304-310, peripheral protrusions 312-318 disposed in periphery 320, and contours 340-342. As used herein, like numbered and/or like named elements are assumed to be referencing the same or a substantially similar element having the same or substantially similar function or structure. Differences in function or structure may be described separately with regard to a specific feature described. In some examples, an interpositional saddle (i.e., “channel”) is provided by interpositional surface 302 and saddle channel openings 304 and 308, which is configured to receive a bone (e.g., metacarpal) when surgically implanted into a joint (e.g., CMC joint). Disposition within a joint (not shown) may include peripheral protrusions 312-318 being positioned to prevent expulsion of device 300 when the joint is articulated. Specifically, one or more of peripheral protrusions 312-318 (which are shown extending both to the upper and lower surfaces of device 300) may be configured to interact with one or more bones, bone structures, heads, ends, or portions thereof when surgically implanted. Further, contours 340-342 may be configured to provide additional surface features or contours that are also shaped or formed to interact with other portions of one or more bones, bone structures, heads, ends, or portions thereof when device 300 is surgically implanted within a joint. In other words, when surgically implanted, peripheral protrusions 312-318 and contours 340-342 may be configured for different types of joints or those that are specific to a given individual based on input or attributes determined or defined from techniques such as x-rays, magnetic resonance imaging (MRI), computed tomography (CAT), fluoroscopy, or other types of imaging techniques, without limitation or restriction. Device 300, as shown and described herein, provides peripheral or corner features that may be tailored to provide customization features for individual joints, bones, or other anatomical structures. Additionally, another channel may be provided, as partially indicated by saddle channel openings 306 and 310, which may be used with interpositional saddle channel 330 (FIG. 3B) to provide an opposing or substantially opposing channel on the underside of device 300 that is shaped to receive mutually reciprocal or substantially mutually reciprocal bones, bone structures, or portions thereof in a joint (e.g., trapezium, metacarpal, radial facet of a metacarpal, or others, without restriction or limitation). In other examples, device 300 and the elements shown and described may be designed, configured, formed, modified, or implemented apart from the examples shown or described and are not limited to those provided.

FIG. 3D illustrates a posterior view of an exemplary implantable interpositional orthopedic pain management apparatus. Here, a posterior or rear view of device 300 includes interpositional saddle surface 302, saddle channel openings 304-310, peripheral protrusions 312-318 disposed in periphery 320, and contours 344-346. As used herein, like numbered and/or like named elements are assumed to be referencing the same or a substantially similar element having the same or substantially similar function or structure. Differences in function or structure may be described separately with regard to a specific feature described.

Here, peripheral protrusions 312-318 are shown, including contours 344-346, which are configured in structure and function similarly to contours 340-342 (FIG. 3C). Peripheral protrusions 312-318 and contours 344-346 may be configured to reside in non-articulating regions of a joint (not shown) when device 300 is surgically implanted. In some examples, like contours 340-342 (FIG. 3C), contours 344-346 may be formed with various types of shapes and features (e.g., convex, concave, partially or wholly, or a combination thereof). If one or more bones of the joint (in which device 300 is surgically implanted) are articulated, then peripheral protrusions 312-318 and contours 344-346 are configured to prevent the bones from coming into contact with each other, maintaining dynamic stability, and restoring an ability for the joint to be manipulated while preventing expulsion of device 300 from the joint or dislocation of bones from the joint. In other words, peripheral protrusions 312-318 and contours 344-346 may be formed to provide structures that function to interact with anatomical structures of a joint, including the bones or portions thereof within the joint, to prevent device 300 from expulsion and dislocation. In other examples, device 300 and the elements shown and described may be designed, configured, formed, modified, or implemented apart from the examples shown or described and are not limited to those provided.

FIG. 3E illustrates a left view of an exemplary implantable interpositional orthopedic pain management apparatus. Here, a left side view of device 300 includes saddle channel openings 304-310, peripheral protrusions 312-318 disposed along and within periphery 320, interpositional saddle surface 330, and contours 342-344. As used herein, like numbered and/or like named elements are assumed to be referencing the same or a substantially similar element having the same or substantially similar function or structure. Differences in function or structure may be described separately with regard to a specific feature described. As shown, a left side view reveals interpositional saddle surface 330 on the underside of device 300 forming a channel between saddle channel openings 306 and 310 in a substantially orthogonal or substantially mutually reciprocal orientation to the channel described above in connection with FIG. 3C. In other examples, the channel formed by interpositional saddle surface 330 and saddle channel openings 306 and 310 may be oriented differently (i.e., set at an angle other than orthogonal to a given axis) and implementation of the techniques described herein do not require an axially orthogonal position. Alternatively, a channel may be found on only a single side of device 300 and is not required to be on mutually reciprocal or substantially reciprocal sides of device 300.

In other examples, device 300 and the elements shown and described may be designed, configured, formed, modified, or implemented apart from the examples shown or described and are not limited to those provided.

FIG. 3F illustrates a right view of an exemplary implantable interpositional orthopedic pain management apparatus. Here, a right side view of device 300 includes saddle channel openings 304-310, peripheral protrusions 312-318 disposed along and integrated with periphery 320, interpositional saddle surface 330, and contours 344 and 346. As used herein, like numbered and/or like named elements are assumed to be referencing the same or a substantially similar element having the same or substantially similar function or structure. Differences in function or structure may be described separately with regard to a specific feature described.

In some examples, peripheral protrusions 312-318 may be coupled directly or indirectly with periphery 320 or formed as surface or structural features or components thereof. As shown and described, peripheral protrusions 312-318 may have various sizes, shapes, dimensions, and other attributes, without limitation or restriction. As shown and described, peripheral protrusions 312-318 may have features integrated or formed within them as well, including, but not limited to, contours 340 and 346. Like contours 342-344 (FIG. 3E), contours 344 and 346 may be implemented as concave features, “dimple-like” in appearance, but are configured to provide contouring for peripheral protrusions 312-318 to engage and fit within a joint when device 300 is surgically implanted.

As shown, an “opening” into a saddle-shaped channel formed by interpositional saddle surface 330 and extending between saddle channel openings 310 (from the right side of device 300) and 306 (not shown, but disposed as a saddle-shaped opening from the left side of device (FIG. 3E)). In some examples, interpositional saddle surface 330 may be a mutually reciprocal surface to interpositional saddle surface 302, both of which provides engaging surfaces of device 300 to fit within a joint having, for example, opposing bones, bone structures, or portions thereof. In other examples, peripheral protrusions 312-318 may be implemented with multiple or no contours (e.g., contours 340-346), or varied beyond those examples shown and/or described, without limitation or restriction. In still other examples, device 300 and the elements shown and described may be designed, configured, formed, modified, or implemented apart from the examples shown or described and are not limited to those provided.

FIG. 3G illustrates a perspective view of an exemplary implantable interpositional orthopedic pain management apparatus. Here, a perspective view of device 300 includes interpositional saddle surface 302, saddle channel openings 304-310, peripheral protrusions 312-318 in periphery 320, interpositional saddle surface 330, and contours 340-344 (contour 346 (FIG. 3F) formed as part of peripheral protrusion 318 is not shown). As used herein, like numbered and/or like named elements are assumed to be referencing the same or a substantially similar element having the same or substantially similar function or structure. Differences in function or structure may be described separately with regard to a specific feature described.

As shown, in some examples, device 300 has multiple channel configured to receive the proximal or distal ends of bones within a joint (not shown). Interpositional saddle surfaces 302 and 330 are shown, which are formed and integrated with peripheral protrusions 312-318 from a top and bottom surfaces of device 300. Here, dotted lines are provided to show outlines of peripheral protrusions 312-318, which provide corner structures that “rise” from the corners of interpositional saddle surface 302 to provide structures that, when placed within non-articulating regions of a joint, prevent device 300 from being expulsed, partially or wholly, when the joint is articulated. Further, peripheral protrusions 312-318 may be implemented with additional structures configured to further prevent expulsion of device 300 from a joint or to aid in maintaining position and/or orientation of device 300 when surgically implanted. For example, contours 340-344 may be implemented as substantially concave features that can be configured to “fit” or receive bones, bone structures, or portions thereof in addition to interpositional saddle surfaces 302 and 330 when device 300 is surgically implanted. In other examples, contours 340-344 may also be configured to provide surfaces that are shaped to fit and/or fill with other materials within a synovial capsule such as cartilage, blood, bodily fluids, or other synthetic or organic materials that are injected or otherwise implanted to aid device 300 in maintaining dynamic stability and position within a joint after surgical implantation. In still other examples, device 300 and the elements shown and described may be designed, configured, formed, modified, or implemented apart from the examples shown or described and are not limited to those provided.

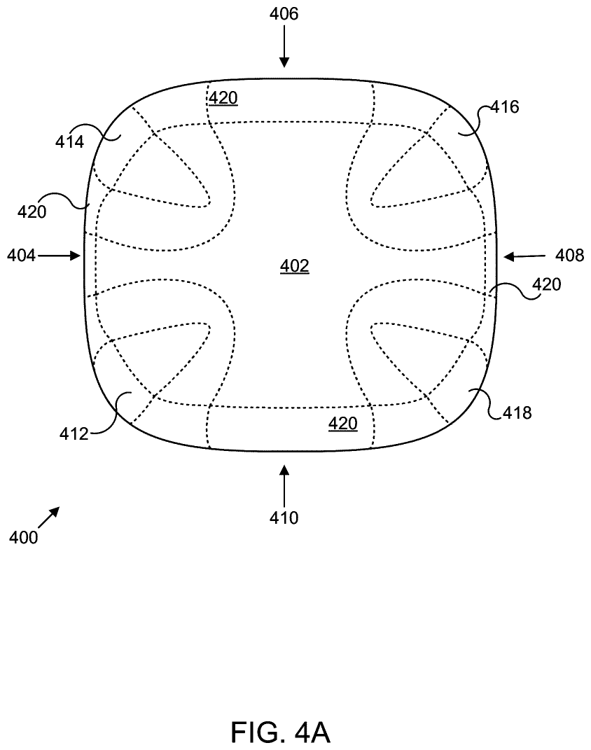

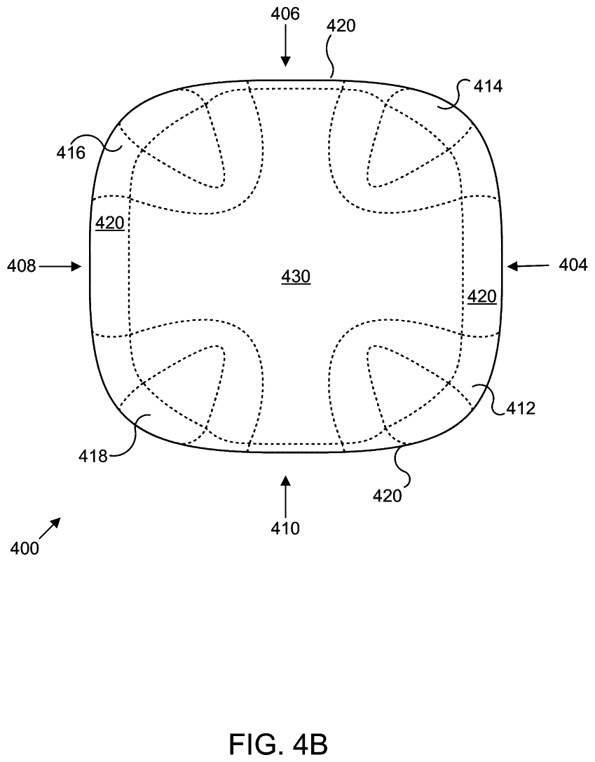

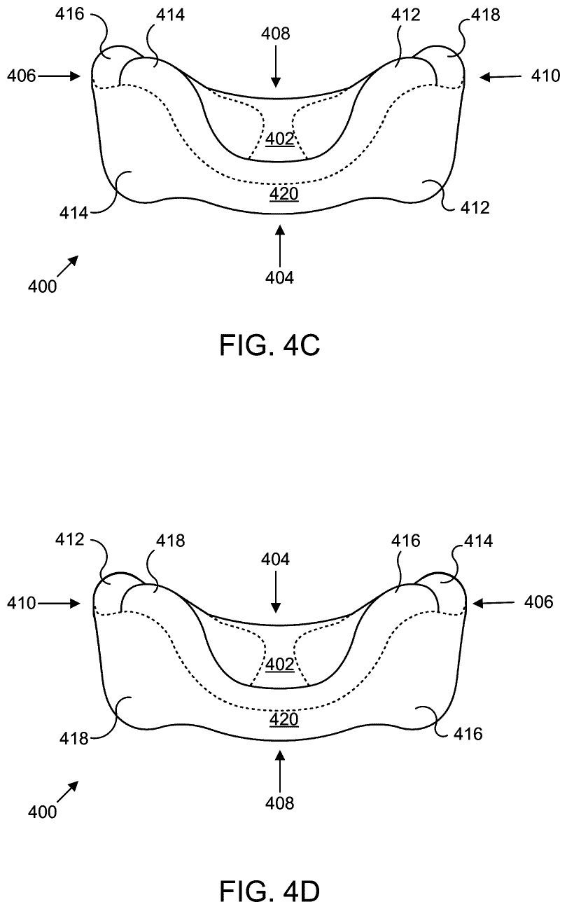

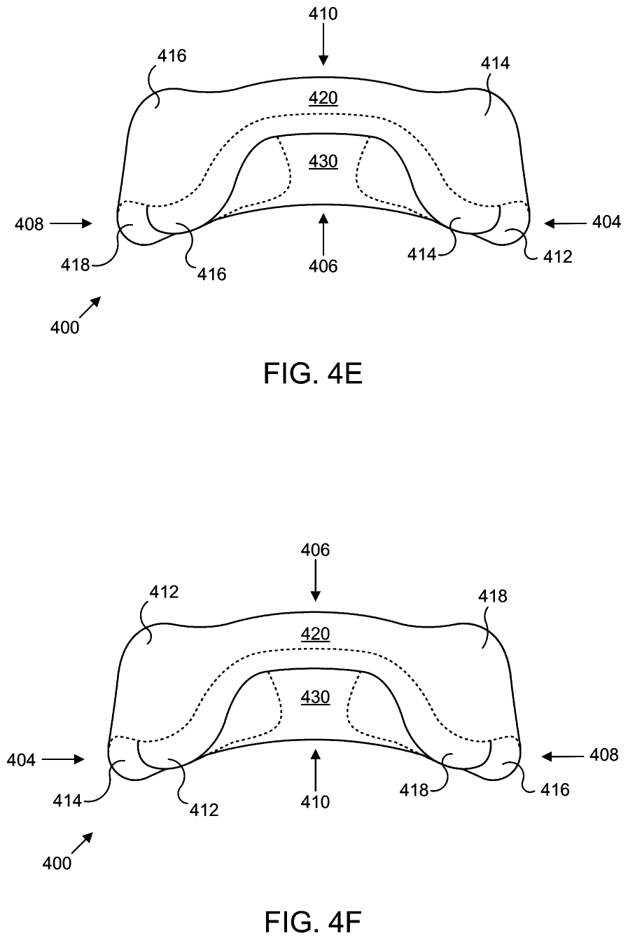

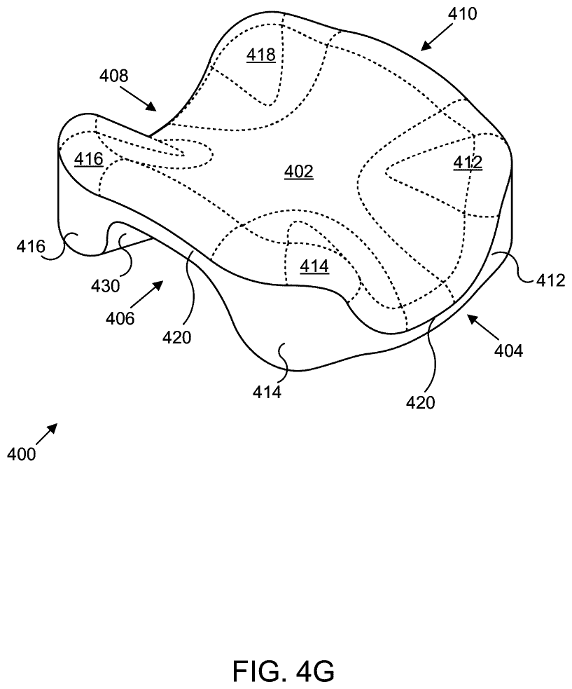

FIG. 4A illustrates a top view of an exemplary implantable interpositional orthopedic pain management apparatus. Here, device 400 includes interpositional saddle surface 402, saddle channel openings 404-410, and peripheral protrusions 412-418 disposed along periphery 420. As used herein, like numbered and/or like named elements are assumed to be referencing the same or a substantially similar element having the same or substantially similar function or structure. Differences in function or structure may be described separately with regard to a specific feature described. In some examples, device 400 may be designed, formed, and implemented as described above. Broken (i.e., dotted) lines are provided for purposes illustrating contours of peripheral protrusions 412-418, which are formed as part of periphery 420 and interpositional saddle surface 402. In some examples, peripheral protrusions 412-418 may be built up as structures that rise in the “corners” of device 400 from a concave recess or cavity (or convex feature) formed from interpositional saddle surface 402 that is configured to maintain dynamic stability of device 400 once surgically implanted in a joint. Peripheral protrusions 412-418 may also be configured in some examples without contours or other features, such as those described above in connection with FIGS. 3A-3G. In other examples, device 400 and the elements shown and described may be designed, configured, formed, modified, or implemented apart from the examples shown or described and are not limited to those provided.

FIG. 4B illustrates a bottom view of an exemplary implantable interpositional orthopedic pain management apparatus. Here, a bottom view of device 400 is shown, including saddle channel openings 404-410, peripheral protrusions 412-418 disposed in periphery 420, and interpositional saddle surface 430. As used herein, like numbered and/or like named elements are assumed to be referencing the same or a substantially similar element having the same or substantially similar function or structure. Differences in function or structure may be described separately with regard to a specific feature described. For example, peripheral protrusions 412-418 may be designed, formed, implemented, and function substantially similar or similar to peripheral protrusions 312-318 (FIG. 3A) or as other examples shown or described.

Here, broken (i.e., dotted) lines are shown for purposes of illustrating the outline of peripheral protrusions 412-418 as shown integrated with device 400 and interpositional saddle surface 430. In other examples, device 400 and the elements shown and described may be designed, configured, formed, modified, or implemented apart from the examples shown or described and are not limited to those provided.

FIG. 4C illustrates an anterior view of an exemplary implantable interpositional orthopedic pain management apparatus. Here, an anterior or front view of device 400 includes interpositional saddle surface 402, saddle channel openings 404-410, peripheral protrusions 412-418 disposed along periphery 420. As used herein, like numbered and/or like named elements are assumed to be referencing the same or a substantially similar element having the same or substantially similar function or structure. Differences in function or structure may be described separately with regard to a specific feature described. In some examples, peripheral protrusions 412-418 may be implemented without contours (e.g., contours 340-346 (FIGS. 3A-3G)) and instead provide substantially vertical walls rising from the bottom to the top of device 300 (i.e., from the bottom to the top of peripheral protrusions 412-418). Device 400, as shown and described here, is an example of another type of implementation that may be used to provide dynamic stability and range of motion while providing pain relief and prevent expulsion due to joint articulation (i.e., one or more bones, bone structures, or portions thereof being manipulated within a joint) after surgical implantation. As shown and described, interpositional saddle surface 402 forms a channel between saddle channel openings 404 and 408 while saddle channel openings 406 and 410 provide another channel with interpositional saddle surface 430 (FIG. 4B). Directional arrows associated with saddle channel openings 404-410 are intended to indicate openings for each of channel formed using interpositional saddle surfaces 402 and 430. For example, saddle channel opening 404 indicates an opening between peripheral protrusions 412 and 414 that is directed into a substantially concave recess formed by interpositional saddle surface 402, which then exits through saddle channel opening 408. Likewise, another channel is indicated at either end by saddle channel openings 406 and 410 that, together with interpositional saddle channel 430, create another channel on the bottom surface of device 400. In other examples, device 400 and the elements shown and described may be designed, configured, formed, modified, or implemented apart from the examples shown or described and are not limited to those provided.

FIG. 4D illustrates a posterior view of an exemplary implantable interpositional orthopedic pain management apparatus. Here, posterior or rear view of device 400 includes interpositional saddle surface 402, saddle channel openings 404-410, peripheral protrusions 412-418 disposed along periphery 420. As used herein, like numbered and/or like named elements are assumed to be referencing the same or a substantially similar element having the same or substantially similar function or structure. Differences in function or structure may be described separately with regard to a specific feature described. In some examples, a rear view of device 400 further illustrates peripheral protrusions 416-418 may be implemented with or without contours (e.g., contours 340-346 (FIGS. 3A-3G)), such as those shown and described above. Peripheral protrusions 412-418 may be varied and are not limited to examples shown and described and may be varied in number, placement, position, shape, configuration, shape, and may be varied in other attributes, without limitation or restriction. In other examples, device 400 and the elements shown and described may be designed, configured, formed, modified, or implemented apart from the examples shown or described and are not limited to those provided.

FIG. 4E illustrates a left view of an exemplary implantable interpositional orthopedic pain management apparatus. Here, a left view of device 400 includes saddle channel openings 404-410, peripheral protrusions 412-418 disposed in periphery 420, and interpositional saddle surface 430. As used herein, like numbered and/or like named elements are assumed to be referencing the same or a substantially similar element having the same or substantially similar function or structure. Differences in function or structure may be described separately with regard to a specific feature described. In some examples, interpositional saddle surface 430 may be implemented on the bottom (i.e., underside, underneath, or the like) of device 400. A channel may be formed with interpositional saddle surface 430 and saddle channel openings 406 and 410. As shown, a channel disposed in device 400 using interpositional saddle surface 430 may be configured to receive a bone, bone structure, or portion thereof when surgical implantation occurs.

In some examples, when device 400 is surgically implanted, peripheral protrusions 412-418 provide structures along periphery 420 that are configured to maintain device 400 in a given joint (i.e., prevent expulsion of device 400), provide dynamic stability in the given joint, and alleviate pain by preventing bones, bone structures, or portions thereof from contacting each other as a replacement for missing, damaged, worn, or otherwise degraded cartilage within a joint or synovial capsule. As shown and described, peripheral protrusions 412-418 may be configured to provide outer perimeter walls that are devoid of contours such as those discussed above in connection with FIGS. 3A-3G. In other examples, peripheral protrusions 412-418 may be implemented with different structures (e.g., contours) apart from those shown and described. For example, peripheral protrusions 412-418 may alternatively have convex or concave structures formed within them to provide features to maintain device 400 within a joint, provide dynamic stability, maintain positioning of device 400 within a synovial capsule and/or joint, increase or protect ranges of motion, alleviate or prevent pain due to bone contact, or others. Still further, peripheral protrusions 412-418 may be varied in quantity, placement, location, position, or other attributes relative to periphery 420. As another example, peripheral protrusions 412-418 may be varied in the number of protrusions disposed and formed with periphery 420. In still other examples, device 400 and the elements shown and described may be designed, configured, formed, modified, or implemented apart from the examples shown or described and are not limited to those provided.

FIG. 4F illustrates a right view of an exemplary implantable interpositional orthopedic pain management apparatus. Here, a right view of device 400 includes saddle channel openings 404-410, peripheral protrusions 412-418 disposed in periphery 420, and interpositional saddle surface 430. As used herein, like numbered and/or like named elements are assumed to be referencing the same or a substantially similar element having the same or substantially similar function or structure. Differences in function or structure may be described separately with regard to a specific feature described. As shown, saddle channel opening 410 indicates an opening to a channel formed with interpositional saddle surface 430 and saddle channel opening 406, the latter of which being indicated by a directional arrow pointed to the rear of device 400. As shown herein (as well as other drawings above and below), a directional arrow for saddle channel opening is directed at an opening having a radius of curvature implemented with interpositional saddle surface 430, which is configured to receive a bone, bone structure, or portion thereof such as a trapezium bone in a CMC joint. As with the other examples throughout this description, various radii of curvature may be used and are not limited to the examples shown and described. For example, an orthopedic surgeon (not shown) may receive a kit having one or more devices similar or substantially similar to device 400. Various devices may be formed using different radii of curvature in order to provide a range of options for a surgeon to determine which device (e.g., device 400) would be suited or appropriately sized for a given joint. Further, as discussed in greater detail below, trial devices (hereafter referred to as “trials”) may be implemented examples of device 400 of different sizes (e.g., having varied or different dimensions), but also have a stem that may be manipulated for testing the placement and position of a device within a given joint. In some examples, a trial may also be used to probe a synovial capsule or joint in order to determine one or more attributes (e.g., size, dimensions, depth, width, tension, resistance, presence or lack of cartilage, and others, without limitation or restriction).

Referring back to FIG. 4F, device 400 may be implemented without contours or other features on the outer most perimeter of periphery 420. In alternative examples, contours or other features may be used and are not limited to those shown and described. In still other examples, device 400 and the elements shown and described may be designed, configured, formed, modified, or implemented apart from the examples shown or described and are not limited to those provided.

FIG. 4G illustrates a perspective view of an exemplary implantable interpositional orthopedic pain management apparatus. Here, device 400 includes interpositional saddle surface 402, saddle channel openings 404-410, peripheral protrusions 412-418 disposed in periphery 420, and interpositional saddle surface 430. As used herein, like numbered and/or like named elements are assumed to be referencing the same or a substantially similar element having the same or substantially similar function or structure. Differences in function or structure may be described separately with regard to a specific feature described. In some examples, device 400 includes peripheral protrusions 412-418, which may be formed with or without features such as contours (e.g., contours 340-346 (FIGS. 3A-3G)). As shown and described herein, peripheral protrusions 412-416 are illustrated with upper and lower portions (peripheral protrusion 418 is shown with an upper portion based on the perspective view angle) that are intended to convey the integration of peripheral protrusions 412-418 with interpositional saddle surfaces 402 and 430 in device 400. Further, broken lines are provided to illustrate exemplary contouring and shaping of peripheral protrusions 412-418 as each is formed shape-wise into interpositional saddle surface 402. While periphery 420 is identified as a separate element, one, some, or all of peripheral protrusions 412-418 may be integrated portions that are part of periphery 420. In other examples, periphery 420 may be formed by directly or indirectly coupling peripheral protrusions 412-418, which may be formed with or apart from device 400. In other examples, device 400 and the elements shown and described may be designed, configured, formed, modified, or implemented apart from the examples shown or described and are not limited to those provided.

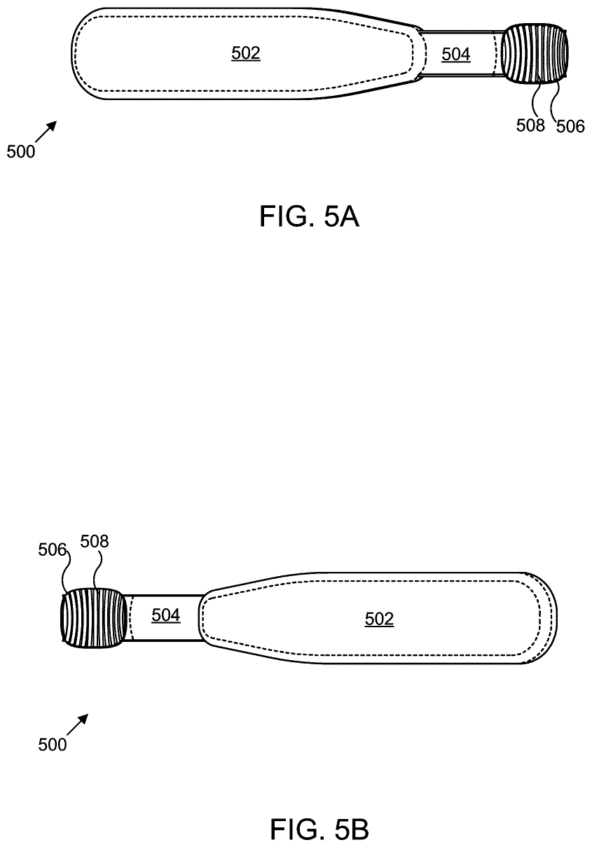

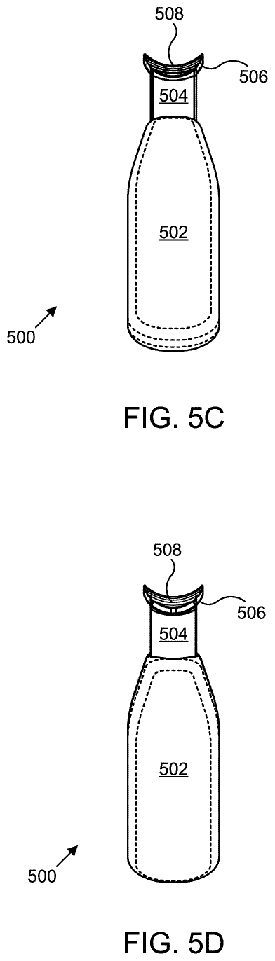

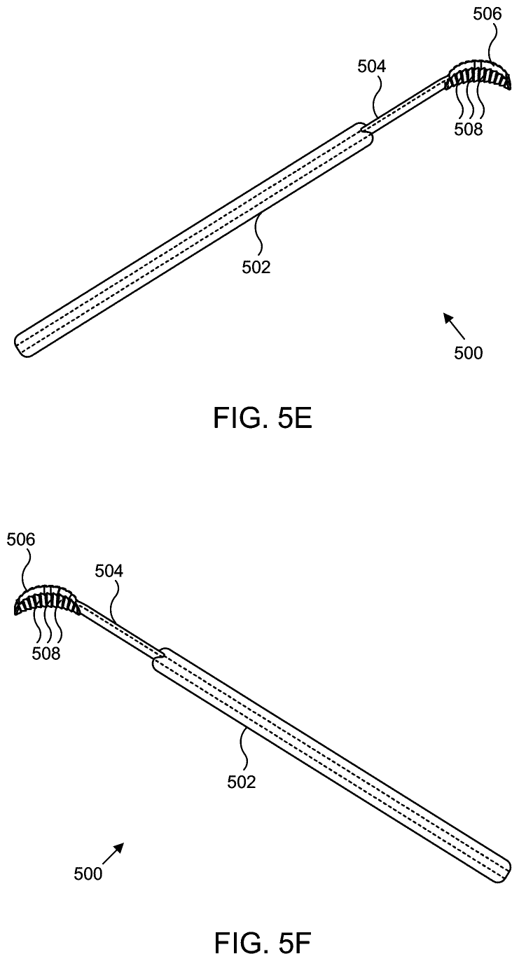

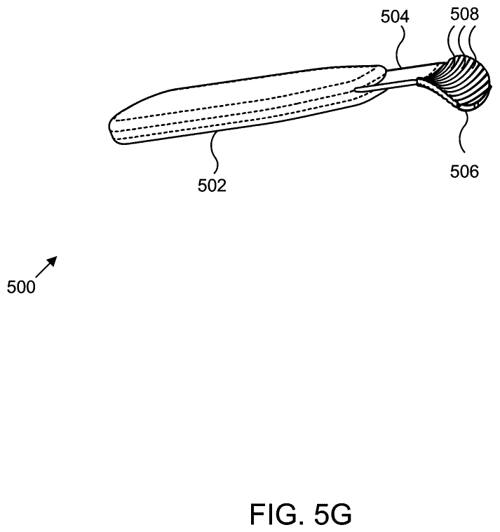

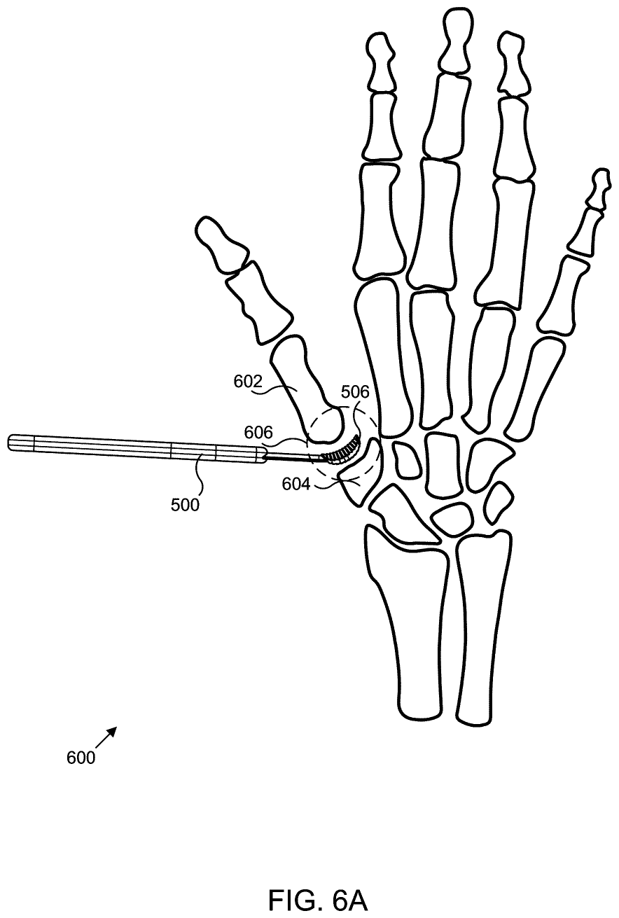

FIG. 5A illustrates a top view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In some examples, instrument 500 may be used to prepare a path for surgical insertion (i.e., implantation) of device 100 (FIGS. 1A-1G), 200 (FIGS. 2A-2G), 300 (FIGS. 3A-3G), 400 (FIGS. 4A-4G), or others. Using handle 502, which is coupled to rasp 506, a user (e.g., orthopedic surgeon, or others) may prepare a joint for insertion of an implantable device, examples of which are provided and discussed herein. As an example, rasp 506 may be implemented (i.e., formed, manufactured, shaped, or otherwise formed) to have a larger radius of curvature than a device (hereafter “device” may refer to any of the exemplary implantable devices shown and described herein). Rasp 506 may be implemented with teeth 508, which may be rasping ridges, cutting ridges, cutting teeth, saw teeth, cutting rows, or the like. As an example, rasp 506 with teeth 508 may be used to “rasp” or modify a joint prior to insertion and implanting of a device. Instrument 500 and the elements may be constructed of various types of materials, such as those described above. In other examples, device 500 and the elements shown and described may be designed, configured, formed, modified, or implemented apart from the examples shown or described and are not limited to those provided.

FIG. 5B illustrates a bottom view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. As shown and described, instrument 500 may be configured with rasp 506, which may have a radius of curvature that is slightly larger than a device to be implanted and may be substantially similar in shape to a device. In other words, rasp 506 may be formed in size, shape, and configuration similar to a device intended for implantation. When instrument 500 is used to modify or rasp a joint, the substantial similarity in size, shape, and configuration aids implantation of a device. In other examples, instrument 500 and the elements shown and described may be designed, configured, formed, modified, or implemented apart from the examples shown or described and are not limited to those provided.

FIG. 5C illustrates an anterior view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In other examples, instrument 500 and the elements shown and described may be designed, configured, formed, modified, or implemented apart from the examples shown or described and are not limited to those provided.

FIG. 5D illustrates a posterior view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In other examples, instrument 500 and the elements shown and described may be designed, configured, formed, modified, or implemented apart from the examples shown or described and are not limited to those provided.

FIG. 5E illustrates a left view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In other examples, instrument 500 and the elements shown and described may be designed, configured, formed, modified, or implemented apart from the examples shown or described and are not limited to those provided.

FIG. 5F illustrates a right view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In other examples, instrument 500 and the elements shown and described may be designed, configured, formed, modified, or implemented apart from the examples shown or described and are not limited to those provided.

FIG. 5G illustrates a perspective view of an exemplary rasping instrument used for implantable interpositional orthopedic pain management. Here, instrument 500 includes handle 502, neck 504, rasp 506, and teeth 508. As used herein, like numbered and/or like named elements are assumed to be referencing the same or a substantially similar element having the same or substantially similar function or structure. Differences in function or structure may be described separately with regard to a specific feature described. In other examples, instrument 500 and the elements shown and described may be designed, configured, formed, modified, or implemented apart from the examples shown or described and are not limited to those provided.

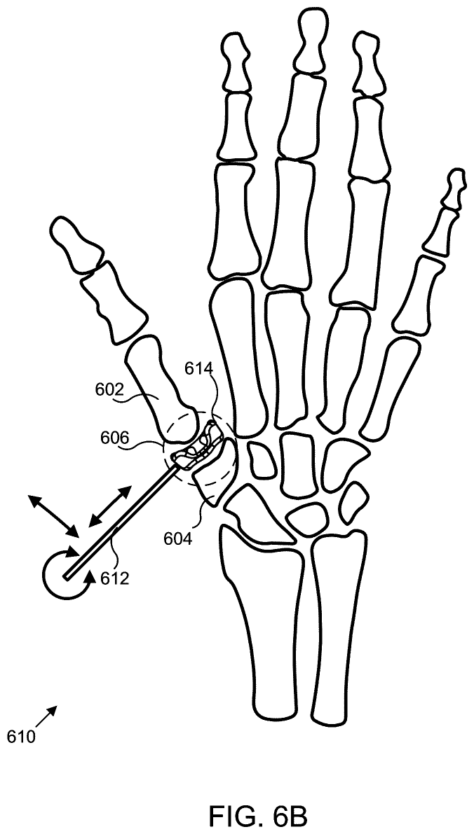

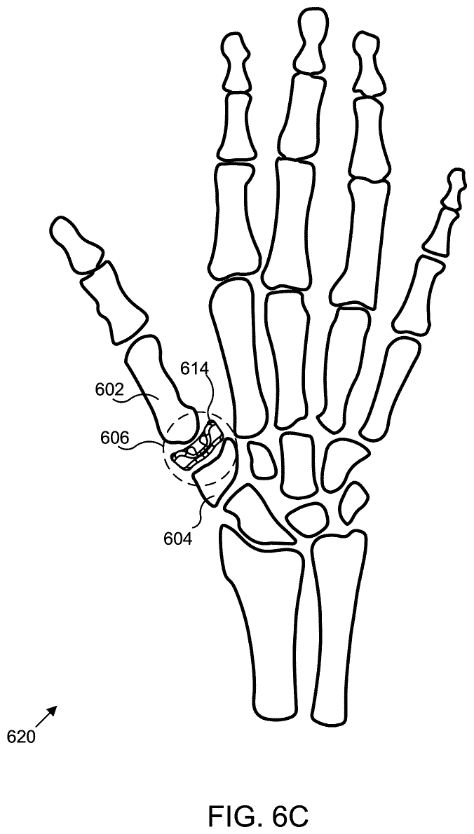

FIG. 6 illustrates an exemplary rasping instrument used for implantable interpositional orthopedic pain management. Here, diagram 600 illustrates a basic skeletal structure of a human hand (in other examples, the skeletal structure of an animal other than Homo sapiens may be used) having metacarpal bone 602, trapezium bone 604, and joint 606. As used herein, like numbered and/or like named elements are assumed to be referencing the same or a substantially similar element having the same or substantially similar function or structure. Differences in function or structure may be described separately with regard to a specific feature described. As described above in connection with FIGS. 5A-5G, rasping instrument 500 with rasp 506 is shown disposed between metacarpal 602 and trapezium 604 forming joint 606. As shown here, rasping instrument 500 may be used to clear, abrade, rasp, or otherwise modify the CMC joint between metacarpal 602 and trapezium 604 for the purpose of path preparation into joint 606 prior to surgical implantation of a device (e.g., device 100 (FIGS. 1A-1G), device 200 (FIGS. 2A-2G), device 300 (FIGS. 3A-3G), device 400 (FIGS. 4A-4G); not shown here). In other examples, rasping instrument 500 may be used to modify joints other than a CMC joint (e.g., joint 606) and is not limited to those shown and described. As presented herein, a joint (e.g., joint 606) targeted for surgical implantation of a device such as those described herein may be prepared (i.e., have a path prepared) by rasping out a path in joint 606 into which a trial or device may be inserted. Surgeons (i.e., users) of rasping instrument 500 may prepare a path for surgical implantation of an interpositional orthopedic pain management device such as those described herein, regardless of the type of joint targeted for modification. In other examples, path preparation may also be performed using more or different tools other than rasping instrument 500 (and rasp 506) and are not limited to the examples shown and described. In other examples, rasping instrument 500 as depicted in diagram 600 and the elements shown and described may be designed, configured, formed, modified, or implemented apart from the examples shown or described and are not limited to those provided.

FIG. 6B illustrates an exemplary placement of an implantable interpositional orthopedic pain management device in a carpometacarpal joint. Here, diagram 610 depicts metacarpal bone (i.e., “metacarpal”) 602, trapezium bone (i.e., “trapezium”) 604, joint 606, trial 612, and trial device 614. As used herein, like numbered and/or like named elements are assumed to be referencing the same or a substantially similar element having the same or substantially similar function or structure. Differences in function or structure may be described separately with regard to a specific feature described. In some examples, after path preparation (as described above in connection with FIG. 6A) has been performed, trial 612 (i.e., a stem coupled to trial device 614, which may be used for purposes of identifying a size of an implantable device targeted for surgical implantation into joint 606, such as those described above) may be inserted into joint 606 to determine whether additional path preparation is desired and, once completed, a size for an implantable device. As shown and described, trial 612 may be formed with trial device 614. In some examples, trial 614 may be welded, integrated with, incorporated into, or otherwise attached to a stem of trial 612. Here, when trial 612 is manipulated by a surgeon or other user, a stem may be provided to formed of various types of materials, such as those described above, and is not limited to the type, manner of formation or manufacturing, or shape. For example, a stem of trial 612 may be an elongated handle having a rounded, circular, oval, square, rectangular, or other type of cross section. Further, a stem of trial 612 may be rigid, flexible, malleable, or have other attributes intended to provide ease of manipulation of trial device 614 during path preparation and fitting (i.e., “sizing” or determining a size for an implantable device based on joint 606). As described herein, trial 612 may be manipulated in various directions to test ranges of motion for trial 614, as indicated by the various arrows provided for generally indicating directional manipulation of device 612, without limitation or restriction to degree, angle, or direction.

As used herein, trial 612 may be one of several trials included in a surgical kit having one or more implantable devices (e.g., device 100 (FIGS. 1A-1G), device 200 (FIGS. 2A-2G), device 300 (FIGS. 3A-3G), device 400 (FIGS. 4A-4G); not shown here). Trial 612 and other trials not shown may be included in a kit intended for reuse or disposal, but after being used to identify a suitable size of an implantable device (not shown) for insertion into joint 606. As shown, trial device 614 may be shaped, configured, or otherwise formed similarly or substantially similarly to a device (not shown) intended for surgical implantation. Trial 612 and other trials used provide the ability to determine a suitable size for an implantable device without requiring excessive trial and error (i.e., attempting to surgically implant disparate implantable devices), which can not only be time and cost-consuming, but also incur risks associated with unnecessary tissue, ligament and ligature, and tendon stress or damage. In other examples, trial 612 as depicted in diagram 610 and the elements shown and described may be designed, configured, formed, modified, or implemented apart from the examples shown or described and are not limited to those provided.

FIG. 6C illustrates an exemplary placement of an implantable interpositional orthopedic pain management device in a carpometacarpal joint. Here, diagram 620 depicts metacarpal bone (i.e., “metacarpal”) 602, trapezium bone (i.e., “trapezium”) 604, joint 606, trial 612, and device 622. As used herein, like numbered and/or like named elements are assumed to be referencing the same or a substantially similar element having the same or substantially similar function or structure. Differences in function or structure may be described separately with regard to a specific feature described. In some examples, device 622 may be surgically implanted in joint 606 between metacarpal 602 and trapezium 606. However, in other examples, device 622 and others as described above (e.g., device 100 (FIGS. 1A-1G), device 200 (FIGS. 2A-2G), device 300 (FIGS. 3A-3G), device 400 (FIGS. 4A-4G); not shown here) may be used for surgical implantation in other human or animal joints, without limitation or restriction, using the techniques and tools described herein. In other examples, device 622 as depicted in diagram 610 and the elements shown and described may be designed, configured, formed, modified, or implemented apart from the examples shown or described and are not limited to those provided.

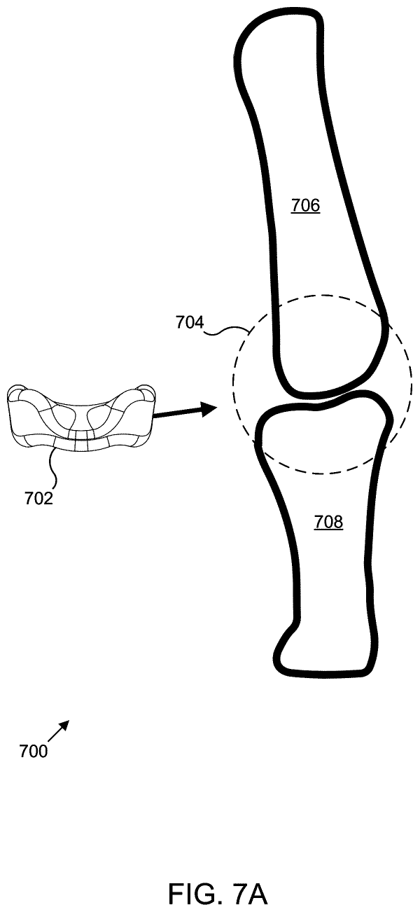

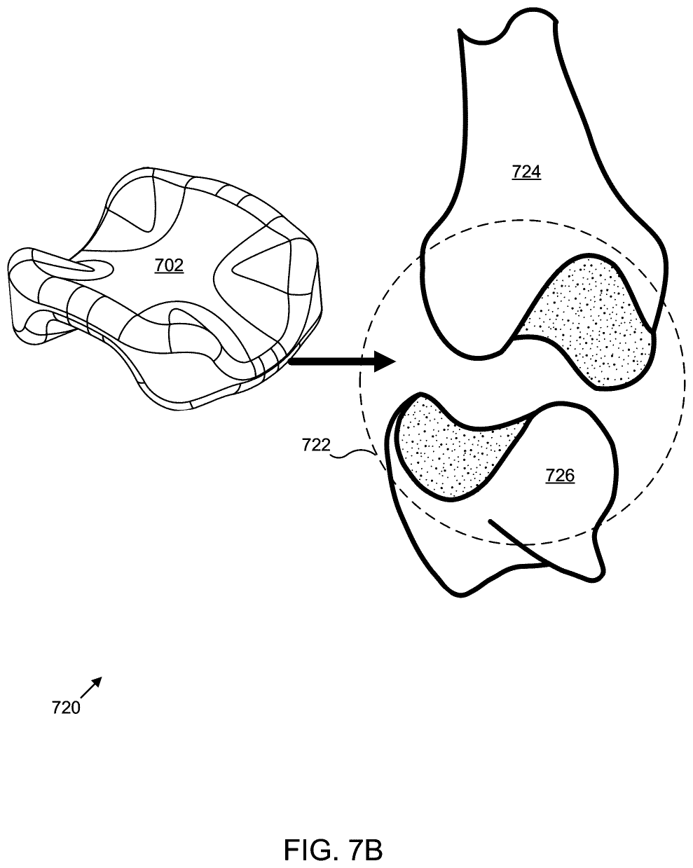

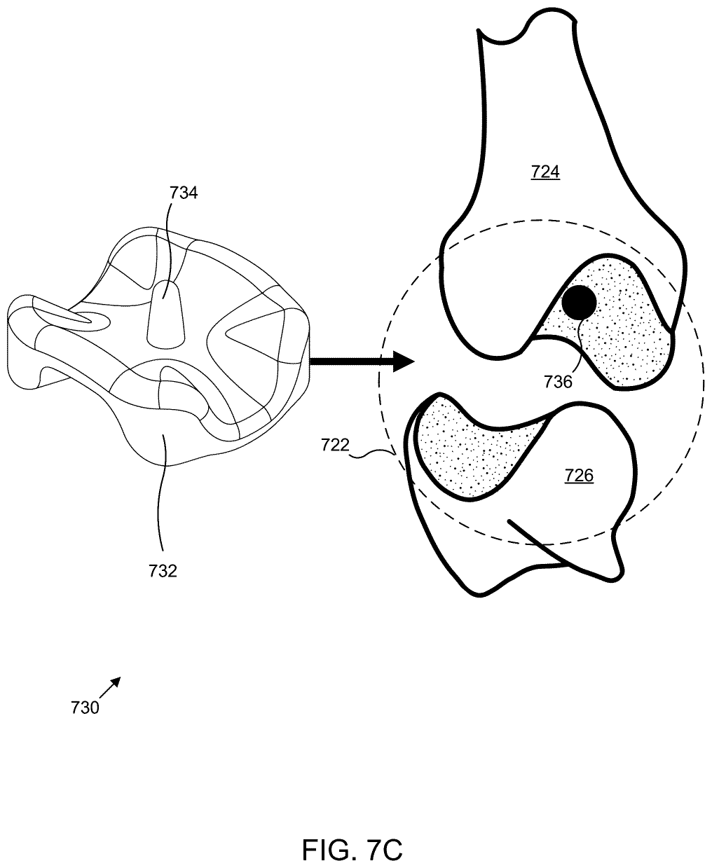

FIG. 7A illustrates another exemplary placement of an implantable interpositional orthopedic pain management device in a bone joint. Here, diagram 700 illustrates a general illustration of device 702 being configured for insertion into joint 704 between bones 706-708. As used herein, like numbered and/or like named elements are assumed to be referencing the same or a substantially similar element having the same or substantially similar function or structure. Differences in function or structure may be described separately with regard to a specific feature described. In some examples, device 702 may be of varying dimensions, shapes, sizes, or other attributes, including modification for joints other than a CMC joint. Here, joint 704 is provided as a basic illustration of a joint in which multiple bones (e.g., bones 706-708) are positioned against each other, but without any intervening material (e.g., cartilage or other bone structures). Device 702 may be formed of varying size and shape to be used, as described herein, for insertion between bones 706-708 to provide the functions provided and described elsewhere. Although bones 706-708 are shown, joint 704 may include other bones or bone structures beyond those shown and described.

As shown, joint 704 between bones 706-708 may lack cartilage, in which case, device 702 may be surgically implanted to prevent contact resulting in pain (i.e., pain relief and management). In other examples, device 702 may be surgically implanted into joint 704 between bones 706-708 to prevent dislocation and, once inserted, features such as those described above in connection with various implementations of an implantable device may be used to prevent expulsion of device 702 from joint 704. In other examples, device 702 as depicted in diagram 700 and the elements shown and described may be designed, configured, formed, modified, or implemented apart from the examples shown or described and are not limited to those provided.

FIG. 7B illustrates a further exemplary placement of an implantable interpositional orthopedic pain management device in a bone joint. Here, diagram 720 provides a three-dimensional oriented view (i.e., perspective view) of surgically implanting device 702 into joint 722 between bones 724 and 726, which are not limited or restrictive to only those of the CMC joint. As used herein, like numbered and/or like named elements are assumed to be referencing the same or a substantially similar element having the same or substantially similar function or structure. Differences in function or structure may be described separately with regard to a specific feature described. For example, bones 724 and 726 may be representative of those found in a knee joint (e.g., femur and tibia) and device 722 may be used to replace worn, damaged, aged, missing, or otherwise incapacitated cartilage that is no longer sufficiently structured or functionally able to provide dynamic stability to joint 722, enable range of motion, or alleviate pain when bones 724-726 physically contact each other. In other examples, device 702 as depicted in diagram 720 and the elements shown and described may be designed, configured, formed, modified, or implemented apart from the examples shown or described and are not limited to those provided.

FIG. 7C illustrates yet another exemplary placement of an implantable interpositional orthopedic pain management device in a bone joint. Here, diagram 730 provides a three-dimensional oriented view (i.e., perspective view) of surgically implanting device 732 into joint 722 between bones 724 and 726, anchor structure 734 and anchor recess 736. As described above, joint 722 is not limited to a specific joint or type of joints, examples of which are provided for purposes of illustration and explanation. As used herein, like numbered and/or like named elements are assumed to be referencing the same or a substantially similar element having the same or substantially similar function or structure. Differences in function or structure may be described separately with regard to a specific feature described.

As described above, device 732 may be implemented similar to other devices (e.g., device 100 (FIGS. 1A-1G), device 200 (FIGS. 2A-2G), device 300 (FIGS. 3A-3G), device 400 (FIGS. 4A-4G)). In some examples, device 732 may also be varied to include elements other than those described herein (e.g., peripheral protrusions, periphery, opposing interpositional saddle surfaces, or others). As shown here, device 732 may be implemented with anchor structure 734, which may be an extra or different element configured to be placed, positioned, or otherwise inserted into anchor recess 736 to provide, among other uses, a point of fixation to bone 724 within joint 722. In some examples, when inserted into anchor recess 736, anchor structure 734 may be used to prevent device 732 from a loss or degradation of position or orientation, dislodgement, or expulsion from joint 722. Further, anchor structure 734 may also be used to prevent one or both of bones 724-726 (which may be any type of bone surrounding any type of joint, as used herein) from becoming dislocated from joint 722. In other examples, anchor structure 734 may be designed, configured, or implemented differently and is not limited to the shape, structure, or function as described herein. For example, anchor structure 734 may have different shapes or other structures, such as protrusions, corners, recesses, or other elements that are configured to fit within anchor recess 736. In still other examples, anchor recess 736 may not be present and bone 724 may instead be configured to receive anchor structure 734 (e.g., which may be implemented as a bump, protrusion, nub, or other type of raised surface or structure (which may or may not be coupled directly or indirectly or formed with device 73)), which have a penetrating or sharp edge that may be pressed into bone 724 without a pre-configured or pre-cut recess or receptacle such as anchor recess 736. In still other examples, device 732 and/or anchor structure 734 may be configured differently and is not limited to the examples shown or described herein.

For example, device 732 may be designed, configured, or implemented differently. In some examples, a stem or substrate, which may be disposed on one or more sides of a joint, may be implemented with one or more peripheral protrusions to be used with or without anchor structure 734 or anchor recess 736. A stem (not shown) may be a substantially flat, smooth, sharp, shortened, elongated, or other shape apart from those shown and described, but when coupled (directly or indirectly) to one or more peripheral protrusions (e.g., peripheral protrusions 112-118 (FIGS. 1A-1G), 212-218 (FIGS. 2A-2G), 312-318 (FIGS. 3A-3G), 412-418 (FIGS. 4A-4G), or others, without limitation or restriction) or other structures (e.g., anchor structure 734) may be used to provide the functions as described herein, including, but not limited to pain relief, pain management, cartilaginous replacement/supplement/augmentation, prevention of dislocation, and others. In other examples, device 732 as depicted in diagram 730 and the elements shown and described may be designed, configured, formed, modified, or implemented apart from the examples shown or described and are not limited to those provided.

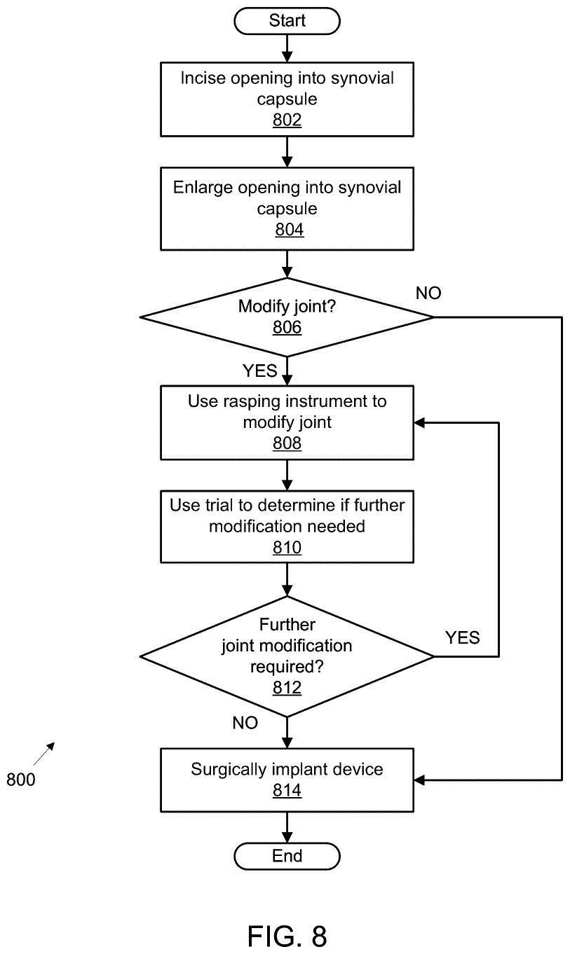

FIG. 8 illustrates an exemplary surgical technique for implantable interpositional orthopedic pain management. Here, process 800 begins by incising (i.e., making an incision) into a synovial capsule surrounding a joint (802). Once an incision (i.e., an opening) has been made, the opening into a synovial capsule may be enlarged using, for example, a trocar or other cutting tool designed for surgically enlarging an incision (804). A determination is then made to determine whether the joint require modification due to obstructions or restrictions such as an osteophyte, abnormal bone growth, or other skeletal structure or sub-structure that may obstruct the articulation of an implantable device (e.g., device 100 (FIGS. 1A-1G), device 200 (FIGS. 2A-2G), device 300 (FIGS. 3A-3G), device 400 (FIGS. 4A-4G), or any other implementation of said devices having one or more peripheral protrusions and other features as described herein) once positioned within a joint (806). If modification is desired, a rasping instrument (e.g., rasping instrument 500 (FIGS. 5A-5G)) may be used to rasp or remove/cut away obstructions within a joint in order to permit insertion, articulation, and non-obstruction of a device once surgically implanted (808).

After rasping or modifying a joint, a trial (e.g., trial 612 (FIG. 6B)) may be inserted into the joint through the enlarged opening to determine if further modification (e.g., additional modification using, for example, rasping instrument 500 (FIGS. 5A-5G)) is desired (810). A determination is then made using a trial as to whether further modification of the joint is required (812). In some examples, if further modification is desired, then rasping instrument 500 (FIGS. 5A-5G) may be used (808) and trial 612 (FIG. 6B) is again inserted to determine if various attributes are within tolerances and/or thresholds for surgical implantation of a device (e.g., device 100 (FIGS. 1A-1G), device 200 (FIGS. 2A-2G), device 300 (FIGS. 3A-3G), device 400 (FIGS. 4A-4G)) (810). However, if modification of a joint is not required, then a device may be surgically implanted and the process ends (814). In other examples, process 800 and the sub-processes or processes shown and described may be designed, configured, performed, ordered, reordered, or otherwise implemented differently than the examples shown or described and are not limited to those provided.

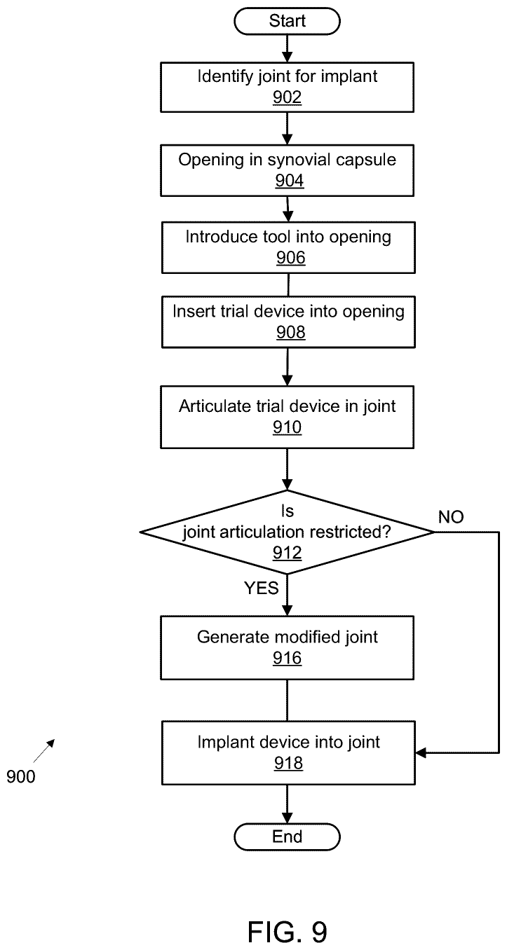

FIG. 9 illustrates another exemplary surgical technique for implantable interpositional orthopedic pain management. Here, alternate process 900 begins by identifying a joint for implantation using a device (e.g., device 100 (FIGS. 1A-1G), device 200 (FIGS. 2A-2G), device 300 (FIGS. 3A-3G), device 400 (FIGS. 4A-4G)) (902). After identifying a joint for implantation, an opening is made into a synovial capsule surrounding the joint (e.g., CMC joint) (904). Next, a tool may be introduced into the opening (e.g., scalpel, trocar, or other cutting tools) to enlarge the initial incision (906). Once the opening/incision has been enlarged, a trial (e.g., trial 612 (FIG. 6B)) is inserted into the joint to determine one or more attributes (e.g., size, position, orientation, and others, without limitation or restriction) (908). In order to aid determination of the one or more factors, the trial device is articulated. In some examples, trial 612 may be articulated using, for example, a stem molded, adhered, attached, or otherwise coupled, directly or indirectly, to a trial device (e.g., trial device 614 (FIG. 6B)) (910). By articulating trial 612, a determination may be made to identify whether any obstructions, restrictions, or limitations are present in the joint (912). For example, osteophytes (i.e., abnormal bone growth or bone structures within a joint) may form into a joint that could prohibit an implantable device (e.g., device 100 (FIGS. 1A-1G), device 200 (FIGS. 2A-2G), device 300 (FIGS. 3A-3G), device 400 (FIGS. 4A-4G)) from articulating fully within a given range of motion, or prohibit articulation of bones adjacent to the joint once surgical implantation has occurred.

In some examples, if a determination is made that a joint is restricted in its ability to articulate, then modification of the joint may be desired and performed using, for example, surgical instruments such as rasping instrument 500 (FIGS. 5A-5G) (916). Once modified or, in the alternative, if no obstruction, restriction, or limitation to articulation of a target joint was detected in step 912, then a device may be surgically implanted into the joint (918) and process 900 ends. In other examples, process 900 and the sub-processes or processes shown and described may be designed, configured, performed, ordered, reordered, or otherwise implemented differently than the examples shown or described and are not limited to those provided.

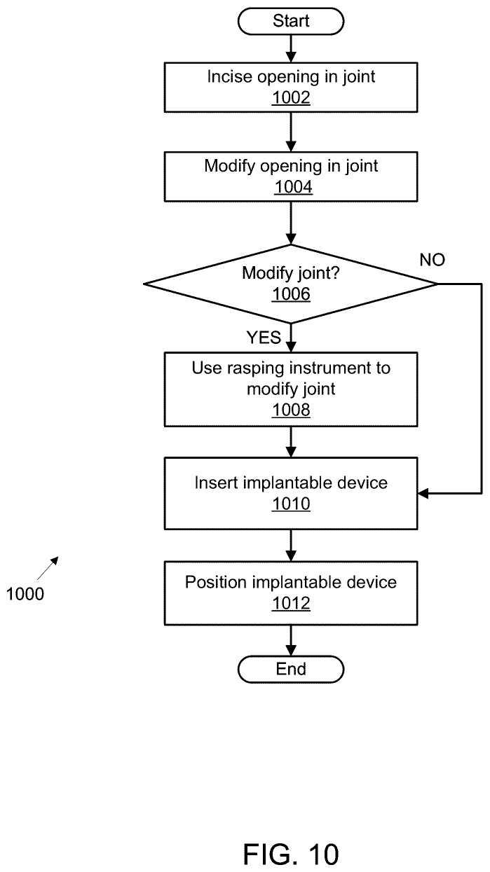

FIG. 10 illustrates an exemplary surgical technique for implantable interpositional orthopedic pain management. Here, process 1000 begins by incising an opening into a joint (1002). Once incised, the opening is modified (i.e., enlarged) (1004). As described herein, various surgical tools and techniques may be used to enlarge the incision, without limitation or restriction, to any particular technique. In some examples, a determination is made as to whether a joint or bones forming the joint need to be modified in order to permit implantation, positioning, and unimpeded (i.e., unobstructed, unrestricted) articulation of a device (e.g., device 100 (FIGS. 1A-1G), device 200 (FIGS. 2A-2G), device 300 (FIGS. 3A-3G), device 400 (FIGS. 4A-4G)) once surgically implanted in a joint (1006). If modification of a joint is required, then a rasping instrument (e.g., rasping instrument 500 (FIGS. 5A-5G)) may be used to modify the joint by removing obstructions, rasping or cutting down osteophytes or other bone growth or bone structures (1008). Once modified or if no modification is required, a device may be surgically implanted (1010). Once inserted, a device may be positioned in order to position peripheral protrusions, such as those described above, to permit articulation of the joint, demonstration of dynamic stability, and restoration (either immediately or a gradual restoration) of strength and range of motion, among other benefits (1012). In other examples, process 1000 and the sub-processes or processes shown and described may be designed, configured, performed, ordered, reordered, or otherwise implemented differently than the examples shown or described and are not limited to those provided.

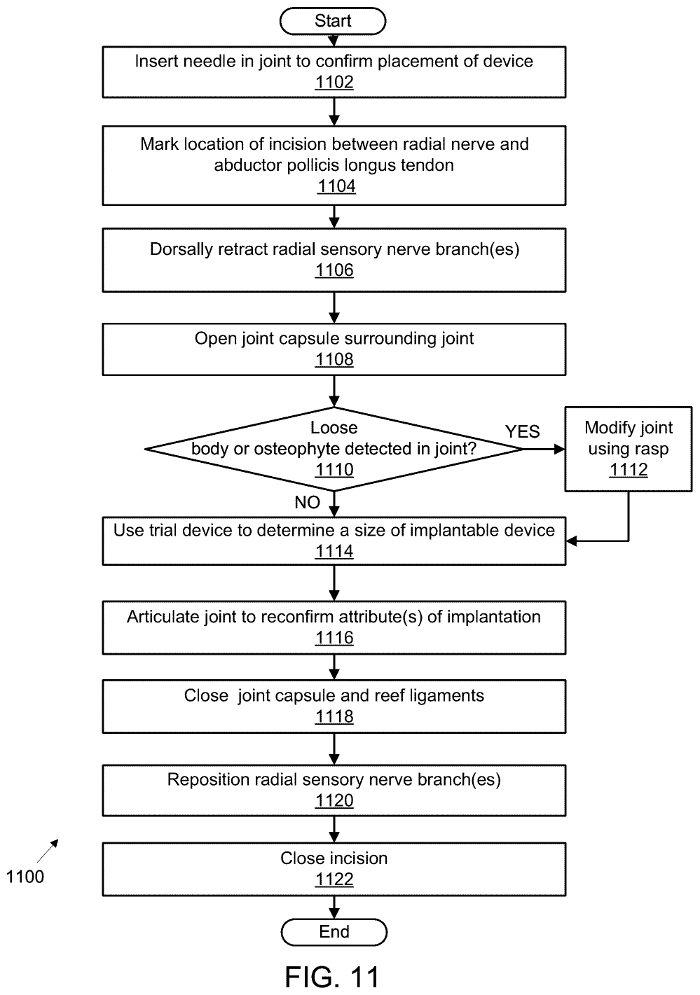

FIG. 11 illustrates yet another exemplary surgical technique for implantable interpositional orthopedic pain management. Here, process 1100 begins by inserting a needle into a joint to confirm placement of a device (1102). After determining the intended position of a surgically implanted device (e.g., device 100 (FIGS. 1A-1G), device 200 (FIGS. 2A-2G), device 300 (FIGS. 3A-3G), device 400 (FIGS. 4A-4G)), a location for an initial incision is marked (1104). In some examples, an incision performed for purposes of inserting a device (e.g., device 100 (FIGS. 1A-1G), device 200 (FIGS. 2A-2G), device 300 (FIGS. 3A-3G), device 400 (FIGS. 4A-4G)) into a CMC joint may be marked as running between the radial nerve and the abductor pollicis longus tendon (“APLT”), to avoid cutting, slicing, incising, or otherwise damaging either the radial nerve or the APLT. Generally, an incision should lie or fall between the radial nerve and the APLT. In other words, the radial nerve and the APLT may bracket either side of an incision as marked in this step. Once marked, one or more radial sensory nerve branches are dorsally retracted (1106). Next, a joint capsule (e.g., synovial capsule) surround a joint is opened (1108). A determination is then made as to whether a loose body (e.g., bone shard, loose cartilage, or other undesired tissue or bone remnants) or osteophytes are detected within a joint (1110). If detected, then rasping instrument 500 (FIGS. 5A-5G) may be used to rasp or cut osteophytes or remove loose objects within the joint (1112). In other examples, different types of surgical tools may be used and are not limited to the examples presented and discussed herein. If no loose bodies (i.e., objects) or osteophytes are detected within a joint, then a trial device may be used to determine a size of an implantable device (1114). Once inserted into a joint through an enlarged opening incised into a joint capsule, trial 612 (FIG. 6B) may be articulated to determine a size of a device (e.g., device 100 (FIGS. 1A-1G), device 200 (FIGS. 2A-2G), device 300 (FIGS. 3A-3G), device 400 (FIGS. 4A-4G)) ultimately intended for surgical implantation in a joint (1114).

After determining a size of a device to be surgically implanted into a joint, articulation may be performed with a trial inserted into the joint in order to reconfirm one or more attributes of implantation (1116). In some examples, attributes of implantation may include size, scale, orientation, position, or other factors for consideration when surgically implanting a device (e.g., device 100 (FIGS. 1A-1G), device 200 (FIGS. 2A-2G), device 300 (FIGS. 3A-3G), device 400 (FIGS. 4A-4G)). After reconfirming attributes associated with the surgically implanted device (i.e., “device”), the joint capsule may be closed and one or more ligaments reefed (1118). Radial sensory nerve branches may then be repositioned prior to closure (1120) and the initial incision is closed (1122). In other examples, process 1000 and the sub-processes or processes shown and described may be designed, configured, performed, ordered, reordered, or otherwise implemented differently than the examples shown or described and are not limited to those provided.

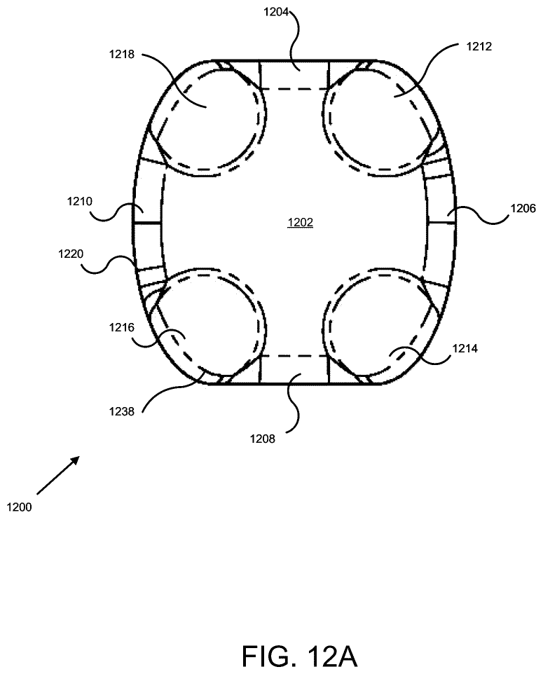

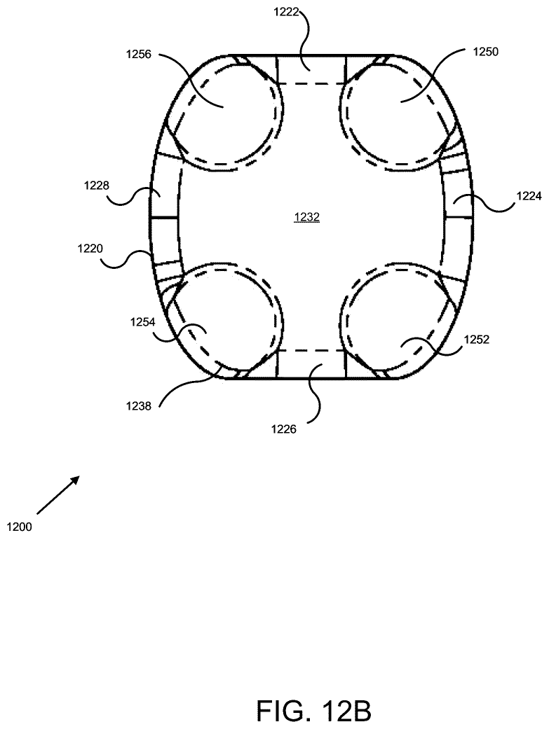

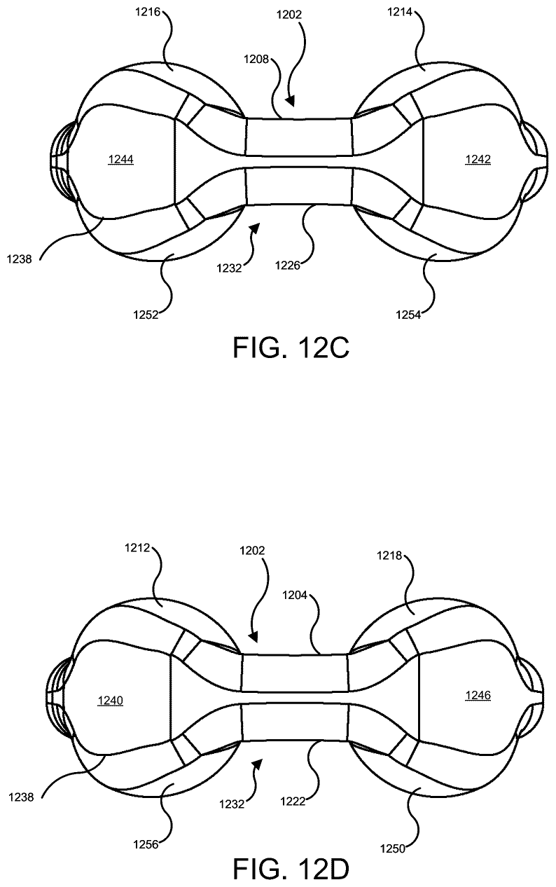

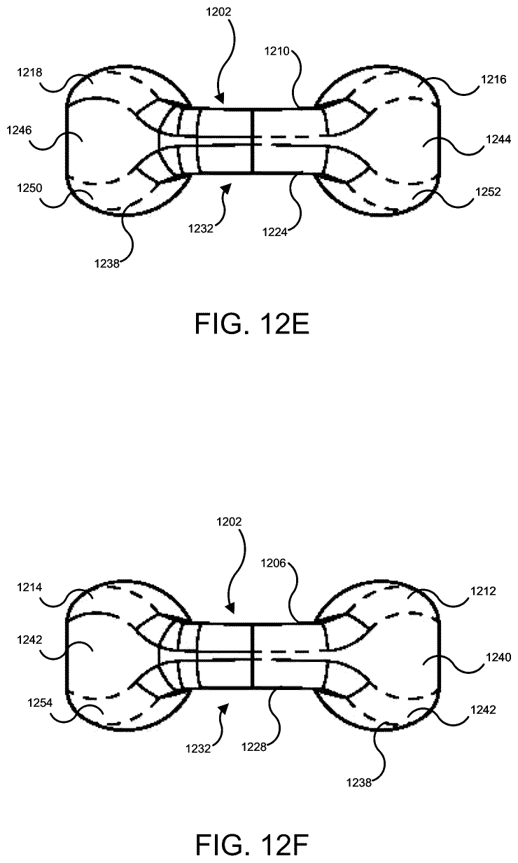

FIG. 12A illustrates a top view of an exemplary implantable interpositional orthopedic pain management apparatus. Here, device 1200 includes a top surface 1202, channel openings 1204-1210, peripheral protrusions 1212-1218, perimeter 1220, and contours 1238. As used herein, like numbered and/or like named elements are assumed to be referencing the same or a substantially similar element having the same or substantially similar function or structure. Differences in function or structure may be described separately with regard to a specific feature described. In some examples, top surface 1202 has at least a portion with a radius of curvature selected to fit with or against an end of a bone (e.g., metacarpal, trapezium, tibia, femur, or others, without limitation or restriction). In some examples, top surface 1202 may have at least a portion having a radius of curvature such that top surface 1202 is substantially concave to receive (or be shaped to substantially fit) a distal or proximal end of a bone. In other examples, top surface 1202 may have at least a portion with a radius of curvature such that top surface 1202 is substantially convex to receive an end of a bone. The radius of curvature of top surface 1202 may be larger than that of a bone intended to be received. In some examples, channel openings 1204-1210 may constitute part of top surface 1202 of device. A channel (e.g., as formed by top surface 1202 and channel openings 1204 and 1208) may be disposed on top surface 1202 of device 1200. Peripheral protrusions 1212-1218 may be disposed on top surface 1202 with outward facing vertical surfaces 1240-1246, to be later described in reference to FIGS. 12C-12G. In some examples, peripheral protrusions 1212-1218 may be disposed substantially about perimeter 1220 of device 1200, and aid in preventing expulsion of device from a joint in which device is implanted. Peripheral protrusions 1212-1218 may be formed and integrated with top surface 1202 to provide pronounced structures that, when device 1200 is surgically implanted in a joint, may be configured to interact within articulating or non-articulating regions (or, in some examples, in a combination of articulating and non-articulating regions) of a joint. The positions of peripheral protrusions 1212-1218 may be selected such that they do not substantially interfere with motion of the joint once implanted within a joint. Peripheral protrusions 1212-1218 may be disposed between proximally-positioned channel openings 1204-1210. For example, peripheral protrusion 1212 may be disposed between proximally-positioned channel openings 1204 and 1206. Peripheral protrusions 1212-1218 may be configured to maintain anatomical alignment of device 1200 when inserted into the joint (not shown). Peripheral protrusions 1212-1218 may also be disposed on opposing sides of a channel opening 1204-1210. For example, peripheral protrusions 1212 and 1214 are disposed on opposing sides of channel opening 1206. The positions of channel openings 1204-1210 and the one or more channels may be configured and selected to have a single channel or dual channels to receive the head or torus of a single bone of a joint.

As described herein, device 1200 may be surgically implanted to achieve stability and regain hand strength in a carpometacarpal (“CMC”), basal, or other type of joint and is not limited to any particular joint. When implanted, device 1200 using peripheral protrusions 1212-1218 may be used to prevent expulsion of device 1200 from a joint while also providing dynamic stability and pain relief in cases where cartilage has been worn away, destroyed, damaged, or is otherwise missing from a joint. Further, peripheral protrusions 1212-1218 are configured to not interfere with motion of joint in order to provide maximum range of extension and motion associated with individual bones forming a joint, and prevents migration of device 1200 from a joint. As described herein, device 1200 may be surgically implanted (i.e., placed) in a joint at a point, position, and/or orientation where device 1200 is least likely to be expulsed when the joint is articulated. As described above, device 1200 is configured to permit motion or articulation of a joint without dislocation or expulsion of device 1200 from the joint. In other words, when bones in a given joint are articulated, device 1200 when implanted may be configured and implemented to prevent one or more bones from dislocation or expulsing device 1200 from the joint. In some examples, device 1200 with peripheral protrusions 1212-1218 is configured to permit motion such as pivoting about one or more axes (e.g., abduction-adduction, flexion-extension, supination-pronation, and others) without dislocation of bones or displacement of device 1200 from a joint. Device 1200 may include additional surface elements as generally identified by contours 1238. For example, device 1200 may include an additional curved surface element in between peripheral protrusion 1216 and top surface 1202, as illustrated by contours 1238. In other examples, device 1200 and the elements shown and described may be designed, configured, formed, modified, or implemented apart from the examples shown or described and are not limited to those provided.

FIG. 12B illustrates a bottom view of an exemplary implantable interpositional orthopedic pain management apparatus. Here, device 1200 includes perimeter 1220, bottom surface 1232, channel openings 1222-1228, contours 1238, and peripheral protrusions 1250-1256. As used herein, like numbered and/or like named elements are assumed to be referencing the same or a substantially similar element having the same or substantially similar function or structure. Differences in function or structure may be described separately with regard to a specific feature described. Elements described herein are similar to those illustrated and described in reference to FIG. 12A. In some examples, bottom surface 1232 has a radius of curvature such that bottom surface 1232 is substantially concave. Bottom surface 1232 may also be substantially convex. The radius of curvature of bottom surface 1232 may be selected such that it is configured to receive a distal or proximal end, or a portion (e.g., central ridge, radial facet volar tubercle, or other structure disposed toward the proximal end of a metacarpal) of a bone. In other examples, top and bottom surfaces 1202, 1232 may be implemented differently and are not limited to the examples shown and described. Peripheral protrusions 1250-1256 may be disposed on bottom surface 1232 with outward facing vertical surfaces 1240-1246, to be later described in reference to FIGS. 12C-12G. In some examples, peripheral protrusions 1250-1256 may be disposed substantially about perimeter 1220 of device 1200, and aid in preventing expulsion of device 1200 from a joint in which device 1200 is implanted. Peripheral protrusions 1250-1256 may be formed and integrated with bottom surface 1232 to provide pronounced structures that, when device 1200 is surgically implanted in a joint, may be configured to interact within articulating or non-articulating regions (or, in some examples, in a combination of articulating and non-articulating regions) of a joint. Peripheral protrusions 1250-1256 may also be disposed on opposing sides of a channel opening 1222-1228. For example, peripheral protrusions 1250 and 1252 are disposed on opposing sides of channel opening 1224. The positions of the channel openings 1222-1228 and the one or more channels may be configured and selected to have a single channel or dual channels to receive the head or torus of a single bone of a joint. In still other examples, device 1200 and the elements shown and described may be designed, configured, formed, modified, or implemented apart from the examples shown or described and are not limited to those provided.

FIG. 12C illustrates an anterior view of an exemplary implantable interpositional orthopedic pain management apparatus. Here, an anterior or front view of device 1200 is shown, including top surface 1202, bottom surface 1232, channel openings 1208 and 1226, protrusions 1214, 1216, 1252, and 1254, contours 1238, and outward facing vertical surfaces 1242 and 1244. As used herein, like numbered and/or like named elements are assumed to be referencing the same or a substantially similar element having the same or substantially similar function or structure. Differences in function or structure may be described separately with regard to a specific feature described. Here, channel openings 1208 and 1226 may be openings into channels formed with top surface 1202, and bottom surface, 1232, respectively. Channel openings 1208 and 1226 may be disposed substantially proximate to perimeter 1220. The channel formed in top surface 1202 with channel opening 1208 and 1204 (not shown) may be shaped to receive a bone (e.g., metacarpal, trapezium, femur, tibia, or the like). Similarly, the channel formed on bottom surface 1232 with channel opening 1226 and 1222 (not shown) may be shaped to receive a bone (e.g., metacarpal, trapezium, femur, tibia, or the like). In some examples, peripheral protrusions 1204-1210 and 1250-1256 may be shaped with a rounded or spherical portion. In some examples, peripheral protrusions 1204-1210 and 1250-1256 may include outward facing vertical surfaces 1240-1246. In some examples, peripheral protrusions 1204-1210 and 1250-1256 may be configured to provide structures on one side (not shown) of device 1200 or on multiple sides (as shown and described). In other examples, peripheral protrusions 1204-1210 and 1250-1256 and outward facing vertical surfaces 1240-1246 may be formed differently and are not limited to the examples shown and described. Device 1200 may include additional surface elements as generally identified by contours 1238. For example, device 1200 may include an additional curved surface element showing a change in curvature between peripheral protrusion 1252 and outward facing vertical surface 1244, as illustrated by contours 1238. In still other examples, device 1200 and the elements shown and described may be designed, configured, formed, modified, or implemented apart from the examples shown or described and are not limited to those provided.

FIG. 12D illustrates a posterior view of an exemplary implantable interpositional orthopedic pain management apparatus. Here, a posterior or rear view of device 1200 is shown, including top surface 1202, bottom surface 1232, channel openings 1204 and 1222, protrusions 1212, 1218, 1250, and 1256, contours 1238, and outward facing vertical surfaces 1240 and 1246. As used herein, like numbered and/or like named elements are assumed to be referencing the same or a substantially similar element having the same or substantially similar function or structure. Differences in function or structure may be described separately with regard to a specific feature described. In some examples, channel openings 1204 and 1208 (not shown) form a channel configured to receive a bone in top surface 1202 when device 1200 is surgically implanted into a synovial capsule and joint. In some examples, channel openings 1222 and 1226 (not shown) form a channel configured to receive a bone in bottom surface 1232 when device 1200 is surgically implanted into a synovial capsule and joint. In still other examples, device 1200 and the elements shown and described may be designed, configured, formed, modified, or implemented apart from the examples shown or described and are not limited to those provided.

FIG. 12E illustrates a left view of an exemplary implantable interpositional pain management apparatus. Here, a left view of the device 1200 is shown, including top surface 1202, bottom surface 1232, channel openings 1210 and 1224, protrusions 1216, 1218, 1250, and 1252, contours 1238, and outward facing vertical surfaces 1244 and 1246. As used herein, like numbered and/or like named elements are assumed to be referencing the same or a substantially similar element having the same or substantially similar function or structure. Differences in function or structure may be described separately with regard to a specific feature described. In some examples, top surface 1202 is configured, with channel openings 1210 and 1206, to receive a bone or a portion thereof when device 1200 is surgically implanted. Bottom surface 1232 may also be configured, with channel openings 1224 and 1228, to receive a bone or a portion thereof when device 1200 is surgically implanted. For example, if device 1200 is surgically implanted into a CMC joint, top surface 1202 may be configured, with channel openings 1204 and 1208, to receive (i.e., top surface 1202 may have a radius of curvature that is larger than that of a trapezium bone) a trapezium bone, or other bone adjacent to the joint. In some examples, outward facing vertical surfaces 1240-1246 (not shown) may be surfaces generally in between a peripheral protrusion on top surface 1202 (e.g., 1216) and a peripheral protrusion on bottom surface 1232 (e.g., 1252). These peripheral protrusions (e.g., 1216 and 1252) may be opposing one another. Outward facing vertical surface 1240-1246 may have a bulb shape, such that that they have a rounded portion and a pointed portion. However, the shape of outward facing vertical surfaces is not limited to those shown and described herein and may be based on the shape of adjacent peripheral protrusions. In still other examples, device 1200 and the elements shown and described may be designed, configured, formed, modified, or implemented apart from the examples shown or described and are not limited to those provided.

FIG. 12F illustrates a right view of an exemplary implantable interpositional pain management apparatus. Here, a right view of the device 1200 is shown, including top surface 1202, bottom surface 1232, channel openings 1210 and 1224, protrusions 1216, 1218, 1250, and 1252, contours 1238, and outward facing vertical surfaces 1244 and 1246. As used herein, like numbered and/or like named elements are assumed to be referencing the same or a substantially similar element having the same or substantially similar function or structure. In some examples, top surface 1202 is configured, with channel openings 1206 and 1210 (not shown), to receive a bone or a portion thereof when device 1200 is surgically implanted. Bottom surface 1232 may also be configured, with channel openings 1228 and 1224 (not shown), to receive a bone or a portion thereof when device 1200 is surgically implanted. Outward facing vertical surfaces 1240 and 1242 may have a curvature element that aligns with perimeter 1220 (not shown), such that device 1200 minimally interferes with lateral tissue or bones once surgically implanted within a joint. Differences in function or structure may be described separately with regard to a specific feature described. In other examples, device 1200 and the elements shown and described may be designed, configured, formed, modified, or implemented apart from the examples shown or described and are not limited to those provided.

FIG. 12G illustrates a perspective view of an exemplary implantable interpositional orthopedic pain management apparatus. Here, device 1200 includes top surface 1202, channel openings 1204-1210, peripheral protrusions 1212-1218 and 1254, perimeter 1220, contours 1238, and outward facing vertical surfaces 1240-1244. As used herein, like numbered and/or like named elements are assumed to be referencing the same or a substantially similar element having the same or substantially similar function or structure. Differences in function or structure may be described separately with regard to a specific feature described. Top and bottom surfaces 1202 and 1232 may be together referred to as the body of the device. As shown, top surface 1202 has a substantially concave shape. In some examples, top surface 1202 may be substantially four sided, with peripheral protrusions 1212-1218 disposed on four corners. Perimeter 1220 may constitute the outermost portion of top or bottom surfaces 1202, 1232. Peripheral protrusions 1212-1218 may be disposed along perimeter 1220, and peripheral protrusions 1250-1256 (not shown) may be similarly disposed along perimeter 1220, on bottom surface 1232, opposite peripheral protrusions 1212-1218. In other examples, however, peripheral protrusions may not be disposed opposite peripheral protrusions on an opposite side of device 1200. Outward facing vertical surface 1242 may be disposed between opposing peripheral protrusions 1214 and 1254, for example, and be curved, as to not extend outward beyond perimeter 1220. In some examples, outward facing vertical surfaces 1240-1244 (not shown) may be formed with perimeter 1220 and associated peripheral protrusions 1212-1218 and 1250-1256 (not shown). Channel opening 1204 indicates an opening to a channel formed with top surface 1202 and channel opening 1208. Similarly, channel opening 1206 indicates an opening to a channel formed with top surface 1202 and channel opening 1210. Device 1200 may include additional surface elements as illustrated and identified generally as contours 1238. For example, device 1200 may include a convex curved portion as a transition between perimeter 1220 and top surface 1220. As with the other examples throughout this description, various radii of curvature may be used and are not limited to the examples shown and described. In other examples, device 1200 and the elements shown and described may be designed, configured, formed, modified, or implemented apart from the examples shown or described and are not limited to those provided.

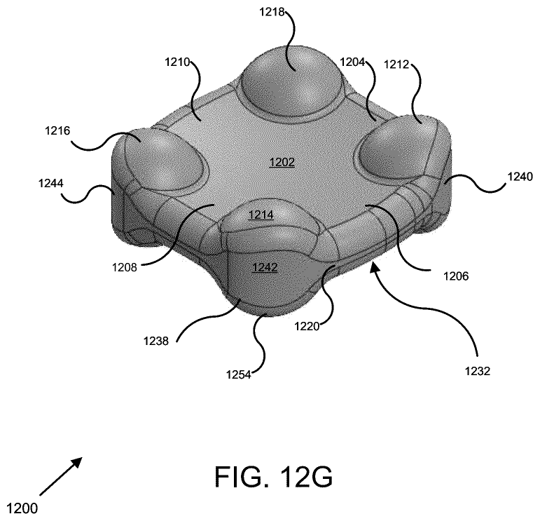

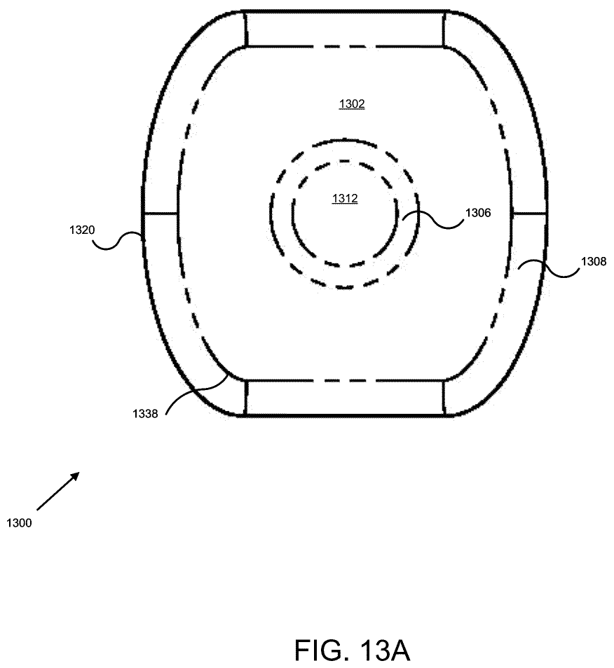

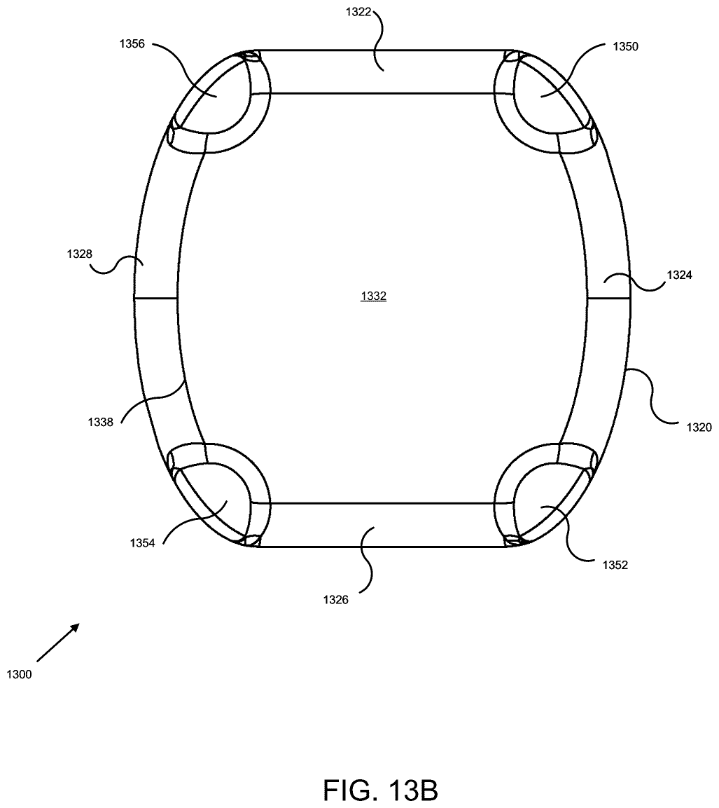

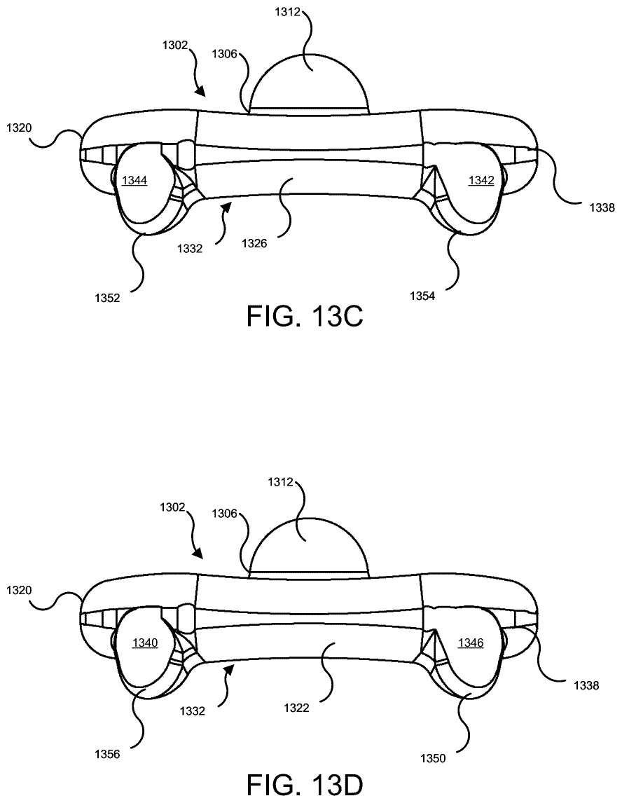

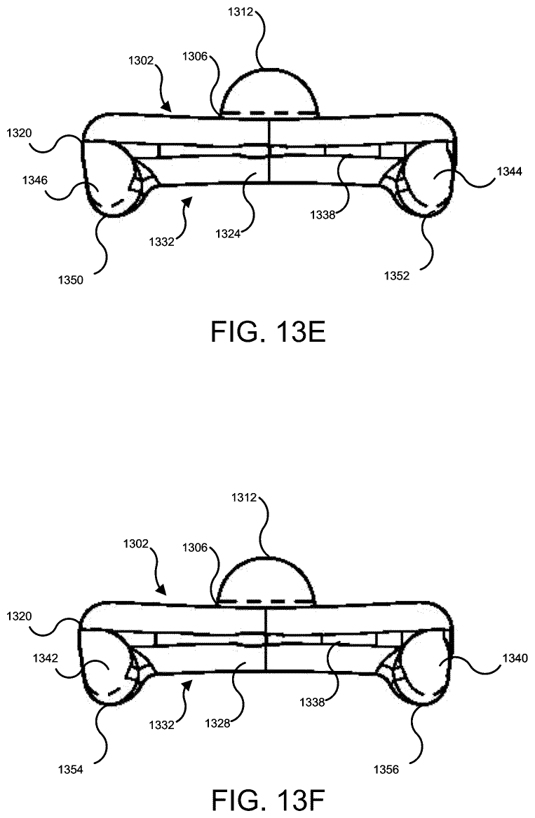

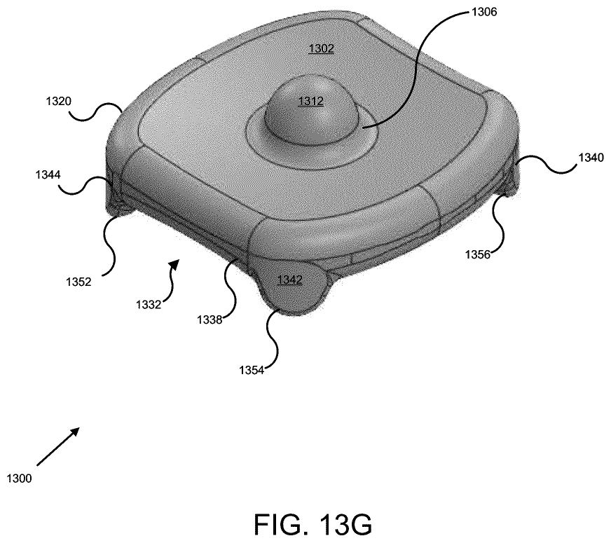

FIG. 13A illustrates a top view of an exemplary implantable interpositional orthopedic pain management apparatus. Here, device 1300 includes top surface 1302, peripheral protrusion 1312, protrusion slope 1306, perimeter wall 1308, perimeter 1320, and contours 1338. As used herein, like numbered and/or like named elements are assumed to be referencing the same or a substantially similar element having the same or substantially similar function or structure. Differences in function or structure may be described separately with regard to a specific feature described. Here, dotted lines are used to illustrate contours 1338 of top surface 1302, and in some examples, changes in the radius of curvature of portions of top surface 1302. For example, there may be a change of radius of curvature of a surface of perimeter wall 1308 to top surface 1302, and there may be change of radius of curvature of top surface 1302 to protrusion slope 1306. Top surface 1302, in some examples, may be substantially concave. As shown, peripheral protrusion 1312 is located in a substantially central position of top surface 1302. However, the position of peripheral protrusion 1312 is not limited to a central position and may be formed with or disposed on top surface 1302 at a position that is spaced from the perimeter 1320. As shown, peripheral protrusion 1312 is partially spherical, however, the shape and size of the protrusion may be varied and are not limited to those shown and described. In some examples, the position and shape of peripheral protrusion 1312 along with the shape of protrusion slope 1306 and perimeter wall 1308 are selected to improve placement, positioning, and expulsion resistance when device 1300 is surgically implanted. Cavities formed within the proximal or distal ends of bones may have structures that device 1300 is contoured to fit. The dotted lines shown in device 1300 may represent contours of peripheral protrusion 1312 that are configured to be disposed within non-articulating portions of a synovial capsule and/or joint and, when motion occurs that articulates a given joint. Once surgically implanted, device 1300 may alleviate pain by preventing distal and proximal ends of bones in a joint from contact while peripheral protrusion 1312 also prevents dislocation (i.e., one or more bones distracting or dislocating from a joint) and expulsion of device 1300. In other examples, device 1300 and the elements shown and described may be designed, configured, formed, modified, or implemented apart from the examples shown or described and are not limited to those provided.

FIG. 13B illustrates a bottom view of an exemplary implantable interpositional orthopedic pain management apparatus. Here, device 1300 includes perimeter 1320, channel openings 1322-1328, contours 1328, peripheral protrusions 1350-1356, and bottom surface 1332. As used herein, like numbered and/or like named elements are assumed to be referencing the same or a substantially similar element having the same or substantially similar function or structure. Differences in function or structure may be described separately with regard to a specific feature described. As shown herein, the bottom view of device 1300 is shown with bottom surface 1332 forming a channel that is configured to be disposed over or fit within a cavity formed with or within a bone, bone structure, bone joint, or portion(s) thereof, functioning similar or substantially similar to the other examples shown and described above and below in connection with various drawings throughout. In some examples, bottom surface 1332 may be convex, concave, or having a radius of curvature that is selected to receive an end or portion of a bone. Peripheral protrusions 1350-1356 may be disposed at positions near perimeter 1320 of device 1300 and may be used to stabilize device 1300 within a synovial capsule of a joint, once implanted, without hindering movement of the joint. One device 1300 is implanted, peripheral protrusions 1350-1356 may maintain the position of device 1300 within the joint, in between at least two bones, without being bonded or otherwise coupled to the bone. In still other examples, device 1300 and the elements shown and described may be designed, configured, formed, modified, or implemented apart from the examples shown or described and are not limited to those provided.

FIG. 13C illustrates an anterior view of an exemplary implantable interpositional orthopedic pain management apparatus. Here, an anterior or frontal view of device 1300 includes a top surface 1302, protrusion slope 1306, peripheral protrusions 1312, 1352, and 1354, perimeter 1320, channel opening 1326, bottom surface 1332, contours 1338, and outward facing vertical surfaces 1344 and 1342. As used herein, like numbered and/or like named elements are assumed to be referencing the same or a substantially similar element having the same or substantially similar function or structure. Differences in function or structure may be described separately with regard to a specific feature described. In some examples, top surface 1302 may have a radius of curvature that is one or two degrees greater than that of an adjacent bone surface of a targeted joint. In other examples, top surface 1302 may be substantially concave. Alternatively, top surface 1302 may be substantially convex. In the example shown, protrusion 1312 has a shape that is partially round or spherical. The size and slope of protrusion slope 1306 and size and shape of peripheral protrusion 1312 is not limited to the example shown herein. For example, peripheral protrusion 1312 may be located substantially close to perimeter 1320 (not shown), on top surface 1302 of device 1300. Outward facing vertical surfaces 1342 and 1344 may be substantially flat, but may also be curved to follow the curvature of perimeter 1320. Outward facing vertical surfaces 1342 and 1344 may be shaped and positioned in a manner to minimize interference of device 1300 with neighboring tissue or bones, thereby managing pain that may be involved in implantation of device 1300. In some examples, a channel is provided by bottom surface 1332 and channel openings 1326 and 1322, which is configured to receive a bone (e.g., metacarpal) when surgically implanted into a joint (e.g., CMC joint). Disposition within a joint (not shown) may include peripheral protrusions 1312 and 1350-1358 being positioned to prevent expulsion of device 1300 when the joint is articulated. Specifically, one or more of peripheral protrusions 1312 and 1350-1358 may be configured to interact with one or more bones, bone structures, heads, ends, or portions thereof when surgically implanted. The curved shapes of peripheral protrusion 1312 and 1350-1358 allows for dynamic movement or adjustment of device 1300, once implanted between two bone surfaces, by allowing for smooth or uninterrupted movement between peripheral protrusions 1312 and 1350-1358 and adjacent anatomical surfaces. Device 1300, as shown and described herein, provides protrusion or corner features that may be tailored to provide customization features for individual joints, bones, or other anatomical structures. Additionally, another channel may be provided by channel openings 1324 and 1328 (not shown) to provide an opposing or substantially opposing channel on the bottom surface 1332 that is shaped to receive mutually reciprocal or substantially mutually reciprocal bones, bone structures, or portions thereof in a joint. In other examples, device 1300 and the elements shown and described may be designed, configured, formed, modified, or implemented apart from the examples shown or described and are not limited to those provided.

FIG. 13D illustrates a posterior view of an exemplary implantable interpositional orthopedic pain management apparatus. Here, a posterior or rear view of device 1300 includes a top surface 1302, protrusion slope 1306, peripheral protrusions 1312, 1350, and 1356, perimeter 1320, channel opening 1322, bottom surface 1332, contours 1338, and outward facing vertical surfaces 1340 and 1346. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described in reference to FIG. 13C. Here, peripheral protrusions 1350 and 1356 are shown, including outward facing vertical surfaces 1340 and 1346 which are configured in structure and function similarly to peripheral protrusion 1352 and 1354 (FIG. 13C) and outward facing vertical surfaces 1342 and 1344 (FIG. 13C). Peripheral protrusions 1350-1356 and outward facing vertical surfaces 1340-1346 may be configured to reside in non-articulating regions of a joint (not shown) when device 1300 is surgically implanted. In some examples, peripheral protrusions 1350-1356 and outward facing vertical surfaces 1340-1346 may be formed with various types of shapes and features (e.g., convex, concave, partially or wholly, or a combination thereof). If one or more bones of the joint (in which device 1300 is surgically implanted) are articulated, then peripheral protrusions 1350-1356 and outward facing vertical surfaces 1340-1346 are configured to prevent the bones from coming into contact with each other, maintaining dynamic stability, and restoring an ability for the joint to be manipulated while preventing expulsion of device 1300 from the joint or dislocation of bones from the joint. In the example shown, top surface 1302 has a single peripheral protrusion 1312, and bottom surface has four peripheral protrusions 1350-1356, however, the configurations of protrusions is not limited to the examples provided herein. In other examples, device 1300 and the elements shown and described may be designed, configured, formed, modified, or implemented apart from the examples shown or described and are not limited to those provided.

FIG. 13E illustrates a left view of an exemplary implantable interpositional orthopedic pain management apparatus. Here, a left side view of device 1300 includes a top surface 1302, protrusion slope 1306, peripheral protrusions 1312, 1350, and 1352, perimeter 1320, channel opening 1324, bottom surface 1332, contours 1338, and outward facing vertical surfaces 1344 and 1346. As used herein, like numbered and/or like named elements are assumed to be referencing the same or a substantially similar element having the same or substantially similar function or structure. Differences in function or structure may be described separately with regard to a specific feature described. A left side view shows bottom surface 1332 forming a channel between channel openings 1324 and 1328 in a substantially orthogonal or substantially mutually reciprocal orientation to the channel described above in connection with FIG. 13C. In other examples, the channel formed by channel openings 1324 and 1328 may be oriented differently (i.e., set at an angle other than orthogonal to a given axis) and implementation of the techniques described herein do not require an axially orthogonal position. In still other examples, device 1300 and the elements shown and described may be designed, configured, formed, modified, or implemented apart from the examples shown or described and are not limited to those provided.

FIG. 13F illustrates a right view of an exemplary implantable interpositional orthopedic pain management apparatus. Here, a right side view of device 1300 includes a top surface 1302, protrusion slope 1306, peripheral protrusions 1312, 1354, and 1356, perimeter 1320, channel opening 1328, bottom surface 1332, contours 1338, and outward facing vertical surfaces 1340 and 1342. As used herein, like numbered and/or like named elements are assumed to be referencing the same or a substantially similar element having the same or substantially similar function or structure. Differences in function or structure may be described separately with regard to a specific feature described. In some examples, peripheral protrusions 1350-1356 may be coupled directly or indirectly with perimeter 1320 or formed as surface or structural features or components thereof. In some examples, the top and bottom surfaces 1302 and 1332 may provide engaging surfaces of device 1300 to fit within a joint having, for example, opposing bones, bone structures, or portions thereof. In still other examples, device 1300 and the elements shown and described may be designed, configured, formed, modified, or implemented apart from the examples shown or described and are not limited to those provided.

FIG. 13G illustrates a perspective view of an exemplary implantable interpositional orthopedic pain management apparatus. Here, a perspective view of device 1300 includes the top surface 1302, protrusion slope 1306, peripheral protrusions 1312 and 1352-1356, perimeter 1320, contours 1338, and outward facing vertical surfaces 1340-1344. As used herein, like numbered and/or like named elements are assumed to be referencing the same or a substantially similar element having the same or substantially similar function or structure. Differences in function or structure may be described separately with regard to a specific feature described. Top and bottom surfaces 1302 and 1332 are formed and integrated with peripheral protrusions 1312 and 1350-1356 to provide structures that, when placed within non-articulating regions of a joint, prevent device 1300 from being expulsed, partially or wholly, when the joint is articulated. In some examples, peripheral protrusions 1312 and 1350-1356 may be shaped with additional surface texture to aid positioning of device 1300 once surgically implanted in between adjacent bone structures. In another example, peripheral protrusions 1350-1356 may be positioned a distance away from perimeter 1320 to support positioning within a joint. Portions of device 1300, once implanted, may be in intermittent or otherwise non-permanent contact with adjacent anatomical or bone surfaces. By being in intermittent contact, device 1300 once implanted is shaped to fit and/or fill with other materials within a synovial capsule such as cartilage, blood, bodily fluids, or other synthetic or organic materials that are injected or otherwise implanted to aid device 1300 in maintaining dynamic stability and position within a joint after surgical implantation. In still other examples, device 1300 and the elements shown and described may be designed, configured, formed, modified, or implemented apart from the examples shown or described and are not limited to those provided.

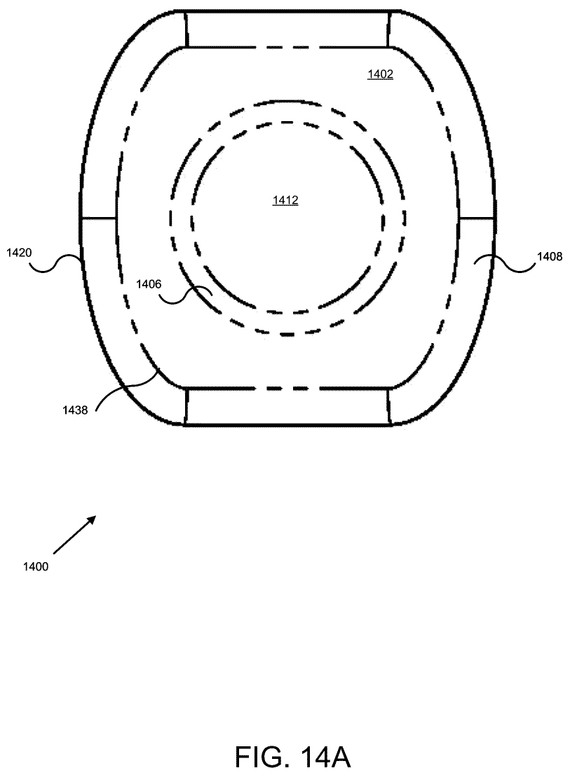

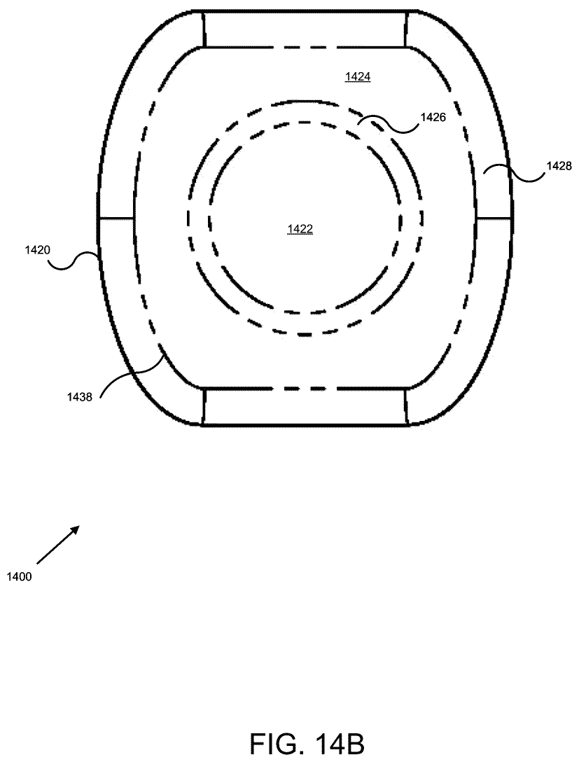

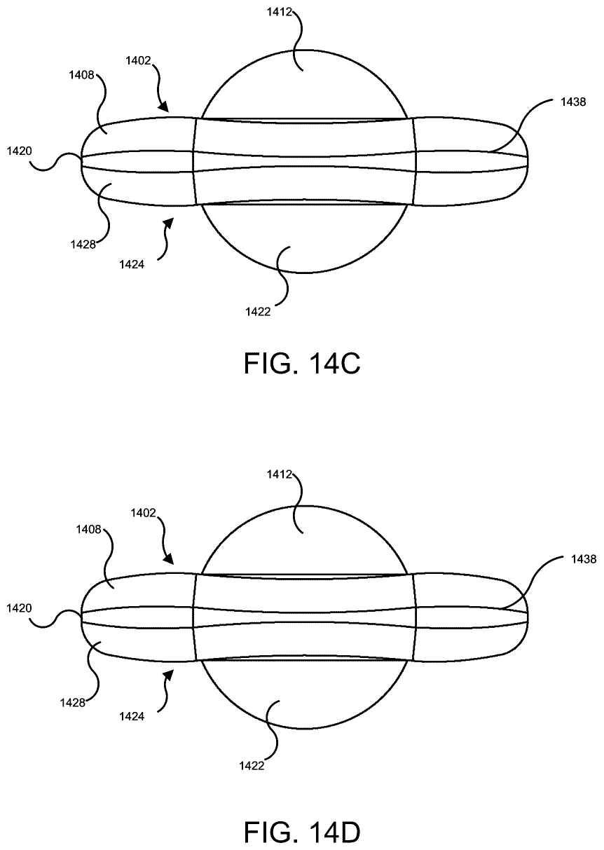

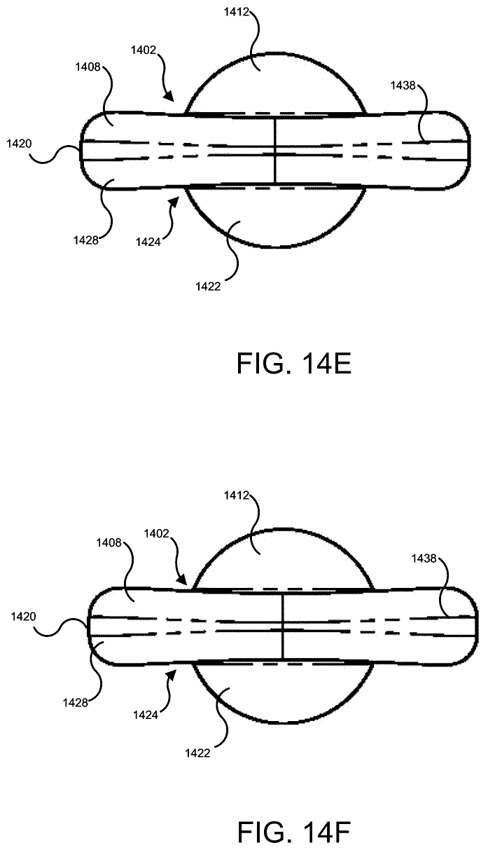

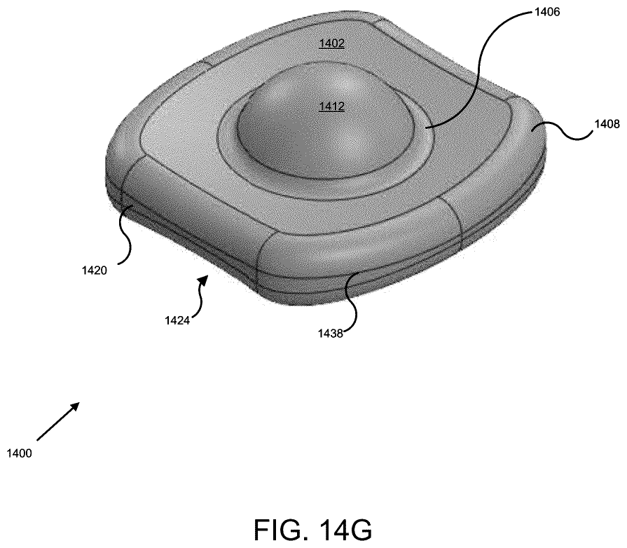

FIG. 14A illustrates a top view of an exemplary implantable interpositional orthopedic pain management apparatus. Here, device 1400 includes top surface 1402, protrusion slope 1406, perimeter wall 1408, peripheral protrusion 1412, perimeter 1420, and contours 1438. As used herein, like numbered and/or like named elements are assumed to be referencing the same or a substantially similar element having the same or substantially similar function or structure. Differences in function or structure may be described separately with regard to a specific feature described. Broken (i.e., dotted) lines are provided for purposes illustrating contours 1438 of device 1400, which, in the example shown, is formed as part of the top surface 1402. In some examples, peripheral protrusion 1412 may be disposed as a structure in a different location on the top surface 1402 that is configured to maintain dynamic stability of device 1400 once surgically implanted in a joint. In the example shown, protrusion slope 1406 encircles peripheral protrusion 1412. Protrusion slope 1406 may have the same or a different radius of curvature of top surface 1402 and may function as a transition slope surface between peripheral protrusion 1412 and top surface 1402. In some examples, top surface 1402 may be substantially concave, and in other examples, top surface 1402 may be substantially convex. The shape or radius of curvature of top surface 1402 may be selected to allow the surface to fit the end of a bone once device 1400 is implanted into a joint. Perimeter wall 1408 may be a portion adjacent to perimeter 1420 that may have the same radius of curvature as top surface 1402, or a different radius of curvature. In some examples, perimeter wall 1408 may have a height element to position device 1400 in a synovial capsule in a joint (not shown). In other examples, device 1400 and the elements shown and described may be designed, configured, formed, modified, or implemented apart from the examples shown or described and are not limited to those provided.

FIG. 14B illustrates a bottom view of an exemplary implantable interpositional orthopedic pain management apparatus. Here, a bottom view of device 1400 is shown, including perimeter 1420, peripheral protrusion 1422, bottom surface 1424, protrusion slope 1426, perimeter wall 1428, and contours. As used herein, like numbered and/or like named elements are assumed to be referencing the same or a substantially similar element having the same or substantially similar function or structure. Differences in function or structure may be described separately with regard to a specific feature described. Structure and function of elements of in the bottom view are substantially similar to elements in reference to FIG. 14A. Broken (i.e., dotted) lines are provided for purposes illustrating contours 1438 of peripheral protrusion 1422, protrusion slope 1426, and perimeter wall 1428, which, in the example shown, are formed as parts of bottom surface 1424. Top and bottom surfaces 1402 (not shown) and 1424, may be together referred to as the body of device 1400. In other examples, device 1400 and the elements shown and described may be designed, configured, formed, modified, or implemented apart from the examples shown or described and are not limited to those provided.

FIG. 14C illustrates an anterior view of an exemplary implantable interpositional orthopedic pain management apparatus. Here, an anterior or front view of device 1400 includes top surface 1402, perimeter walls 1408 and 1428, peripheral protrusions 1412 and 1422, perimeter 1420, and contours 1438. As used herein, like numbered and/or like named elements are assumed to be referencing the same or a substantially similar element having the same or substantially similar function or structure. Differences in function or structure may be described separately with regard to a specific feature described. In the example shown herein, top and bottom surfaces 1402 and 1424 are substantially concave, however, in other examples, top and bottom surfaces 1402 and 1424 may be substantially convex, selected to position device 1400 in between two bones of a joint (not shown). Perimeter walls 1408 and 1428 may be a portion of top and bottom surfaces 1402 and 1424, respectively, that is closest to perimeter 1420, and may be substantially coextensive with top and bottom surfaces 1402 and 1424. Peripheral protrusions 1412 and 1422 may be positioned in a central portion of top and bottom surfaces, 1402 and 1424. However, their positions should not be limited to the central location, for example, peripheral protrusions 1412 and 1422 may both be located off-center, or otherwise closer to perimeter 1420. Peripheral protrusions 1412, 1422 may be partially spherical in shape to position device 1400 between opposing ends, or the torii, or two bones in a joint. The sizes of peripheral protrusions 1412, 1422 may allow for uninterrupted and dynamic movement and shifting of the adjacent bone surfaces on around peripheral protrusions 1412, 1422 as the joint is used. Device 1400, as shown and described here, is an example of another type of implementation that may be used to provide dynamic stability and range of motion while providing pain relief and prevent expulsion due to joint articulation (i.e., one or more bones, bone structures, or portions thereof being manipulated within a joint) after surgical implantation. In still other examples, device 1400 and the elements shown and described may be designed, configured, formed, modified, or implemented apart from the examples shown or described and are not limited to those provided.

FIG. 14D illustrates a posterior view of an exemplary implantable interpositional orthopedic pain management apparatus. Here, a posterior or back view of device 1400 includes top surface 1402, perimeter walls 1408 and 1428, peripheral protrusions 1412 and 1422, perimeter 1420, and contours. As used herein, like numbered and/or like named elements are assumed to be referencing the same or a substantially similar element having the same or substantially similar function or structure. Differences in function or structure may be described separately with regard to a specific feature described. The elements shown herein are substantially similar to show identified and described with reference to FIG. 14C. Peripheral protrusions 1412 and 1422 may be varied and are not limited to examples shown and described and may be varied in number, placement, position, shape, configuration, shape, and may be varied in other attributes, without limitation or restriction. In other examples, device 1400 and the elements shown and described may be designed, configured, formed, modified, or implemented apart from the examples shown or described and are not limited to those provided.

FIG. 14E illustrates a left view of an exemplary implantable interpositional orthopedic pain management apparatus. Here, a left view of device 1400 includes top surface 1402, perimeter walls 1408 and 1428, peripheral protrusions 1412 and 1422, perimeter 1420, bottom surface 1424, and contours 1438. As used herein, like numbered and/or like named elements are assumed to be referencing the same or a substantially similar element having the same or substantially similar function or structure. Differences in function or structure may be described separately with regard to a specific feature described. In the example shown, peripheral protrusions 1412 and 1422 are located centrally on top and bottom surfaces 1402 and 1422, and have rounded or curved shapes. These positions and shapes may be selected based on the target joint for which device 1400 is to be surgically implanted, one purpose being to optimize the device stability within the joint and reduce or minimize discomfort associated with the implant. In other examples, however, peripheral protrusions 1412 and 1422 may be located at different positions on the surface and may have shapes that are selected based on the topography and radii of curvature of the bones in the target joint. For example, peripheral protrusions 1412 and 1422 may alternatively have convex or concave structures formed within them to provide features to maintain device 1400 within a joint, provide dynamic stability, maintain positioning of device 1400 within a synovial capsule and/or joint, increase or protect ranges of motion, alleviate or prevent pain due to bone contact, or others. Still further, peripheral protrusions 1412 and 1422 may be varied in quantity, placement, location, position, or other attributes relative to perimeter 1420, determined in part by the joint to be used with. In some examples, when device 1400 is surgically implanted, peripheral protrusions 1412 and 1422 may provide structures that are configured to maintain device 1400 in a given joint (i.e., prevent expulsion of device 1400), provide dynamic stability in the given joint, and alleviate pain by preventing bones, bone structures, or portions thereof from contacting each other as a replacement for missing, damaged, worn, or otherwise degraded cartilage within a joint or synovial capsule. Device 1400 may include additional elements as generally identified by contours 1438. For example, device 1400 may include an additional curved surface element in between perimeter walls 1408 and 1428, as illustrated by contours 1438. In still other examples, device 1400 and the elements shown and described may be designed, configured, formed, modified, or implemented apart from the examples shown or described and are not limited to those provided.

FIG. 14F illustrates a right view of an exemplary implantable interpositional orthopedic pain management apparatus. Here, a right view of device 1400 includes top surface 1402, perimeter walls 1408 and 1428, peripheral protrusions 1412 and 1422, perimeter 1420, bottom surface 1424, and contours 1438. As used herein, like numbered and/or like named elements are assumed to be referencing the same or a substantially similar element having the same or substantially similar function or structure. Differences in function or structure may be described separately with regard to a specific feature described. The elements shown herein are substantially similar to show identified and described with reference to FIG. 14E. In the example shown, peripheral protrusions 1412 and 1422 are located centrally on the top and bottom surfaces 1402 and 1422, and have rounded or curved shapes. These positions and shapes may be selected based on the target joint for which device 1400 is to be surgically implanted, one purpose being to optimize the device stability within the joint and reduce or minimize pain associated with the implant. In other examples, however, the peripheral protrusions 1412 and 1422 may be located at different positions on the surface and may have shapes that are selected based on the topography of the bones in the target joint.

FIG. 14G illustrates a perspective view of an exemplary implantable interpositional orthopedic pain management apparatus. Here, device 1400 includes top surface 1402, protrusion slope 1406, peripheral protrusion 1412, perimeter wall 1408, perimeter 1420, bottom surface 1424, and contours 1438. As used herein, like numbered and/or like named elements are assumed to be referencing the same or a substantially similar element having the same or substantially similar function or structure. Differences in function or structure may be described separately with regard to a specific feature described. As shown herein, top surface 1402 may have a radius of curvature such that top surface 1402 is substantially concave. In other examples, top surface 1402 may have a radius of curvature such that the top surface 1402 is substantially convex. Top and bottom surfaces 1402 and 1424 may also have different radii of curvature. Perimeter wall 1408 defines a sloped outside portion of top surface 1402, the size and shape of which may be selected based on the bone surface that device 1400 is intended to be adjacent to, once surgically implanted. Device 1400, or portions thereof, may be intended to be, once surgically implanted, in intermittent contact with adjacent bones. In other examples, device 1400 and the elements shown and described may be designed, configured, formed, modified, or implemented apart from the examples shown or described and are not limited to those provided.

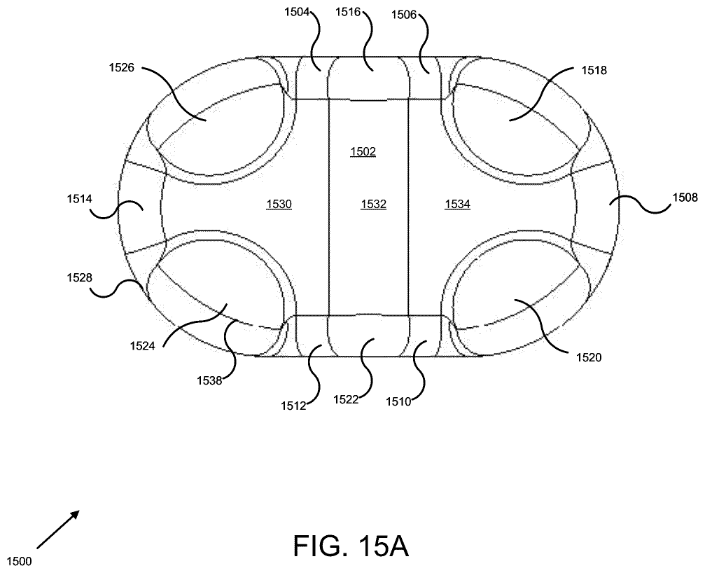

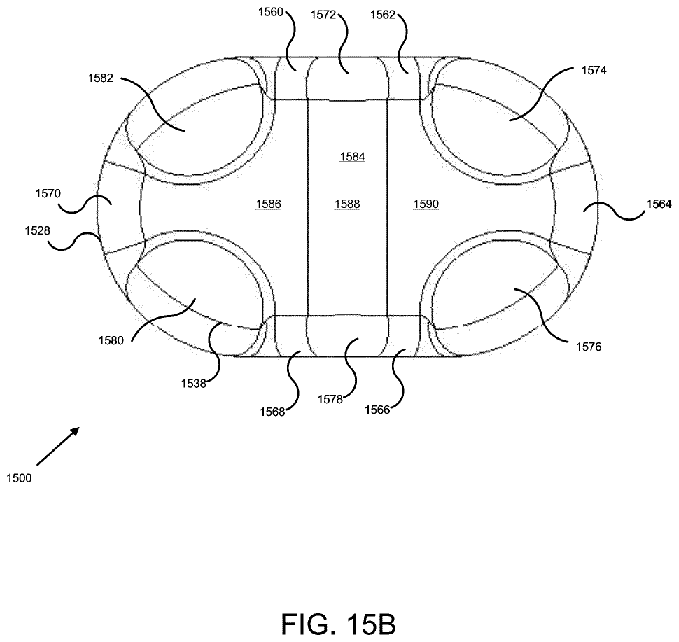

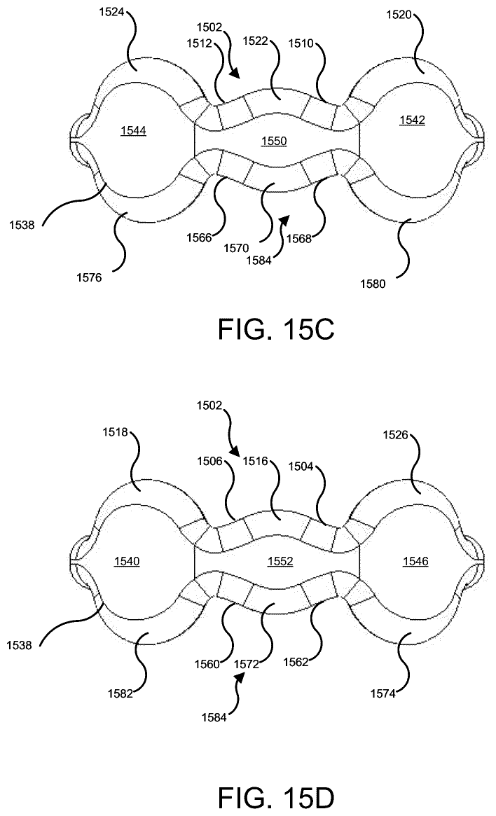

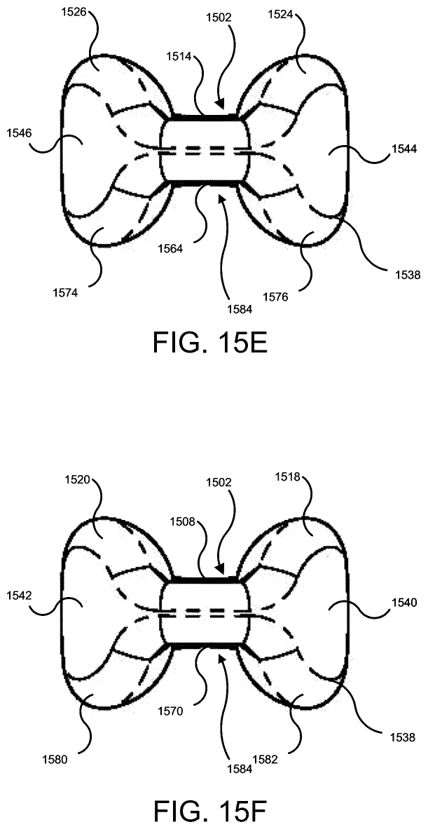

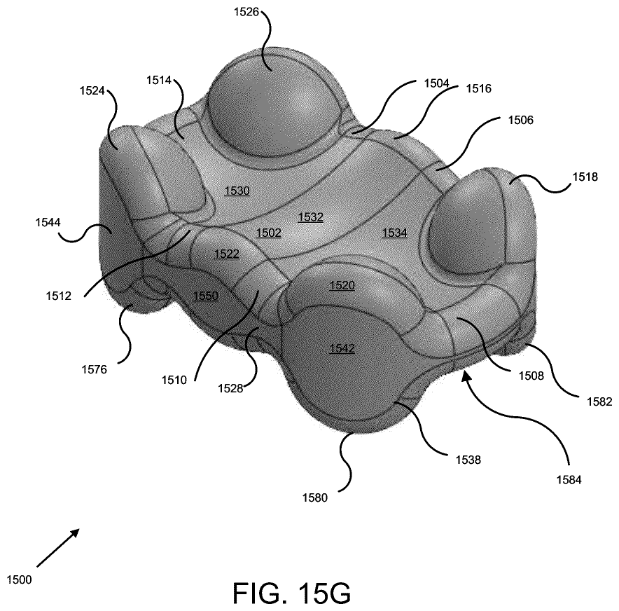

FIG. 15A illustrates a top view of an exemplary implantable interpositional orthopedic pain management apparatus. Here, device 1500 includes top surface 1502, channel openings 1504-1514, notches 1516 and 1522, peripheral protrusions 1518, 1520, 1524, and 1526, perimeter 1528, top surface regions 1530-1534, and contours 1538. As used herein, like numbered and/or like named elements are assumed to be referencing the same or a substantially similar element having the same or substantially similar function or structure. Differences in function or structure may be described separately with regard to a specific feature described. Top surface, 1502, in some examples, may include multiple regions such as top surface regions 1530-1534. Each of regions 1530-1534 may have similar or different radii of curvature, selected to fit with an end of a bone (e.g., metacarpal, trapezium, tibia, femur, or others, without limitation or restriction). In some examples, one or more region 1530-1534 may be substantially concave. In some examples, on or more regions 1530-1534 may be substantially convex. Top surface region 1532 may be configured to define an elongate portion with dual notches 1516 and 1522 disposed on or near perimeter 1528 and formed with region 1532. While formed as part or integrated with device 1500, perimeter 1528 may be an outermost portion of device 1500 into which features are shaped, such as peripheral protrusions 1518, 1520, 1524, and 1526. In the example herein, peripheral protrusions 1518, 1520, 1524, and 1526 are disposed adjacent to perimeter 1528. Channel openings 1508 and 1514 may be disposed between two adjacent protrusions, such as 1518 and 1520, and 1524 and 1526, respectively. In other examples, channel openings may be disposed between a peripheral protrusion 1526 and a notch 1516. A channel may be formed between channel openings 1506 and 1510, and another channel may me formed in between channel openings 1504 and 1512. The formed mentioned channels may be substantially parallel to one another. Channel openings 1508 and 1514 may also define openings to channels on top surface 1502 that may be part of regions 1534 and 1514, respectively, and overlap or intersect parallel channels defined by channel openings 1504 and 1512, and 1506 and 1510. In other examples, as in the previously and following described figures, the number, shape, type, quantity, and disposition of peripheral protrusions 1518, 1520, 1524, and 1526 may be varied and are not limited to those shown and described. The number, shape, type, quantity, and disposition of peripheral protrusions 1518, 1520, 1524, and 1526 may be selected to be positioned within non-articulating portions of a synovial capsule and/or joint, and alleviates pain by preventing contact between distal and proximal ends of bones in a joint, and peripheral protrusions prevent dislocation and expulsion of device 1500 from the joint. Device 1500 may include additional surface elements as generally identified by contours 1538. For example, device 1500 may include an additional curved surface element in between peripheral protrusion 1518 and top surface 1502, as illustrated by contours 1538. In other examples, device 1500 and the elements shown and described may be designed, configured, formed, modified, or implemented apart from the examples shown or described and are not limited to those provided.

FIG. 15B illustrates a bottom view of an exemplary implantable interpositional orthopedic pain management apparatus. Here, device 1500 includes perimeter 1528, contours 1538, bottom surface 1584, channel openings 1560-1570, notches 1572 and 1578, peripheral protrusions 1574, 1576, 1580, and 1582, and bottom surface regions 1586-1590. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described in reference to FIG. 15A. Bottom surface 1584, in some examples, may have three regions 1586-1590, each region having similar or different radii of curvature. The radii of curvature of three regions 1586-1590 may be selected to fit an end of a distal or proximal end of a bone in a joint, or a portion thereof. In some examples, the radii of curvature may be one or two degrees greater than radii of curvature of portions of an end of a bone. In some examples, region 1586 and region 1590 may be substantially convex, and region 1588 is an elongate portion, the ends of which define notches 1572 and 1578. Along with peripheral protrusions 1574, 1576, 1580, and 1582, notches 1572 and 1578 may function to maintain the position of device 1500, once implanted into a joint (not shown), such that they prevent expulsion device 1500 from the joint, while allowing for intermittent contact between peripheral protrusions 1574, 1576, 1580, and 1582, notches 1572 and 1578 and adjacent bone surfaces. Peripheral protrusions 1574, 1576, 1580, and 1582, and notches 1572 and 1578 may be in contact, or not be in contact with adjacent bone surfaces depending on the extension and/or flexion of the joint. While the various examples shown herein may exhibit symmetry around various axes, in other examples device 1500 and others described above and below may be asymmetrically formed or off-axially aligned, including when surgically implanted within a joint. In still other examples, device 1500 and the elements shown and described may be designed, configured, formed, modified, or implemented apart from the examples shown or described and are not limited to those provided.

FIG. 15C illustrates an anterior view of an exemplary implantable interpositional orthopedic pain management apparatus. Here, an anterior or front view of device 1500 includes top surface 1502, channel openings 1510, 1512, 1566, and 1568, peripheral protrusions 1520, 1524, 1576, and 1580, notches 1522 and 1570, contours 1538, and outward facing vertical surfaces 1542, 1544, and 1550. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 1502 in between channel openings 1510 and 1506 (not shown). A channel may also be formed on top surface 1502 in between channel openings 1512 and 1504 (not shown). In some examples, a channel is formed on bottom surface 1584 in between channel openings 1566 and 1562 (not shown). A channel may also be formed on bottom surface 1584 in between channel openings 1568 and 1560 (not shown). Notch 1522 may, for example, define a side portion of a channel, for example, the channel formed in between channel openings 1512 and 1504 (not shown). The position of channels on top surface 1502 may be defined by transitions or changes of radii of curvature between regions 1530-1534. Top and bottom surfaces 1502 and 1584 may each be formed to receive mutually reciprocal or substantially mutually reciprocal bones, bone structures, or portions thereof in a joint, and to align device 1500 with a bone surface of a joint. Peripheral protrusions 1524 and 1576 may be disposed on opposing sides of outward facing vertical surface 1544. Peripheral protrusions 1520 and 1580 may similarly be disposed on opposing sides of outward facing vertical surface 1542. Opposing notches 1522 and 1570 may be disposed on opposing sides of outward facing vertical surface 1550. Outward facing vertical surfaces 1542, 1544, and 1550 may be substantially flat and/or curved in shape such that they substantially align with the shape of perimeter 1528 (not shown) and that they minimize interaction with adjacent bone or tissue once implanted in a joint. In still other examples, device 1500 and the elements shown and described may be designed, configured, formed, modified, or implemented apart from the examples shown or described and are not limited to those provided.

FIG. 15D illustrates a posterior view of an exemplary implantable interpositional orthopedic pain management apparatus. Here, a posterior or back view of device 1500 includes top surface 1502, channel openings 1504, 1506, 1560, and 1562, peripheral protrusions 1518, 1526, 1574, and 1582, notches 1516 and 1572, contours 1538, and outward facing vertical surfaces 1540, 1546, and 1552. As used herein, like numbered and/or like named elements are assumed to be referencing the same or a substantially similar element having the same or substantially similar function or structure. Differences in function or structure may be described separately with regard to a specific feature described. Here a channel is formed on top surface 1502 in between channel openings 1506 and 1510 (not shown) A channel may also be formed on top surface 1502 in between channel openings 1504 and 1512 (not shown). In some examples, a channel is formed on bottom surface 1584 in between channel openings 1560 and 1568 (not shown). A channel may also be formed on bottom surface 1584 in between channel openings 1562 and 1566 (not shown). Notch 1516 may, for example, define a side portion of a channel, for example, the channel formed in between channel openings 1504 and 1512 (not shown). The radii of curvature of top surface regions 1530-1534 (not shown) may, in part, be defined by the channels formed between channel openings 1504, and 1512 (not shown), and 1506 and 1510 (not shown), and notches 1516 and 1522 (not shown). Similarly, the radii of curvature of bottom surface regions 1586-1590 may, in part, be defined by the channels formed between channel openings 1560 and 1568 (not shown), and 1562 and 1566 (not shown), and notches 1572 and 1578 (not shown). Peripheral protrusions 1518, 1526, 1574, and 1582 may be formed along perimeter 1528 of device 1500 and include outward facing vertical surfaces 1540 and 1546. Outward facing vertical surfaces 1540 and 1546 may, in some examples, have a bulb shape with a rounded portion and a pointed portion. Outward facing vertical surface 1552 may be disposed in between notches 1516 and 1572. Peripheral protrusions 1518, 1526, 1574, and 1582, and notches 1516 and 1572 may be configured to reside in non-articulating regions of a joint (not shown) when device 1500 is surgically implanted. In some examples, top surface 1502 and 1584 may be formed with various types of shapes and features (e.g., convex, concave, partially or wholly, or a combination thereof). If one or more bones of the joint (in which device 1500 is surgically implanted) are articulated, then peripheral protrusions 1518, 1526, 1574, and 1582, and notches 1516 and 1572 are configured to prevent the bones from coming into contact with each other, maintaining dynamic stability, and restoring an ability for the joint to be manipulated while preventing expulsion of device 1500 from the joint or dislocation of bones from the joint. In other examples, device 1500 and the elements shown and described may be designed, configured, formed, modified, or implemented apart from the examples shown or described and are not limited to those provided.

FIG. 15E illustrates a left view of an exemplary implantable interpositional orthopedic pain management apparatus. Here, a left side of device 1500 includes top surface 1502, channel openings 1514 and 1564, peripheral protrusions 1524, 1526, 1574, and 1576, contours 1538. outward facing vertical surfaces 1544 and 1546, and bottom surface 1584. As used herein, like numbered and/or like named elements are assumed to be referencing the same or a substantially similar element having the same or substantially similar function or structure. Differences in function or structure may be described separately with regard to a specific feature described. As shown, channel opening 1514 is positioned between peripheral protrusions 1524 and 1526. Channel opening 1564 is positioned between peripheral protrusions 1574 and 1576. Channel opening 1514 may be an opening to a channel on top surface 1502 that may intersect with the channel between channel openings 1504 and 1512 (not shown), and be disposed on region 1530 (not shown). Channel opening 1564 may be an opening to a channel on bottom surface 1584 that may intersect or be partially coextensive with the channel formed between channel openings 1562 and 15622 (not shown), and be disposed on region 1590 (not shown). Peripheral protrusions 1524 and 1526 are substantially disposed on the top side of outward facing vertical surfaces 1544 and 1546, and peripheral protrusions 1574 and 1576 are substantially disposed on the bottom side of outward facing vertical surfaces 1544 and 1546. Depending on the joint in which device 1500 is to be implanted within (not shown), outward facing vertical surfaces 1544, 1546 may be shaped in a manner that minimizes disturbance of any tissue or bone, thus decreasing potential pain or irritation caused by the implanted device 1500. In other examples, the positions and shapes of peripheral protrusions 1524, 1526, 1574, and 1576 and outward facing vertical surfaces 1544 and 1546 may be selected so that device 1500 may best fit within a targeted joint and minimize likelihood of expulsion of device 1500 from the joint (not shown). Device 1500 may include additional surface elements as generally identified by contours 1538. For example, contours 1538 may define a change in shape or radius of curvature between outward facing vertical surface 1544 and peripheral protrusion 1524. In still other examples, device 1500 and the elements shown and described may be designed, configured, formed, modified, or implemented apart from the examples shown or described and are not limited to those provided.

FIG. 15F illustrates a right view of an exemplary implantable interpositional orthopedic pain management apparatus. Here, a right side view of device 1500 includes top surface 1502, channel openings 1508 and 1570, peripheral protrusions 1518, 1520, 1580, and 1582, contours 1538, outward facing vertical surfaces 1540 and 1542, and bottom surface 1584. As used herein, like numbered and/or like named elements are assumed to be referencing the same or a substantially similar element having the same or substantially similar function or structure. Differences in function or structure may be described separately with regard to a specific feature described. In some examples, peripheral protrusions 1518, 1520, 1580, and 1582 may be coupled directly or indirectly with perimeter 1528 or formed as surface or structural features or components thereof. As shown and described, peripheral protrusions 1518, 1520, 1580, and 1582 may have various sizes, shapes, dimensions, and other attributes, without limitation or restriction. Here, channel opening 1508 is positioned between peripheral protrusions 1518 and 1520. Channel opening 1570 is positioned between peripheral protrusions 1580 and 1582. A channel may be formed on region 1534 (not shown) with channel opening 1508, such that the channel may partially overlap with the channel between channel openings 1506 and 1510 (not shown). A channel may be formed on region 1586 (not shown) with channel opening 1570, such that the channel may partially overlap with the channel between channel openings 1560 and 1568 (not shown). In still other examples, device 1500 and the elements shown and described may be designed, configured, formed, modified, or implemented apart from the examples shown or described and are not limited to those provided.

FIG. 15G illustrates a perspective view of an exemplary implantable interpositional orthopedic pain management apparatus. Here, a perspective view of device 1500 includes top surface 1502, channel openings 1504-1514, notches 1516, and 1522, peripheral protrusions 1518, 1520, 1524, 1526, 1576, 1580, and 1582, perimeter 1528, top surface regions 1530-1534, contours 1538, outward facing vertical surfaces 1542, 1544, and 1550, and bottom surface 1584. As used herein, like numbered and/or like named elements are assumed to be referencing the same or a substantially similar element having the same or substantially similar function or structure. Differences in function or structure may be described separately with regard to a specific feature described. As shown, the configurations of top surface regions 1530-1534 are each different. Shown here, regions 1530 and 1534 and both substantially concave, and include the channels formed between channel openings 1504 and 1512, and 1506 and 1510, respectively. Region 1530 includes the channel originating from channel opening 1514. Region 1534 includes the channel originating from channel opening 1508. Notches 1516 and 1522 define the end portions of region 1532, such that notches 1516 and 1522 are substantially adjacent to perimeter 1528, or formed with a region of top surface 1502. Region 1532 slopes on dual ends toward each notch 1516 and 1522. Regions 1530, 1532, and 1534 define the shape of top surface 1502 and in some examples, is based on the shape of an end of a bone of a target joint (not shown). Regions 1586-1590 (not shown) may be similar to regions 1530-1534. In the example shown, peripheral protrusions 1518, 1520, 1524, and 1526 are partial-spherical in shape. However, examples of device 1500 is not limited to the shown peripheral protrusion shape, such that the shape of the protrusion may be selected based on the specific shape of the adjacent bone that device 1500 is to be implanted next to in the joint. Device 1500, once surgically implanted within a synovial capsule and/or a joint, functions to prevent contact between a bone received by top surface 1502 and bottom surface 1584, and is configured to anatomically align device 1500 within a joint. Device 1500 may not be permanently coupled or otherwise adhered to a bone surface, to allow for dynamic movement of device 1500 within the joint. In other words, device 1500 may be in intermittent contact with bones that make up a joint. Peripheral protrusions 1518, 1520, 1524, 1526, 1574, 1576, 1580, and 1582 may in part function to hold device 1500 within the joint (not shown) by contacting substantially outer portions of received bones, thus preventing expulsion of device 1500 from the joint. In other examples, peripheral protrusions 1518, 1520, 1524, 1526, 1574, 1576, 1580, and 1582, and top and bottom surfaces 1502 and 1584 may also be configured to provide surfaces that are shaped to fit and/or fill with other materials within a synovial capsule such as cartilage, blood, bodily fluids, or other synthetic or organic materials that are injected or otherwise implanted to aid device 1500 in maintaining dynamic stability and position within a joint after surgical implantation. In still other examples, device 1500 and the elements shown and described may be designed, configured, formed, modified, or implemented apart from the examples shown or described and are not limited to those provided.

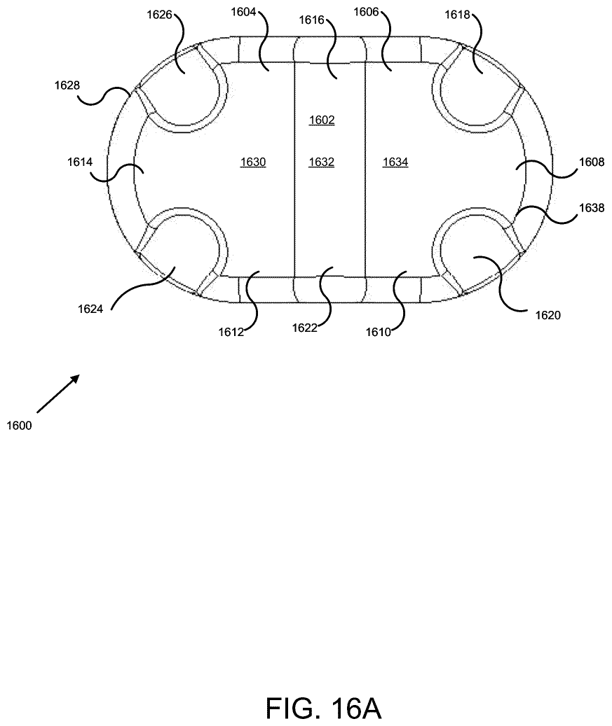

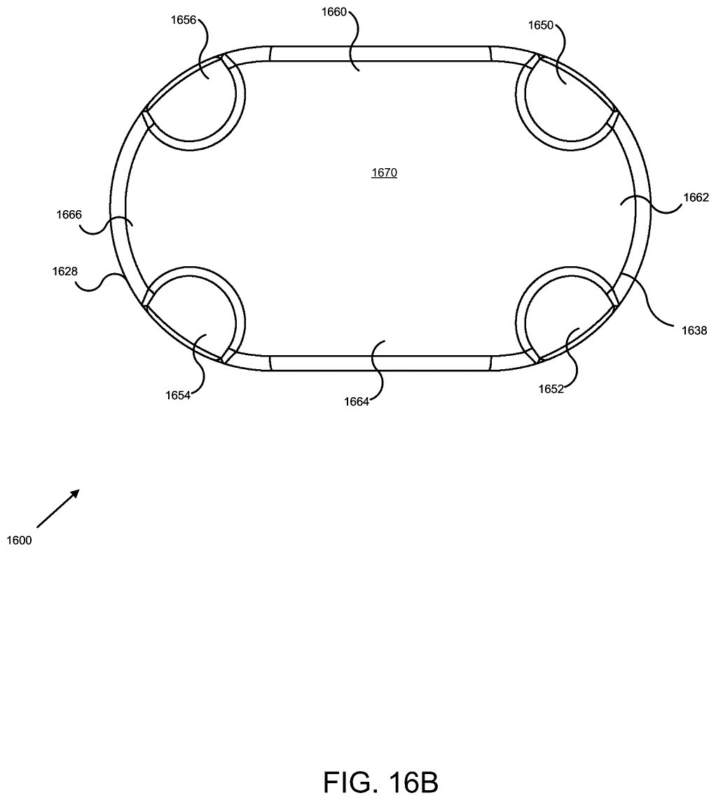

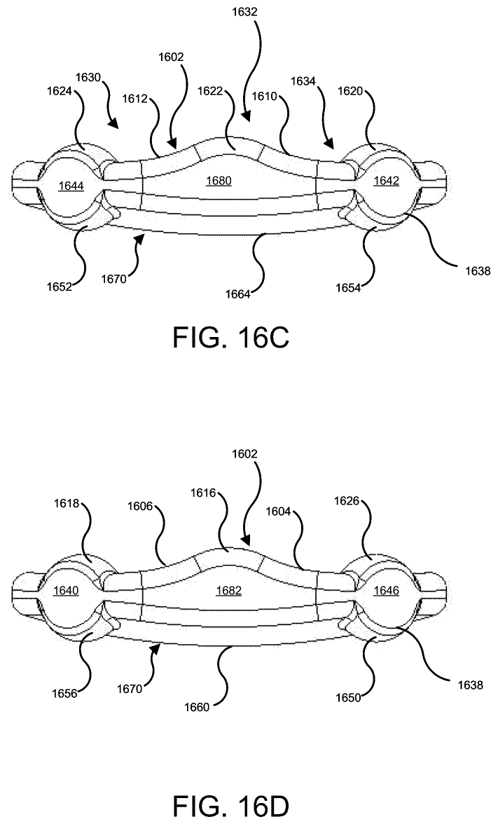

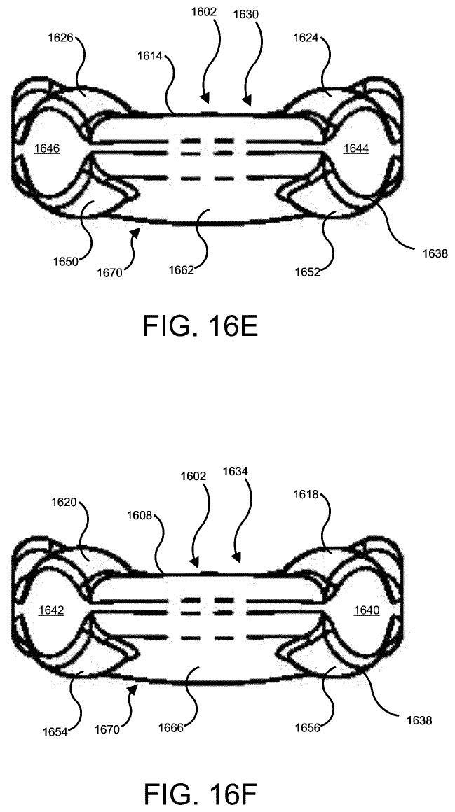

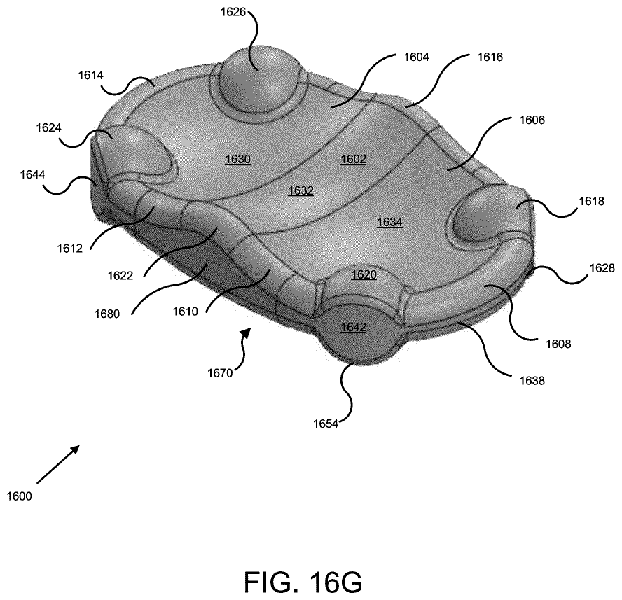

FIG. 16A illustrates a top view of an exemplary implantable interpositional orthopedic pain management apparatus. Here, device 1600 includes top surface 1602, channel openings 1604-1614, notches 1616 and 1622, peripheral protrusions 1618, 1620, 1624, and 1626, perimeter 1628, and top surface regions 1630-1634. As used herein, like numbered and/or like named elements are assumed to be referencing the same or a substantially similar element having the same or substantially similar function or structure. Differences in function or structure may be described separately with regard to a specific feature described. As shown, top surface 1602 may have multiple regions, 1630, 1632, and 1634. Each of the regions may have a different radius of curvature and shape depending on the joint in which the device 1600 is intended to be surgically inserted into. Each radius of curvature may be selected to fit with a portion of or an end of a bone, and to align the device 1600 with a bone or bone surface. In some examples, one or more of regions 1630-1634 may be substantially concave. In some examples, one or more of regions 1630-1634 may be substantially convex. Region 1632 may be disposed in between regions 1630 and 1634, and configured to define an elongated portion with dual notches 1616 and 1622 disposed on or near perimeter 1628, or otherwise formed with a region of top surface 1602. While formed as part or integrated with device 1600, perimeter 1628 may be an outermost perimeter of device 1600 into which features are shaped, such as peripheral protrusions 1618, 1620, 1624, and 1626, and outward facing vertical surfaces 1640-1646, 1680, and 1682 (to be described in reference to FIGS. 16C-16G). In the example herein, peripheral protrusions 1618, 1620, 1624, and 1626 are disposed adjacent to perimeter 1628. Channel openings 1608 and 1614 may be disposed between two adjacent protrusions, such as 1618 and 1620, and 1624 and 1626, respectively. In other examples, a channel opening 1604 may be disposed between a peripheral protrusion 1626 and a notch 1616. A channel may be formed in region 1630 in between channel openings 1604 and 1612. A substantially parallel channel may be formed in region 1634 in between channel openings 1606 and 1610. Channel opening 1614 may be an opening to a channel in region 1630, such that the channel may overlap or be partially coextensive with the channel between channel openings 1604 and 1612. Similarly, channel opening 1608 may be an opening to a channel in region 1634, such that the channel may overlap or be partially coextensive with the channel between channel openings 1606 and 1610. Channels on top surface 1602 generally have a curvature element that aligns with the curvatures of associated regions 1630-1634. In other examples, as in the previously and following described figures, the number, shape, type, quantity, and disposition of peripheral protrusions 1618, 1620, 1624, and 1626 may be varied and are not limited to those shown and described. Device 1600 may include additional surface elements as generally identified by contours 1638. For example, device 1600 may include an additional curved surface element in between peripheral protrusion 1620 and region 1634, as illustrated by contours 1238. The number, shape, type, quantity, and disposition of peripheral protrusions 1618, 1620, 1624, and 1626 may be selected to be positioned within non-articulating portions of a synovial capsule and/or joint, and alleviates pain by preventing contact between distal and proximal ends of bones in a joint, and peripheral protrusions prevent dislocation and expulsion of device 1600 from the joint. In other examples, device 1600 and the elements shown and described may be designed, configured, formed, modified, or implemented apart from the examples shown or described and are not limited to those provided.

FIG. 16B illustrates a bottom view of an exemplary implantable interpositional orthopedic pain management apparatus. Here, device 1600 includes perimeter 1628, contours 1638, peripheral protrusions 1650-1656, channel openings 1660-1666, and bottom surface 1670. As used herein, like numbered and/or like named elements are assumed to be referencing the same or a substantially similar element having the same or substantially similar function or structure. Differences in function or structure may be described separately with regard to a specific feature described. Top and bottom surfaces 1602 and 1670 may together be referred to as the body of device 1600. In some examples, a channel may be formed on bottom surface 1670 in between channel openings 1660 and 1664, or in between channel openings 1662 and 1666. Bottom surface 1670 may have a radius of curvature so that surface 1670 substantially fits the end of a bone within a joint. In other examples, bottom surface 1670 may have a radius of curvature that is one or two degrees greater than the radius of curvature of a bone. Peripheral protrusions 1650-1656 may be in contact, or not be in contact with adjacent bone surfaces depending on movement or flexion and extension of the joint. While the various examples shown herein may exhibit symmetry around various axes, in other examples device 1600 and others described above and below may be asymmetrically formed or off-axially aligned, including when surgically implanted within a joint. In still other examples, device 1600 and the elements shown and described may be designed, configured, formed, modified, or implemented apart from the examples shown or described and are not limited to those provided.

FIG. 16C illustrates an anterior view of an exemplary implantable interpositional orthopedic pain management apparatus. Here, an anterior or front view of device 1600 includes top surface 1602, channel openings 1610, 1612, and 1664, peripheral protrusions 1620, 1624, 1652, and 1654, notch 1622, regions 1630-1634, contours 1638, outward facing vertical surfaces 1642, 1644, and 1680, and bottom surface 1670. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 1602 in between channel openings 1610 and 1606 (not shown). A channel may also be formed on top surface 1602 in between channel openings 1612 and 1604 (not shown). In some examples, a channel is formed on bottom surface 1670 in between channel openings 1664 and 1660 (not shown). Notch 1622 and region 1632 (not shown) may define a side portion of a channel, for example, the channel formed in between channel openings 1612 and 1604 (not shown). Region 1632 may be a boundary between the positions of channels on top surface 1602. The boundary, however, may not be definite, such that the channel may overlap or be otherwise be disposed on region 1632. In the example shown, regions 1630 and 1634 (shown with floating arrows) are substantially concave. Region 1632 is substantially convex and may generally function as a portion of top surface 1602 to transition between curvatures of regions 1630 to 1634. Top and bottom surfaces 1602 and 1670 may each be formed to receive mutually reciprocal or substantially mutually reciprocal bones, bone structures, or portions thereof in a joint (e.g., trapezium, metacarpal, radial facet of a metacarpal, or others, without restriction or limitation). Peripheral protrusions 1624 and 1652 may also be disposed on substantially opposite sides of outward facing vertical surface 1644. Peripheral protrusions 1620 and 1654 may be disposed on substantially opposite sides of outward facing vertical surface 1642. Notch 1622 may be disposed on one side of outward facing vertical surface 1680. Outward facing vertical surface 1680 may be disposed in between outward facing vertical surfaces 1642 and 1644. Outward facing vertical surfaces 1642, 1644, and 1680 may have curvatures and topographies such that they minimize interaction with adjacent bone or tissue once implanted in a joint. In still other examples, device 1600 and the elements shown and described may be designed, configured, formed, modified, or implemented apart from the examples shown or described and are not limited to those provided.

FIG. 16D illustrates a posterior view of an exemplary implantable interpositional orthopedic pain management apparatus. Here, a posterior or back view of device 1600 includes top surface 1602, channel openings 1604, 1606, and 1660, peripheral protrusions 1618, 1626, 1650, and 1656, notch 1616, contours 1638, outward facing vertical surfaces 1640, 1646, and 1682, and bottom surface 1670. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described with reference to FIG. 16C. Here a channel is formed on top surface 1602 in between channel openings 1606 and 1610 (not shown) A channel may also be formed on top surface 1602 in between channel openings 1604 and 1612 (not shown). In some examples, a channel is formed on bottom surface 1670 in between channel openings 1660 and 1668 (not shown). Notch 1616 and region 1632 (not shown) may define a side portion of a channel, for example, the channel formed in between channel openings 1612 and 1604 (not shown). Peripheral protrusions 1618, 1626, 1650, and 1656 may be formed along perimeter 1628 of device 1600, and include, or be formed with, outward facing vertical surfaces 1640 and 1646. Peripheral protrusions 1618, 1626, 1650, and 1656, and notch 1616 may be configured to reside in non-articulating regions of a joint (not shown) when device 1600 is surgically implanted. In some examples, top surface 1602 and bottom surface 1670 may be formed with various types of shapes and features (e.g., convex, concave, partially or wholly, or a combination thereof). If one or more bones of the joint (in which device 1600 is surgically implanted) are articulated, then peripheral protrusions 1618, 1626, 1650, and 1656, and notch 1616 are configured to prevent the bones from coming into contact with each other, maintaining dynamic stability, and restoring an ability for the joint to be manipulated while preventing expulsion of device 1600 from the joint or dislocation of bones from the joint. In other words, peripheral protrusions 1618, 1626, 1650, and 1656, and notch 1616 may be formed to provide structures that function to interact with anatomical structures of a joint, including the bones or portions thereof within the joint, to prevent device 1600 from expulsion and dislocation. In other examples, device 1600 and the elements shown and described may be designed, configured, formed, modified, or implemented apart from the examples shown or described and are not limited to those provided.

FIG. 16E illustrates a left view of an exemplary implantable interpositional orthopedic pain management apparatus. Here, a left side of device 1600 includes top surface 1602, channel openings 1614 and 1662, peripheral protrusions 1624, 1626, 1650, and 1652, region 1630, contours 1638, outward facing vertical surfaces 1644 and 1646, and bottom surface 1670. As used herein, like numbered and/or like named elements are assumed to be referencing the same or a substantially similar element having the same or substantially similar function or structure. Differences in function or structure may be described separately with regard to a specific feature described. Top surface 1602 (or region 1630) may be substantially concave, and bottom surface 1670 may be substantially convex, for example. Here, channel opening 1614 is disposed between peripheral protrusions 1624 and 1626. Channel opening 1662 is disposed between peripheral protrusions 1650 and 1652. Peripheral protrusions 1624 and 1626 are formed on the top sides of outward facing vertical surfaces 1644 and 1646, and peripheral protrusions 1650 and 1652 are formed on the bottom sides of outward facing vertical surfaces 1644 and 1646. Depending on the joint in which device 1600 is to be implanted within (not shown), the outward facing vertical surfaces may be shaped in a manner that minimizes disturbance of any adjacent tissue or bone, thus decreasing potential pain or irritation caused by the implanted device 1600. In other examples, the positions and shapes of peripheral protrusions 1624, 1626, 1650, and 1652, outward facing vertical surfaces 1644 and 1646 may be selected so that device 1600 may best fit within a targeted joint and minimize likelihood of expulsion of device 1600 from the joint (not shown). In other examples, device 1600 and the elements shown and described may be designed, configured, formed, modified, or implemented apart from the examples shown or described and are not limited to those provided.

FIG. 16F illustrates a right view of an exemplary implantable interpositional orthopedic pain management apparatus. Here, a right side of device 1600 includes top surface 1602, channel openings 1608 and 1666, peripheral protrusions 1618, 1620, 1654, and 1656, region 1634, contours 1638, outward facing vertical surfaces 1640 and 1640, and bottom surface 1670. As used herein, like numbered and/or like named elements are assumed to be referencing the same or a substantially similar element having the same or substantially similar function or structure. Differences in function or structure may be described separately with regard to a specific feature described. In some examples, peripheral protrusions 1618, 1620, 1654, and 1656 may be coupled directly or indirectly with perimeter 1628 or formed as surface or structural features or components thereof. As shown and described, peripheral protrusions 1618, 1620, 1654, and 1656 may have various sizes, shapes, dimensions, and other attributes, without limitation or restriction. Here, channel opening 1608 is defined by a space between peripheral protrusions 1618 and 1620. Channel opening 1666 is defined by a space between peripheral protrusions 1654 and 1656. A portion of top surface 1602, such as region 1634, may be substantially concave, when viewed from a right side of the device 1600. A portion of bottom surface 1670 may be substantially convex, when viewed from the right side of device 1600. However, the shapes and radii of curvatures of top and bottom surfaces 1602 and 1670 are generally selected based on the corresponding shapes of the ends of the bones in a targeted joint (not shown). In other examples, device 1600 and the elements shown and described may be designed, configured, formed, modified, or implemented apart from the examples shown or described and are not limited to those provided.

FIG. 16G illustrates a perspective view of an exemplary implantable orthopedic pain management apparatus. Here, a perspective view of device 1600 includes top surface 1602, channel openings 1604-1614, notches 1616 and 1622, peripheral protrusions 1618, 1620, 1624, 1626, and 1654, top surface regions 1630-1634, contours 1638, outward facing vertical surfaces 1642, 1644, 1680, and bottom surface 1670. As used herein, like numbered and/or like named elements are assumed to be referencing the same or a substantially similar element having the same or substantially similar function or structure. Differences in function or structure may be described separately with regard to a specific feature described. As shown, the configurations of regions 1630-1634 are each different. Regions 1630 and 1634 and both substantially concave, and include the channels formed between channel openings 1604 and 1612, and 1606 and 1610, respectively, and channel openings 1614 and 1608, respectively. Notches 1616 and 1622 define the end portions of region 1632, the end portions being adjacent to perimeter 1628 and formed with top surface 1602. Region 1632 may, in some examples, separate regions 1630 and 1634, and have a radius of curvature that transitions from the curvatures of region 1630 to region 1634. In some examples, region 1632 slopes on both ends toward each notch 1616 and 1622. Regions 1630-1634 define the shape of top surface 1602, and in some examples, is based on the shape of an end of a bone of a target joint (not shown). In the example shown, peripheral protrusions 1618, 1620, 1624, and 1626 are partial-spherical in shape. In some examples, the rounded shapes or peripheral protrusions 1618, 1620, 1624, 1626, and 650-1656 allow for smooth or uninterrupted movement or interaction between peripheral protrusions and adjacent bone surfaces. Outward facing vertical surfaces 1640-1646 (not shown) connect peripheral protrusions 1618, 1620, 1624, and 1626 to peripheral protrusions 1650-1656 (not shown). Outward facing vertical surfaces 1680 and 1682 (not shown) may be defined as the surface in between notch 1622 and 1612 and channel openings 1664 and 1660 (not shown). The shape of outward facing vertical surfaces 1640-1646, 1680, and 1682 may be selected to be aligned with the shape of perimeter 1628. The shape of outward facing vertical surfaces 1640-1646, 1680, and 1682 may also be configured to minimize interaction of device 1600 with adjacent tissue or bone, once surgically implanted within a synovial capsule and/or joint (not shown). However, examples of the device 1600 is not limited to the shapes described herein, such that the shape of the protrusions and outward facing vertical surfaces may be selected based on the specific shape of the adjacent bone that device 1600 is to be implanted next to. Device 1600, once surgically implanted within a synovial capsule and/or a joint, functions to prevent contact between a bone received by top surface 1602 and bottom surface 1670, and is configured to maintain anatomical alignment of device 1600 within the joint. Device 1600 may not be permanently coupled or otherwise adhered to a bone surface, to allow for dynamic movement of device 1600 within the joint. In other words, device 1600 may be in intermittent contact with bones that make up a joint. Peripheral protrusions 1618, 1620, 1624, 1626, and 1650-1654 may in part function to hold device 1600 within the joint (not shown) by contacting substantially outside portions of the received bones, thus preventing expulsion of device 1600 from the joint. In other examples, peripheral protrusions 1618, 1620, 1624, 1626, and 1650-1654 and top and bottom surfaces 1602 and 1670 may also be configured to provide surfaces that are shaped to fit and/or fill with other materials within a synovial capsule such as cartilage, blood, bodily fluids, or other synthetic or organic materials that are injected or otherwise implanted to aid device 1600 in maintaining dynamic stability and within a joint after surgical implantation. In still other examples, device 1600 and the elements shown and described may be designed, configured, formed, modified, or implemented apart from the examples shown or described and are not limited to those provided.

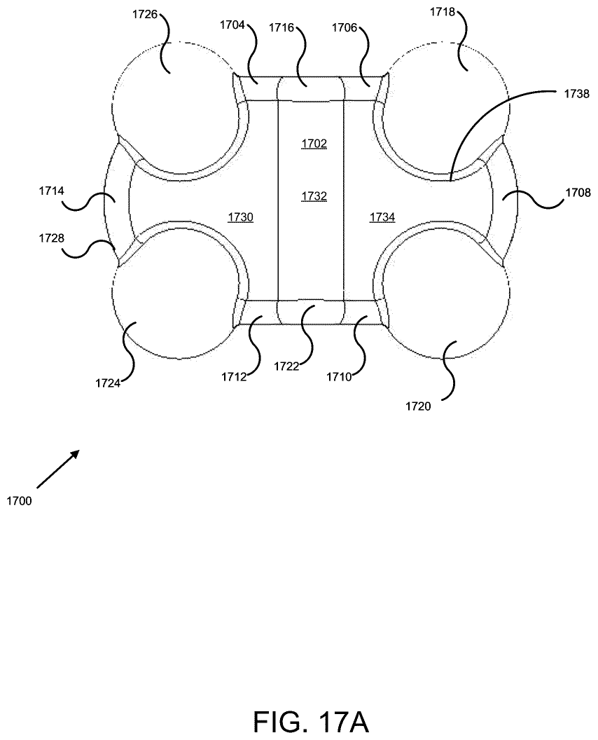

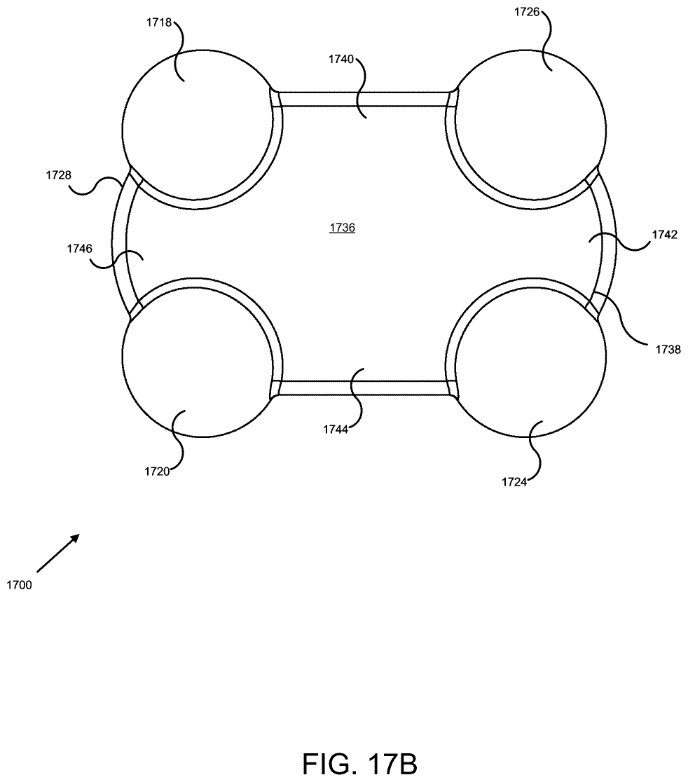

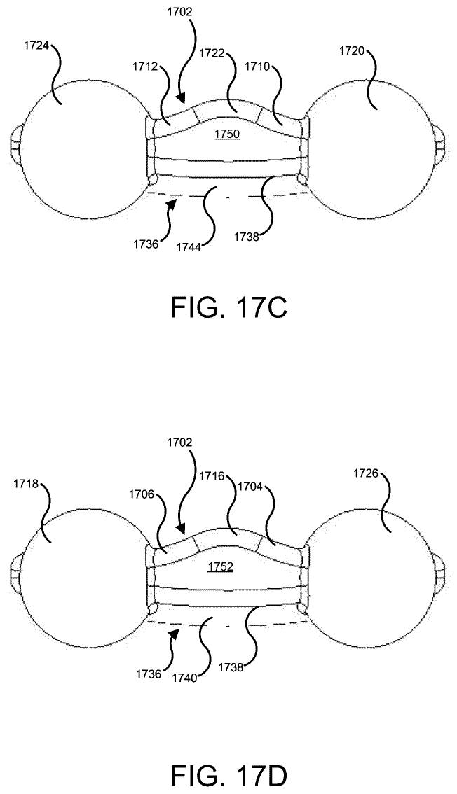

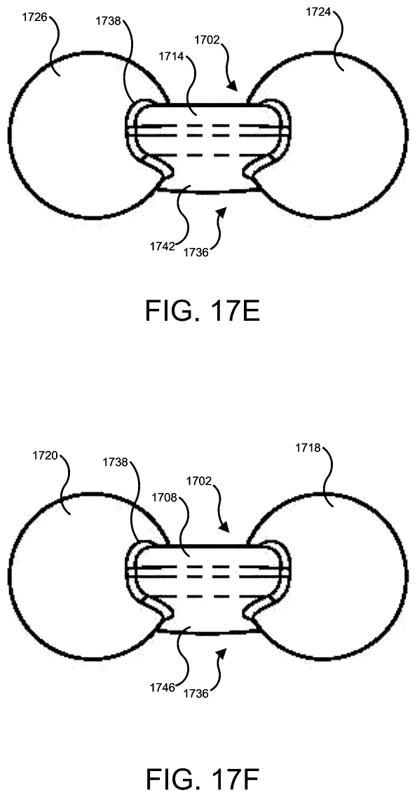

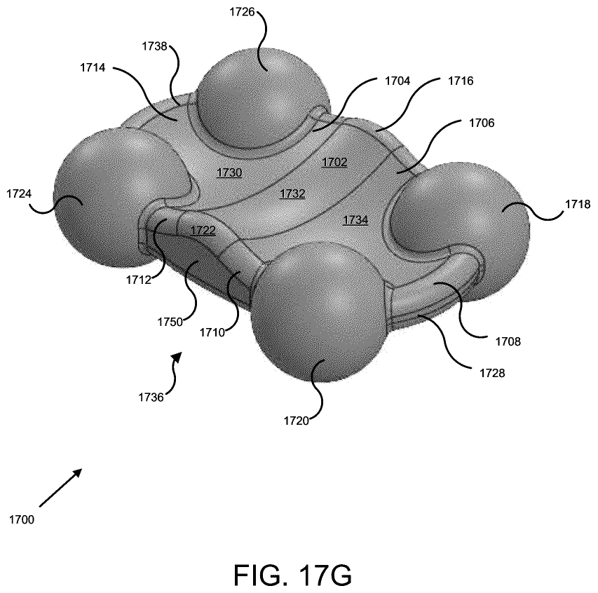

FIG. 17A illustrates a top view of an exemplary implantable interpositional orthopedic pain management apparatus. Here, device 1700 includes top surface 1702, channel openings 1704-1714, notches 1716 and 1722, peripheral protrusions 1718, 1720, 1724, and 1726, perimeter 1728, top surface regions 1730-1734, and contours 1738. As used herein, like numbered and/or like named elements are assumed to be referencing the same or a substantially similar element having the same or substantially similar function or structure. Differences in function or structure may be described separately with regard to a specific feature described. As shown, top surface 1702 may have multiple regions 1730-1734, each of which may be defined by different radii of curvature. The surface curvature transitions between regions 1730-1734 may be gradual or abrupt. Region 1732 may be disposed in between regions 1730 and 1734. The radius of curvature of each region 1730-1734 may in part depend on the shape of the bone against which the top surface 1702 is intended to be implanted against. Each of the regions may have a different radius of curvature, selected to fit with an end of a bone (e.g., metacarpal, trapezium, tibia, femur, or others, without limitation or restriction). In some examples, one or more of regions 1730-1734 may be substantially concave. In some examples, one or more of regions 1730-1734 may be substantially convex. Region 1732 may be configured to define an elongate portion with dual notches 1716 and 1722 disposed substantially on perimeter 1728. While formed as part or integrated with device 1700, perimeter 1728 may be an outermost perimeter of device 1700 into which features are shaped, such as peripheral protrusions 1718, 1720, 1724, and 1726, and notches 1716 and 1722. In this example, peripheral protrusions 1718, 1720, 1724, and 1726 are disposed along perimeter 1628 and are spherical in shape and may have circular cross sections. Channel openings 1708 and 1714 may be disposed between two adjacent protrusions, such as 1718 and 1720, and 1724 and 1726, respectively. In other examples, a channel opening 1704 may be disposed between a peripheral protrusion 1726 and a notch 1716. A channel may be formed in between channel openings 1704 and 1712. A channel may also be formed in between channels 1706 and 1710. The channels between 1704 and 1712, and 1706 and 1710 may be substantially parallel, and/or be disposed on opposing sides of region 1732. Region 1730 may include a channel formed with channel opening 1714, the channel formed may be curved and overlap or be otherwise coextensive with the channel formed between channel openings 1704 and 1712. Region 1734 may include a channel formed with channel opening 1708, the channel formed may be curved and overlap or be otherwise coextensive with the channel formed between channel openings 1706 and 1710. Device 1700 may include additional surface elements as generally identified by contours 1738. For example, device 1700 may include a change in surface shape between region 1734 and peripheral protrusion 1718, as identified generally by contours 1738. In other examples, as in the previously and following described figures, the number, shape, type, quantity, and disposition of peripheral protrusions 1718, 1720, 1724, and 1726 may be varied and are not limited to those shown and described. The number, shape, type, quantity, and disposition of peripheral protrusions 1718, 1720, 1724, and 1726 may be selected to be positioned within non-articulating portions of a synovial capsule and/or joint, and alleviates pain by preventing contact between distal and proximal ends of bones in a joint, and peripheral protrusions prevent dislocation and expulsion of device 1700 from the joint. In other examples, device 1700 and the elements shown and described may be designed, configured, formed, modified, or implemented apart from the examples shown or described and are not limited to those provided.

FIG. 17B illustrates a bottom view of an exemplary implantable interpositional orthopedic pain management apparatus. Here, device 1700 includes peripheral protrusions 1718-1726, perimeter 1728, bottom surface 1736, contours 1738, and channel openings 1740-1746. As used herein, like numbered and/or like named elements are assumed to be referencing the same or a substantially similar element having the same or substantially similar function or structure. Differences in function or structure may be described separately with regard to a specific feature described. Here, the bottom view shows the bottom sides of spherical peripheral protrusions 1718-1726 (top sides of peripheral protrusions are viewable in FIG. 17A). Peripheral protrusions 1718-1726 may have outermost walls that extend outward past perimeter 1728. A channel may be defined by the area on the bottom surface 1736 in between opposing channel openings, such as 1740 and 1744, and 1742 and 1746. Bottom surface 1736 may have a radius of curvature so that surface 1736 substantially fits the end of a bone within a joint. In other examples, bottom surface 1736 may have a radius of curvature that is one or two degrees greater than the radius of curvature of a bone. In some examples, bottom surface 1736 may be substantially convex in nature. In other examples, bottom surface 1736 may be substantially concave. While the various examples shown herein may exhibit symmetry around various axes, in other examples device 1700 and others described above and below may be asymmetrically formed or off-axially aligned, including when surgically implanted within a joint. In still other examples, device 1700 and the elements shown and described may be designed, configured, formed, modified, or implemented apart from the examples shown or described and are not limited to those provided.

FIG. 17C illustrates an anterior view of an exemplary implantable interpositional orthopedic pain management apparatus. Here, an anterior or front view of device 1700 includes top surface 1702, channel openings 1710, 1712 and 1744, peripheral protrusions 1720 and 1724, notch 1722, bottom surface 1736, contours 1738 and outward facing vertical surface 1750. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 1702 in between channel openings 1710 and 1706 (not shown). A channel may also be formed on top surface 1702 in between channel openings 1712 and 1704 (not shown). A channel may be formed on bottom surface 1736 in between channel openings 1744 and 1740. Notch 1722 and region 1734 (not shown) may define a side portion of a channel, for example, the channel formed in between channel openings 1612 and 1604 (not shown). Notch 1722 and region 1732 (not shown) may have an elevated component, as compared to channel openings 1710 and 1712. In this example and view, part of the curvature of top surface 1702 is viewable and may be partially concave and partially convex. Part of the curvature of bottom surface 1736 is viewable and is generally convex in nature. Outward facing vertical surface 1750 may be shaped with perimeter 1728 (not shown) and be in between peripheral protrusions 1720 and 1724. In some examples, outward facing vertical surface 1750 may be substantially in between notch 1722 and channel opening 1744. Top and bottom surfaces 1702 and 1736 may each be formed to receive mutually reciprocal or substantially mutually reciprocal bones, bone structures, or portions thereof in a joint. In still other examples, device 1700 and the elements shown and described may be designed, configured, formed, modified, or implemented apart from the examples shown or described and are not limited to those provided.

FIG. 17D illustrates a posterior view of an exemplary implantable interpositional orthopedic pain management apparatus. Here, a posterior or back view of device 1700 includes top surface 1702, channel openings 1704, 1706 and 1740, peripheral protrusions 1718 and 1726, notch 1716, bottom surface 1736, contours 1738, and outward facing vertical surface 1752. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described with reference to FIG. 17D. Here, a channel is formed on top surface 1702 in between channel openings 1706 and 1710 (not shown) A channel may also be formed on top surface 1702 in between channel openings 1704 and 1712 (not shown). In some examples, a channel is formed on bottom surface 1736 in between channel openings 1740 and 1744 (not shown). Notch 1716 may be disposed in between channel openings 1704 and 1706. The radii of curvature of regions 1730-1734 may, in part, be defined by the channels formed between channel openings 1704, 1706, 1710, and 1712. Conversely, the channels formed between channel openings 1704, 1706, 1710, and 1712 may in part define the radii of curvature of regions 1730-1734. Peripheral protrusions 1718 and 1726, and notch 1716 may be configured to reside in non-articulating regions of a joint (not shown) when device 1700 is surgically implanted. If one or more bones of the joint (in which device 1700 is surgically implanted) are articulated, then peripheral protrusions 1718 and 1726, and notch 1716 are configured to prevent the bones from coming into contact with each other, maintaining dynamic stability, and restoring an ability for the joint to be manipulated while preventing expulsion of device 1700 from the joint or dislocation of bones from the joint. In other examples, device 1700 and the elements shown and described may be designed, configured, formed, modified, or implemented apart from the examples shown or described and are not limited to those provided.

FIG. 17E illustrates a left view of an exemplary implantable interpositional orthopedic pain management apparatus. Here, a left side of device 1700 includes top surface 1702, channel openings 1714 and 1742, peripheral protrusions 1724 and 1726, bottom surface 1736, and contours 1738. As used herein, like numbered and/or like named elements are assumed to be referencing the same or a substantially similar element having the same or substantially similar function or structure. Differences in function or structure may be described separately with regard to a specific feature described. As shown, channel opening 1714 is disposed on top surface 1702 between peripheral protrusions 1724 and 1726. Channel opening 1742 is disposed on bottom surface 1736 between peripheral protrusions 1724 and 1726. Peripheral protrusions 1724 and 1726 are substantially spherical in shape and are formed on perimeter 1728 of the device. A portion of peripheral protrusions 1724 and 17246 may be disposed on or formed with top surface 1702, and a portion may be disposed on or formed with bottom surface 1736. Peripheral protrusions 1724 and 1726 may also be formed to each envelop a portion of the body of device 1700 (the body being the top and bottom surfaces 1702 and 1736). The outermost surfaces of peripheral protrusions 1724 and 1726 spatially extend further than top surface 1702, bottom surface 1736, and perimeter 1728 of device 1700. However, the size, shape, and positions of peripheral protrusions 1724 and 1726 depend on the joint in which device 1700 is to be implanted within (not shown). Once implanted, peripheral protrusions 1724, 1726 may in some examples cradle an end of a bone against top and bottom surfaces 1702, 1736, and reduce lateral (as measured against an axis of the bone) movement of device 1700. In other examples, the positions and shapes of peripheral protrusions 1724 and 1726 may be selected so that device 1700 may best fit within a targeted joint and minimize likelihood of expulsion of device 1700 from the joint (not shown). In other examples, device 1700 and the elements shown and described may be designed, configured, formed, modified, or implemented apart from the examples shown or described and are not limited to those provided.

FIG. 17F illustrates a right view of an exemplary implantable interpositional orthopedic pain management apparatus. Here, a right side of device 1700 includes top surface 1702, channel openings 1708 and 1746, peripheral protrusions 1718 and 1720, bottom surface 1736, and contours 1738. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described in reference to FIG. 17E. In some examples, peripheral protrusions 1718 and 1720 may be coupled directly or indirectly with perimeter 1728 or formed as surface or structural features or components thereof. As shown and described, peripheral protrusions 1718 and 1720 may have various sizes, shapes, dimensions, and other attributes, without limitation or restriction. Here, channel opening 1708 is defined by a space on the top surface 1702 between peripheral protrusions 1718 and 1720. Channel opening 1746 is defined by a space on the bottom surface 1746 between peripheral protrusions 1718 and 1720. A portion of top surface 1702, such as region 1734, may be substantially concave, when viewed from a right side of device 1700. A portion of bottom surface 1736 may be substantially convex, when viewed from a right side of device 1700. However, the shapes and radii of curvatures of top and bottom surfaces 1702 and 1736 are generally selected based on the corresponding shapes of the ends of the bones in a targeted joint (not shown). In other examples, device 1700 and the elements shown and described may be designed, configured, formed, modified, or implemented apart from the examples shown or described and are not limited to those provided.

FIG. 17G illustrates a perspective view of an exemplary implantable orthopedic pain management apparatus. Here, a perspective view of device 1700 includes top surface 1702, channel openings 1704-1714, notches 1716 and 1722, peripheral protrusions 1718, 1720, 1724, and 1726, perimeter 1728, top surface regions 1730-1734, bottom surface 1736, contours 1738, and outward facing vertical surface 1750. As used herein, like numbered and/or like named elements are assumed to be referencing the same or a substantially similar element having the same or substantially similar function or structure. Differences in function or structure may be described separately with regard to a specific feature described. As shown, the radii of curvature and orientations of top surface regions 1730-1734 are each different. Here, regions 1730 and 1734 and both substantially concave, and include the channels formed between channel openings 1704 and 1712, and 1706 and 1710, respectively. Notches 1716 and 1722 define the end portions of region 1732, notches 1716, 1722 being adjacent to perimeter 1728 and formed with top surface 1702. In some examples, region 1732 is substantially four-sided and slopes on two ends toward each notch 1716 and 1722. Regions 1730-1734 define the shape of top surface 1702, and in some examples, is based on the shape of an end of a bone of a target joint (not shown). Additional surface elements are illustrated, for examples, by contours 1738. For example, device 1700 may include an additional curved surface element in between peripheral protrusion 1726 and region 1730, as illustrated by contours 1738. In the example shown, peripheral protrusions 1718, 1720, 1724, and 1726 are substantially spherical in shape. However, examples of device 1700 is not limited to the shown peripheral protrusion shape, such that the shape of the protrusion may be selected based on the specific shape of the adjacent bone that device 1700 is to be implanted next to. In some examples, the spherical shape of peripheral protrusions 1718-1726 may allow for smooth or uninterrupted adjustments or movements between device 1700 and adjacent bone surfaces. Device 1700, once surgically implanted within a synovial capsule and/or a joint, functions to prevent contact between a bone received by top surface 1702 and bottom surface 1736. Device 1700 may not be permanently coupled or otherwise adhered to a bone surface, to allow for dynamic movement of device 1700 within the joint. In other words, device 1700 may be in intermittent contact with bones that make up a joint. Peripheral protrusions 1718, 1720, 1724, and 1726 may in part function to hold device 1700 within the joint (not shown) by contacting substantially outside portions of the received bones, thus preventing expulsion of device 1700 from the joint when lateral forces are applied to top or bottom surfaces 1702 and 1736. In other examples, peripheral protrusions 1718, 1720, 1724, and 1726 and top and bottom surfaces 1702 and 1736 may also be configured to provide surfaces that are shaped to fit and/or fill with other materials within a synovial capsule such as cartilage, blood, bodily fluids, or other synthetic or organic materials that are injected or otherwise implanted to aid device 1700 in maintaining dynamic stability and position within a joint after surgical implantation. In still other examples, device 1700 and the elements shown and described may be designed, configured, formed, modified, or implemented apart from the examples shown or described and are not limited to those provided.

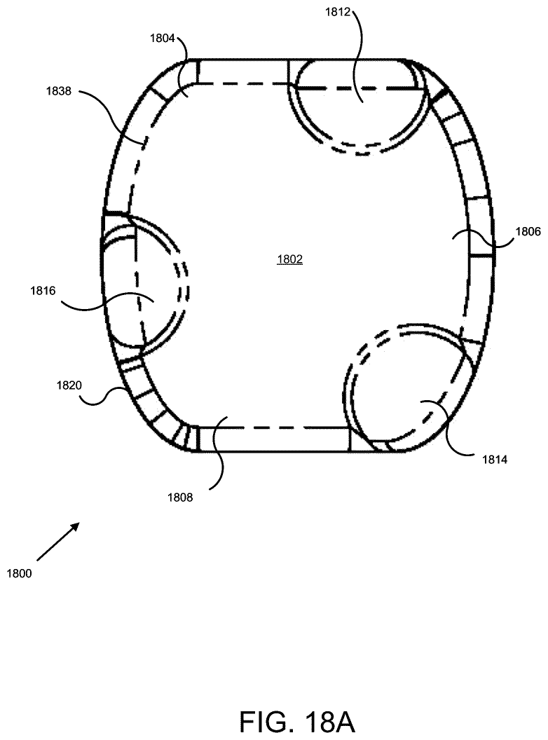

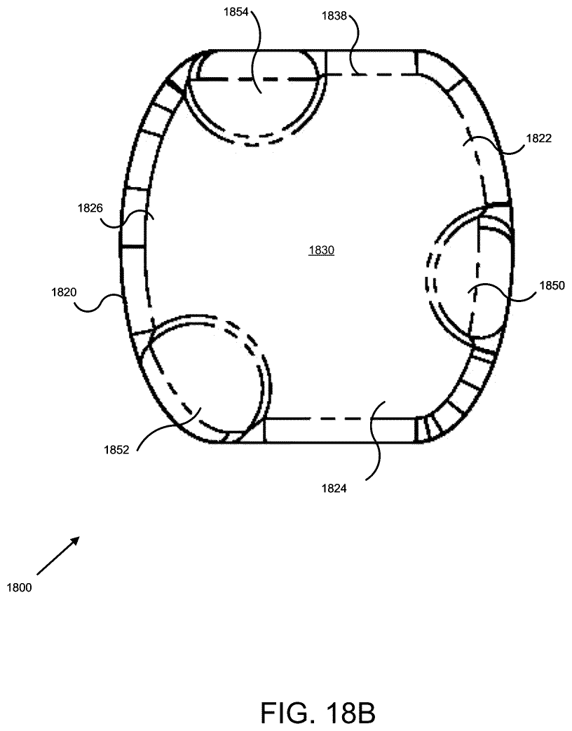

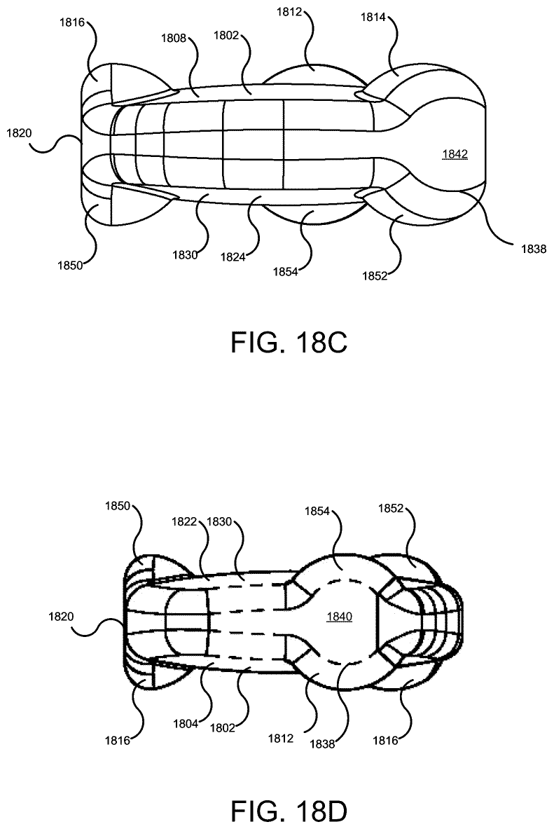

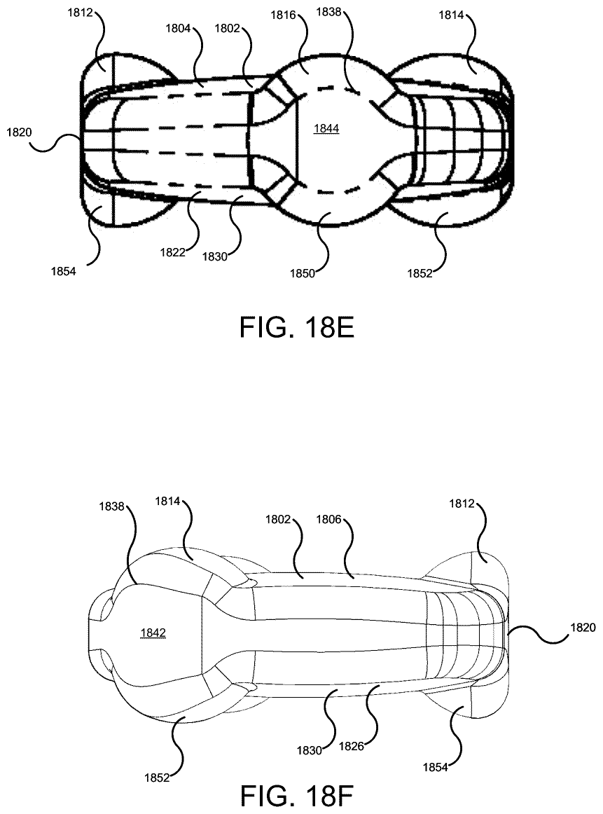

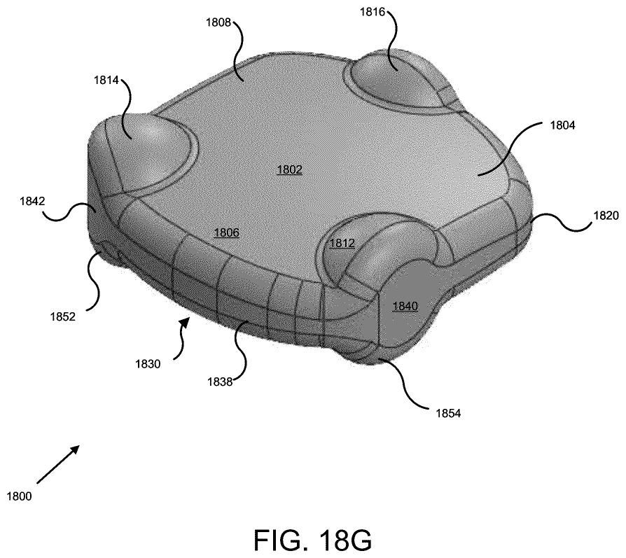

FIG. 18A illustrates a top view of an exemplary implantable interpositional orthopedic pain management apparatus. Here, device 1800 includes top surface 1802, channel openings 1804-1808, peripheral protrusions 1812-1816, perimeter 1820, and contours 1838. As used herein, like numbered and/or like named elements are assumed to be referencing the same or a substantially similar element having the same or substantially similar function or structure. Differences in function or structure may also be described separately without limitation, restriction, or regard to a specific feature previously described. In the present example, top surface 1802 is substantially four-sided with three peripheral protrusions 1812-1816. In some examples, peripheral protrusions 1812-1816 are formed with perimeter 1820 and disposed at substantially equal distances from one another on perimeter 1820. However, the positions of peripheral protrusions 1812-1816 are not limited to the perimeter or having positions equidistant from each other. In some examples, top surface 1802 may be substantially convex and, together with peripheral protrusions 1812-1816 and channel openings 1804-1808, is configured to receive a bone (e.g., metacarpal, trapezium, tibia, femur, or others, without limitation or restriction). Peripheral protrusions 1812-1816 may provide structures that, when implanted into a joint (not shown), are configured to interact within articulating or non-articulating regions of a joint. Peripheral protrusions 1812-1816 may be configured to maintain anatomical alignment of device 1800 within the joint, once implanted. Peripheral protrusions 1812-1816 are configured to not interfere with motion of joint in order to provide maximum range of extension and motion associated with individual bones forming a joint, and prevents migration of device 1800 from joint. As described above, device 1800 is configured to permit motion or articulation of a joint without dislocation or expulsion of device 1800 from the joint. Device 1800 may include additional surface elements as generally identified by contours 1838. For example, device 1800 may include an additional curved surface element on top surface 1802 indicating a change of curvature, as illustrated by contours 1838. In other examples, device 1800 and the elements shown and described may be designed, configured, formed, modified, or implemented apart from the examples shown or described and are not limited to those provided.

FIG. 18B illustrates a bottom view of an exemplary implantable interpositional orthopedic pain management apparatus. Here, device 1800 includes perimeter 1820, bottom surface 1830, channel openings 1822-1826, contours 1838, and peripheral protrusions 1850-1854. As used herein, like numbered and/or like named elements are assumed to be referencing the same or a substantially similar element having the same or substantially similar function or structure. Differences in function or structure may also be described separately without limitation, restriction, or regard to a specific feature previously described. Elements shown herein are similar to those described in reference to FIG. 18A. In the example herein, bottom surface 1830 includes three peripheral protrusions 1850-1854 spaced substantially equidistant from each other, formed with perimeter 1820. In some examples, bottom surface 1830 may be substantially convex and, together with peripheral protrusions 1850-1854 and channel openings 1822-1826, is configured to receive a bone. Top and bottom surfaces 1802 (not shown) and 1830, may be together referred to as the body of device 1800. In other examples, top and bottom surfaces 1802 (FIG. 18A) and 1830 may be configured differently and are not limited to the examples shown and described.

FIG. 18C illustrates an anterior view of an exemplary interpositional orthopedic pain management apparatus. Here, an anterior or front view of device 1800 is shown, including top surface 1802, channel openings 1808 and 1824, perimeter 1820, bottom surface 1830, contours 1838, peripheral protrusions 1812-1816 and 1850-1854, and outward facing vertical surface 1842. As used herein, like numbered and/or like named elements are assumed to be referencing the same or a substantially similar element having the same or substantially similar function or structure. Differences in function or structure may be described separately with regard to a specific feature described. Here, both top surface 1802 and bottom surface 1830 are substantially convex, with three peripheral protrusions 1812-1816 on top surface 1802, and three peripheral protrusions 1850-1854 on bottom surface 1830. Peripheral protrusions 1812-1816 and 1850-1854 are disposed along and formed with perimeter 1820. In other examples, peripheral protrusions 1812-1816 and 1850-1854 may be formed differently and are not limited to the examples shown and described. The configurations of surfaces 1802 and 1830 and peripheral protrusions 1812-1816 and 1850-1854 are selected to receive a bone within a joint or synovial capsule. In some examples, an outward facing vertical surface 1842 is disposed between opposing (i.e., disposed on opposite surfaces 1802, 1830) peripheral protrusions 1814 and 1852. Outward facing vertical surface 1842 may have a curvature element that is similar to the curvature of perimeter 1820, the curvature being best viewable in FIGS. 18A and 18B. In some examples, a channel may be formed on top surface 1802, in between peripheral protrusions 1814 and 1816, with channel opening 1808. The channel may be a portion of top surface 1802 that includes channel opening 1808, and may, in some examples, intersect or include channels originating at channel openings 1804 and 1806 (not shown). A channel may be formed on bottom surface 1830, in between peripheral protrusions 1850 and 1852 with channel opening 1824. The channel may be a portion of bottom surface 1830 that includes channel opening 1824, and may, in some examples, intersect or include channels originating at channel openings 1822 and 1826 (not shown). In other examples, device 1800 and the elements shown and described may be designed, configured, formed, modified, or implemented apart from the examples shown or described and are not limited to those provided.

FIG. 18D illustrates a posterior view of an exemplary implantable interpositional orthopedic pain management apparatus. Here, a posterior or rear view of device 1800 is shown, including top surface 1802, channel openings 1804 and 1822, perimeter 1820, bottom surface 1830, contours 1838, peripheral protrusions 1812-1816 and 1850-1854, and outward facing vertical surface 1840. As used herein, like numbered and/or like named elements are assumed to be referencing the same or a substantially similar element having the same or substantially similar function or structure. Differences in function or structure may be described separately with regard to a specific feature described. Here, top and bottom surfaces 1802 and 1830 are substantially convex. In other examples, top and bottom surfaces 1802 and 1830 may be substantially concave, or one may be substantially convex and the other may be substantially concave. Top and bottom surfaces 1802 and 1830 may have radii of curvature that are one or two degrees less than the radii of curvature of opposing bones in a joint, so that device 1800 may fit within the joint (not shown). In some examples, outward facing vertical surface 1840 is disposed between peripheral protrusions 1812 and 1854. Outward facing vertical surface 1840 may have a curvature that is similar to the curvature of perimeter 1820. Outward facing vertical surface 1840 may have a curvature that aligns with perimeter 1820, such that once device 1800 is surgically implanted within a synovial capsule in a joint (not shown), there is minimal undesirable interaction between formations disposed about perimeter 1820 with adjacent tissue or bones. Contours 1838 may define, for example, a change in surface curvature between outward facing vertical surface 1840 and peripheral protrusion 1812. Channel opening 1804 may be disposed between peripheral protrusions 1812 and 1816, and be disposed on the opposite side of channel opening 1822. Channel opening 1822 may be disposed in between peripheral protrusions 1850 and 1854, and be disposed on the opposite side of channel opening 1804. The position of channel openings, however, may vary depending on the surface structure and topography of the joint bones that device 1800 is intended to be implanted within. In other examples, device 1800 and the elements shown and described may be designed, configured, formed, modified, or implemented apart from the examples shown or described and are not limited to those provided.

FIG. 18E illustrates a left view of an exemplary implantable interpositional orthopedic pain management apparatus. Here, a left view of device 1800 is shown, including top surface 1802, peripheral protrusions 1812-1816 and 1850-1854, channel openings 1808 and 1822, perimeter 1820, bottom surface 1830, contours 1838, and outward facing vertical surface 1844. As used herein, like numbered and/or like named elements are assumed to be referencing the same or a substantially similar element having the same or substantially similar function or structure. Differences in function or structure may be described separately with regard to a specific feature described. Here, both top surface 1802 and bottom surface 1830 are substantially convex, with three peripheral protrusions 1812-1816 on top surface 1802, and three peripheral protrusions 1850-1854 on bottom surface 1830. Top and bottom surfaces 1802 and 1830 may be configured to receive a bone adjacent to the joint. The positions of the peripheral protrusions 1812-1816 and 1850-1854 are selected in a matter that, once implanted in a joint, peripheral protrusions 1812-1816 and 1850-1854 aid in maintaining the position of device 1800 within the joint, for example during flexion or extension when various forces are applied on device 1800. Top and bottom surfaces 1802 and 1830, together with the peripheral protrusions 1812-1816 and 1850-1854 prevent contact between opposing bone surfaces within a joint, thus minimizing pain by absorbing a portion of the forces that may be applied to device 1800. In other examples, device 1800 and the elements shown and described may be designed, configured, formed, modified, or implemented apart from the examples shown or described and are not limited to those provided.

FIG. 18F illustrates a right view of an exemplary implantable interpositional orthopedic pain management apparatus. Here, a right view of device 1800 is shown including top surface 1802, peripheral protrusions 1812, 1814, 1852, and 1854, channel openings 1806 and 1826, perimeter 1820, bottom surface 1830, contours 1838, and outward facing vertical surface 1842. As used herein, like numbered and/or like named elements are assumed to be referencing the same or a substantially similar element having the same or substantially similar function or structure. Differences in function or structure may be described separately with regard to a specific feature described. In some examples, a channel may be formed on top surface 1802, in between peripheral protrusions 1814 and 1812, with channel opening 1806. The channel may be a portion of top surface 1802 that includes channel opening 1806, and may, in some examples, extend to and include channel openings 1804 and/or 1808 (not shown). A channel may be formed on bottom surface 1830, in between peripheral protrusions 1852 and 1854 with channel opening 1826. The channel may be a portion of bottom surface 1830 that includes channel opening 1826, and may, in some examples, extend to and include channel openings 1822 and 1824 (not shown). In other examples, device 1800 and the elements shown and described may be designed, configured, formed, modified, or implemented apart from the examples shown or described and are not limited to those provided.

FIG. 18G illustrates a perspective view of an exemplary implantable interpositional orthopedic pain management apparatus. Here, device 1800 includes top surface 1802, channel openings 1804-1808, peripheral protrusions 1812-1816, 1852, and 1854, perimeter 1820, outward facing vertical surfaces 1840 and 1842, bottom surface 1830, and contours 1838. As used herein, like numbered and/or like named elements are assumed to be referencing the same or a substantially similar element having the same or substantially similar function or structure. Differences in function or structure may be described separately with regard to a specific feature described. In some examples, a channel opening is disposed in between two adjacent peripheral protrusions, such as channel opening 1806 and peripheral protrusions 1812 and 1814, or channel opening 1808 and peripheral protrusions 1814 and 1816. A channel may be a portion of top surface 1802 that includes a channel opening such as 1806, and may overlap with other channels such as a channel that includes channel opening 1804. Outward facing vertical surfaces 1840-1844 (not shown) may be formed in part as portions of perimeter 1820, and/or be formed as a portion of peripheral protrusions 1812-1816, 1850-1854. Outward facing vertical surfaces 1840-1844 (not shown) may be substantially flat and/or be curved and positioned a manner to minimize interference of device 1800 with neighboring tissue or bones, thereby managing pain that may be involved in retention of device 1800 within a joint. Outward facing vertical surface 1840 may have a shape that is partially curved to align with adjacent peripheral protrusions 1812, 1854, and may have a bulb shape, with a somewhat rounded portion between peripheral protrusions 1812, 1854, and a somewhat pointed portion. When device 1800 is surgically implanted in a joint (e.g., through an incision or opening in a synovial capsule), peripheral protrusions 1812-1816 and 1850-1854 may be oriented in a joint to provide dynamic stability to a joint with weakened, degraded, or missing cartilage while preventing dislocation of bones from a joint and expulsion of device 1800 when a given joint is articulated. The rounded shapes of peripheral protrusions 1812-1816 and 1850-1854 may aid in uninterrupted dynamic contact and movement between bone surfaces and device 1800. Device 1800 and the elements shown and described may be designed, configured, formed, modified, or implemented apart from the examples shown or described and are not limited to those provided

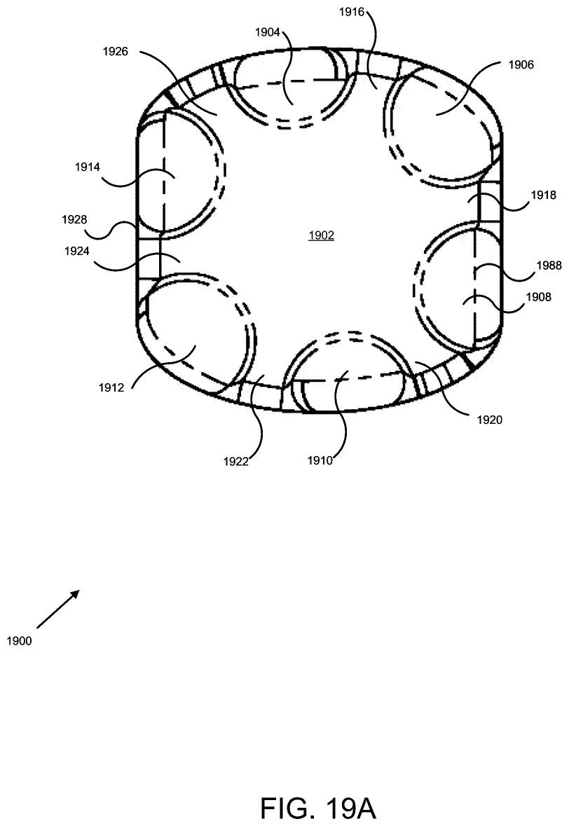

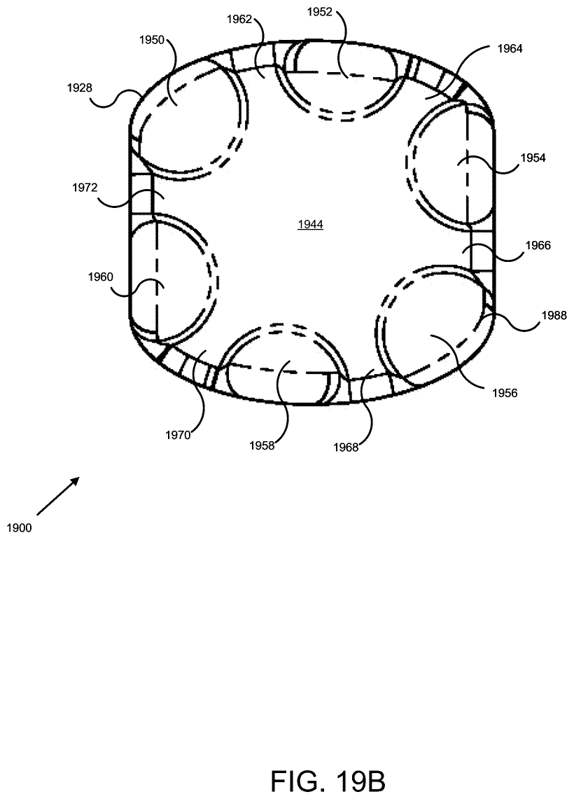

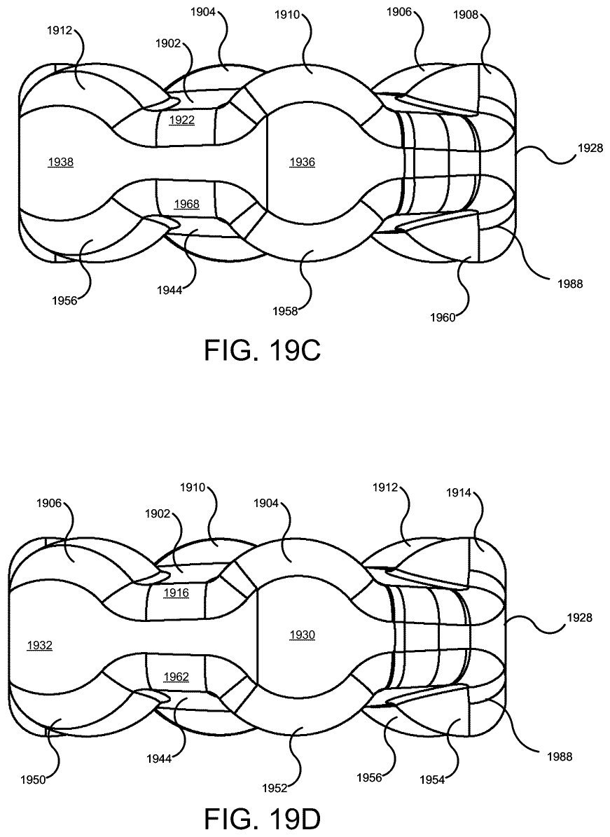

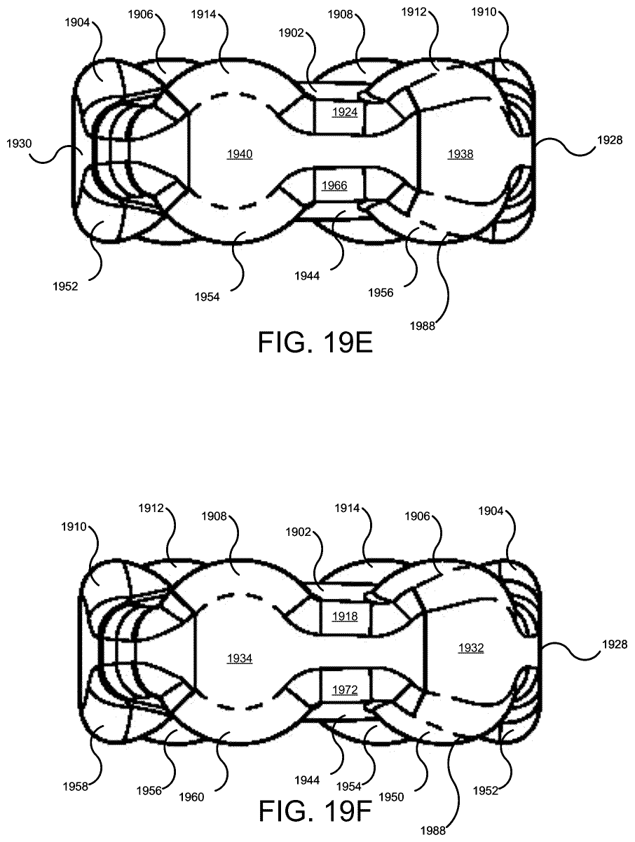

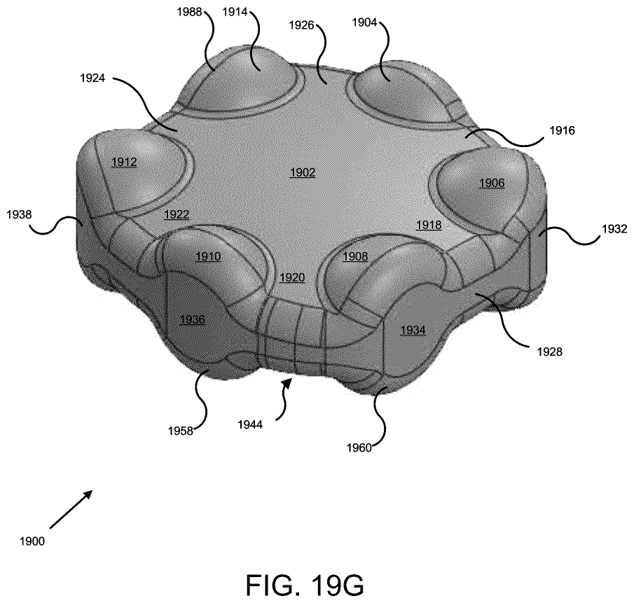

FIG. 19A illustrates a top view of an exemplary implantable interpositional orthopedic pain management apparatus. Here, device 1900 includes top surface 1902, peripheral protrusions 1904-1914, channel openings 1916-1926, perimeter 1928, and contours 1988. As used herein, like numbered and/or like named elements are assumed to be referencing the same or a substantially similar element having the same or substantially similar function or structure. Differences in function or structure may be described separately with regard to a specific feature described. In the present example, top surface 1902 is substantially four-sided with six peripheral protrusions 1904-1914. In some examples, peripheral protrusions 1904-1914 are formed with perimeter 1928 and disposed at substantially equal distances from one another on perimeter 1928. However, the positions of peripheral protrusions 1904-1914 are not limited to perimeter 1928 or having positions equidistant from each other. In some examples, top surface 1902 may be substantially concave. In some examples, top surface 1902 may be substantially convex. The radius of curvature of top surface 1902 may be selected to fit with an end of a bone (e.g., metacarpal, trapezium, tibia, femur, or others, without limitation or restriction). Channel openings 1916-1926 may be disposed in between peripheral protrusions 1904-1914. Conversely, peripheral protrusions 1904-1914 may be disposed in between channel openings 1916-1926. The number, shape, type, quantity, and disposition of peripheral protrusions 1904-1914 may be selected to be positioned within non-articulating portions of a synovial capsule and/or joint, and alleviates pain by preventing contact between distal and proximal ends of bones in a joint. Device 1900 may include additional surface elements as generally identified by contours 1988. For example, device 1900 may include an additional curved surface element in as a transition from peripheral protrusion 1908 to outward facing vertical surface 1938 (not shown) as illustrated by contours 1988. In other examples, device 1900 and the elements shown and described may be designed, configured, formed, modified, or implemented apart from the examples shown or described and are not limited to those provided.

FIG. 19B illustrates a bottom view of an exemplary implantable interpositional orthopedic pain management apparatus. Here, device 1900 includes perimeter 1928, bottom surface 1944, peripheral protrusions 1950-1960, channel openings 1962-1972, and contours 1988. As used herein, like numbered and/or like named elements are assumed to be referencing the same or a substantially similar element having the same or substantially similar function or structure. Differences in function or structure may be described separately with regard to a specific feature described. Elements shown herein are similar to those illustrated and described with reference to FIG. 19A. In the example herein, bottom surface 1944 includes six peripheral protrusions 1950-1960 spaced substantially equidistant from each other, formed with perimeter 1928. Channel openings 1962-1972 may be disposed at or substantially proximate to perimeter 1928. In some examples, bottom surface 1944 may be substantially convex and, together with peripheral protrusions 1950-1960 and channel openings 1962-1972, is configured to receive an end of a bone. In other examples, top and bottom surfaces 1902 (FIG. 19A) and 1944 may be implemented differently and are not limited to the examples shown and described.

FIG. 19C illustrates an anterior view of an exemplary interpositional orthopedic pain management apparatus. Here, an anterior or front view of device 1900 is shown, including top surface 1902, peripheral protrusions 1904-1912 and 1956-1960, perimeter 1928, bottom surface 1944, channel openings 1922 and 1968, outward facing vertical surfaces 1936 and 1938, and contours 1988. As used herein, like numbered and/or like named elements are assumed to be referencing the same or a substantially similar element having the same or substantially similar function or structure. Differences in function or structure may be described separately with regard to a specific feature described. Here, both top surface 1902 and bottom surface 1944 are substantially convex, with six peripheral protrusions 1904-1914 on top surface 1902, and six peripheral protrusions 1950-1960 on bottom surface 1944. Peripheral protrusions 1904-1914 and 1950-1960 are disposed along and formed with perimeter 1928. In other examples, peripheral protrusions 1904-1914 and 1950-1960 may be formed differently and are not limited to the examples shown and described. The configurations of surfaces 1902 and 1944 and peripheral protrusions 1904-1914 and 1950-1960 are selected to receive a bone within a joint or synovial capsule. In some examples, outward facing vertical surface 1936 is disposed between peripheral protrusions 1910 and 1958. Outward facing vertical surface 1936 may have a curvature element that is similar to the curvature of perimeter 1928. In some examples, a channel may be formed on top surface 1902, in between peripheral protrusions 1910 and 1912, with channel opening 1922. The channel may be a portion of top surface 1902 that includes channel opening 1922, and may, in some examples, include other channel openings on top surface 1902. A channel may be formed on bottom surface 1944, in between peripheral protrusions 1956 and 1958 with channel opening 1968. The channel may be a portion of bottom surface 1944 that includes channel opening 1968, and may, in some examples, include other channel openings on bottom surface 1944 (not shown). In other examples, device 1900 and the elements shown and described may be designed, configured, formed, modified, or implemented apart from the examples shown or described and are not limited to those provided.

FIG. 19D illustrates a posterior view of an exemplary implantable interpositional orthopedic pain management apparatus. Here, a posterior or back view of device 1900 is shown, including top surface 1902, peripheral protrusions 1904-1914 and 1950-1956, channel openings 1916 and 1962, perimeter 1928, outward facing vertical surfaces 1930 and 1936, bottom surface 1944, and contours 1988. As used herein, like numbered and/or like named elements are assumed to be referencing the same or a substantially similar element having the same or substantially similar function or structure. Differences in function or structure may be described separately with regard to a specific feature described. Here, top and bottom surfaces 1902 and 1944 are substantially convex. In some examples, top and bottom surfaces 1904 and 1944 may have radii of curvature that are substantially similar, or substantially opposite to one another (e.g. the bottom surface may be substantially concave with a similar radius of curvature as top surface). Top and bottom surfaces 1902 and 1944 may have radii of curvature that are one or two degrees greater than or less than the radii of curvature of opposing bones in a joint. In some examples, outward facing vertical surfaces 1930 and 1932 may be surfaces in between opposing peripheral protrusions. For example, outward facing vertical surface 1930 is in between peripheral protrusion 1904 and 1952. However, in other examples, the positions of peripheral protrusions are not limited to be opposing one another, such as the opposing peripheral protrusions 1904 and 1952. Outward facing vertical surfaces 1930 and 1932 may have a curvature element that aligns with perimeter 1928, such that once device 1900 is surgically implanted within a synovial capsule in a joint (not shown), there is minimal undesirable interaction between formations disposed about perimeter 1928 with adjacent tissue or bones. In other examples, device 1900 and the elements shown and described may be designed, configured, formed, modified, or implemented apart from the examples shown or described and are not limited to those provided.

FIG. 19E illustrates a left view of an exemplary implantable interpositional orthopedic pain management apparatus. Here, a left view of device 1900 is shown, including top surface 1902, peripheral protrusions 1904-1914 and 1954-1956, channel openings 1924 and 1966, perimeter 1928, outward facing vertical surfaces 1938 and 1940, bottom surface 1944, and contours 1988. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described with reference to FIGS. 19C and 19D. Here, both top surface 1902 and bottom surface 1944 are substantially convex, with six peripheral protrusions 1904-1914 on top surface 1902, and six peripheral protrusions 1950-1960 on bottom surface 1944. Top and bottom surfaces 1902 and 1944 may be configured to receive a bone adjacent to a joint. Top and bottom surfaces 1902 and 1944, together with peripheral protrusions 1904-1914 and 1950-1960 prevent contact between opposing bone surfaces within a joint, thus managing pain by absorbing a portion of the forces that may be applied to device 1900. In other examples, device 1900 and the elements shown and described may be designed, configured, formed, modified, or implemented apart from the examples shown or described and are not limited to those provided.

FIG. 19F illustrates a right view of an exemplary implantable interpositional orthopedic pain management apparatus. Here, a right view of device 1900 is shown, including top surface 1902, peripheral protrusions 1904-1914 and 1950-1960, channel openings 1918 and 1972, perimeter 1928, outward facing vertical surfaces 1932 and 1934, bottom surface 1944, and contours 1988. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described with reference to FIGS. 19C and 19D. In some examples, a channel may be formed on top surface 1902 in between adjacent peripheral protrusions 1906 and 1908, with channel opening 1918. The channel may be a portion of top surface 1902 that includes channel opening 1918, and may, in some examples, include other channel openings on the top surface (not shown). A channel may be formed on bottom surface 1944, in between adjacent peripheral protrusions 1950 and 1960 with channel opening 1972. The channel may be a portion of bottom surface 1944 that includes channel opening 1972, and may, in some examples, include other channel openings on bottom surface (not shown). In other examples, device 1900 and the elements shown and described may be designed, configured, formed, modified, or implemented apart from the examples shown or described and are not limited to those provided.

FIG. 19G illustrates a perspective view of an exemplary implantable interpositional orthopedic pain management apparatus. Here, device 1900 includes top surface 1902, peripheral protrusions 1904-1914, 1958, and 1960, perimeter 1928, channel openings 1916-1926, outward facing vertical surfaces 1932-1938, bottom surface 1944, and contours 1988. As used herein, like numbered and/or like named elements are assumed to be referencing the same or a substantially similar element having the same or substantially similar function or structure. Differences in function or structure may be described separately with regard to a specific feature described. As shown herein, top surface 1902 may be substantially convex, and have six peripheral protrusions 1904-1914 formed with top surface 1902 around perimeter 1928. Peripheral protrusions 1904-1914 have a partial-spherical portion. However, the positions and the shapes of peripheral protrusions 1904-1914 are not limited to those shown herein. Outward facing vertical surfaces 1930-1940 (not shown) may be disposed in between two peripheral protrusions on opposite sides of device 1900. For example, outward facing vertical surface 1934 is disposed in between peripheral protrusions 1908 and 1960, on top surface 1902 and bottom surface 1944, respectively. When device 1900 is surgically implanted in a joint (e.g., through an incision or opening in a synovial capsule), peripheral protrusions 1904-1914 and 1950-1960 may be oriented in a joint to provide dynamic stability to a joint with weakened, degraded, or missing cartilage while preventing dislocation of bones from a joint and expulsion of device 1900 when a given joint is articulated. By retaining device 1900 in a joint after surgical implantation, device 1900 and peripheral protrusions 1904-1914 and 1950-1960 aid in minimizing pain associated with dislocation of device 1900 from the joint. In some examples, two peripheral protrusions are on two sides of a channel opening. For example, peripheral protrusions 1908 and 1910 are disposed on two sides of channel opening 1920. A channel may be defined as a portion of top surface 1902 that includes a channel opening, such as 1902, and may also include other channels formed in part by other channel openings. In still other examples, device 1900 and the elements shown and described may be designed, configured, formed, modified, or implemented apart from the examples shown or described and are not limited to those provided.

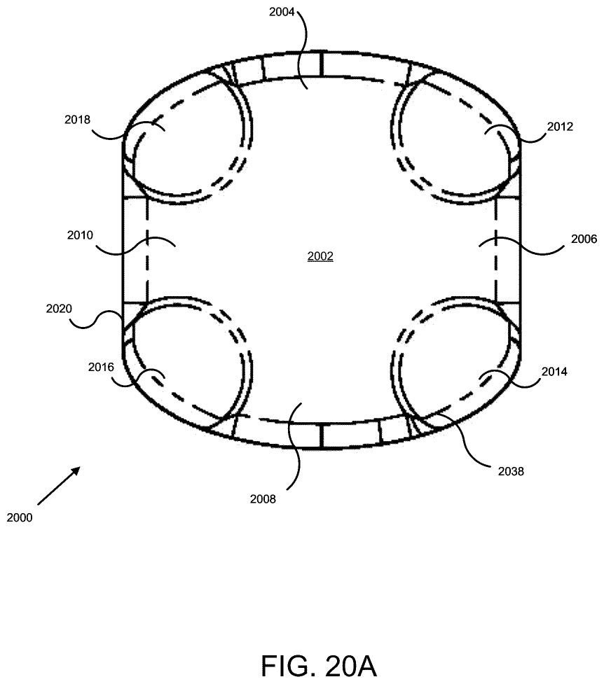

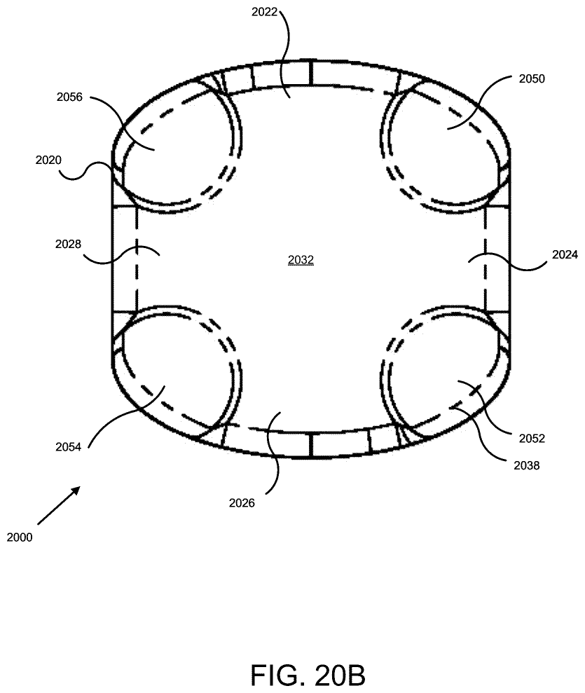

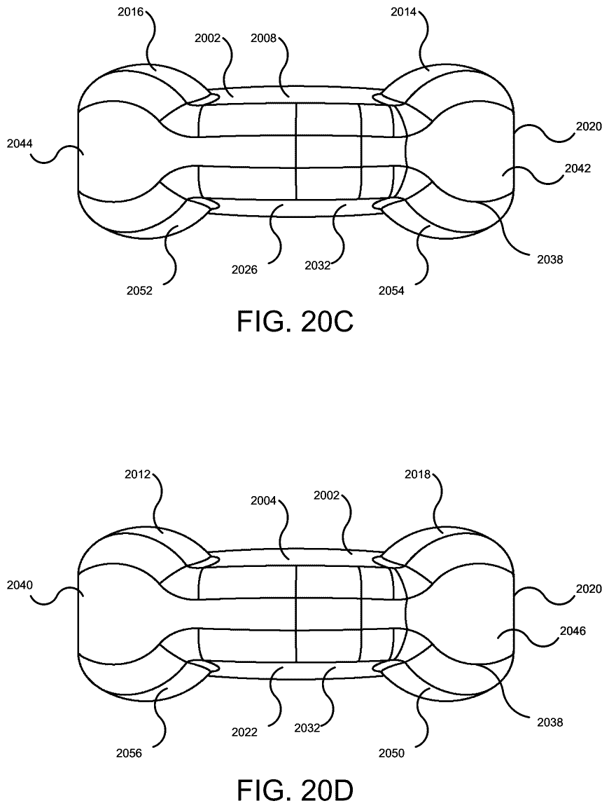

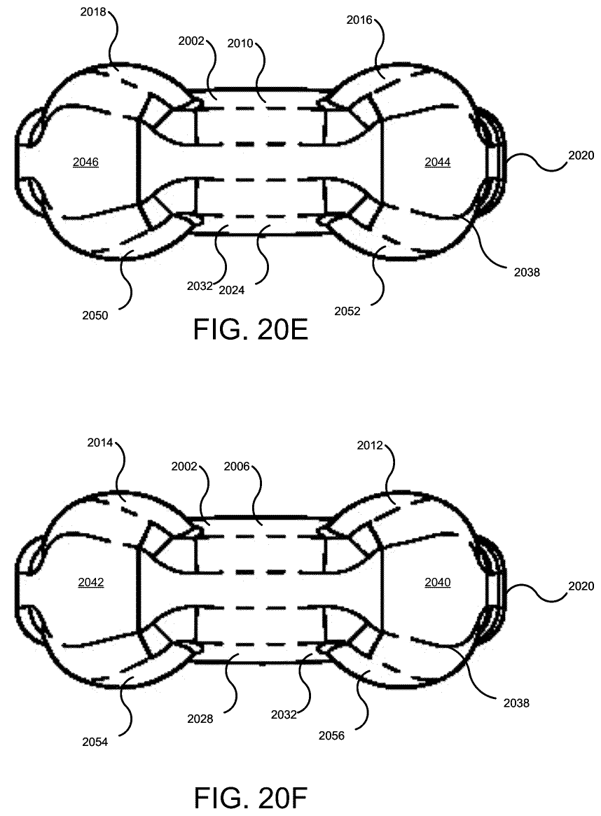

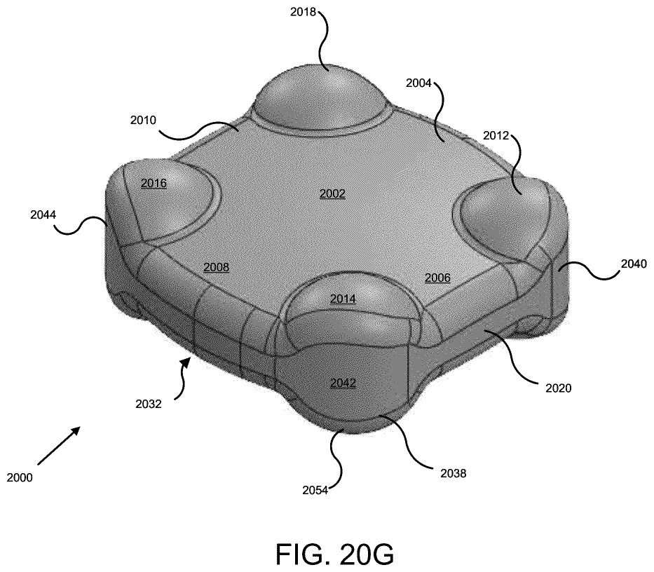

FIG. 20A illustrates a top view of an exemplary implantable interpositional orthopedic pain management apparatus. Here, device 2000 includes a top surface 2002, channel openings 2004-2010, peripheral protrusions 2012-2018, perimeter 2020, and contours 2038. As used herein, like numbered and/or like named elements are assumed to be referencing the same or a substantially similar element having the same or substantially similar function or structure. Differences in function or structure may be described separately with regard to a specific feature described. In some examples, top surface 2002 has at least a portion with a radius of curvature selected to fit with a portion of or an end of a bone (e.g., metacarpal, trapezium, tibia, femur, or others, without limitation or restriction). In some examples, at least a portion of top surface 2002 may have a radius of curvature such that top surface 2002 is substantially convex to receive (or otherwise be shaped to substantially fit) an end of a bone. Channel openings 2004-2010 may constitute part of top surface 2002 of device 2000. Channel openings 2004-2010 may be individually disposed in between peripheral protrusions 2012-2018. For example, channel opening 2006 is disposed in between peripheral protrusions 2012 and 2014. A channel (e.g., as formed by top surface 2002 and channel openings 2006 and 2010) may be disposed on top surface 2002 of the device 2000. Peripheral protrusions 2012-2018 may be disposed on top surface 2002 with outward facing vertical surfaces 2040-2046, to be later described in reference to FIGS. 20C-20G. Device 2000 may include additional surface elements as generally identified by contours 2038. In some examples, peripheral protrusions 2012-2018 may be disposed substantially about perimeter 2002 of device 2000, and aid in preventing expulsion of device 2000 from a joint in which the device is implanted. Peripheral protrusions 2012-2018 may be formed and integrated with top surface 2002 to provide pronounced structures that, when device 2000 is surgically implanted in a joint, may be configured to interact within articulating or non-articulating regions (or, in some examples, in a combination of articulating and non-articulating regions) of a joint. The positions of peripheral protrusions 2012-2018 may be selected such that they do not substantially interfere with motion of the joint once implanted within a joint. The positions of channel openings 2004-2010 and the one or more channels may be configured and selected to have a single channel or dual channels to receive the head or torus of a single bone of a joint. Device 2000 is configured to permit motion or articulation of a joint without dislocation or expulsion of device 2000 from the joint. In other words, when bones in a given joint are articulated, device 2000 when implanted may be configured and implemented to prevent one or more bones from dislocation or expulsing device 2000 from the joint. In some examples, device 2000 with peripheral protrusions 2012-2018 is configured to permit motion such as pivoting about one or more axes (e.g., abduction-adduction, flexion-extension, supination-pronation, and others) without dislocation of bones or displacement of device 2000 from a joint. In other examples, device 2000 and the elements shown and described may be designed, configured, formed, modified, or implemented apart from the examples shown or described and are not limited to those provided.

FIG. 20B illustrates a bottom view of an exemplary implantable interpositional orthopedic pain management apparatus. Here, device 2000 includes a bottom surface 2032, channel openings 2024-2028, peripheral protrusions 2050-2056, perimeter 2020, and contours 2038. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described in reference to FIG. 20A. In some examples, bottom surface 2032 has at least a portion with a radius of curvature selected to fit with a portion of or an end of a bone. In some examples, at least a portion of bottom surface 2032 may have a radius of curvature such that bottom surface 2032 is substantially convex to receive (or otherwise be shaped to substantially fit) an end of a bone. Peripheral protrusions 2050-2056 may be disposed on bottom surface 2032 with outward facing vertical surfaces 2040-2046, to be later described in reference to FIGS. 20C-20G. In some examples, peripheral protrusions 2040-2046 may be disposed substantially about perimeter 2020 of device 2000, and aid in preventing expulsion of device 2000 from a joint in which the device is implanted. Peripheral protrusions 2040-2046 may be formed and integrated with bottom surface 2032 to provide pronounced structures that, when device 2020 is surgically implanted in a joint, may be configured to interact within articulating or non-articulating regions (or, in some examples, in a combination of articulating and non-articulating regions) of a joint. The positions of peripheral protrusions 2050-2056 may be selected such that they do not substantially interfere with motion of the joint once implanted within a joint. Peripheral protrusions 2050-2056 may also be disposed on opposing sides of a channel opening 2022-2028. For example, peripheral protrusions 2052 and 2054 are disposed on opposing sides of channel opening 2026. In other examples, device 2000 and the elements shown and described may be designed, configured, formed, modified, or implemented apart from the examples shown or described and are not limited to those provided.

FIG. 20C illustrates an anterior view of an exemplary implantable interpositional orthopedic pain management apparatus. Here, an anterior or front view of device 2000 is shown, including top surface 2002, channel openings 2008 and 2026, peripheral protrusions 2014, 2016, 2052, and 2054, perimeter 2020, bottom surface 2032, contours 2038, and outward facing vertical surfaces 2042 and 2044. As used herein, like numbered and/or like named elements are assumed to be referencing the same or a substantially similar element having the same or substantially similar function or structure. Differences in function or structure may be described separately with regard to a specific feature described. Here, channel openings 2008 and 2026 are shown disposed on top and bottom surfaces, 2002 and 2032, respectively. Channel opening 2008 may be disposed in between two adjacent peripheral protrusions 2014 and 2016. The channel formed on top surface 2002 with channel openings 2008 and 2004 (not shown) may be shaped to receive a bone. Similarly, the channel formed on bottom surface 2032 with channel opening 2026 and 2022 (not shown) may be shaped to receive an adjacent bone in a joint. In some examples, peripheral protrusions 2004-2010 and 2050-2056 may be shaped with a rounded or spherical portion. The rounded or spherical shape of peripheral protrusions 2004-2010 and 2050-2056 may support uninterrupted or smooth dynamic adjustments or minor shifts of device 2000 within the joint once implanted. Such dynamic adjustment may in part constitute the intermittent contact between portions of device 2000 and/or peripheral protrusions 2004-2010 and 2050-2056 with bones in a joint. In some examples, peripheral protrusions 2004-2010 and 2050-2056 may include outward facing vertical surfaces 2040-2046. In other examples, peripheral protrusions 2004-2010 and 2050-2056 and outward facing vertical surfaces 2004-2010 and 2050-2056 may be formed differently and are not limited to the examples shown and described. Device 2000 may include additional curved surface elements, for example, a change in curvature between peripheral protrusion 2014 and outward facing vertical surface 2042, as illustrated by contours 2038. In still other examples, device 2000 and the elements shown and described may be designed, configured, formed, modified, or implemented apart from the examples shown or described and are not limited to those provided.

FIG. 20D illustrates a posterior view of an exemplary implantable interpositional orthopedic pain management apparatus. Here, a posterior or rear view of the device 2000 is shown, including top surface 2002, channel openings 2004 and 2022, peripheral protrusions 2012, 2018, 2050, and 2056, perimeter 2020, bottom surface 2032, contours 2038, and outward facing vertical surfaces 2040 and 2046. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described in reference to FIG. 20C. In some examples, channel openings 2004 and 2008 (not shown) form a channel configured to receive a bone in top surface 2002 when device 2000 is surgically implanted into a synovial capsule and joint. In some examples, channel openings 2022 and 2026 (not shown) form a channel configured to receive a bone in bottom surface 2032 when device 2000 is surgically implanted into a synovial capsule and joint. In other examples, device 2000 and the elements shown and described may be designed, configured, formed, modified, or implemented apart from the examples shown or described and are not limited to those provided.

FIG. 20E illustrates a left view of an exemplary implantable interpositional pain management apparatus. Here, a left view of the device 2000 is shown, including top surface 2002, channel openings 2010 and 2024, peripheral protrusions 2016, 2018, 2050, and 2052, perimeter 2020, bottom surface 2032, contours 2038, and outward facing vertical surfaces 2044 and 2046. As used herein, like numbered and/or like named elements are assumed to be referencing the same or a substantially similar element having the same or substantially similar function or structure. Differences in function or structure may be described separately with regard to a specific feature described. In some examples, top surface 2002 is configured, with channel openings 2010 and 2006 (not shown), to receive a bone or a portion thereof when device 2000 is surgically implanted. Bottom surface 2032 may also be configured, with channel openings 2024 and 2028 (not shown), to receive a bone or a portion thereof when device 2000 is surgically implanted. For example, if device 2000 is surgically implanted into a CMC joint, top surface 2002 may be configured, with channel openings 2004 and 2008, to receive (i.e., top surface 2002 may have a radius of curvature that is larger than that of a trapezium bone) a trapezium bone, or other bone adjacent to the joint. Outward facing vertical surfaces 2044 and 2046 may have a curvature that aligns with perimeter 2020, such that device 2000 minimally interferes with lateral tissue or bones once surgically implanted within a joint. Outward facing vertical surfaces 2044 and 2046 may have a shape with curved portions adjacent to peripheral protrusions 2016, 2018, 2050, and 2052. However, the shape and orientation of outward facing vertical surfaces 2040-2046 are not limited to those shown and described herein. In other examples, device 2000 and the elements shown and described may be designed, configured, formed, modified, or implemented apart from the examples shown or described and are not limited to those provided.

FIG. 20F illustrates a right view of an exemplary implantable interpositional pain management apparatus. Here, a right view of device 2000 is shown, including top surface 2002, channel openings 2006 and 2028, peripheral protrusions 2012, 2014, 2054, and 2056, perimeter 2020, bottom surface 2032, contours 2038, and outward facing vertical surfaces 2040 and 2042. As used herein, like numbered and/or like named elements are assumed to be referencing the same or a substantially similar element having the same or substantially similar function or structure. Differences in function or structure may be described separately with regard to a specific feature described. Elements herein are similar to those described in reference to FIG. 20D. In some examples, top surface 2002 is configured, with channel openings 2006 and 2010 (not shown), to receive a bone or a portion thereof when device 2000 is surgically implanted. Bottom surface 2032 may also be configured, with channel openings 2028 and 2024 (not shown), to receive a bone or a portion thereof when device 2000 is surgically implanted. Outward facing vertical surfaces 2040 and 2042 may be substantially flat have a curvature that aligns with perimeter 2020, such that device 2000 minimally interferes with lateral tissue or bones once surgically implanted within a joint. In other examples, device 2000 and the elements shown and described may be designed, configured, formed, modified, or implemented apart from the examples shown or described and are not limited to those provided.

FIG. 20G. illustrates a perspective view of an exemplary implantable interpositional orthopedic pain management apparatus. Here, device 200 includes top surface 2002, channel openings 2004-2010, peripheral protrusions 2012-2018 and 2054, perimeter 2020, outward facing vertical surfaces 2040-2044, bottom surface 2032, and contours 2038. As used herein, like numbered and/or like named elements are assumed to be referencing the same or a substantially similar element having the same or substantially similar function or structure. Differences in function or structure may be described separately with regard to a specific feature described. In the example herein, top surface 2002 may have a radius of curvature such that top surface 2002 is substantially convex. Peripheral protrusions 2012-2018, and similarly 2050-2056, may have a partial spherical shape, but the shape is not limited to the example shown herein. The configurations of top surface 2002 and peripheral protrusions 2012-2018 are selected based on the shape of an end of a bone which device 2000 is implanted against. The configurations of bottom surface 2032 and peripheral protrusions 2050-2056 (not shown) are also selected based on the shape of an end of a bone in the same joint, once surgically implanted. When device 2000 is surgically implanted in a joint (e.g., through an incision or opening in a synovial capsule), peripheral protrusions 2012-2018 and 2050-2056 (not shown) may be oriented in a joint to provide dynamic stability to a joint with weakened, degraded, or missing cartilage while preventing dislocation of bones from a joint and expulsion of device 2000 when a given joint is articulated. In some examples, outward facing vertical surfaces 2040-2046 have a curvature that aligns with the curvature of perimeter 2020, such that the vertical surfaces, when seen from a top view (e.g. FIG. 20A), are not viewable. In some examples, outward facing vertical surfaces 2040-2046 are coextensive with perimeter 2020, such that outward facing vertical surfaces 2040-2046 are portions of perimeter 2020. Once surgically implanted within a joint, top and bottom surfaces 2002, 2032 and peripheral protrusions 2012-2018 and 2050-2056 (not shown) may be in dynamic contact with the adjacent bone surfaces. Top and bottom surfaces 2002, 2032 and peripheral protrusions 2012-2018 and 2050-2056 (not shown) may be in intermittent contact with adjacent bone surfaces, in part to dynamically distribute forces applied during movement (e.g. flexion, extension) through different portions of device 2000, while the peripheral protrusions 2012-2018 and 2050-2056 are oriented to retain device 2000 within the joint. In other examples, device 2000 and the elements shown and described may be designed, configured, formed, modified, or implemented apart from the examples shown or described and are not limited to those provided.

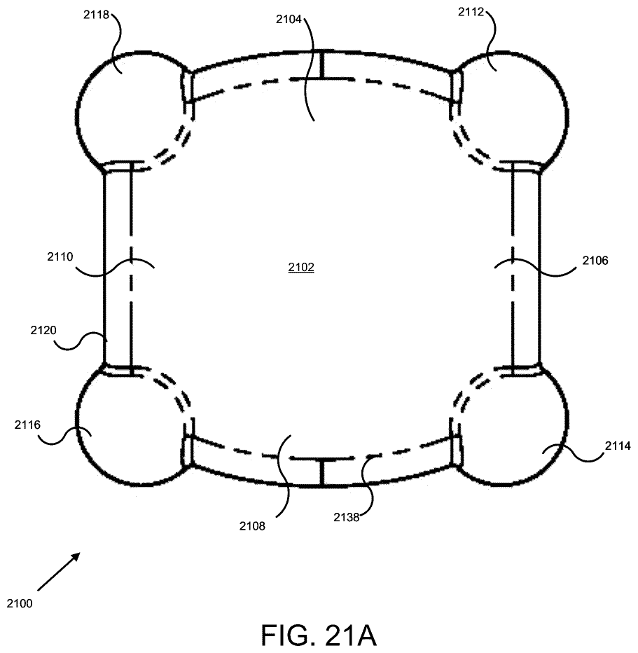

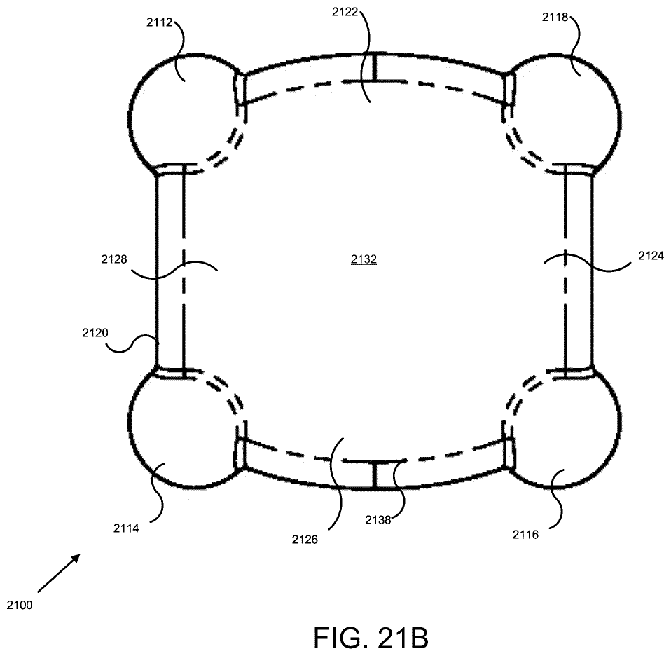

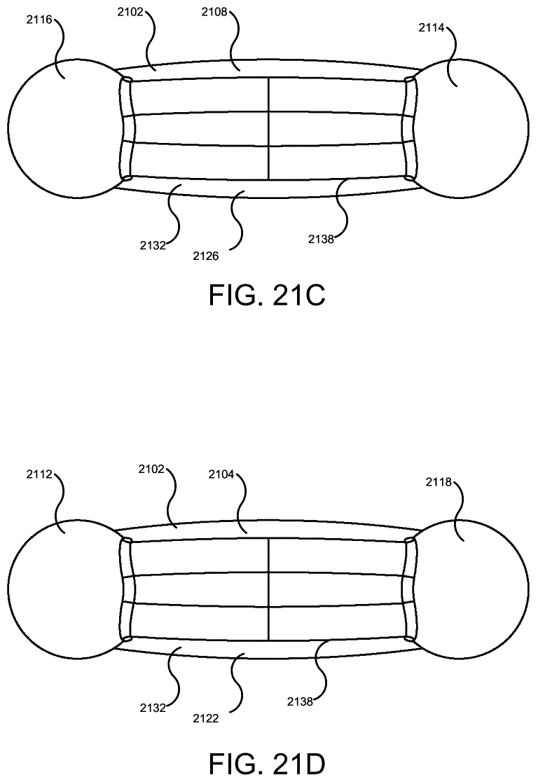

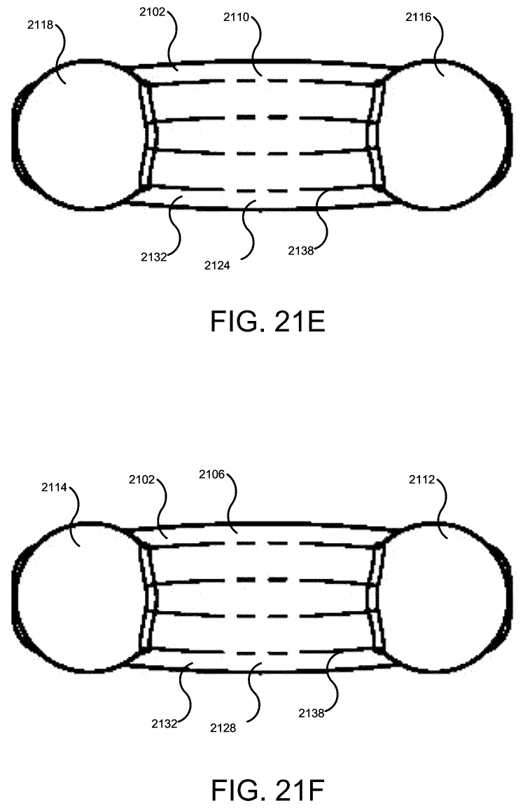

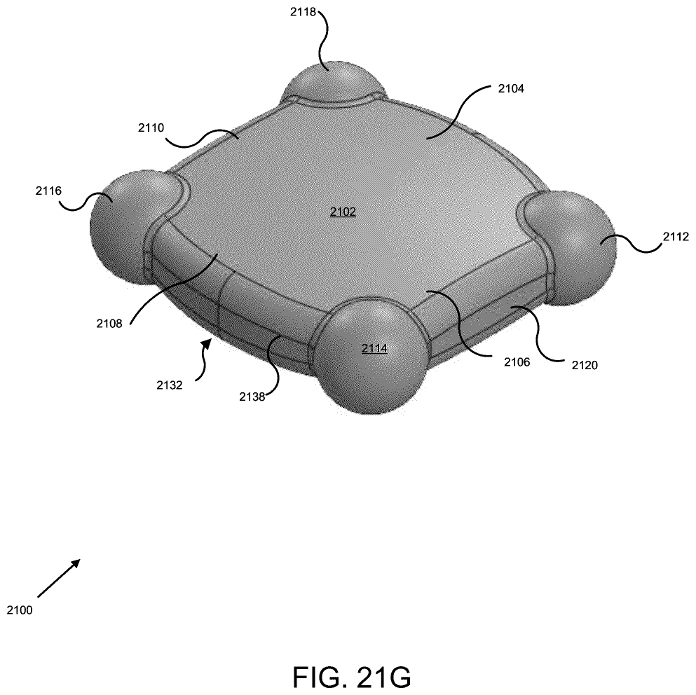

FIG. 21A illustrates a top view of an exemplary implantable interpositional orthopedic pain management apparatus. Here, device 2100 includes a top surface 2102, channel openings 2104-2110, peripheral protrusions 2112-2118, perimeter 2020, and contours 2038. As used herein, like numbered and/or like named elements are assumed to be referencing the same or a substantially similar element having the same or substantially similar function or structure. Differences in function or structure may be described separately with regard to a specific feature described. In some examples, top surface 2102 has at least a portion with a radius of curvature selected to fit with an end of a bone (e.g., metacarpal, trapezium, tibia, femur, or others, without limitation or restriction). In some examples, at least a portion of top surface 2102 may have a radius of curvature such that top surface 2102 is substantially convex to receive (or otherwise be shaped to substantially fit) an end of a bone. Channel openings 2104-2110 may constitute part of top surface 2102 of device 2100. Channel openings 2104-2110 may be individually disposed in between peripheral protrusions 2112-2118. For example, channel opening 2108 is disposed in between peripheral protrusions 2114 and 2116. A channel (e.g., as formed by the top surface 2102 and channel openings 2106 and 2110) may be disposed on top surface 2102 of the device. Peripheral protrusions 2112-2118 may be disposed on top surface 2102. In some examples, top surface 2102 may be substantially four-sided, with each peripheral protrusion 2112-2118 disposed at a corner of top surface 2102. Peripheral protrusions 2112-2118 may also be substantially spherical in shape and may have a circular cross section. Peripheral protrusions 2112-2118 may be formed and integrated with top surface 2102 to provide pronounced structures that, when device 2100 is surgically implanted in a joint, may be configured to interact within articulating or non-articulating regions (or, in some examples, in a combination of articulating and non-articulating regions) of a joint. The positions of channel openings 2104-2110 and the one or more channels may be configured and selected to have a single channel or dual channels to receive the head or torus of a single bone of a joint. Device 2100 is configured to permit motion or articulation of a joint without dislocation or expulsion of device 2100 from the joint. In other words, when bones in a given joint are articulated, device 2100 when implanted may be configured and implemented to prevent one or more bones from dislocation or expulsing device 2100 from the joint. In some examples, device 2100 with peripheral protrusions 2112-2118 is configured to permit motion such as pivoting about one or more axes (e.g., abduction-adduction, flexion-extension, supination-pronation, and others) without dislocation of bones or displacement of device 2100 from a joint. In some examples, surgical clamps or any type of implant holder may be configured for surgical insertion of implantable devices such as device 2100 in human or animal joints, which may include carpometacarpal, trapeziometacarpal, or others, without limitation or restriction. In other examples, device 2100 and the elements shown and described may be designed, configured, formed, modified, or implemented apart from the examples shown or described and are not limited to those provided.

FIG. 21B illustrates a bottom view of an exemplary implantable interpositional orthopedic pain management apparatus. Here, device 2100 includes a bottom surface 2132, peripheral protrusions 2112-2118, channel openings 2124-2128, perimeter 2020, and contours 2138. As used herein, like numbered and/or like named elements are assumed to be referencing the same or a substantially similar element having the same or substantially similar function or structure. Differences in function or structure may be described separately with regard to a specific feature described. In some examples, bottom surface 2132 has at least a portion with a radius of curvature selected to fit with an end of a bone. In some examples, at least a portion of bottom surface 2132 may have a radius of curvature such that bottom surface 2132 is substantially convex to receive (or otherwise be shaped to substantially fit) an end of a bone. Bottom sides of peripheral protrusions 2112-2118 (as compared with top view shown in FIG. 21A) are viewable and formed with corners of bottom surface 2132 and may be formed with and along perimeter 2020. In some examples, a channel may be formed in between channel openings 2124 and 2128 and may constitute a portion of bottom surface 2132. A channel may be formed in between channel openings 2122 and 2126 and may also constitute a portion of bottom surface 2132. Device 2100 may include additional surface elements as generally identified by contours 2138. For example, device 2100 may include an additional curved surface element in at a portion of bottom surface 2132 near perimeter 2020, as illustrated by contours 2138. In other examples, device 2100 and the elements shown and described may be designed, configured, formed, modified, or implemented apart from the examples shown or described and are not limited to those provided.

FIG. 21C illustrates an anterior view of an exemplary implantable interpositional orthopedic pain management apparatus. Here, an anterior or front view of device 2100 is shown, including top surface 2102, channel openings 2108 and 2126, peripheral protrusions 2114 and 2116, bottom surface 2132, and contours 2138. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2102 in between channel openings 2108 and 2104 (not shown). A channel may also be formed on bottom surface 2132 in between channel openings 2126 and 2122 (not shown). Here, the channels may have a curvature is coextensive with the convex curvature of top surface 2102, and bottom surface 2132. Peripheral protrusions 2114 and 2116 may be disposed on opposite sides of channel openings 2108 and 2126. Top and bottom surfaces 2102 and 2132 may each be formed to receive mutually reciprocal or substantially mutually reciprocal bones, bone structures, or portions thereof in a joint (e.g., trapezium, metacarpal, radial facet of a metacarpal, or others, without restriction or limitation). In still other examples, device 2100 and the elements shown and described may be designed, configured, formed, modified, or implemented apart from the examples shown or described and are not limited to those provided.

FIG. 21D illustrates a posterior view of an exemplary implantable interpositional orthopedic pain management apparatus. Here, a posterior or back view of device 2100 is shown, including top surface 2102, channel openings 2104 and 2122, peripheral protrusions 2112 and 2118, bottom surface 2132, and contours 2138. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2102 in between channel openings 2104 and 2108 (not shown). A channel may also be formed on bottom surface 2132 in between channel openings 2122 and 2126 (not shown). Here, the channels may have a curvature is coextensive with the convex curvature of top surface 2102, and bottom surface 2132. Peripheral protrusions 2122 and 2118 may be disposed on opposite sides of channel openings 2104 and 2122. In still other examples, device 2100 and the elements shown and described may be designed, configured, formed, modified, or implemented apart from the examples shown or described and are not limited to those provided.

FIG. 21E illustrates a left view of an exemplary implantable interpositional orthopedic pain management apparatus. Here, a left view of device 2100 is shown, including top surface 2102, channel openings 2110 and 2124, peripheral protrusions 2116 and 2118, bottom surface 2132, and contours 2138. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2102 in between channel openings 2110 and 2106 (not shown). A channel may also be formed on bottom surface 2132 in between channel openings 2124 and 2128 (not shown). Here, the channels may have a curvature is coextensive with the convex curvature of top surface 2102, and bottom surface 2132. Peripheral protrusions 2116 and 2118 may be disposed on opposite sides of channel openings 2110 and 2124. Peripheral protrusions 2116 and 2118 may be substantially spherical and have circular cross sections. Once surgically implanted within a joint (not shown), top and bottom surfaces 2102 and 2132 and portions of peripheral protrusions 2112-2118 may be in dynamic contact with opposing bone surfaces, device 2100 being configured to prevent bones from coming into contact with each other. In still other examples, device 2100 and the elements shown and described may be designed, configured, formed, modified, or implemented apart from the examples shown or described and are not limited to those provided.

FIG. 21F illustrates a right view of an exemplary implantable interpositional orthopedic pain management apparatus. Here, a right view of device 2100 is shown, including top surface 2102, channel openings 2106 and 2128, peripheral protrusions 2112 and 2114, bottom surface 2132, and contours 2138. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2102 in between channel openings 2106 and 2110 (not shown). A channel may also be formed on bottom surface 2132 in between channel openings 2128 and 2124 (not shown). Here, the channels may have a curvature is coextensive with the convex curvature of the top surface 2102, and bottom surface 2132. Peripheral protrusions 2112 and 2114 may be disposed on opposite sides of channel openings 2110 and 2124. In still other examples, device 2100 and the elements shown and described may be designed, configured, formed, modified, or implemented apart from the examples shown or described and are not limited to those provided.

FIG. 21G illustrates a perspective view of an exemplary implantable interpositional orthopedic pain management apparatus. Here, a perspective view of device 2100 includes top surface 2102, channel openings 2104-2110, peripheral protrusions 2112-2118, perimeter 2120, bottom surface, and contours 2138. As used herein, like numbered and/or like named elements are assumed to be referencing the same or a substantially similar element having the same or substantially similar function or structure. Differences in function or structure may be described separately with regard to a specific feature described. As shown, top surface 2102 may be substantially convex and have a substantially four-sided shape. Peripheral protrusions 2112-2118 may be located on the corners of four-sided device 2100. In some examples, peripheral protrusions 2112-2118 may be coupled directly or indirectly with periphery 2120 or formed as surface or structural features or components thereof. As shown and described, peripheral protrusions 2112-2118 may have various sizes, shapes, dimensions, and other attributes, without limitation or restriction. Peripheral protrusions 2112-2118 may have features integrated or formed within them as well, to engage and fit within a joint when device 2100 is surgically implanted. Channel openings 2104-2110 may be disposed near or at perimeter 2120, and opposing channel openings (e.g. 2106 and 2110) may form a channel between them, the channel constituting a portion of top surface 2102. In other examples, top and bottom surfaces 2102 and 2132, and peripheral protrusions 2112-2118 may also be configured to provide surfaces that are shaped to fit and/or fill with other materials within a synovial capsule such as cartilage, blood, bodily fluids, or other synthetic or organic materials that are injected or otherwise implanted to aid device 2100 in maintaining dynamic stability and position within a joint after surgical implantation. In still other examples, device 2100 and the elements shown and described may be designed, configured, formed, modified, or implemented apart from the examples shown or described and are not limited to those provided.

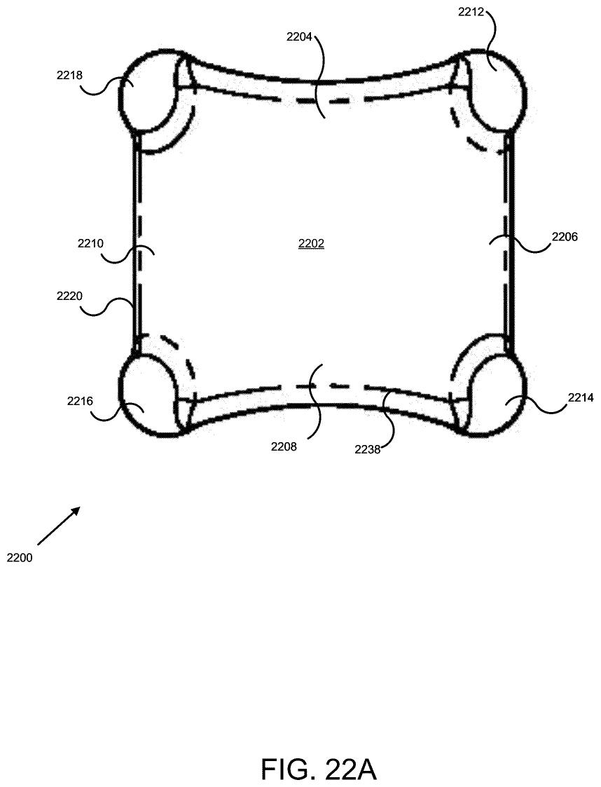

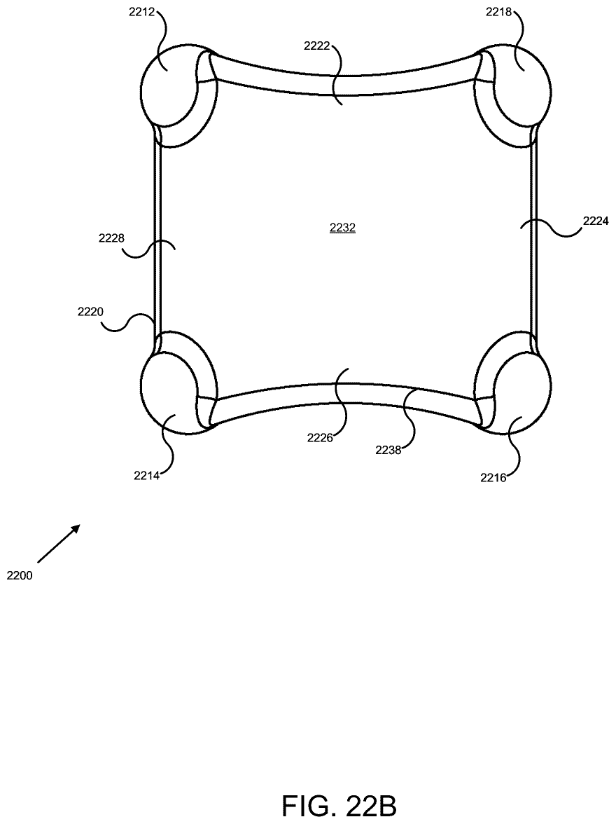

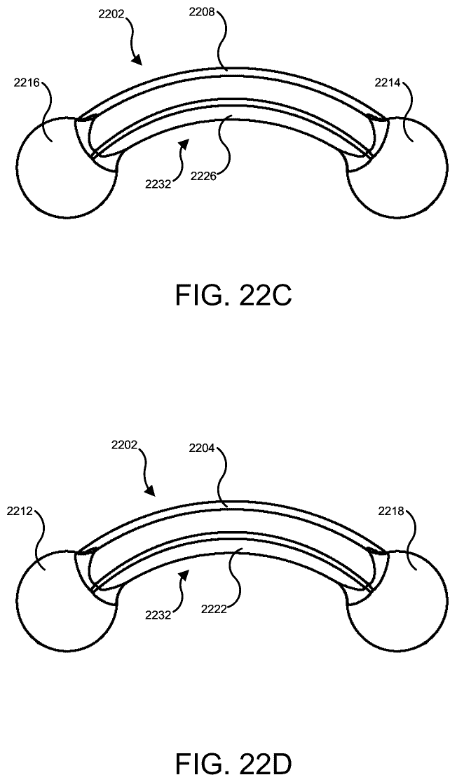

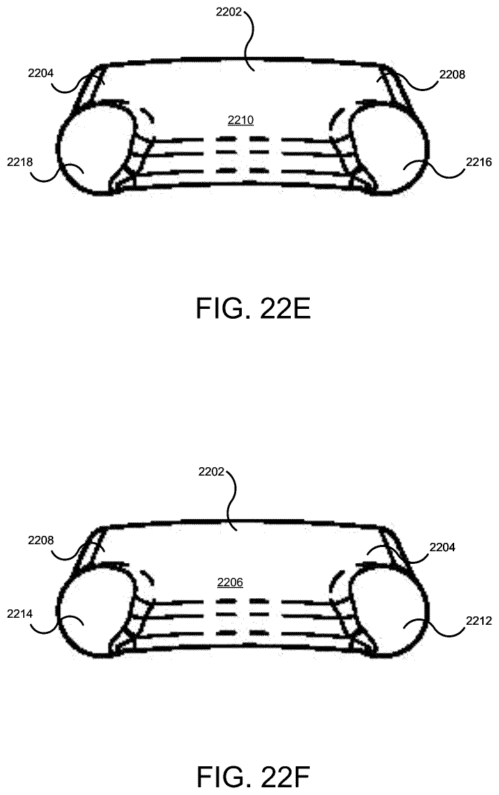

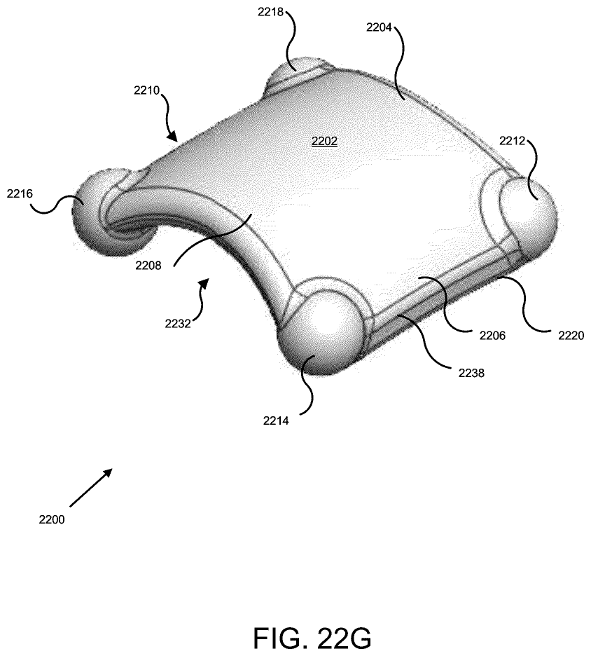

FIG. 22A illustrates a top view of an exemplary implantable interpositional orthopedic pain management apparatus. Here, device 2200 includes a top surface 2202, channel openings 2204-2210, peripheral protrusions 2212-2218, perimeter 2220, and contours 2238. As used herein, like numbered and/or like named elements are assumed to be referencing the same or a substantially similar element having the same or substantially similar function or structure. Differences in function or structure may be described separately with regard to a specific feature described. In some examples, top surface 2202 has at least a portion with a radius of curvature selected to fit with an end of a bone (e.g., capitate, lunate, metacarpal, trapezium, tibia, femur, or others, without limitation or restriction). The radius of curvature of top surface 2202 may be one or two degrees less than that of a bone within a joint. Top surface 2202 may be substantially four sided, with peripheral protrusions 2212-2218 disposed at each of four corners. Peripheral protrusions 2212-2218 may be substantially spherical in shape, with substantially circular cross sections. In some examples, channel openings 2204-2210 may constitute part of top surface 2202 of device 2200. Device 2200 may include additional surface elements as generally identified by contours 2238. For example, device 2200 may include a curved surface element in between peripheral protrusion 2214 and top surface 2202, as illustrated by contours 2238, indicating a change in surface curvature between top surface 2202 and peripheral protrusion 2214. A channel (e.g., as formed by top surface 2202 and channel openings 2206 and 2206) may be disposed on top surface 2202 of device 2200. In some examples, peripheral protrusions 2212-2218 may be disposed substantially about perimeter 2220 of device 2200, and aid in preventing expulsion of the device from a joint in which the device is implanted. Peripheral protrusions 2212-2218 may be formed and integrated with top surface 2202 to provide pronounced structures that, when device 2200 is surgically implanted in a joint, may be configured to interact within articulating or non-articulating regions (or, in some examples, in a combination of articulating and non-articulating regions) of a joint. The positions of peripheral protrusions 2212-2218 may be selected such that they do not substantially interfere with motion of the joint once implanted within a joint. As described above, device 2200 is configured to permit motion or articulation of a joint without dislocation or expulsion of device 2200 from the joint. In other words, when bones in a given joint are articulated, device 2200 when implanted may be configured and implemented to prevent one or more bones from dislocation or expulsing device 2200 from the joint. In some examples, device 2200 with peripheral protrusions 2212-2218 are configured to permit motion such as pivoting about one or more axes (e.g., abduction-adduction, flexion-extension, supination-pronation, and others) without dislocation of bones or displacement of device 2200 from a joint. In some examples, device 2200 may be implanted and used as a spacer in between two adjacent bones, such as between lunate and capitate bones. In other examples, device 2200 and the elements shown and described may be designed, configured, formed, modified, or implemented apart from the examples shown or described and are not limited to those provided.

FIG. 22B illustrates a bottom view of an exemplary implantable interpositional orthopedic pain management apparatus. Here, device 2200 includes perimeter 2220, bottom surface 2232, peripheral protrusions 2212-2218, channel openings 2222-2224, and contours 2238. As used herein, like numbered and/or like named elements are assumed to be referencing the same or a substantially similar element having the same or substantially similar function or structure. Differences in function or structure may be described separately with regard to a specific feature described. In the view of the example shown herein, bottom surface 2232 of device 2200 is substantially similar to the top view FIG. 22A. Bottom sides of peripheral protrusions 2212-2218 are disposed on perimeter 2220. Peripheral protrusions 2212-2218 are substantially spherical in shape. In the example herein, bottom surface 2232 has a radius of curvature that is substantially opposite that of top surface 2202 (not shown). A joint within which device 2200 may be surgically implanted into may have opposing bone surfaces with substantially opposite radii of curvature, such that a surface of a first bone fits into the surface of the adjacent bone. Once implanted, peripheral protrusions 2212-2218 may maintain device 2200 between two bone surfaces by resisting any dislodging forces applied to device 2200. In other examples, device 2200 and the elements shown and described may be designed, configured, formed, modified, or implemented apart from the examples shown or described and are not limited to those provided.

FIG. 22C illustrates an anterior view of an exemplary implantable interpositional orthopedic pain management apparatus. Here, an anterior or front view of device 2200 is shown, including top surface 2202, channel openings 2208 and 2226, peripheral protrusions 2214 and 2216, and bottom surface 2232. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2202 in between channel openings 2208 and 2204 (not shown). A channel may also be formed on bottom surface 2232 in between channel openings 2226 and 2222 (not shown). Here, the channels may have a curvature is coextensive with the curvatures of top surface 2202, and bottom surface 2232, respectively. In the example shown, top surface 2202 may have a curvature element that is substantially opposite of a curvature element of bottom surface 2232. Peripheral protrusions 2214 and 2216 may be disposed on opposite sides of channel openings 2208 and 2226. Top and bottom surfaces 2202 and 2236 may each be formed to receive mutually reciprocal or substantially mutually reciprocal bones, bone structures, or portions thereof in a joint. In still other examples, device 2200 and the elements shown and described may be designed, configured, formed, modified, or implemented apart from the examples shown or described and are not limited to those provided.

FIG. 22D illustrates a posterior view of an exemplary implantable interpositional orthopedic pain management apparatus. Here, a posterior or rear view of device 2200 is shown, including top surface 2202, channel openings 2204 and 2222, and peripheral protrusions 2212 and 2218, and bottom surface 2232. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2202 in between channel openings 2204 and 2208 (not shown). A channel may also be formed on bottom surface 2232 in between channel openings 2222 and 2226 (not shown). Here, the channels may have a curvature is coextensive with the curvatures of top surface 2202, and bottom surface 2232, respectively. Peripheral protrusions 2212 and 2218 may be disposed on opposite sides of channel openings 2204 and 2222. Top and bottom surfaces 2202 and 2236 may each be formed to receive mutually reciprocal or substantially mutually reciprocal bones, bone structures, or portions thereof in a joint. In still other examples, device 2200 and the elements shown and described may be designed, configured, formed, modified, or implemented apart from the examples shown or described and are not limited to those provided.

FIG. 22E illustrates a left view of an exemplary implantable interpositional orthopedic pain management apparatus. Here, a left view of device 2200 is shown, including top surface 2202, channel openings 2204, 2208, and 2210, and peripheral protrusions 2216 and 2218. As used herein, like numbered and/or like named elements are assumed to be referencing the same or a substantially similar element having the same or substantially similar function or structure. Differences in function or structure may be described separately with regard to a specific feature described. As shown, top surface 2202 in this example has a radius of curvature that obscures bottom surface 2232 (not shown) from a left view. In some examples, a channel is formed on top surface 2202 in between channel openings 2204 and 2208. A substantially orthogonal channel may also be formed on top surface 2202 in between channel openings 2210 and 2206 (not shown). Here, the channels may have a curvature is coextensive with the convex curvature of top surface 2202. Peripheral protrusions 2216 and 2218 may be disposed on opposite sides of channel opening 2210. Peripheral protrusions 2216 and 2218 may be substantially spherical and have circular cross sections. Once surgically implanted within a joint (not shown), top and bottom surfaces 2202 and 2232 (not shown) and portions of peripheral protrusions 2212-2218 may be in dynamic or intermittent contact with opposing bone surfaces, device 2200 being configured to prevent bones from coming into contact with each other and alleviating pain associated with wear or degradation between bone surfaces. In still other examples, device 2200 and the elements shown and described may be designed, configured, formed, modified, or implemented apart from the examples shown or described and are not limited to those provided.

FIG. 22F illustrates a right view of an exemplary implantable interpositional orthopedic pain management apparatus. Here, a right view of device 2200 is shown, including top surface 2202, channel openings 2204-2208, and peripheral protrusions 2212 and 2214. As used herein, like numbered and/or like named elements are assumed to be referencing the same or a substantially similar element having the same or substantially similar function or structure. Differences in function or structure may be described separately with regard to a specific feature described. In some examples, a channel is formed on top surface 2202 in between channel openings 2204 and 2208. A substantially orthogonal channel may also be formed on top surface 2202 in between channel openings 2206 and 2210 (not shown). Here, the channels may have a curvature is coextensive with the convex curvature of top surface 2202. Peripheral protrusions 2212 and 2214 may be disposed on opposite sides of channel openings 2206. Peripheral protrusions 2212 and 2214 may be substantially spherical and have circular cross sections. In still other examples, device 2200 and the elements shown and described may be designed, configured, formed, modified, or implemented apart from the examples shown or described and are not limited to those provided.

FIG. 22G illustrates a perspective view of an exemplary implantable interpositional orthopedic pain management apparatus. Here, a perspective view of device 2200 includes top surface 2202, channel openings 2204-2210, peripheral protrusions 2212-2218, perimeter 2220, bottom surface 2232, and contours 228. As used herein, like numbered and/or like named elements are assumed to be referencing the same or a substantially similar element having the same or substantially similar function or structure. Differences in function or structure may be described separately with regard to a specific feature described. In the example shown, top surface 2202 is substantially curved and is four-sided. The curvature of bottom surface 2232 is substantially the opposite of the curvature of top surface 2202. In some examples, device 2200 may include four peripheral protrusions 2212-2218 disposed on four corners on a four-sided top surface 2202. Peripheral protrusions 2212-2218 may be formed with both top and bottom surfaces 2202, 2232, such that a portion of a peripheral protrusion is substantially disposed on top surface 2202 and a portion is substantially disposed on bottom surface 2232. The shape and curvature of top surface 2202, and bottom surface 2232, and positions and sizes of peripheral protrusions 2212-2218 are selected to fit in between two adjacent bones in a joint. Top and/or bottom surface(s) 2202, 2232, once device 2200 is surgically implanted within a joint, may be in dynamic contact with adjacent bone surfaces, which allows for movement of device 2200 within the joint. In other words, portions of device 2200 such as top and bottom surfaces 2202 and 2232, and peripheral protrusions 2212-2218 may be in intermittent contact with adjacent bones. The rounded or curved nature of peripheral protrusions 2212-2218 may, in some examples, aid in allowing for dynamic adjustment of device 2200 once implanted, such that the surfaces allow for smooth movement against adjacent bone surfaces, without abrupt corners. In other examples, device 2200 may be implanted in between capitate and lunate bones to alleviate discomfort associated with bone degradation and wear. In other examples, device 2200 and the elements shown and described may be designed, configured, formed, modified, or implemented apart from the examples shown or described and are not limited to those provided.

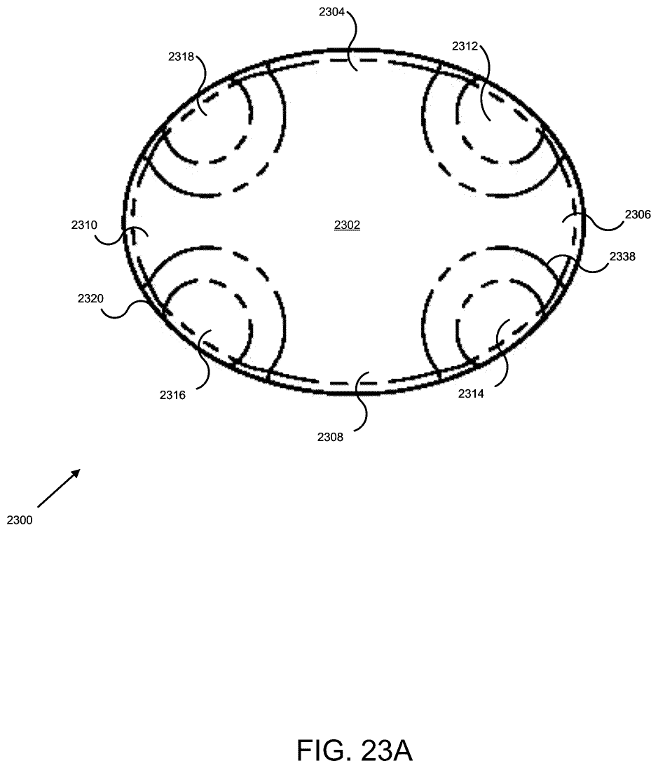

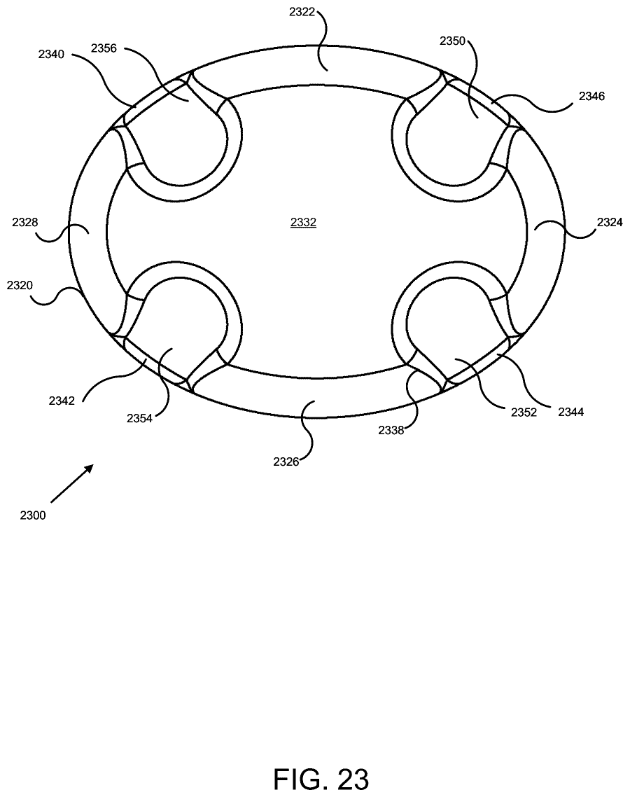

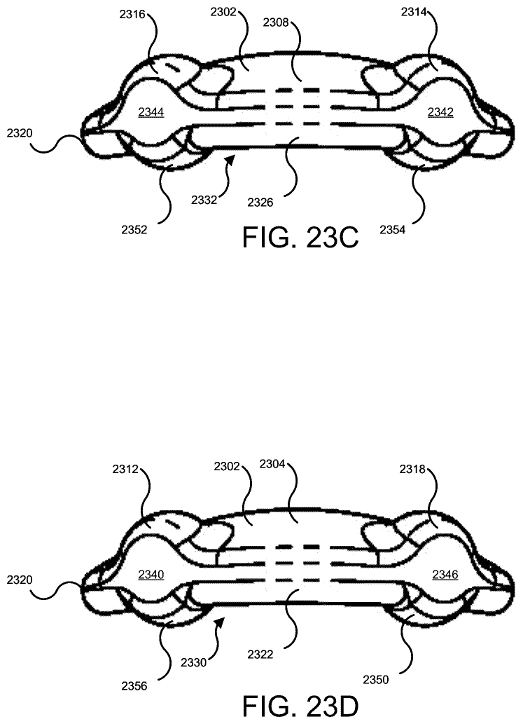

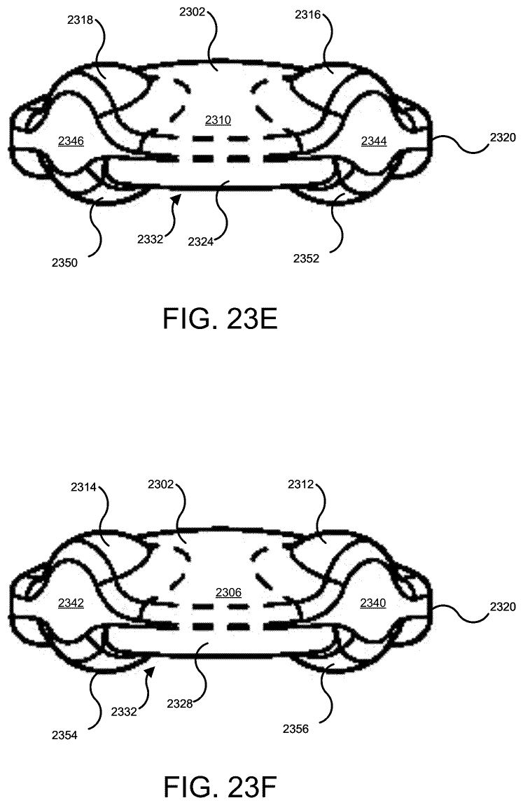

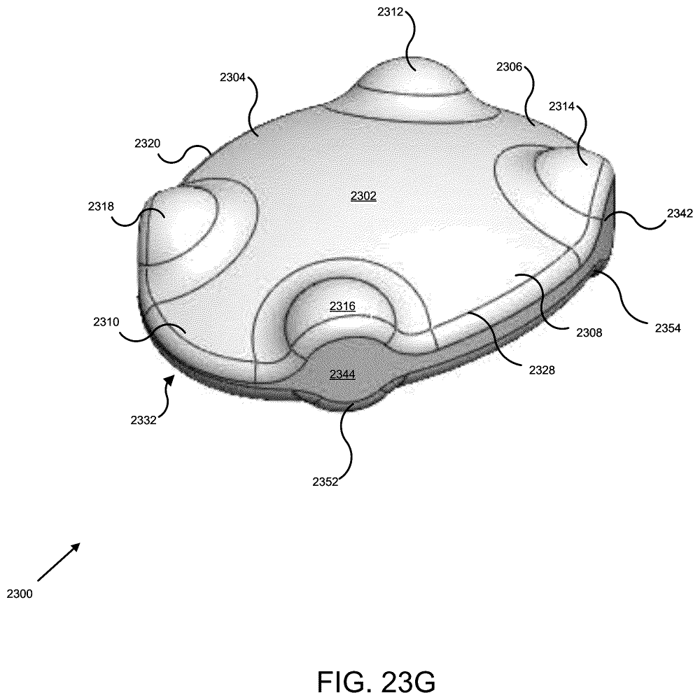

FIG. 23A illustrates a top view of an exemplary implantable interpositional orthopedic pain management apparatus. Here, device 2300 includes top surface 2302, channel openings 2304-2310, peripheral protrusions 2312-2318, perimeter 2320, and contours 2338. As used herein, like numbered and/or like named elements are assumed to be referencing the same or a substantially similar element having the same or substantially similar function or structure. Differences in function or structure may be described separately with regard to a specific feature described. Top surface 2302 may have a substantially rounded or ovular shape, with peripheral protrusions 2312-2318 disposed substantially on perimeter 2320. Top surface 2302 may be substantially convex, and shaped to fit, or be positioned against, a bone surface in a joint. In other examples, top surface 2302 may be substantially concave. Peripheral protrusions 2312-2318 may have rounded or partial-spherical dimensions, and in some examples, function to retain device 2300 in a synovial capsule and/or joint, once surgically implanted. In some examples, peripheral protrusions 2312-2318 are disposed in their entirety on top surface 2302. Channel openings 2304-2310 may be disposed in between two peripheral protrusions 2312-2318. Peripheral protrusions 2312-2318 may be positioned in between two proximally-positioned channel openings 2304-2310. Channel openings on opposite sides of top surface 2302 such as 2304 and 2308 may define a channel across top surface 2302. Similarly, channel openings 2306 and 2310 may define a channel substantially orthogonal to the channel between channel openings 2304 and 2308. A channel may include a portion of top surface 2302 between channel openings, such as channel openings 2304 and 2308. A channel opening 2304-2310 may be substantially disposed at or near perimeter 2320. Device 2300 may include additional surface elements as generally identified by contours 2338. For example, device 2300 may include an additional curved surface element in between peripheral protrusion 2314 and top surface 2302, as illustrated by contours 2338. In other examples, as in the previously and following described figures, the number, shape, type, quantity, and disposition of peripheral protrusions 2312-2318 may be varied and are not limited to those shown and described. The number, shape, type, quantity, and disposition of peripheral protrusions 2312-2318 may be selected to be positioned within non-articulating portions of a synovial capsule and/or joint, and alleviates pain by preventing contact between distal and proximal ends of bones in a joint, and peripheral protrusions prevent dislocation and expulsion of device 2300 from the joint. In other examples, device 2300 and the elements shown and described may be designed, configured, formed, modified, or implemented apart from the examples shown or described and are not limited to those provided.

FIG. 23B illustrates a bottom view of an exemplary implantable interpositional orthopedic pain management apparatus. Here, device 2300 includes perimeter 2020, channel openings 2322-2328, bottom surface 2332, outward facing vertical surfaces 2340-2346, peripheral protrusions 2350-2356, and contours 2338. As used herein, like numbered and/or like named elements are assumed to be referencing the same or a substantially similar element having the same or substantially similar function or structure. Differences in function or structure may be described separately with regard to a specific feature described. In the example herein, bottom surface 2332 may have a radius of curvature such that it is substantially concave to receive (or otherwise be shaped to substantially fit) an end of a bone. In other examples, bottom surface 2332 may have a radius of curvature such that it is substantially convex. Bottom surface 2332 may have channel openings 2322-2328, from which channels on bottom surface 2332 may be defined, similar to channel openings 2304-2310 (FIG. 23A). In some examples, peripheral protrusions 2350-2356 may be formed with bottom surface 2332 and be shaped to support device 2300 within a joint once surgically implanted. In other examples, outward facing vertical surfaces 2340-2346 are disposed on a side of each peripheral protrusion 2350-2356, the surfaces being generally faced away from a central region of top or bottom surfaces 2302, 2332, and may have a curvature element similar to the curvature of perimeter 2320. In other examples, device 2300 and the elements shown and described may be designed, configured, formed, modified, or implemented apart from the examples shown or described and are not limited to those provided.

FIG. 23C illustrates an anterior view of an exemplary implantable interpositional orthopedic pain management apparatus. Here, an anterior or front view of device 2300 includes top surface 2302, channel openings 2308 and 2326, peripheral protrusions 2314, 2316, 2352, and 2354, perimeter 2320, and outward facing vertical surfaces 2342 and 2344. As used herein, like numbered and/or like named elements are assumed to be referencing the same or a substantially similar element having the same or substantially similar function or structure. Differences in function or structure may be described separately with regard to a specific feature described. Here, top surface 2302 is substantially convex, and bottom surface 2332 is substantially concave. However, the curvatures of such surfaces are not limited to those shown herein. Channel opening 2308 is shown disposed opposite of channel opening 2326. Channel opening 2308 may be disposed in between peripheral protrusions 2314 and 2316. The channel formed on top surface 2302 with channel openings 2308 and 2304 (not shown) may be shaped to receive an end of a bone. Channel opening 2326 may be disposed between peripheral protrusions 2352 and 2354. The channel formed on bottom surface 2332 with channel openings 2326 and 2322 may be shaped to receive an end of an opposing bone in the joint. In some examples, outward facing vertical surfaces 2340-2346 (not shown) may be disposed in between adjacent peripheral protrusions 2312-2318 (not shown) and 2350-2356 (not shown). For example, outward facing vertical surface 2342 is in between, or connects, peripheral protrusions 2314 and 2354, that are on opposite sides of device 2300. Outward facing vertical surfaces 2340-2346 (not shown) may have a shape that in part conforms to a portion of an adjacent peripheral protrusion 2312-2318 (not shown). In this view, outward facing vertical surface 2346 has curved portions that are substantially similar to the shape of adjacent peripheral protrusions 2318 and 2350, and pointed portions where the curved portions intersect. In some examples, outward facing vertical surfaces 2340-2346 (not shown) may be partially bulb-shaped, with a rounded portion and a pointed portion. In other examples, device 2300 and the elements shown and described may be designed, configured, formed, modified, or implemented apart from the examples shown or described and are not limited to those provided.

FIG. 23D illustrates a posterior view of an exemplary implantable interpositional orthopedic pain management apparatus. Here, a posterior view of device 2300 includes top surface 2302, channel openings 2304 and 2322, peripheral protrusions 2312, 2318, 2350, and 2356, perimeter 2320, and outward facing vertical surfaces 2340 and 2346. As used herein, like numbered and/or like named elements are assumed to be referencing the same or a substantially similar element having the same or substantially similar function or structure. Differences in function or structure may be described separately with regard to a specific feature described. Elements herein are similar to those illustrated and described with reference to FIG. 23C. In some examples, channel openings 2304 and 2308 (not shown) form a channel configured to receive a bone in top surface 2302 when device 2300 is surgically implanted into a synovial capsule and joint. In some examples, channel openings 2322 and 2326 (not shown) form a channel configured to receive a bone in bottom surface 2332 when device 2300 is surgically implanted into a synovial capsule and joint. In other examples, device 2300 and the elements shown and described may be designed, configured, formed, modified, or implemented apart from the examples shown or described and are not limited to those provided.

FIG. 23E illustrates a left view of an exemplary implantable interpositional pain management apparatus. Here, a left view of device 2300 is shown, including top surface 2302, channel openings 2310 and 2324, peripheral protrusions 2316, 2318, 2350, and 2352, perimeter 2320, and outward facing vertical surfaces 2344 and 2346. As used herein, like numbered and/or like named elements are assumed to be referencing the same or a substantially similar element having the same or substantially similar function or structure. Differences in function or structure may be described separately with regard to a specific feature described. Here, top surface 2302 is substantially convex, and bottom surface 2332 is substantially concave. However, the curvatures of such surfaces are not limited to those shown herein. Channel opening 2310 is shown disposed opposite of channel opening 2324. Channel opening 2310 may be disposed in between peripheral protrusions 2316 and 2318. The channel formed on top surface 2302 with channel openings 2310 and 2306 (not shown) may be shaped to receive an end of a bone. Channel opening 2324 may be disposed between peripheral protrusions 2352 and 2350. The channel formed on bottom surface 2332 with channel openings 2324 and 2328 may be shaped to receive an end of an opposing bone in the joint. In some examples, outward facing vertical surfaces 2340-2346 (not shown) may be disposed in between adjacent peripheral protrusions 2312-2318 (not shown) and 2350-2356 (not shown). For example, outward facing vertical surface 2344 is in between, or connects, peripheral protrusions 2316 and 2352. Device 2300 may be surgically implanted in a joint, and oriented such that it prevents contact between surfaces of adjacent bones. Outward facing vertical surfaces 2344 and 2346 may have a curvature that aligns with perimeter 2320, such that device 2300 minimally interferes with lateral tissue or bones once surgically implanted within a joint. In other examples, device 2300 and the elements shown and described may be designed, configured, formed, modified, or implemented apart from the examples shown or described and are not limited to those provided.

FIG. 23F illustrates a right view of an exemplary implantable interpositional pain management apparatus. Here, a right view of device 2300 is shown, including top surface 2302, channel openings 2306 and 2328, peripheral protrusions 2312, 2314, 2354, and 2356, perimeter 2320, bottom surface 2332, and outward facing vertical surfaces 2342 and 2340. As used herein, like numbered and/or like named elements are assumed to be referencing the same or a substantially similar element having the same or substantially similar function or structure. Differences in function or structure may be described separately with regard to a specific feature described. In some examples, top surface 2302 is configured, with channel openings 2306 and 2310 (not shown), to receive a bone or a portion thereof when device 2300 is surgically implanted. Bottom surface 2332 may also be configured, with channel openings 2328 and 2324 (not shown), to receive a bone or a portion thereof when device 2300 is surgically implanted. For example, if device 2300 is surgically implanted into a CMC joint, top surface 2302 may be configured, with channel openings 2306 and 2310, to receive (i.e., top surface 2302 may have a radius of curvature that is similar to that of a trapezium bone) a trapezium bone, or other bone adjacent to the joint. Outward facing vertical surfaces 2340 and 2342 may have a curvature that aligns with perimeter 2320, such that device 2300 minimally interferes with lateral tissue or bones once surgically implanted within a joint. In other examples, device 2300 and the elements shown and described may be designed, configured, formed, modified, or implemented apart from the examples shown or described and are not limited to those provided.

FIG. 23G illustrates a perspective view of an exemplary implantable interpositional orthopedic pain management apparatus. Here, device 2300 includes top surface 2302, channel openings 2304-2310, peripheral protrusions 2312-2318, 2352, and 2354, perimeter 2320, bottom surface 2332, outward facing vertical surfaces 2342 and 2344, and contours 2328. As used herein, like numbered and/or like named elements are assumed to be referencing the same or a substantially similar element having the same or substantially similar function or structure. Differences in function or structure may be described separately with regard to a specific feature described. As shown, top surface 2302 is substantially convex, and has an ovular shape. Perimeter 2320 encompasses the ovular shape of top surface 2302. Peripheral protrusions 2312-2318 are disposed along perimeter 2320, and peripheral protrusions 2350-2356 (not shown) are similarly disposed along perimeter 2320, on bottom surface 2332, opposite peripheral protrusions 2312-2318. Outward facing vertical surface 2344 may be disposed between opposing peripheral protrusions 2316 and 2352, for example. In some examples, outward facing vertical surfaces 2340-2344 may be formed with perimeter 2320 and associated peripheral protrusions 2312-2318 and 2350-2356. Channel opening 2304 indicates an opening to a channel formed with top surface 2302 and channel opening 2308. Similarly, channel opening 2306 indicates an opening to a channel formed with top surface 2302 and channel opening 2310. Contours 2328 may indicate additional surface features such as a change in curvature of top surface 2302 near perimeter 2320. Once, implanted, outward facing vertical surface 2340-2346 (not shown) and peripheral protrusions 2312-2318 may be in intermittent contact with adjacent bone surfaces and function to oppose dislodging forces applied to device 2300. By being in intermittent contact with adjacent bone surfaces, device 2300 may be dynamically adjusted as the joint is moved. As with the other examples throughout this description, various radii of curvature may be used and are not limited to the examples shown and described. In other examples, device 2300 and the elements shown and described may be designed, configured, formed, modified, or implemented apart from the examples shown or described and are not limited to those provided.

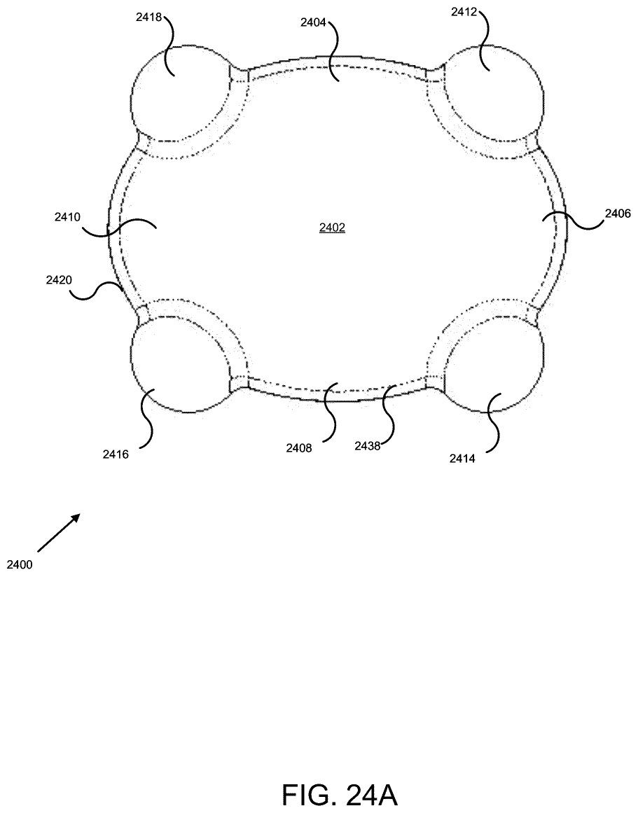

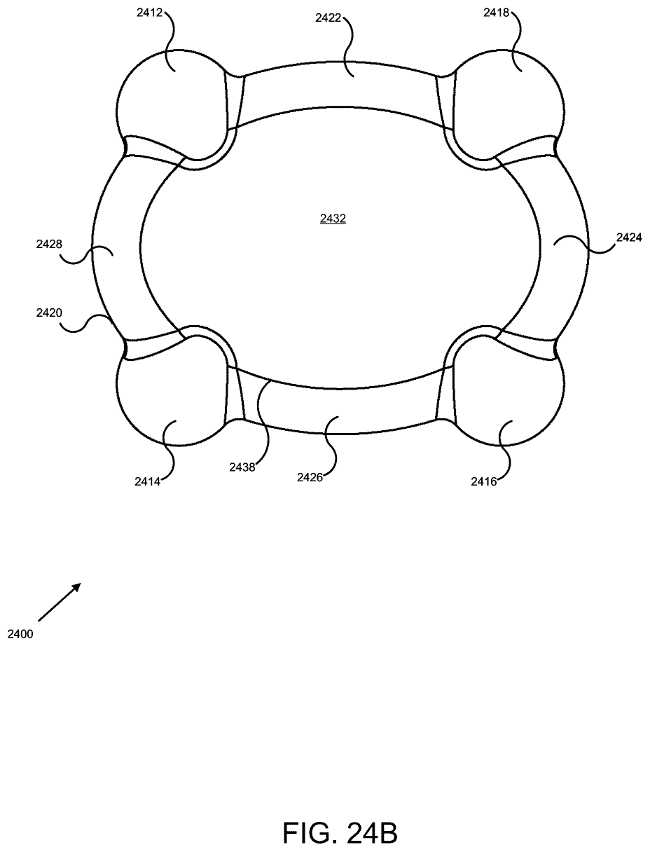

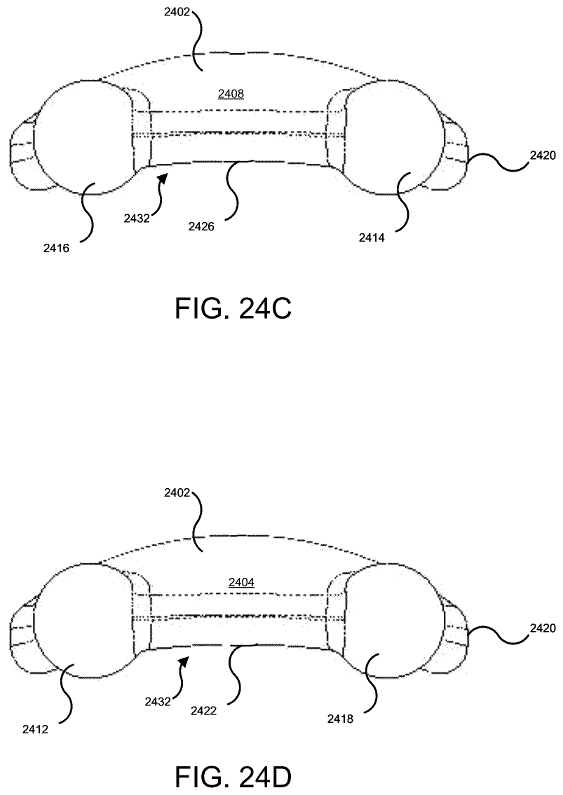

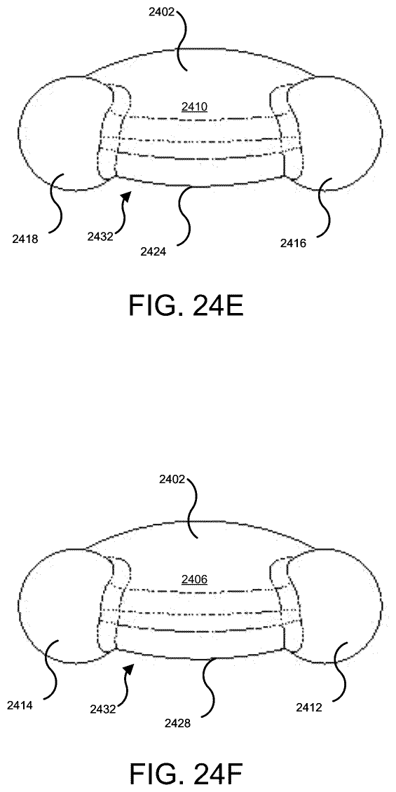

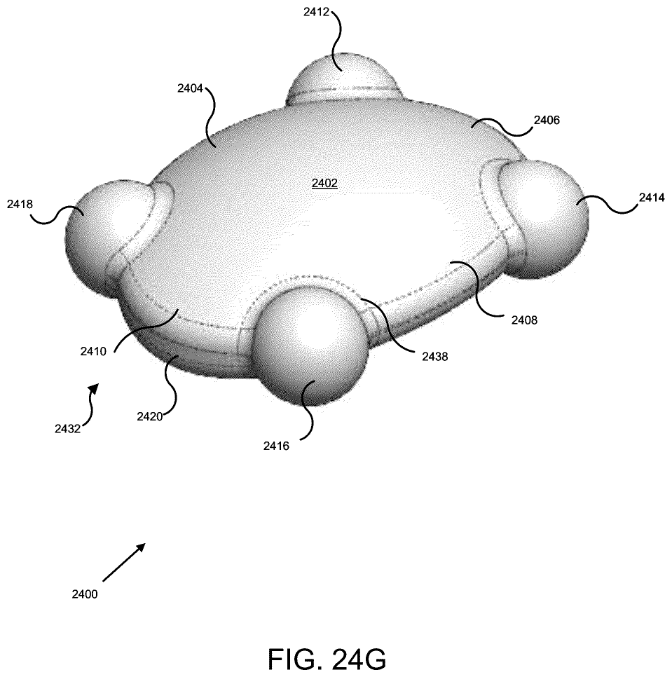

FIG. 24A illustrates a top view of an exemplary implantable interpositional orthopedic pain management apparatus. Here, device 2400 includes top surface 2402, channel openings 2402-2410, peripheral protrusions 2412-2418, perimeter 2420, and contours. As used herein, like numbered and/or like named elements are assumed to be referencing the same or a substantially similar element having the same or substantially similar function or structure. Top surface 2402 may have a substantially rounded or ovular shape, with peripheral protrusions 2412-2418 disposed substantially on perimeter 2420. Top surface 2402 may be substantially convex, and shaped to fit, or be positioned against, a bone surface in a joint. In other examples, top surface 2402 may be substantially concave. Peripheral protrusions 2412-2418 may be spherical in shape or have rounded portions, and in some examples, function to retain device 2400 in a synovial capsule and/or joint, once surgically implanted. Peripheral protrusions 2412-2418 may have substantially circular cross sections. In some examples, peripheral protrusions 2412-2418 may be formed with top surface 2402 and have portions that extend beyond perimeter 2420, from a top view. Channel openings 2404-2410 may be disposed in between peripheral protrusions 2412-2418. Channel openings on opposite sides of top surface 2402, such as 2404 and 2408, may define a channel across top surface 2402. Similarly, channel openings 2406 and 2410 may define a channel substantially orthogonal to the channel between channel openings 2404 and 2408. Device 2400 may include additional surface elements as generally identified by contours 2438. For example, device 2400 may include an additional curved surface element in between peripheral protrusion 2414 and top surface 2402, as illustrated generally by contours 2438. In other examples, as in the previously and following described figures, the number, shape, type, quantity, and disposition of peripheral protrusions 2412-2418 may be varied and are not limited to those shown and described. The number, shape, type, quantity, and disposition of peripheral protrusions 2412-2418 may be selected to be positioned within non-articulating portions of a synovial capsule and/or joint, and alleviates pain by preventing contact between distal and proximal ends of bones in a joint, and peripheral protrusions prevent dislocation and expulsion of device 2400 from the joint. In other examples, device 2400 and the elements shown and described may be designed, configured, formed, modified, or implemented apart from the examples shown or described and are not limited to those provided.

FIG. 24B illustrates a bottom view of an exemplary implantable interpositional orthopedic pain management apparatus. Here, device 2400 includes perimeter 2420, channel openings 2422-2428, bottom surface 2432, peripheral protrusions 2412-2418, and contours. As used herein, like numbered and/or like named elements are assumed to be referencing the same or a substantially similar element having the same or substantially similar function or structure. Differences in function or structure may be described separately with regard to a specific feature described. In the example herein, bottom surface 2432 may have at least a portion with a radius of curvature such that it is substantially concave to receive (or otherwise be shaped to substantially fit) an end of a bone. In other examples, bottom surface 2432 may have a radius of curvature such that it is substantially convex. Bottom surface 2432 may have channel openings 2422-2428, from which channels on bottom surface 2432 may be defined, similar to channel openings 2404-2410 (FIG. 24A). In some examples, peripheral protrusions 2412-2418 may be formed with bottom surface 2432, in part be formed with and disposed on top surface 2402 (not shown), and be shaped to support device 2400 within a joint once surgically implanted. In other examples, device 2400 and the elements shown and described may be designed, configured, formed, modified, or implemented apart from the examples shown or described and are not limited to those provided.

FIG. 24C illustrates an anterior view of an exemplary implantable interpositional orthopedic pain management apparatus. Here, an anterior or front view of device 2400 includes top surface 2402, channel openings 2408 and 2426, peripheral protrusions 2414 and 2416, and perimeter 2420. As used herein, like numbered and/or like named elements are assumed to be referencing the same or a substantially similar element having the same or substantially similar function or structure. Differences in function or structure may be described separately with regard to a specific feature described. Here, top surface 2402 is substantially convex, and bottom surface 2432 is substantially concave. However, the curvatures of such surfaces are not limited to those shown herein. Top surface 2402 and bottom surface 2432 may be configured and shaped to fit in between two opposing bone surfaces in a joint, thus preventing the bone surfaces from coming into contact. In some examples, top surface 2402 and bottom surface 2432 may be in dynamic contact with each bone surface, and not be directly coupled or permanently or semi-permanently joined to either bone surface. In other words, after device 2400 is surgically implanted in a joint, different portions of device 2400 may be in contact with the bone surfaces of the joint depending on movement of the joint (e.g., flexion and extension). Channel opening 2408 is shown disposed opposite of channel opening 2426. Channel opening 2406 may be disposed in between peripheral protrusions 2414 and 2416. The channel formed on top surface 2402 with channel openings 2408 and 2404 (not shown) may be shaped to receive an end of a bone. Channel opening 2426 may be disposed between peripheral protrusions 2414 and 2416. The channel formed on bottom surface 2432 with channel openings 2426 and 2422 may be shaped to receive an end of an opposing bone in the joint. Peripheral protrusions 2412-2418 may be at least partially spherical and shape, and span top and bottom surfaces 2402 and 2432. In other words, a portion of peripheral protrusions 2412-2418 may be disposed on and protrude out of top surface 2402, and a portion may be disposed on and protrude out of bottom surface 2432. In other examples, device 2400 and the elements shown and described may be designed, configured, formed, modified, or implemented apart from the examples shown or described and are not limited to those provided.

FIG. 24D illustrates a posterior view of an exemplary implantable interpositional orthopedic pain management apparatus. Here, a posterior view of device 2400 includes top surface 2402, channel openings 2402 and 2422, peripheral protrusions 2412 and 2418, and perimeter 2420. As used herein, like numbered and/or like named elements are assumed to be referencing the same or a substantially similar element having the same or substantially similar function or structure. Differences in function or structure may be described separately with regard to a specific feature described. Elements shown herein are substantially similar to those described and illustrated in reference to FIG. 24C. In some examples, channel openings 2404 and 2408 (not shown) form a channel configured to receive a bone in top surface 2402 when device 2400 is surgically implanted into a synovial capsule and joint. In some examples, channel openings 2422 and 2426 (not shown) form a channel configured to receive a bone in bottom surface 2432 when device 2400 is surgically implanted into a synovial capsule and joint. In other examples, device 2400 and the elements shown and described may be designed, configured, formed, modified, or implemented apart from the examples shown or described and are not limited to those provided.

FIG. 24E illustrates a left view of an exemplary implantable interpositional pain management apparatus. Here, a left view of device 2400 is shown, including top surface 2402, channel openings 2410 and 2424, and peripheral protrusions 2416 and 2418. As used herein, like numbered and/or like named elements are assumed to be referencing the same or a substantially similar element having the same or substantially similar function or structure. Differences in function or structure may be described separately with regard to a specific feature described. Here, top surface 2402 is substantially convex, and bottom surface 2432 is substantially concave. However, the curvatures of such surfaces are not limited to those shown herein. Channel opening 2410 is shown disposed opposite of channel opening 2424. Channel opening 2410 may be disposed in between peripheral protrusions 2416 and 2418. The channel formed on top surface 2402 with channel openings 2410 and 2406 (not shown) may be shaped to receive an end of a bone. Channel opening 2424 may be disposed between peripheral protrusions 2416 and 2418. The channel formed on bottom surface 2432 with channel openings 2424 and 2428 may be shaped to receive an end of an opposing bone in the joint. Device 2400 may be surgically implanted in a joint, and oriented such that it prevents contact between surfaces of adjacent bones. Peripheral protrusions 2412-2418 may function to retain device 2400 within the joint by interacting with adjacent bone surfaces, thereby preventing expulsion of device 2400 from the joint. In other examples, device 2400 and the elements shown and described may be designed, configured, formed, modified, or implemented apart from the examples shown or described and are not limited to those provided.

FIG. 24F illustrates a right view of an exemplary implantable interpositional pain management apparatus. Here, a right view of device 2400 is shown, including top surface 2402, channel openings 2406 and 2428, peripheral protrusions 2412 and 2412, and bottom surface 2432. As used herein, like numbered and/or like named elements are assumed to be referencing the same or a substantially similar element having the same or substantially similar function or structure. Differences in function or structure may be described separately with regard to a specific feature described. Elements shown herein are substantially similar to those described and illustrated in reference to FIG. 24E. In some examples, top surface 2402 is configured, with channel openings 2406 and 2410 (not shown), to receive a bone or a portion thereof when device 2400 is surgically implanted. Bottom surface 2432 (indicated with a floating arrow) may also be configured, with channel openings 2428 and 2424 (not shown), to receive a bone or a portion thereof when device 2400 is surgically implanted. For example, if device 2400 is surgically implanted into a CMC joint, top surface 2402 may be configured, with channel openings 2406 and 2410, to receive (i.e., top surface 2402 may have a radius of curvature that is similar to that of a trapezium bone) a trapezium bone, or other bone adjacent to the joint. In other examples, device 2400 and the elements shown and described may be designed, configured, formed, modified, or implemented apart from the examples shown or described and are not limited to those provided.

FIG. 24G illustrates a perspective view of an exemplary implantable interpositional orthopedic pain management apparatus. Here, device 2400 includes top surface 2402, channel openings 2404-2410, peripheral protrusions 2412-2418, perimeter 2420, bottom surface 2432, and contours 2438. As used herein, like numbered and/or like named elements are assumed to be referencing the same or a substantially similar element having the same or substantially similar function or structure. Differences in function or structure may be described separately with regard to a specific feature described. As shown, top surface 2402 is substantially convex, and has a rounded, or ovular shape. Peripheral protrusions 2412-2418 may be disposed along perimeter 2420, and may span, or otherwise be formed with, top surface 2402 and bottom surface 2432. Channel opening 2404 indicates an opening to a channel formed with top surface 2402 and channel opening 2404. Similarly, channel opening 2406 indicates an opening to a substantially orthogonal or intersecting channel (to the channel formed between channel openings 2404 and 2408) formed with top surface 2402 and channel opening 2410. Contours 2438 may include portions of device 2400 having a change in surface shape, such as between peripheral protrusion 2416 and top surface 2402. As with the other examples throughout this description, various radii of curvature may be used and are not limited to the examples shown and described. Once implanted into a joint, peripheral protrusions 2412-2418 may function to retain device 2400 in the joint by resisting forces applied to device 2700, and be physical barriers to prevent lateral expulsion by resisting movement against adjacent bone surfaces. In other examples, device 2400 and the elements shown and described may be designed, configured, formed, modified, or implemented apart from the examples shown or described and are not limited to those provided.

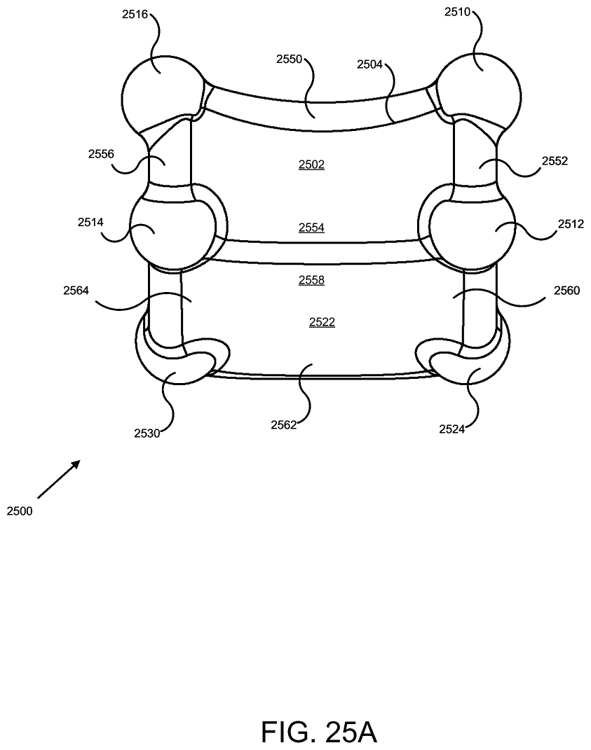

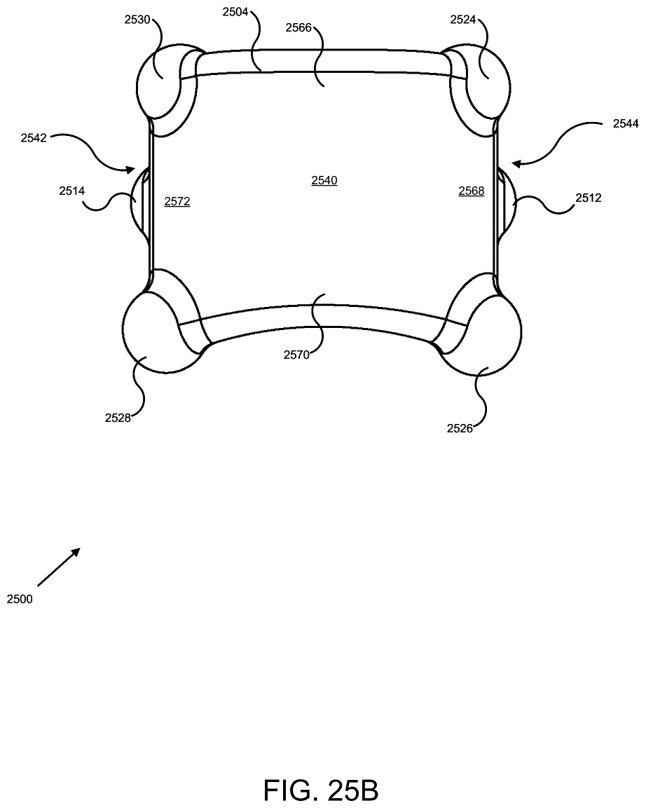

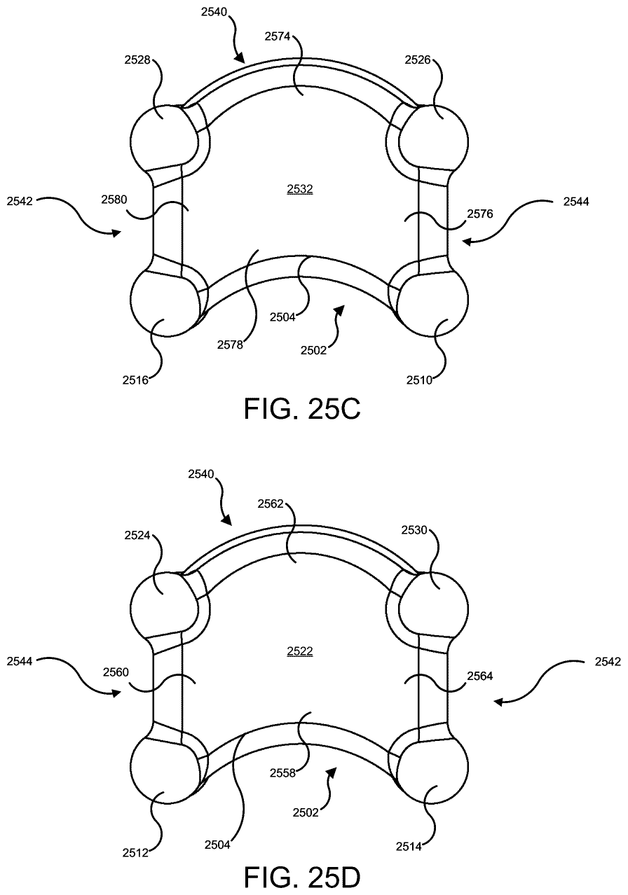

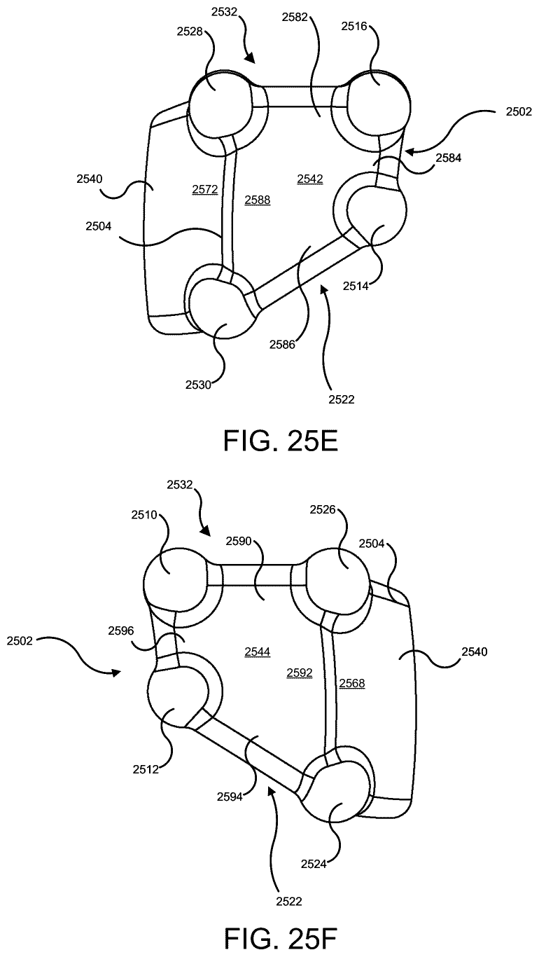

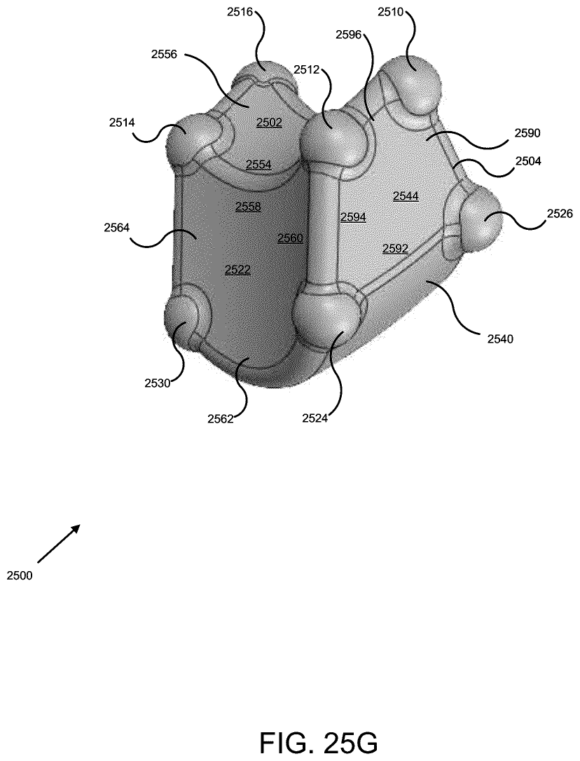

FIG. 25A illustrates a top view of an exemplary implantable interpositional orthopedic pain management apparatus. Here, device 2500 includes a top surface 2502, contours 2504, front surface 2522, peripheral protrusions 2510-2516, 2524, and 2530, and channel openings 2550-2564. As used herein, like numbered and/or like named elements are assumed to be referencing the same or a substantially similar element having the same or substantially similar function or structure. Differences in function or structure may be described separately with regard to a specific feature described. Here, top surface 2502 is substantially concave and four-sided. In other examples, top surface 2502 may be substantially convex, or have a radius of curvature different to that shown herein. Front surface 2522, shown adjacent to top surface 2502, may be substantially concave, convex, or be substantially flat. Front surface 2522 may be substantially four sided, and may share one side with a side of top surface 2502. The radii of curvature or surface characteristics of top and front surfaces 2502 and 2522 may be selected to substantially fit a surface of a bone in a joint, once device 2500 is surgically implanted. Peripheral protrusions 2510-2516 may be disposed or formed with top surface 2502, and may be disposed in part on the corners of top surface 2502. Peripheral protrusions 2512, 2514, 2524, and 2530 may be disposed or formed with front surface 2522, and may be disposed in part on the corners of front surface 2522. Peripheral protrusions 2510-2516, 2524, and 2530 may be disposed on intersections of adjacent surfaces, such as peripheral protrusions 2512 and 2514 disposed on the intersection of top surface 2502 and front surface 2522. In some examples, top and front surfaces 2502 and 2522 may include channel openings 2550-2564, which may be disposed in between two peripheral protrusions, and indicate an opening to a channel on the surface. Peripheral protrusions may also be disposed in between proximally-positioned channel openings, such as peripheral protrusion 2512 disposed in between channel openings 2552 and 2560. On top surface 2502, a channel may be formed in between channel openings 2550 and 2554, and a substantially orthogonal channel may be formed in between channel openings 2552 and 2556. On front surface 2522, a channel may be formed in between channel openings 2558 and 2562, and a substantially orthogonal channel may be formed in between channel openings 2560 and 2564. Contours 2504 may be generally indicated as additional surface elements of device 2500. For example, a change in curvature or shape may be in between top surface 2502 and front surface 2522, may be generally shown by contours 2504, indicating a change in curvature or shape. Peripheral protrusions 2512, 2514, 2524, and 2530 may be formed and integrated with surfaces 2502, 2522 to provide pronounced structures that, when device 2500 is surgically implanted in a joint, may be configured to interact within articulating or non-articulating regions (or, in some examples, in a combination of articulating and non-articulating regions) of a joint. Device 2500 is configured to permit motion or articulation of a joint without dislocation or expulsion of device 2500 from the joint. In some examples, device 2500 may be implanted and used replacement of a bone in a joint, in between two adjacent bones. Once implanted, peripheral protrusions 2512, 2514, 2524, and 2530, top surface 2502, and front surface 2522 may be configured to be in intermittent contact with adjacent bone surfaces, allowing for dynamic interaction of device 2500 within a joint. In other examples, device 2500 and the elements shown and described may be designed, configured, formed, modified, or implemented apart from the examples shown or described and are not limited to those provided.

FIG. 25B illustrates a bottom view of an exemplary implantable interpositional orthopedic pain management apparatus. Here, device 2500 includes contours 2504, bottom surface 2540, peripheral protrusions 2512, 2514, and 2524-2530, side surfaces 2542 and 2544, and channel openings 2566-2572. As used herein, like numbered and/or like named elements are assumed to be referencing the same or a substantially similar element having the same or substantially similar function or structure. Differences in function or structure may be described separately with regard to a specific feature described. Here, bottom surface 2540 is has a radius of curvature such that it is substantially convex. In other examples, bottom surface 2540 may be substantially concave, or has at least a portion with a radius of curvature selected to fit with a portion of or an end of a bone. Bottom surface 2540 may be substantially four-sided, with peripheral protrusions disposed on or formed with four corners of bottom surface 2540. Portions of peripheral protrusions 2512 and 2514, disposed substantially on the intersection of top surface 2502 and front surface 2522, are also viewable. Bottom surface 2540 may include channel openings 2566-2572. A channel may be formed on bottom surface 2540 in between channel openings 2566 and 2570. A substantially intersecting channel may be formed on bottom surface 2540 in between channel openings 2568 and 2572. Locations of side surfaces 2542 and 2544 are indicated by floating arrows and will be described in greater detail with reference to FIGS. 25E and 25F. Side surfaces 2542 and 2544 may be substantially parallel to one another and may have substantially similar radii of curvature. In other examples, device 2500 and the elements shown and described may be designed, configured, formed, modified, or implemented apart from the examples shown or described and are not limited to those provided.

FIG. 25C illustrates an anterior view of an exemplary implantable interpositional orthopedic pain management apparatus. Here, an anterior or front view of device 2500 includes top surface 2502, contours 2504, peripheral protrusions 2510, 2516, 2526, and 2528, back surface 2532, bottom surface 2540, side surfaces 2542 and 2544, and channel openings 2574-2580. As used herein, like numbered and/or like named elements are assumed to be referencing the same or a substantially similar element having the same or substantially similar function or structure. Differences in function or structure may be described separately with regard to a specific feature described. Here, back surface 2532 may be substantially concave, or otherwise have a radius of curvature selected to fit a portion of or an end of a bone. Back surface 2532 may be substantially four-sided, with peripheral protrusions 2510, 2516, 2526, and 2528 disposed at least partially on the corners of back surface 2532. Peripheral protrusions 2510, 2516, 2526, and 2528 may also be substantially disposed at an intersection of at least two surfaces. Here, peripheral protrusion 2526 may be substantially disposed at an intersection of back surface 2532, bottom surface 2540, and side surface 2544 (bottom surface 2540 and side surface 2544 are identified generally by floating arrows). The intersection of back surface 2532, bottom surface 2540, and side surface 2544 may also be referred to as a three-dimensional corner of device 2500. In another example, peripheral protrusion 2528 may be substantially disposed or formed with an intersection of back surface 2532, bottom surface 2540, and side surface 2542. Back surface 2532 may also include channel openings 2574-2580. A channel may be formed on back surface 2532 between channel openings 2574 and 2478. A channel may also be formed on back surface 2532 between channel openings 2576 and 2580. A channel may be shaped to fit against, or be in intermittent contact with, a bone surface in a target joint. Once device 2500 is implanted, peripheral protrusions 2510, 2516, 2526, and 2528 may be positioned to cradle or hold device 2500 in between adjacent bone surfaces. In some examples, side surfaces 2542 and 2544 may be substantially parallel to one another, or have radii of curvature that are substantially similar. In other examples, device 2500 and the elements shown and described may be designed, configured, formed, modified, or implemented apart from the examples shown or described and are not limited to those provided.

FIG. 25D illustrates a posterior view of an exemplary implantable interpositional orthopedic pain management apparatus. Here, a posterior or back view of device 2500 includes top surface 2502, contours 2504, peripheral protrusions 2512, 2514, 2524, and 2530, front surface 2522, bottom surface 2540, side surfaces 2542 and 2544, and channel openings 2558-2562. As used herein, like numbered and/or like named elements are assumed to be referencing the same or a substantially similar element having the same or substantially similar function or structure. Differences in function or structure may be described separately with regard to a specific feature described. Here, front surface 2522 may have a radius of curvature such that it may substantially fit a portion of or an end of a bone in a joint. In some examples, front surface 2522 may have a portion that is substantially concave. Front surface 2522 may be substantially four-sided with peripheral protrusions 2512, 2514, 2524, and 2530, formed with or disposed on corners of front surface 2522. Peripheral protrusions 2512, 2514, 2524, and 2530 may be formed with or disposed on an intersection of surfaces. For example, peripheral protrusion 2530 may be disposed on an intersection of front surface 2522, bottom surface 2540, and side surface 2542. Front surface 2522 may include channel openings 2558-2562. Channel openings 2558-2562 may be formed in between adjacent peripheral protrusions, such as peripheral protrusions 2524 and 2530. A channel may be formed in between channel openings 2562 and 2558. An intersecting channel may be formed in between channel openings 2560 and 2564. Channels formed between channel openings 2558-2564 may be coextensive with front surface 2522 and define a portion of front surface 2522 against which a bone may fit against once device 2500 is surgically implanted. In other examples, device 2500 and the elements shown and described may be designed, configured, formed, modified, or implemented apart from the examples shown or described and are not limited to those provided.

FIG. 25E illustrates a left view of an exemplary implantable interpositional orthopedic pain management apparatus. Here, a left side view of device 2500 includes top surface 2502, contours 2504, peripheral protrusions 2514, 2516. 2528, and 2530, front surface 2522, back surface 2532, bottom surface 2540, side surface 2542, and channel openings 2572 and 2582-2588. As used herein, like numbered and/or like named elements are assumed to be referencing the same or a substantially similar element having the same or substantially similar function or structure. Differences in function or structure may be described separately with regard to a specific feature described. Here, side surface 2542 is substantially trapezoidally shaped and may be substantially parallel to side surface 2544 (not shown). Side surface 2542 may be generally four-sided with peripheral protrusions 2514, 2516, 2528, and 2530 disposed on or formed with corners of side surface 2542. As described with respect to other examples, peripheral protrusions 2514, 2516, 2528, and 2530 may be formed with or disposed on the intersection of surfaces. For example, peripheral protrusion 2528 may be formed with or disposed on the intersection of side surface 2542, bottom surface 2540, and back surface 2532 (indicated by floating arrow). The position of peripheral protrusions 2514, 2516, 2528, and 2530 may be selected such that device 2500, once implanted in a joint, is in intermittent or non-permanent contact with adjacent bones. In some examples, peripheral protrusions 2514, 2516, 2528, and 2530 may be in dynamic contact with adjacent bones depending on movement of the joint (e.g., flexion, extension, rotation, etc.). Device 2500 may also be configured in a manner to minimize likelihood of dislodgement or expulsion of device, particularly when joint movement causes adjacent bone surfaces to apply forces to 2500. Device 2500 may include channel openings 2582-2588, each disposed substantially in between two peripheral protrusions. For example, channel opening 2584 is disposed between peripheral protrusions 2514 and 2516. Side surface 2542 may include dual intersecting channels, a first between channel openings 2582 and 2586, a second in between channel openings 2584 and 2588. Channel opening 2572, disposed on bottom surface 2540, may be separated from channel opening 2588 by an intersection of bottom surface 2540 and side surface 2542, and may be identified by contours 2504. Contours 2504, in this example, may include a change in surface curvature or shape in between bottom surface 2540 and side surface 2542. Contours 2504 may also indicate a change in surface curvature between a peripheral protrusion (such as peripheral protrusion 2514) and a surface (such as side surface 2542). In other examples, device 2500 and the elements shown and described may be designed, configured, formed, modified, or implemented apart from the examples shown or described and are not limited to those provided.

FIG. 25F illustrates a right view of an exemplary implantable interpositional orthopedic pain management apparatus. Here, a right side view of device 2500 includes top surface 2502, contours 2504, peripheral protrusions 2510, 2512, 2524, and 2526, front surface 2522, back surface 2532, bottom surface 2540, side surface 2544, and channel openings 2568 and 2590-2596 As used herein, like numbered and/or like named elements are assumed to be referencing the same or a substantially similar element having the same or substantially similar function or structure. Differences in function or structure may be described separately with regard to a specific feature described. Elements herein are substantially similar those illustrated and described in reference to FIG. 25E. Side surface 2544 is trapezoidally shaped and substantially parallel to side surface 2542 and has a substantially similar radius of curvature as side surface 2542 (FIG. 25E). Side surface 2544 may have substantially similar dimensions as side surface 2542. In some examples, side surface 2544 has the same or substantially similar trapezoidal shape as side surface 2542 (FIG. 25E). Side surface 2544 may be, or have a portion that is, substantially concave, substantially convex, or substantially flat. Side surface 2544 may include dual intersecting channels, a first disposed in between channel openings 2590 and 2594, and a second disposed in between channel openings 2592 and 2596. A peripheral protrusion may be disposed in between two channel openings (for example, peripheral protrusion 2526 is disposed in between channel openings 2590 and 2592). In some examples, a boundary between surfaces, such as between side surface 2544 and bottom surface 2540, may be defined by a change in surface characteristics, such a change of radii of curvature between side surface 2544 and bottom surface 2540. In other examples, device 2500 and the elements shown and described may be designed, configured, formed, modified, or implemented apart from the examples shown or described and are not limited to those provided.

FIG. 25G illustrates a perspective view of an exemplary implantable orthopedic pain management apparatus. Here, a perspective view of device 2500 includes top surface 2502, contours 2504, front surface 2522, bottom surface 2540, side surface 2544, peripheral protrusions 2510-2516, 2524, 2526, and 2530, channel openings 2554-2564, and 2590-2596. As used herein, like numbered and/or like named elements are assumed to be referencing the same or a substantially similar element having the same or substantially similar function or structure. Differences in function or structure may be described separately with regard to a specific feature described. Here, top surface 2502 is substantially concave, and is substantially four sided, with peripheral protrusions 2510-2516 disposed in part on corners of top surface 2502. Top surface 2502 may have a radii of curvature that is similar to, and directionally opposite that of bottom surface 2540. Peripheral protrusions 2512 and 2514 are disposed on and/or formed an intersection of top surface 2502 with front surface 2522. Peripheral protrusions 2510 and 2512 are disposed on and/or formed with an intersection of top surface 2502 and side surface 2544. Here, front surface 2522 is substantially concave, with channel openings 2558-2564. Side surface 2544 is substantially four-sided or trapezoidally shaped, with four channel openings 2590-2596. As partially seen herein, bottom surface may be substantially convex, and has an intersection with side surface 2544, where peripheral protrusions 2524 and 2526 are partially disposed, and an intersection with front surface 2522, where peripheral protrusions 2524 and 2530 are partially disposed. In some examples, contours 2504 may illustrate additional device 2500 elements such as a change in surface curvature between surfaces (such as between side surface 2544 and front surface 2522). Peripheral protrusions may be substantially spherical, with circular cross sections. However, the size, shape, number, and positions of peripheral protrusions is not limited to those shown herein, the quantity of the peripheral protrusions being determined by a type associated with the joint. In some examples, the rounded characteristic of peripheral protrusions 2510-2516, 2524, 2526, and 2530 permits smooth adjustment of device 2500 within the joint when the joint is moved or articulated. Peripheral protrusions 2510-2516, 2524, 2526, and 2530 may be configured to maintain anatomical alignment of device 2500 within the joint, once implanted and configured to move device 2500 in a direction that is substantially opposite to a force transmitted by a bone surface. In other examples, elements of device 2500 including top surface 2502, contours 2504, front surface 2522, bottom surface 2540, side surface 2544, peripheral protrusions 2510-2516, 2524, 2526, and 2530, channel openings 2554-2564, and 2590-2596 may also be configured to provide surfaces that are shaped to fit and/or fill with other materials within a synovial capsule such as cartilage, blood, bodily fluids, or other synthetic or organic materials that are injected or otherwise implanted to aid device 2500 in maintaining dynamic stability and position within a joint after surgical implantation. In other examples, device 2500 may be configured and used to replace a bone in a joint. In other examples, device 2500 and the elements shown and described may be designed, configured, formed, modified, or implemented apart from the examples shown or described and are not limited to those provided.

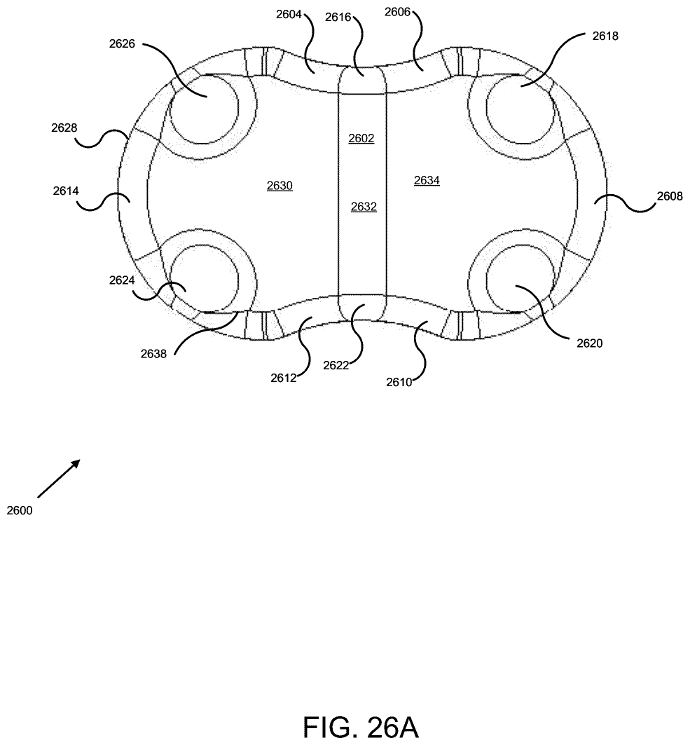

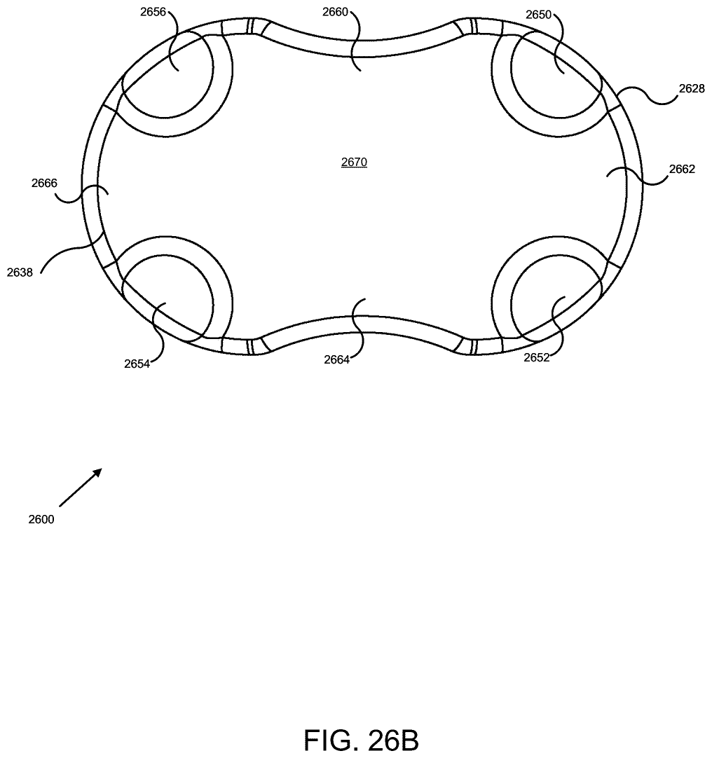

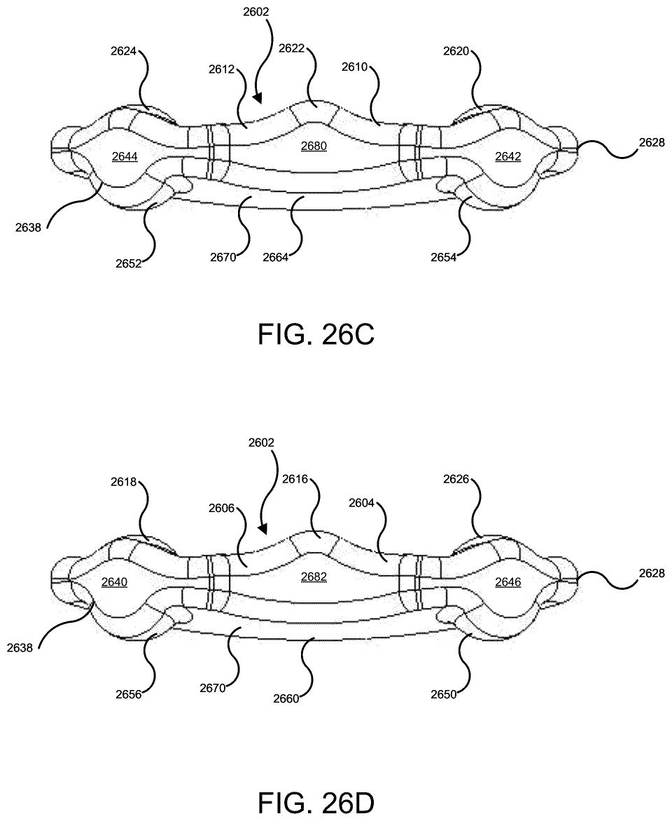

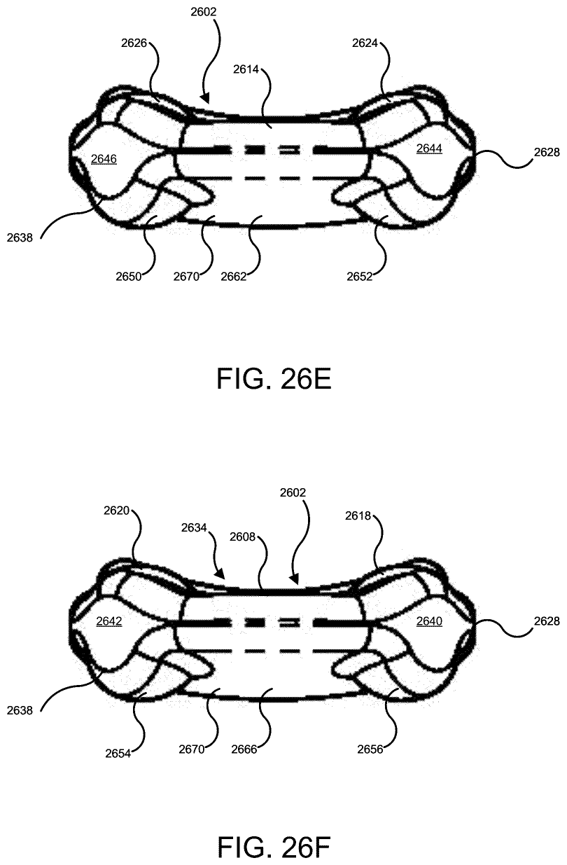

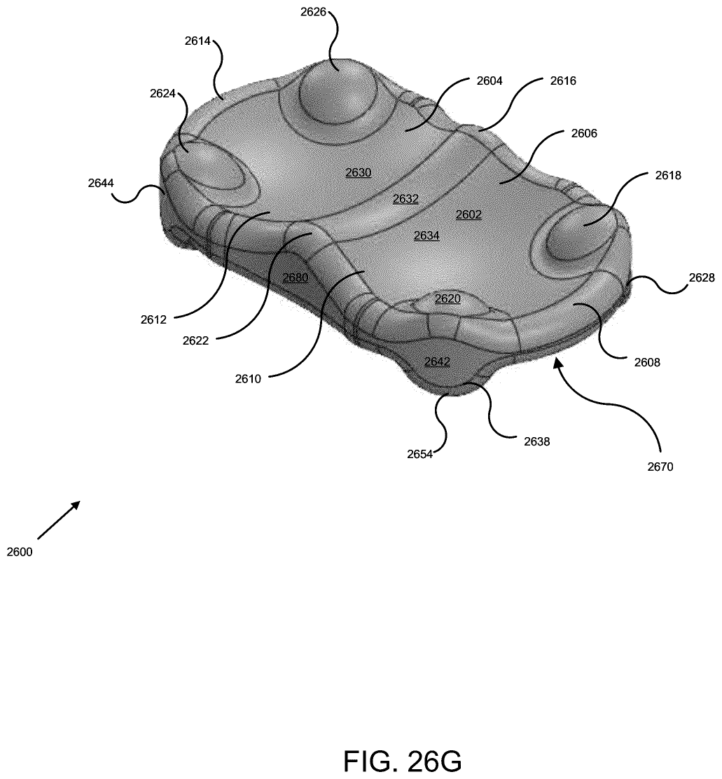

FIG. 26A illustrates a top view of an exemplary implantable interpositional orthopedic pain management apparatus. Here, device 2600 includes top surface 2602, channel openings 2604-2614, notches 2616 and 2622, peripheral protrusions 2618, 2620, 2624, and 2626, perimeter 2628, top surface regions 2630-2634, and contours 2638. As used herein, like numbered and/or like named elements are assumed to be referencing the same or a substantially similar element having the same or substantially similar function or structure. Differences in function or structure may be described separately with regard to a specific feature described. As shown, top surface 2602 may be include multiple regions 2630-2634 each of which may be defined by a different radius of curvature. The radius of curvature of each region 2630-2634 may in part depend on the shape of the bone against which top surface 2602 is intended to be implanted against. Each of regions 2630-2634 may have a different radius of curvature, selected to fit with an end of a bone (e.g., metacarpal, trapezium, tibia, femur, or others, without limitation or restriction). In some examples, one or more of regions 2630-2634 may be substantially concave. In some examples, one or more of regions 2630-2634 may be substantially convex. Region 2632 may be configured to define, in some examples, an elongate central portion, in between regions 2630 and 2634, with notches 2616 and 2622 on opposing sides of region 2632, disposed substantially on perimeter 2628, and formed with top surface 2602. While formed as part or integrated with device 2600, perimeter 2628 may be an outermost portion of device 2600 into which features are shaped, such as peripheral protrusions 2618, 2620, 2624, and 2626, and notches 2616 and 2622. In the example herein, peripheral protrusions 2618, 2620, 2624, and 2626 are disposed along perimeter 2628 and have a rounded portion or element. Channel openings 2608 and 2614 may be disposed between two adjacent protrusions, such as 2618 and 2620, and 2624 and 2626, respectively. In other examples, a channel opening 2604 may be disposed between a peripheral protrusion 2626 and a notch 2616. A channel may be formed in between channel openings 2604 and 2612. A channel may also be formed in between channels 2606 and 2610. The channels between 2604 and 2612, and 2606 and 2610 may be substantially parallel, and/or be disposed on opposing sides of region 2632. Region 2630 may include a channel formed with channel opening 2614, the channel formed may be curved and overlap or be otherwise coextensive with the channel formed between channel openings 2604 and 2612. Region 2634 may include a channel formed with channel opening 2608, the channel formed may be curved and overlap or be otherwise coextensive with the channel formed between channel openings 2606 and 2610. In other examples, as in the previously and following described figures, the number, shape, type, quantity, and disposition of peripheral protrusions 2618, 2620, 2624, and 2626 may be varied and are not limited to those shown and described. Device 2600 may be further shaped as shown by contour lines (identified generally as contours 2638), to accommodate the shape of bone surface of a target joint. In other examples, device 2600 and the elements shown and described may be designed, configured, formed, modified, or implemented apart from the examples shown or described and are not limited to those provided.

FIG. 26B illustrates a bottom view of an exemplary implantable interpositional orthopedic pain management apparatus. Here, device 2600 includes perimeter 2628, contours 2638, peripheral protrusions 2650-2656, channel openings 2660-2666, and bottom surface 2670. As used herein, like numbered and/or like named elements are assumed to be referencing the same or a substantially similar element having the same or substantially similar function or structure. Differences in function or structure may be described separately with regard to a specific feature described. In some examples, a channel may be formed in between channel openings 2660 and 2664, or in between channel openings 2662 and 2666. Bottom surface 2670 may have a radius of curvature so that surface 2670 substantially fits the end of a bone within a joint. In other examples, bottom surface 2670 may have a radius of curvature that is one or two degrees greater than the radius of curvature of a bone. Once device 2600 is surgically implanted in a joint, peripheral protrusions 2650-2656 may be in contact, or not be in contact with adjacent bone surfaces depending on the flexion or extension of the joint. While the various examples shown herein may exhibit symmetry around various axes, in other examples device 2600 and others described above and below may be asymmetrically formed or off-axially aligned, including when surgically implanted within a joint. In other examples, contours 2638 may define additional curvature or surface elements of device 2600 that may be selected to configure device 2600 to fit in a joint. In still other examples, device 2600 and the elements shown and described may be designed, configured, formed, modified, or implemented apart from the examples shown or described and are not limited to those provided.

FIG. 26C illustrates an anterior view of an exemplary implantable interpositional orthopedic pain management apparatus. Here, an anterior or front view of device 2600 includes top surface 2606, channel openings 2610, 2612, and 2662, peripheral protrusions 2620, 2624, 2652, and 2654, notch 2622, perimeter 2628, contours 2638, outward facing vertical surfaces 2642, 2644, and 2680, and bottom surface 2670. As used herein, like numbered and/or like named elements are assumed to be referencing the same or a substantially similar element having the same or substantially similar function or structure. Differences in function or structure may be described separately with regard to a specific feature described. Channel openings 2610, 2612, and 2664, for examples, define openings to channels on top surface 2602 or bottom surface 2670. Channels may constitute portions of surfaces 2602 and 2670. Channels may be curved and overlap one another, and may have indefinite boundaries. In some examples, a channel is formed on top surface 2602 in between channel openings 2610 and 2606 (not shown). A channel may also be formed on top surface 2602 in between channel openings 2612 and 2604 (not shown). In some examples, a channel is formed on bottom surface 2670 in between channel openings 2664 and 2660 (not shown). Notch 2622 and region 2632 may, for example, define a side portion of a channel, such as a side portion of the channel formed in between channel openings 2612 and 2604 (not shown). The position of channels on top surface 2602 may in part be defined by curvature changes between regions 2630-2634. For example, the channel formed in between channel openings 2612 and 2604 may be a part of region 2630 (not shown), and the channel formed in between channel openings 2610 and 2606 (not shown) may be a part of region 2634. Region 2632 may define a change a curvature between 2630 and 2634 and also separate the aforementioned channels on top surface 2602. Top and bottom surfaces 2602 and 2670 may each be formed to receive mutually reciprocal or substantially mutually reciprocal bones, bone structures, or portions thereof in a joint. Peripheral protrusions 2620 and 2654 may also be substantially disposed on two sides of outward facing vertical surface 2642. Peripheral protrusions 2624 and 2652 may be substantially disposed on two sides of outward facing vertical surface 2644. Notch 2622 may be disposed on a side of outward facing vertical surface 2680. The shape of outward facing vertical surfaces 2642, 2644, and 2680 may be selected to be aligned with the shape of perimeter 2628. Outward facing vertical surfaces 2642, 2644, and 2680 may disposed substantially on perimeter 2628 and may have radii of curvature and topographies such that they minimize interaction with adjacent bone or tissue once implanted in a joint. In still other examples, device 2600 and the elements shown and described may be designed, configured, formed, modified, or implemented apart from the examples shown or described and are not limited to those provided.

FIG. 26D illustrates a posterior view of an exemplary implantable interpositional orthopedic pain management apparatus. Here, a posterior or back view of device 2600 includes top surface 2602, channel openings 2604, 2606, and 2660, peripheral protrusions 2618, 2626, 2650, and 2656, notch 2616, perimeter 2628, contours 2638, outward facing vertical surfaces 2640, 2646 and 2682, and bottom surface 2670. As used herein, like numbered and/or like named elements are assumed to be referencing the same or a substantially similar element having the same or substantially similar function or structure. Differences in function or structure may be described separately with regard to a specific feature described. Elements recited herein are similar to those illustrated and described in reference to FIG. 26C. Here a channel is formed on top surface 2602 in between channel openings 2606 and 2610 (not shown) A channel may also be formed on top surface 2602 in between channel openings 2604 and 2612 (not shown). In some examples, a channel is formed on bottom surface 2670 in between channel openings 2660 and 2664 (not shown). Notch 2616 and region 2632 may define a side portion of a channel, for example, the channel formed in between channel openings 2604 and 2612 (not shown). Peripheral protrusions 2618, 2626, 2650, and 2656 may be formed along perimeter 2628 of device 2600, and be formed with outward facing vertical surfaces 2640 and 2646. Once surgically implanted within a joint, peripheral protrusions 2618, 2626, 2650, and 2656, and notch 2616 may be configured to reside in non-articulating regions of a joint (not shown). In some examples, top surface 2602 and bottom surface 2670 may be formed with various types of shapes and features (e.g., convex, concave, partially or wholly, or a combination thereof), as may be shown by contours 2638. If one or more bones of the joint (in which device 2600 is surgically implanted) are articulated, peripheral protrusions 2618, 2626, 2650, and 2656, and notch 2616 are configured to prevent the bones from coming into contact with each other, maintaining dynamic stability, and restoring an ability for the joint to be manipulated while preventing expulsion of device 2600 from the joint or dislocation of bones from the joint. In other examples, device 2600 and the elements shown and described may be designed, configured, formed, modified, or implemented apart from the examples shown or described and are not limited to those provided.

FIG. 26E illustrates a left view of an exemplary implantable interpositional orthopedic pain management apparatus. Here, a left side view of device 2600 includes top surface 2602, channel openings 2614 and 2662, peripheral protrusions 2624, 2626, 2650, and 2652, perimeter 2628, contours 2638, outward facing vertical surfaces 2644 and 2646, and bottom surface 2670. As used herein, like numbered and/or like named elements are assumed to be referencing the same or a substantially similar element having the same or substantially similar function or structure. Differences in function or structure may be described separately with regard to a specific feature described. As shown, channel opening 2614 is disposed between peripheral protrusions 2624 and 2626. Channel opening 2662 is disposed between peripheral protrusions 2650 and 2652. Peripheral protrusions 2624 and 2626 are disposed on the top sides of outward facing vertical surfaces 2644 and 2646, and peripheral protrusions 2650 and 2652 are disposed on the bottom sides of outward facing vertical surfaces 2644 and 2646. Depending on the joint in which device 2600 is to be implanted within (not shown), the outward facing vertical surfaces 2644, 2646 may be shaped in a manner that minimizes disturbance of any adjacent tissue or bone, thus decreasing potential pain or irritation caused by the implanted device 2600. Outward facing vertical surfaces 2644 and 2646, may in some examples be bulb shaped, with a rounded portion in between peripheral protrusions (i.e. 2624 and 2652) and a pointed portion (in this view, adjacent to channel openings 2614 and 2662). In other examples, the positions and shapes of peripheral protrusions 2624, 2626, 2650, and 2652, outward facing vertical surfaces 2644 and 2646 may be such that device 2600 may best fit within a targeted joint and minimize likelihood of expulsion of device 2600 from the joint (not shown). Contours 2638 may illustrate potential exemplary structures of portions of device 2600, for example, a curved transition portion between peripheral protrusion 2650 and outward facing vertical surface 2646. In other examples, device 2600 and the elements shown and described may be designed, configured, formed, modified, or implemented apart from the examples shown or described and are not limited to those provided.

FIG. 26F illustrates a right view of an exemplary implantable interpositional orthopedic pain management apparatus. Here, a right side view of device 2600 includes top surface 2602, channel openings 2608 and 2666, peripheral protrusions 2618, 2620, 2654, and 2656, perimeter 2628, top surface region 2634, contours 2638, outward facing vertical surfaces 2640 and 2642, and bottom surface 2670. As used herein, like numbered and/or like named elements are assumed to be referencing the same or a substantially similar element having the same or substantially similar function or structure. Differences in function or structure may be described separately with regard to a specific feature described. Elements recited herein are similar to those described and illustrated in reference to FIG. 26F. In some examples, peripheral protrusions 2618, 2620, 2654, and 2656 may be coupled directly or indirectly with perimeter 2628 or formed as surface or structural features or components thereof. As shown and described, peripheral protrusions 2618, 2620, 2654, and 2656 may have various sizes, shapes, dimensions, and other attributes, without limitation or restriction. Here, channel opening 2608 is defined by a portion of top surface 2602 between peripheral protrusions 2618 and 2620. Channel opening 2666 is defined by a portion of top surface 2602 between peripheral protrusions 2654 and 2656. A portion of top surface 2602, such as region 2634 (identified generally by a floating arrow), may be substantially concave. A portion of bottom surface 2670 may be substantially convex. However, the shapes and radii of curvatures of top and bottom surfaces 2602 and 2670 may be generally selected based on the corresponding shapes of the ends of the bones in a targeted joint (not shown). In other examples, device 2600 and the elements shown and described may be designed, configured, formed, modified, or implemented apart from the examples shown or described and are not limited to those provided.

FIG. 26G illustrates a perspective view of an exemplary implantable orthopedic pain management apparatus. Here, a perspective view of device 2600 includes top surface 2602, channel openings 2604-2614, notches 2616 and 2622, peripheral protrusions 2618, 2620, 2624, 2626, and 2654, perimeter 2628, top surface regions 2630-2634, contours 2638, outward facing vertical surfaces 2642, 2644, and 2680, and bottom surface 2670. As used herein, like numbered and/or like named elements are assumed to be referencing the same or a substantially similar element having the same or substantially similar function or structure. Differences in function or structure may be described separately with regard to a specific feature described. As shown, the configurations of top surface regions 2630-2634 are each different. Here, regions 2630 and 2634 and both substantially concave, are substantially symmetrical in shape, and include the channels formed between channel openings 2604 and 2612, and 2606 and 2610, respectively. Notches 2616 and 2622 define the end portions of region 2632, the end portions being adjacent to perimeter 2628. In some examples, region 2632 includes slopes on surface portions by each notch 2616 and 2622. Regions 2630-2634 define the shape of top surface 2602, and in some examples, is based on the shape of an end of a bone of a target joint (not shown). The number and shape of top surface regions 2630-2634, however, are not limited to those shown herein. In the example shown, peripheral protrusions 2618, 2620, 2624, and 2626 are partial-spherical in shape. However, examples of device 2600 is not limited to the shown peripheral protrusion shape, such that the shape of the protrusion may be selected based on the specific shape of the adjacent bone that device 2600 is to be implanted next to. For example, peripheral protrusions 2618, 2620, 2624, and 2626 may include additional structural or surface portions as illustrated in general by contours 2638. Outward facing vertical surfaces 2642 and 2644 may be formed as part of peripheral protrusions 2620, and 2624. Outward facing vertical surface 2680 may be formed as part of perimeter 2628 and be substantially flat, in some examples. Outward facing vertical surfaces 2642, 2644, and 2680, may generally be shaped or similar to perimeter 2628 and, once implanted, be shaped to not extend past an adjacent bone surface. Device 2600, once surgically implanted within a synovial capsule and/or a joint, functions to prevent contact between a bone received by top surface 2602 and bottom surface 2670. Once implanted, device 2600 is configured to anatomically align within the joint. Device 2600 may not be permanently coupled or semi-permanently joined to a bone surface to allow for dynamic movement of device 2600 within the joint. In other words, device 2600 may be in intermittent contact with bones that make up a joint. Peripheral protrusions 2618, 2620, 2624, 2626, and 2650-2654 may in part function to hold device 2600 within the joint (not shown) by contacting substantially outside, or outside-facing, portions of the received bones, thus preventing expulsion of device 2600 from the joint. In other examples, peripheral protrusions 2618, 2620, 2624, 2626, and 2650-2654 and top and bottom surfaces 2602 and 2670 may also be configured to provide surfaces that are shaped to fit and/or fill with other materials within a synovial capsule such as cartilage, blood, bodily fluids, or other synthetic or organic materials that are injected or otherwise implanted to aid device 2600 in maintaining dynamic stability and position within a joint after surgical implantation. In still other examples, device 2600 and the elements shown and described may be designed, configured, formed, modified, or implemented apart from the examples shown or described and are not limited to those provided.

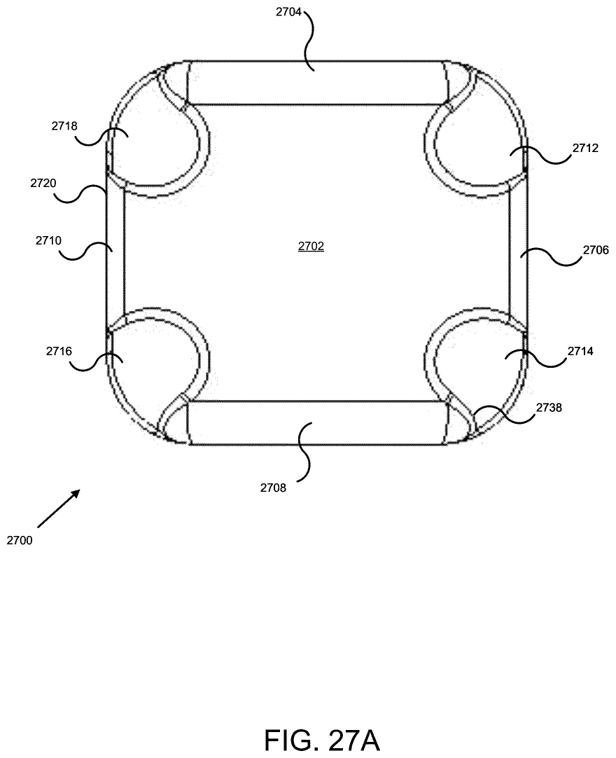

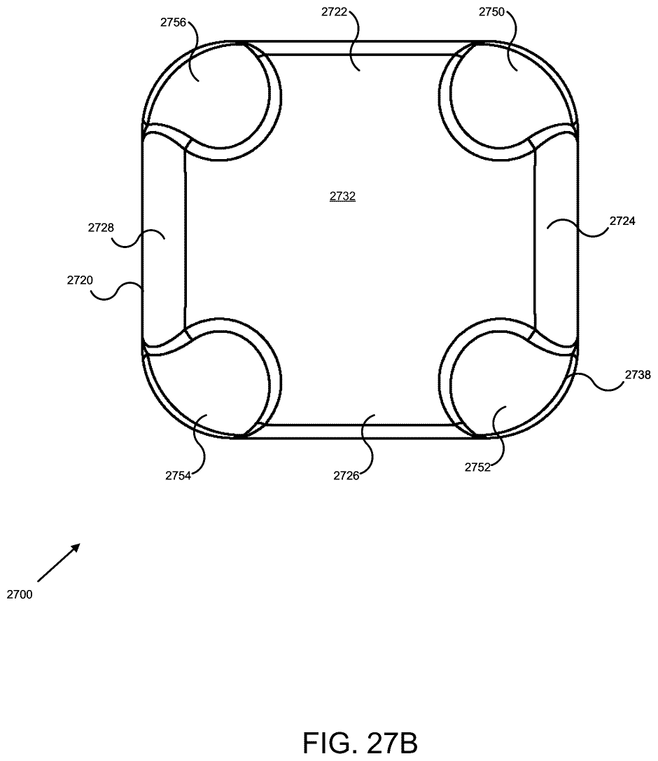

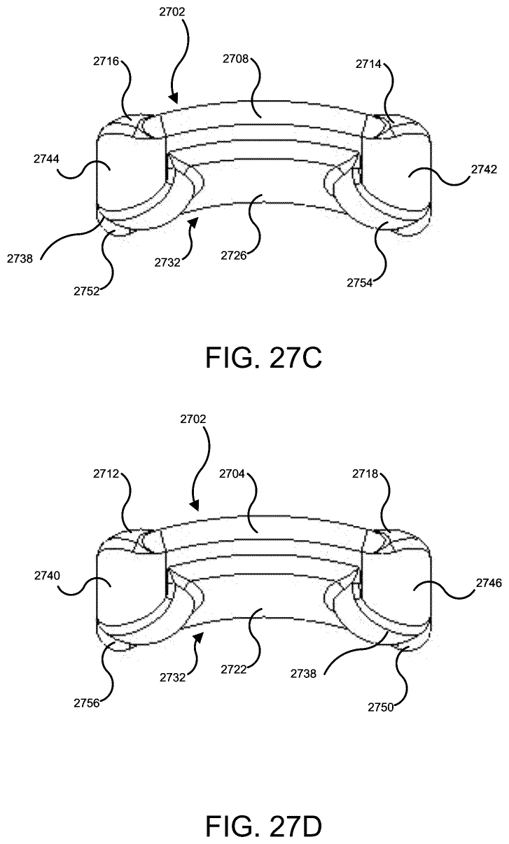

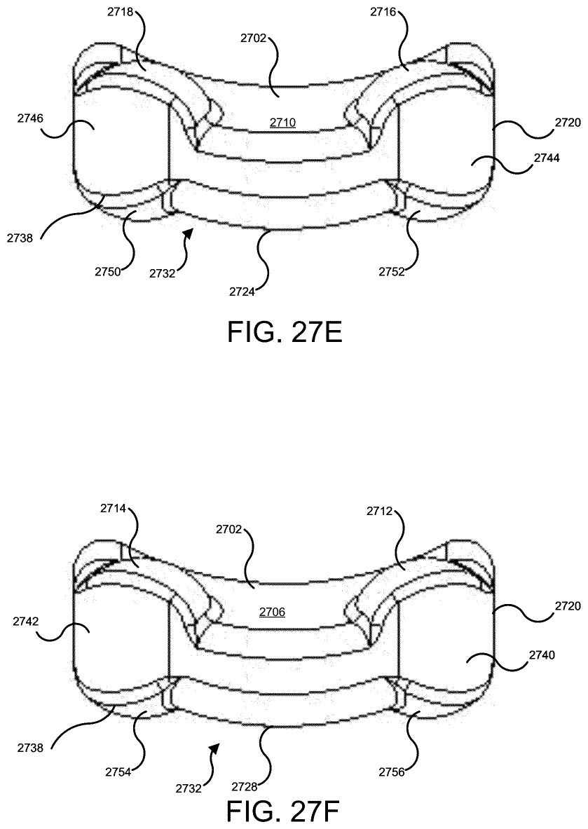

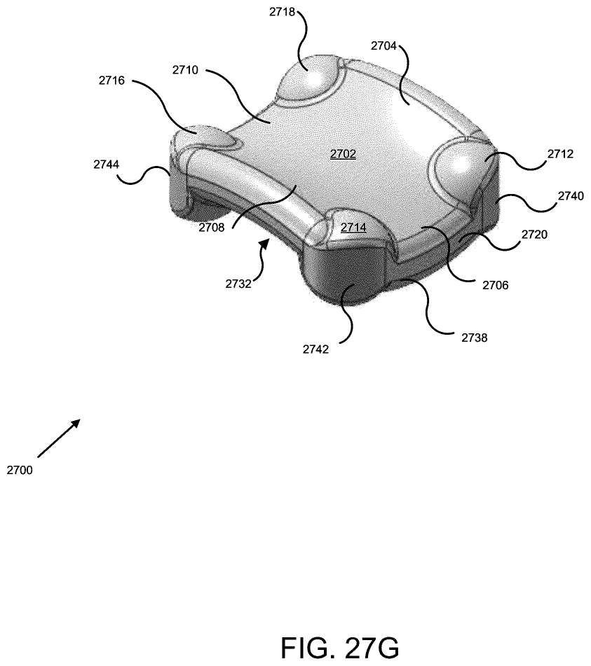

FIG. 27A illustrates a top view of an exemplary implantable interpositional orthopedic pain management apparatus. Here, a top view is shown of device 2700, which includes top surface 2702, channel openings 2704-2710, peripheral protrusions 2712-2718, perimeter 2720, and contours 2738. As used herein, like numbered and/or like named elements are assumed to be referencing the same or a substantially similar element having the same or substantially similar function or structure. Differences in function or structure may be described separately with regard to a specific feature described. As used herein, the term “saddle” may be used to refer to any implementation of device 2700, which is not limited to symmetrical, asymmetrical, off-centered, centered, aligned, or other specific geometric shapes or properties. A “saddle” may be implemented using various shapes that may be concave or convex and are not required to be aligned symmetrically around any dimensional axis (not shown) shown or described throughout, but can be axially aligned, anatomically aligned (i.e., aligned about a joint, bone, or other anatomical structure, without limitation or restriction), or aligned differently, without limitation or restriction. The term “saddle” may be used to refer, in some examples, to a body of device 2700 that is configured to receive, fit, conform, or otherwise be positioned or placed on, over, under, or between one or more bones. Some of these shapes may be varied such that device is saddle-shaped or substantially saddle-shaped implementation. Here, device 2700 is configured structurally to contour to bone ends (e.g., distal or proximal ends of bones that have various contoured surfaces that may be convex, concave, or otherwise). Top surface 2702 may be substantially four sided, with perimeter 2720 defining the outermost edge of device 2700. Top surface 2702 may be substantially concave, or saddle shaped, with a curvature component that is larger than that of a bone surface of the targeted joint. In other examples, top surface 2702 may have a radius of curvature such that it is substantially convex to receive or fit against an end of a bone. In some examples, channel openings 2704-2710 may constitute part of top surface 2702. A channel (e.g., as formed by top surface 2702 and channel openings 2706 and 2710) may be disposed on top surface 2702 and define a portion of top surface 2702 in between channel openings. Peripheral protrusions 2712-2718 may be formed with top surface 2702 with outward facing vertical surfaces 2740-2746, to be later described in reference to FIGS. 27C-27G. In some examples, peripheral protrusions 2712-2718 may be disposed substantially about perimeter 2720, and aid in preventing expulsion of device 2700 from within a joint, after implantation. Peripheral protrusions 2712-2718 may be configured such that they do not substantially interfere with motion of the joint once implanted within a joint and oppose, or apply an opposing force, to force applied to device 2700 once implanted. Device 2700 and peripheral protrusions 2712-2718 may include additional design components, as illustrated by and identified generally by contours 2738. Peripheral protrusions 2712-2718 may also be disposed on opposing sides of a channel opening 2704-2710. The positions of channel openings 2704-2710 and the one or more channels may be configured to have a single channel or dual channels to receive the head or torus of a bone within a joint. As described herein, device 2700 may be surgically implanted (i.e., placed) in a joint at a point, position, and/or orientation where device 2700 is least likely to be expulsed when the joint is articulated. As described above, device 2700 is configured to permit motion or articulation of a joint without dislocation or expulsion of device 2700 from the joint. In other words, when bones in a given joint are articulated, device 2700 when implanted may be configured and implemented to prevent one or more bones from dislocation or expulsing device 2700 from the joint. In still other examples, device 2700 and the elements shown and described may be designed, configured, formed, modified, or implemented apart from the examples shown or described and are not limited to those provided.

FIG. 27B illustrates a bottom view of an exemplary implantable interpositional orthopedic pain management apparatus. Here, device 2700 includes perimeter 2720, channel openings 2722-2728, bottom surface 2732, contours 2738, and peripheral protrusions 2750-2756. As used herein, like numbered and/or like named elements are assumed to be referencing the same or a substantially similar element having the same or substantially similar function or structure. Differences in function or structure may be described separately with regard to a specific feature described. In the example herein, bottom surface 2732 may have radii of curvature such that it is substantially saddle shaped to receive (or otherwise be shaped to substantially fit) an end of a bone. Bottom surface 2732 may have channel openings 2722-2728 from which channels on bottom surface 2732 may be defined, similar to channel openings 2704-2710 (FIG. 23A). In some examples, channels may be substantially orthogonal to one another. In some examples, peripheral protrusions 2750-2756 may be formed with bottom surface 2732 and be shaped to support device 2700 within a joint once surgically implanted. In other examples, outward facing vertical surfaces 2740-2746 (not shown) are disposed on a side of each peripheral protrusion 2750-2756, the surfaces being generally faced away from a central region of top or bottom surfaces 2702, 2732, and may have a curvature element similar to the curvature or shape of perimeter 2720. In other examples, device 2700 and the elements shown and described may be designed, configured, formed, modified, or implemented apart from the examples shown or described and are not limited to those provided.

FIG. 27C illustrates an anterior view of an exemplary implantable interpositional orthopedic pain management apparatus. Here, an anterior or front view of device 2700 includes top surface 2702, channel openings 2708 and 2726, peripheral protrusions 2714, 2716, 2752, and 2754, bottom surface 2732, contours 2738, and outward facing vertical surfaces 2742 and 2744. As used herein, like numbered and/or like named elements are assumed to be referencing the same or a substantially similar element having the same or substantially similar function or structure. Differences in function or structure may be described separately with regard to a specific feature described. From the anterior view, the portion of top surface 2702 shown is substantially convex, and the portion of bottom surface 2732 shown is substantially concave. However, the curvatures of such surfaces are not limited to those shown herein. Channel opening 2708 is shown disposed opposite of channel opening 2726. Channel opening 2708 may be disposed in between peripheral protrusions 2714 and 2716. The channel formed on top surface 2702 with channel openings 2708 and 2704 (not shown) may be shaped to receive a torus or an end of a bone. Channel opening 2726 may be disposed between peripheral protrusions 2752 and 2754. The channel formed on bottom surface 2732 with channel openings 2726 and 2722 may be shaped to receive an end of an opposing bone in the joint. In other words, device 2700 has structural elements such as peripheral protrusions 2712-2718, 2750-2756 and top and bottom surfaces 2702, 2732, to substantially align with adjacent bone surfaces in a joint. In some examples, outward facing vertical surfaces 2740-2746 may be disposed in between adjacent or opposing peripheral protrusions 2712-2718 and 2750-2756. For example, outward facing vertical surface 2742 is in between, or connects, peripheral protrusions 2714 and 2754. Device 2700 may be shaped with additional topographical characteristics, as shown by contours 2738. In other examples, device 2700 and the elements shown and described may be designed, configured, formed, modified, or implemented apart from the examples shown or described and are not limited to those provided.

FIG. 27D illustrates a posterior view of an exemplary implantable interpositional orthopedic pain management apparatus. Here, a posterior view of device 2700 includes top surface 2702, channel openings 2704 and 2722, peripheral protrusions 2712, 2718, 2750, and 2756, bottom surface 2732, contours 2738, and outward facing vertical surfaces 2740 and 2746. As used herein, like numbered and/or like named elements are assumed to be referencing the same or a substantially similar element having the same or substantially similar function or structure. Differences in function or structure may be described separately with regard to a specific feature described. Elements of FIG. 27D are similar to those shown and described in reference to FIG. 27C. In some examples, channel openings 2704 and 2708 (not shown) form a channel configured to receive a bone in top surface 2702 when device 2700 is surgically implanted into a synovial capsule and joint. In some examples, channel openings 2722 and 2726 (not shown) form a channel configured to receive a bone in the bottom surface 2732 when device 2700 is surgically implanted into a synovial capsule and joint. In other examples, device 2700 and the elements shown and described may be designed, configured, formed, modified, or implemented apart from the examples shown or described and are not limited to those provided.

FIG. 27E illustrates a left view of an exemplary implantable interpositional pain management apparatus. Here, a left view of device 2700 includes top surface 2702, channel openings 2710 and 2724, peripheral protrusions 2716, 2718, 2750, and 2752, perimeter 2720, bottom surface 2732, contours 2738, and outward facing vertical surfaces 2744 and 2746. As used herein, like numbered and/or like named elements are assumed to be referencing the same or a substantially similar element having the same or substantially similar function or structure. Differences in function or structure may be described separately with regard to a specific feature described. Here, from a side left view, a portion of top surface 2702 shown is substantially concave, and a portion of bottom surface 2732 shown is substantially convex. However, the curvatures of such surfaces are not limited to those shown herein. Channel opening 2710 is shown disposed opposite of channel opening 2724. Channel opening 2710 may be disposed in between peripheral protrusions 2716 and 2718. The channel formed on top surface 2702 with channel openings 2710 and 2706 (not shown) may be shaped to receive an end of a bone. Channel opening 2724 may be disposed between peripheral protrusions 2752 and 2752. The channel formed on bottom surface 2732 with channel openings 2724 and 2728 may be shaped to receive an end of an opposing bone in the joint. In some examples, outward facing vertical surfaces 2740-2746 may be disposed in between adjacent peripheral protrusions 2712-2718 and 2750-2753. For example, outward facing vertical surface 2744 is in between, or connects, peripheral protrusions 2716 and 2752. Device 2700 may be surgically implanted in a joint, and oriented such that it prevents contact between surfaces of adjacent bones. Outward facing vertical surfaces 2744 and 2746 may have a curvature that aligns with perimeter 2720, such that device 2700 minimally interferes with lateral tissue or bones once surgically implanted within a joint. In other examples, device 2700 and the elements shown and described may be designed, configured, formed, modified, or implemented apart from the examples shown or described and are not limited to those provided.

FIG. 27F illustrates a right view of an exemplary implantable interpositional pain management apparatus. Here, a right view of device 2700 includes top surface 2702, channel openings 2706 and 2728, peripheral protrusions 2712, 2714, 2754, and 2756, perimeter 2720, bottom surface 2732, contours 2738, and outward facing vertical surfaces 2740 and 2742. As used herein, like numbered and/or like named elements are assumed to be referencing the same or a substantially similar element having the same or substantially similar function or structure. Differences in function or structure may be described separately with regard to a specific feature described. Elements of FIG. 27F are similar to those shown and described in reference to FIG. 27E. In some examples, top surface 2702 is configured, with channel openings 2706 and 2710 (not shown), to receive a bone or a portion thereof when device 2700 is surgically implanted. Bottom surface 2732 may also be configured, with channel openings 2728 and 2724 (not shown), to receive a bone or a portion thereof when device 2700 is surgically implanted. For example, if device 2700 is surgically implanted into a CMC joint, top surface 2702 may be configured, with channel openings 2706 and 2710, to receive (i.e., top surface 2302 may have a radius of curvature that is similar to that of a trapezium bone) a trapezium bone, or other bone adjacent to the joint. Outward facing vertical surfaces 2740 and 2742 may have a curvature that aligns with the perimeter 2720, such that device 2700 minimally interferes with lateral tissue or bones once surgically implanted within a joint. In other examples, device 2700 and the elements shown and described may be designed, configured, formed, modified, or implemented apart from the examples shown or described and are not limited to those provided.

FIG. 27G illustrates a perspective view of an exemplary implantable interpositional orthopedic pain management apparatus. Here, device 2700 includes top surface 2702, channel openings 2704-2710, peripheral protrusions 2712-2718, perimeter 2720, bottom surface 2732, contours 2738, and bottom surface 2732. As used herein, like numbered and/or like named elements are assumed to be referencing the same or a substantially similar element having the same or substantially similar function or structure. Differences in function or structure may be described separately with regard to a specific feature described. As shown, top surface 2702 has a saddle shape, with a convex portion and a concave portion. In some examples, top surface 2702 may be substantially four sided, or rectangular, with peripheral protrusions 2712-2718 disposed on four corners. Perimeter 2720 may constitute the outermost portion of top or bottom surfaces 2702, 2732. Peripheral protrusions 2712-2718 may be disposed along perimeter 2720 on top surface 2702, and peripheral protrusions 2750-2756 (not shown) are similarly disposed along perimeter 2720, on bottom surface 2732, opposite peripheral protrusions 2712-2718. In other examples, however, peripheral protrusions may not be disposed opposite peripheral protrusions on an opposite side of device 2700. Peripheral protrusions 2712-2718 and 2750-2756 (not shown) may be dissed in between proximally-positioned channel openings 2704-2710 and 2722-2728 (not shown). For example, peripheral protrusion 2712 is positioned in between proximally-positioned channel openings 2704 and 2706. Outward facing vertical surface 2742 may be disposed between opposing peripheral protrusions 2714 and 2754, for example, and be curved, as to not extend outward beyond perimeter 2728. In some examples, outward facing vertical surfaces 2740-2744 may be formed with, or be coextensive with, perimeter 2728 and associated with peripheral protrusions 2712-2718 and 2750-2756. Channel opening 2704 indicates an opening to a channel formed with top surface 2702 and channel opening 2708. Similarly, channel opening 2706 indicates an opening to a channel formed with top surface 2702 and channel opening 2708. Device 2700 may include additional surface elements as illustrated and identified generally as contours 2738. For example, device 2700 may include a convex curved portion as a transition between perimeter 2720 and top surface 2702. As with the other examples throughout this description, various radii of curvature may be used and are not limited to the examples shown and described. Device 2700 may not be permanently coupled or semi-permanently joined to a bone surface to allow for dynamic movement of device 2700 within the joint. In other words, device 2700 may be in intermittent contact with bones that make up a joint. Once implanted, peripheral protrusions 2712-2718 and 2750-2756 (not shown) may, in part, function to move device 2700 in a direction that is substantially opposite to a force transmitted by a bone surface. In other examples, device 2700 and the elements shown and described may be designed, configured, formed, modified, or implemented apart from the examples shown or described and are not limited to those provided.

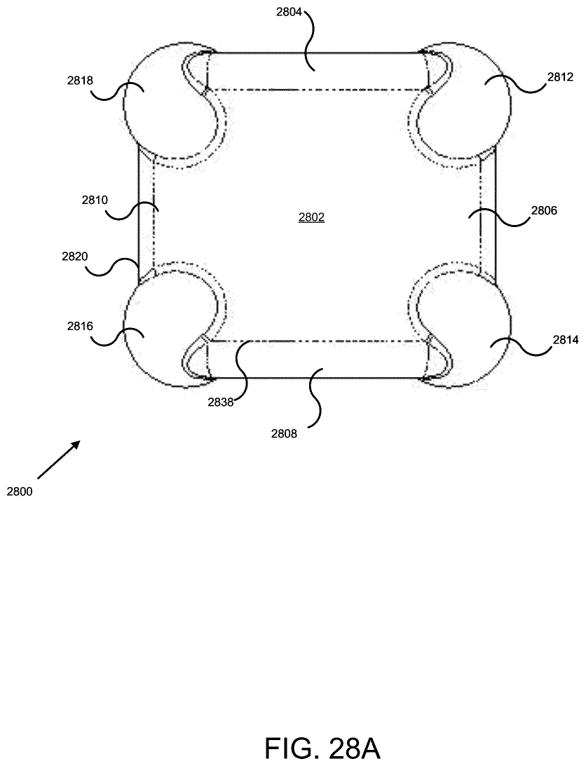

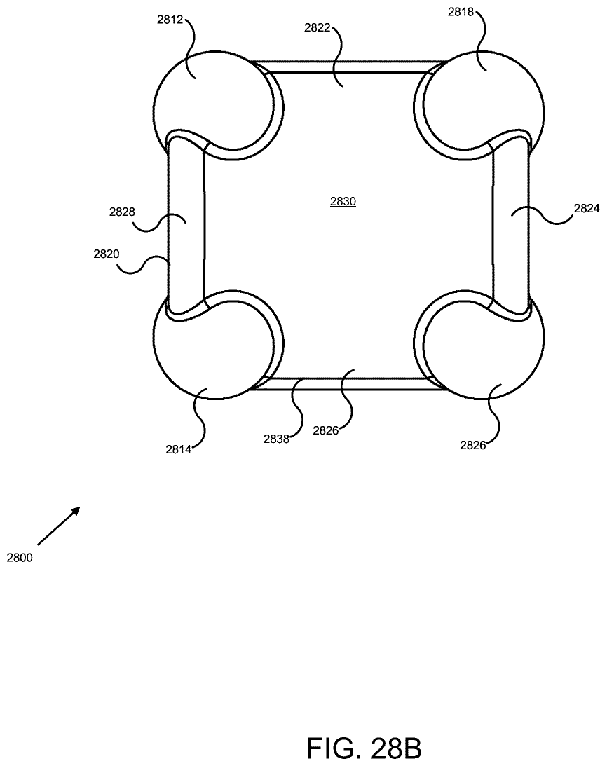

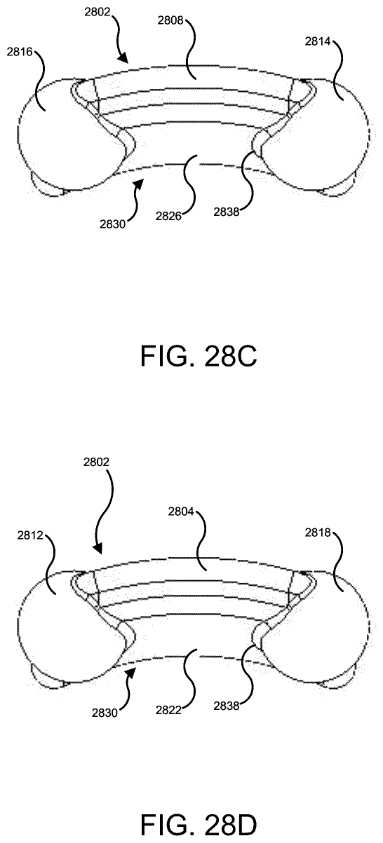

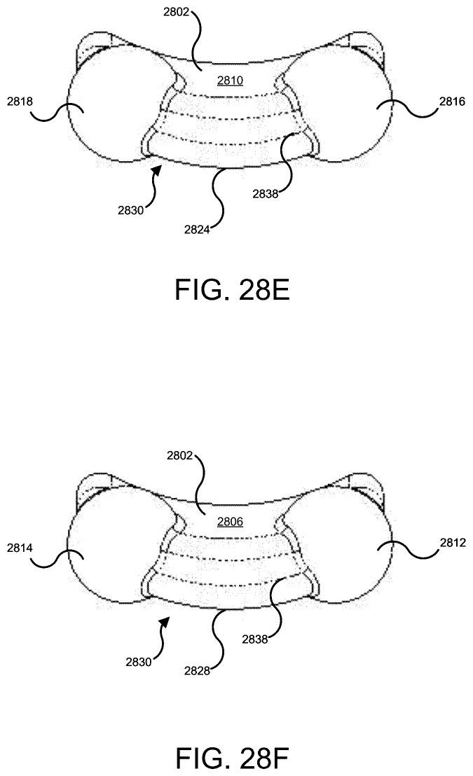

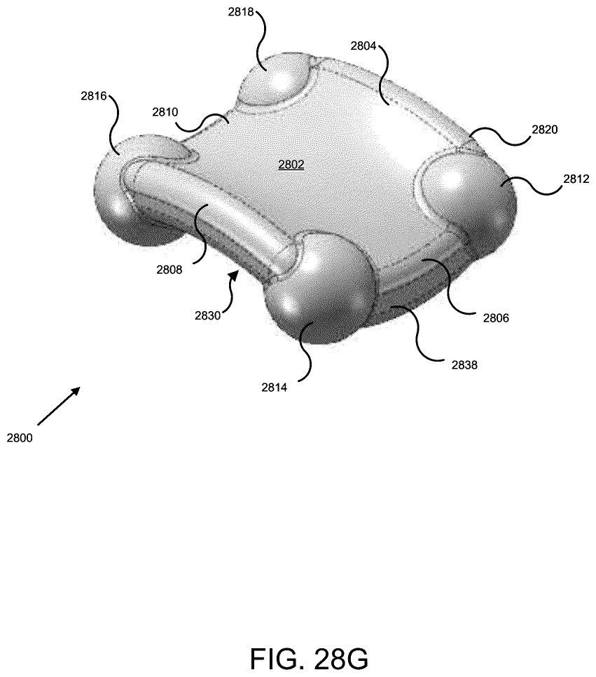

FIG. 28A illustrates a top view of an exemplary implantable interpositional orthopedic pain management apparatus. Here, a top view is shown of device 2800, which includes top surface 2802, channel openings 2804-2810, peripheral protrusions 2812-2818, perimeter 2820, and contours 2838. As used herein, like numbered and/or like named elements are assumed to be referencing the same or a substantially similar element having the same or substantially similar function or structure. Differences in function or structure may be described separately with regard to a specific feature described. As used herein, the term “saddle” may be used to refer to any implementation of device 2800, which is not limited to symmetrical, asymmetrical, off-centered, centered, aligned, or other specific geometric shapes or properties. A “saddle” may be implemented using various shapes that may be concave or convex and are not required to be aligned symmetrically around any dimensional axis (not shown) shown or described throughout, but can be axially aligned, anatomically aligned (i.e., aligned about a joint, bone, or other anatomical structure, without limitation or restriction), or aligned differently, without limitation or restriction. The term “saddle” may be used to refer, in some examples, to a body of device 2800 that is configured to receive, fit, conform, or otherwise be positioned or placed on, over, under, or between one or more bones. Some of these shapes may be varied such that device is saddle-shaped or substantially saddle-shaped implementation. Here, device 2800 is configured structurally to contour to bone ends (e.g., distal or proximal ends of bones that have various contoured surfaces that may be convex, concave, or otherwise). Top surface 2802 may be substantially four sided, with perimeter 2820 defining the outermost edge of device 2800. Top surface 2800 may be substantially saddle shaped, with a curvature component that is larger than that of a bone surface of the targeted joint. In other examples, top surface 2802 may have a radius of curvature such that it is substantially convex to receive an end of a bone. In some examples, channel openings 2804-2810 may constitute part of top surface 2802. A channel (e.g., as formed by top surface 2802 and channel openings 2804 and 2808) may be disposed on top surface 2802 and may generally refer to a portion of top surface 2802 in between channel openings, or may be defined by a common curvature of a portion of top surface 2802 between the channel openings. Peripheral protrusions 2812-2818 may be formed with top surface 2802 and may be substantially spherical in shape. Peripheral protrusions 2812-2818, in some examples, may have an outermost portion that extends beyond perimeter 2820. In some examples, peripheral protrusions 2812-2818 may be disposed substantially about perimeter 2820, and aid in preventing expulsion of device 2800 from within a joint by moving device 2802 in a direction that is substantially opposite to a force transmitted by a bone surface to another bone surface. Peripheral protrusions 2812-2818 may be configured such that they do not substantially interfere with motion of the joint once implanted within a joint. Device 2800 and peripheral protrusions 2812-2818 may include additional design components, as illustrated by and identified generally by contours 2838. For example, there may be an area in between peripheral protrusion 2814 and top surface 2802 with a curvature element that transitions from a curvature associated with peripheral protrusion 2814 to that of top surface 2802. Peripheral protrusions 2812-2818 may also be disposed on opposing sides of a channel opening 2804-2810 The positions of channel openings 2804-2810 and the one or more channels may be configured to have a single channel or dual channels to receive the head or torus of a bone within a joint. As described herein, device 2800 may be surgically implanted (i.e., placed) in a joint at a point, position, and/or orientation where device 2800 is least likely to be expulsed when the joint is articulated. In other words, when bones in a given joint are articulated, device 2800 when implanted may be configured and implemented to prevent one or more bones from dislocation or expulsing device 2800 from the joint. In still other examples, device 2800 and the elements shown and described may be designed, configured, formed, modified, or implemented apart from the examples shown or described and are not limited to those provided.

FIG. 28B illustrates a bottom view of an exemplary implantable interpositional orthopedic pain management apparatus. Here, device 2800 includes peripheral protrusions 2812-2818, perimeter 2820, channel openings 2822-2828, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. In the example herein, bottom surface 2830 may have a radius of curvature such that it is substantially saddle shaped to receive (or otherwise be shaped to substantially fit) an end of a bone. Top and bottom surfaces 2802 (not shown) and 2830 may together be referred to the body of device 2800. Bottom surface 2830 may have channel openings 2822-2828 from which channels on bottom surface 2830 may be defined, similar to channel openings 2804-2810 (FIG. 28A). In some examples, channels may be substantially orthogonal to one another such that they intersect on bottom surface 2830. In some examples, peripheral protrusions 2812-2818 may be formed with bottom surface 2830 and be shaped to support device 2800 within a joint once surgically implanted, while also providing dynamic stability and pain relief in cases where cartilage has been worn away, destroyed, damaged, or is otherwise missing from a joint. Further, peripheral protrusions 2812-2818 are configured to not interfere with motion of joint in order to provide maximum range of extension and motion associated with individual bones forming a joint, and prevents migration of implantable device (i.e., implant or device) from joint. In other examples, device 2800 and the elements shown and described may be designed, configured, formed, modified, or implemented apart from the examples shown or described and are not limited to those provided.

FIG. 28C illustrates an anterior view of an exemplary implantable interpositional orthopedic pain management apparatus. Here, an anterior or front view of device 2800 includes top surface 2802, channel openings 2808 and 2826, peripheral protrusions 2814 and 2816, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. From the anterior view, top surface 2802 is substantially convex, and bottom surface 2830 is substantially concave. However, the curvatures of such surfaces are not limited to those shown herein and are instead based on the joint into which device 2800 is to be implanted within. Channel opening 2808 is shown disposed opposite of channel opening 2826. Channel opening 2808 may be disposed in between peripheral protrusions 2814 and 2816. The channel formed on top surface 2802 with channel openings 2808 and 2804 (not shown) may be shaped to receive a torus or an end of a bone. Channel opening 2826 may be disposed between peripheral protrusions 2814 and 2816. The channel formed on bottom surface 2830 with channel openings 2826 and 2822 may be shaped to receive an end of an opposing bone in the joint. In other words, device 2800 has structural elements such as peripheral protrusions 2812-2818 and top and bottom surfaces 2802, 2830, to substantially align device 2800 with adjacent bone surfaces in a joint. Peripheral protrusions 2814 and 2816 may be substantially spherical, having a portion of which structurally associated or formed with top surface 2802 and a portion of which structurally associated or formed with bottom surface 2830. Device 2800 may be shaped with additional topographical characteristics, as shown by contours 2838. In other examples, device 2800 and the elements shown and described may be designed, configured, formed, modified, or implemented apart from the examples shown or described and are not limited to those provided.

FIG. 28D illustrates a posterior view of an exemplary implantable interpositional orthopedic pain management apparatus. Here, a posterior view of device 2800 includes top surface 2802, channel openings 2804 and 2822, peripheral protrusions 2812 and 2818, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. Elements described herein are similar to those illustrated and described with reference to FIG. 28C. In some examples, channel openings 2804 and 2808 (not shown) form a channel configured to receive a bone in top surface 2802 when device 2800 is surgically implanted into a synovial capsule and joint. In some examples, channel openings 2822 and 2826 (not shown) form a channel configured to receive a bone in bottom surface 2830 when device 2800 is surgically implanted into a synovial capsule and joint. In other examples, device 2800 and the elements shown and described may be designed, configured, formed, modified, or implemented apart from the examples shown or described and are not limited to those provided.

FIG. 28E illustrates a left view of an exemplary implantable interpositional pain management apparatus. Here, a left view of device 2800 includes top surface 2802, channel openings 2810 and 2824, peripheral protrusions 2816 and 2818, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. Here, from a side left view, top surface 2802 is substantially concave, and bottom surface 2830 is substantially convex. However, the curvatures of such surfaces are not limited to those shown herein. Channel opening 2810 is shown disposed opposite of channel opening 2824. Channel opening 2810 may be disposed in between peripheral protrusions 2816 and 2818. The channel formed on top surface 2802 with channel openings 2810 and 2806 (not shown) may be shaped to receive a proximal or distal end of a bone. Channel opening 2824 may be disposed between peripheral protrusions 2816 and 2818. The channel formed on bottom surface 2830 with channel openings 2824 and 2828 may be shaped to receive an end of an opposing bone in the joint. Device 2800 may be surgically implanted in a joint, and oriented such that it prevents contact between surfaces of adjacent bones. In other examples, device 2800 and the elements shown and described may be designed, configured, formed, modified, or implemented apart from the examples shown or described and are not limited to those provided.

FIG. 28F illustrates a right view of an exemplary implantable interpositional pain management apparatus. Here, a right view of device 2800 includes top surface 2802, channel openings 2806 and 2828, peripheral protrusions 2812-2814, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. In some examples, top surface 2802 is configured, with channel openings 2806 and 2810 (not shown), to receive a bone or a portion thereof when device 2800 is surgically implanted. Bottom surface 2830 may also be configured, with channel openings 2828 and 2824 (not shown), to receive a bone or a portion thereof when device 2800 is surgically implanted. For example, if device 2800 is surgically implanted into a CMC joint, top surface 2802 may be configured, with channel openings 2806 and 2810, to receive (i.e., top surface 2802 may have a radius of curvature that is similar to that of a trapezium bone) a trapezium bone, or other bone adjacent to the joint. In other examples, device 2800 and the elements shown and described may be designed, configured, formed, modified, or implemented apart from the examples shown or described and are not limited to those provided.

FIG. 28G illustrates a perspective view of an exemplary implantable interpositional orthopedic pain management apparatus. Here, device 2800 includes top surface 2802, channel openings 2804-2810, peripheral protrusions 2812-2818, perimeter 2820, bottom surface 2830, and contours 2838. As used herein, like numbered and/or like named elements are assumed to be referencing the same or a substantially similar element having the same or substantially similar function or structure. Differences in function or structure may be described separately with regard to a specific feature described. As shown, top surface 2802 has a saddle shape, with a convex portion and a concave portion, as viewable in FIGS. 28C-28F. The concave and convex portions are coextensive, and both form the saddle shape of top surface 2802. In some examples, top surface 2802 may be substantially four sided, or rectangular, with peripheral protrusions 2812-2818 disposed on four corners. Perimeter 2820 may constitute the outermost portion of top or bottom surfaces 2802, 2830. In some examples, peripheral protrusions 2812-2818 may be formed with or disposed along perimeter 2820. In other examples, however, peripheral protrusions may not be disposed at corners of device 2800. Channel opening 2804 indicates an opening to a channel formed with top surface 2802 and channel opening 2802. Similarly, channel opening 2806 indicates an opening to a channel formed with top surface 2802 and channel opening 2808. Device 2800 may include additional surface elements as illustrated and identified generally as contours 2838. For example, device 2800 may include a convex curved portion as a transition between perimeter 2820 and top surface 2802. As with the other examples throughout this description, various radii of curvature may be used and are not limited to the examples shown and described. Device 2800 may not be permanently coupled or semi-permanently joined to a bone surface to allow for dynamic movement of device 2800 within the joint. In other words, device 2800 may be in intermittent contact with bones that make up a joint. In other examples, device 2800 and the elements shown and described may be designed, configured, formed, modified, or implemented apart from the examples shown or described and are not limited to those provided.

Although the foregoing examples have been described in various detail for purposes of clarity of understanding, the above-described inventive techniques and subject matter are not limited to the details provided. There are many alternative ways of implementing the above-described invention techniques. The disclosed examples are illustrative and not restrictive.

What is claimed:

1. A device, comprising:
a body having a top surface and a bottom surface, the top surface having a radius of curvature and the bottom surface having another radius of curvature, the body being configured to be surgically implanted in a joint and to prevent contact between one or more bones comprising the joint, the body not being coupled to the one or more bones; and
a peripheral protrusion disposed substantially about a perimeter of the body, the peripheral protrusion being formed with the body and having a substantially circular cross section, the peripheral protrusion being disposed about the perimeter of the body to prevent expulsion of the body from the joint.

2. The device of claim 1, wherein the peripheral protrusion is substantially spherical in shape.

3. The device of claim 1, wherein the peripheral protrusion is configured to maintain anatomical alignment of the device when inserted into the joint.

4. The device of claim 1, wherein the peripheral protrusion is in intermittent contact with the one or more bones.

5. The device of claim 1, wherein the protrusion is disposed substantially about the perimeter at a location at which the protrusion is configured to move the device in a direction that is substantially opposite to a force transmitted by a bone surface or another bone surface.

6. The device of claim 1, wherein the peripheral protrusion is disposed between proximally-positioned channel openings.

7. The device of claim 1, wherein the top surface is substantially concave to align the body with a bone surface of the one or more bones of the joint.

8. The device of claim 1, wherein the top surface is substantially convex to align the body with a bone surface of the one or more bones of the joint.

9. The device of claim 1, wherein the top surface is substantially saddle shaped.

10. The device of claim 1, wherein the peripheral protrusion is configured to be in intermittent contact with a bone surface of the one or more bones.

11. The device of claim 1, wherein the peripheral protrusion is disposed on the top surface at a distance from the perimeter.

12. The device of claim 1, wherein the peripheral protrusion is formed with the top surface and the bottom surface of the body.

13. A device, comprising:
a body having a top surface and a bottom surface, the top surface having a radius of curvature and the bottom surface having another radius of curvature, the body being configured to be surgically implanted in a joint and to prevent contact between one or more bones comprising the joint, the body not being coupled to the one or more bones; and
a plurality of peripheral protrusions disposed substantially about a perimeter of the body, the plurality of peripheral protrusions being formed with the body, each of the plurality of peripheral protrusions having a substantially circular cross section and being disposed about the perimeter of the body to prevent expulsion of the body from the joint.

14. The device of claim 12, wherein the peripheral protrusions are spherical in shape and configured to retain the device within a joint.

15. The device of claim 12, wherein the top surface radius of curvature is associated with a distal or proximal end of the one or more bones comprising the joint.

16. The device of claim 12, wherein the bottom surface radius of curvature is associated with a distal or proximal end of the one or more bones comprising the joint.

17. The device of claim 12, wherein each of the plurality of the peripheral protrusions having the substantially circular cross section are shaped are configured to substantially retain the device in a synovial capsule in the joint.

18. The device of claim 12, wherein the top surface includes a channel opening disposed substantially proximate to the perimeter of the body.

19. The device of claim 12, wherein the top surface includes two intersecting channels, each channel having two channel openings disposed about the perimeter of the body.

20. A device, comprising:
a body having a top surface and a bottom surface, the top surface having a radius of curvature and the bottom surface having another radius of curvature, the body being configured to be surgically implanted in a joint and to prevent contact between one or more bones comprising the joint, the body not being coupled to the one or more bones; and
a plurality of peripheral protrusions disposed substantially about a perimeter of the body, the plurality of peripheral protrusions being formed with the body, each of the plurality of peripheral protrusions having a substantially circular cross section and being disposed about the perimeter of the body to prevent expulsion of the body from the joint, a quantity of the plurality of peripheral protrusions being determined by a type associated with the joint.
